(19) United States
(12) Patent Application Publication
(10) Pub. No.: US 2025/0166785 A1
(43) Pub. Date: May 22, 2025

(54) METHOD FOR MULTI-SYSTEM INTERACTION

(71) Applicant: Cilag GmbH International, Zug (CH)

(72) Inventors: Frederick E. Shelton, IV, Hillsboro, OH (US); John E. Brady, Liberty Township, OH (US); Shannon L. Jones, Cincinnati, OH (US); Laura S. Downing, Cincinnati, OH (US); Andrew C. Deck, Cincinnati, OH (US); Weston Stanley Hirschfeld, West Chester, OH (US); Scotty A. Chung, Cincinnati, OH (US); Anthony J. Evers, Fairfield, OH (US); Todd M. Dahling, Loveland, OH (US); Aaron Crooker, Florence, KY (US); Christopher Johns, Mason, OH (US); Devin J. Clark, Cincinnati, OH (US); Douglas W. Fisher, Fairfield, OH (US); Jason L. Harris, Lebanon, OH (US); Matthew David Cowperthwait, Cincinnati, OH (US); Shane R. Adams, Lebanon, OH (US); Daniel James Mumaw, Liberty Township, OH (US); Benjamin M. Kaminski, Cincinnati, OH (US); Jacqueline Corrigan Aronhalt, Loveland, OH (US); Madeleine Jayme, Cincinnati, OH (US); Karl W. Mueller, Cincinnati, OH (US); Monica Louise Zeckel Rivard, Cincinnati, OH (US); Carl Joseph Draginoff, Mason, OH (US); Richard Joseph Ackermann, III, Monroe, OH (US); Jeffrey David Messerly, Cincinnati, OH (US); Richard L. Leimbach, Cincinnati, OH (US); Mark Zeiner, Loveland, OH (US); Sarah A. Scully, Cincinnati, OH (US); Nicholas James Ross, Franklin, OH (US); Seth Holdmeyer, Sharonville, OH (US); Joshua R. Ruoff, Loveland, OH (US); Rachel Garima Madhogarhia, Philadelphia, PA (US); Richard W. Timm, Cincinnati, OH (US); Andréas N. Ward, Cincinnati, OH (US); Dylan Rushton, Hyde Park, OH (US); Cole Grabowski, Cincinnati, OH (US); Jesse Hontz, Cincinnati, OH (US)

(21) Appl. No.: 18/954,186

(22) Filed: Nov. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.
(60) Provisional application No. 63/603,031, filed on Nov. 27, 2023.
(60) Provisional application No. 63/603,033, filed on Nov. 27, 2023.

(51) Int. Cl. G16H 20/40; G16H 10/60; G16H 40/63

(52) U.S. Cl. CPC G16H 20/40; G16H 10/60; G16H 40/63

(57) ABSTRACT

One or more data streams may be used for controlling multi-system interaction. For example, a data stream may be received, and a surgical option associated with a surgical instrument may be selected based on the data stream. A control signal associated with the surgical instrument may be generated based on the selected surgical option. A surgical device may receive an external data stream from a source external to the surgical system. The device may derive, based at least on the external data stream, decision contextual information. The device may select a surgical option associated with a surgical instrument based on the decision context information. The device may generate a visual indication of the decision context information associated with selecting the surgical option. The device may generate a control signal associated with the surgical instrument based on the selected surgical option.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023,
Provisional U.S. Patent Application No. 63/602,028, filed Nov. 22, 2023,
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023,
Provisional U.S. Patent Application No. 63/602,007, filed Nov. 22, 2023,
Provisional U.S. Patent Application No. 63/603,031, filed Nov. 27, 2023, and
Provisional U.S. Patent Application No. 63/603,033, filed Nov. 27, 2023.

This application is related to the following, filed contemporaneously, the contents of each of which are incorporated by reference herein:
Attorney Docket No. END9637USNP2, entitled VISUALIZATION OF AN INTERNAL PROCESS OF AN AUTOMATED OPERATION,
Attorney Docket No. END9637USNP3, entitled, VISUALIZATION OF AUTOMATED SURGICAL SYSTEM DECISIONS,
Attorney Docket No. END9637USNP4, entitled VISUALIZATION OF EFFECTS OF DEVICE PLACEMENT IN AN OPERATING ROOM,
Attorney Docket No. END9637USNP5, entitled VISUALIZATION OF EFFECTS OF DEVICE MOVEMENTS IN AN OPERATING ROOM,
Attorney Docket No. END9637USNP6, entitled DISPLAY OF COMPLEX AND CONFLICTING INTERRELATED DATA STREAMS,
Attorney Docket No. END9637USNP7, entitled COLLECTION OF USER CHOICES AND RESULTING OUTCOMES FROM SURGERIES TO PROVIDE WEIGHTED SUGGESTIONS FOR FUTURE DECISIONS,
Attorney Docket No. END9637USNP8, entitled AUGMENTING DATAFLOWS TO REBALANCE THE NUMBER OF UNKNOWNS AND DATAFLOWS,
Attorney Docket No. END9637USNP9, entitled PROBLEM-SOLVING LEVEL BASED ON THE BALANCE OF UNKNOWNS AND DATA STREAMS,
Attorney Docket No. END9637USNP10, entitled DATA STREAMS MULTI-SYSTEM INTERACTION,
Attorney Docket No. END9637USNP11, entitled DATA STREAM RESPONSE REACTION IN A MULTI-SYSTEM INTERACTION,
Attorney Docket No. END9637USNP12, entitled CONFLICTING DATA STREAMS IN MULTI-SYSTEM INTERACTION, and
Attorney Docket No. END9637USNP13, entitled INVALID DATA STREAM IN A MULTI-SYSTEM INTERACTION.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices.

### SUMMARY

Devices and methods for visualizing internal processes of an automated operation. An example device may include a processor configured to perform one or more actions. The device may receive an external data stream from a source external to the surgical system. The device may derive, based at least on the external data stream, decision contextual information. The device may select a surgical option associated with a surgical instrument based on the decision context information. The device may generate a visual indication of the decision context information associated with selecting the surgical option. The device may generate a control signal associated with the surgical instrument based on the selected surgical option.

The surgical option may be associated with stone removal. The external data stream may include a visualization of a patient's organ, and the processor is further configured to identify, based on the visualization of the patient's organ, a potential perimeter of a stone in the patient's organ. The decision context information may include the identified potential perimeter of the stone in the patient's organ. The visual indication of the decision context information may include a visual indication of the identified potential perimeter of the stone in the patient's organ.

The surgical option may be associated with stone removal. The external data stream may include a visualization of a patient's organ. The device may identify, based on the visualization of the patient's organ, a potential perimeter of a stone in the patient's organ. The device may select a stone removal treatment location based on the identified potential perimeter of the stone in the patient's organ. The control signal associated with the surgical instrument may be generated based on the selected stone removal treatment location. The visual indication of the decision context information may include a visual indication of the identified potential perimeter of the stone in the patient's organ.

The device may obtain a plurality of surgical options associated with the surgical instrument. The device may determine, based at least on the external data stream, respective system confidence assessments that correspond to the plurality of surgical options. The decision context information may include the respective system confidence assessments that correspond to the plurality of surgical options. The visual indication of the decision context information may include the system confidence assessment that corresponds to the selected surgical option.

The external data stream may include a visualization of a patient's organ. The device may determine, based on the external data stream and the selected surgical option, a resultant visualization of the patient's organ. The visual indication of the decision context information may include a visualization of the patient's organ pre-therapy and the resultant visualization of the patient's organ post-therapy.

The surgical option may be associated with stone removal. The external data stream may include a visualization of a patient's organ. The device may identify, based on the visualization of the patient's organ, a first potential perimeter of a stone in the patient's organ and a second potential perimeter of the stone in the patient's organ, the second potential perimeter encompassing the first potential perimeter. The device may calculate a first system confidence percentage associated with the first potential perimeter and a second system confidence percentage associated with the second potential perimeter. The decision context information may include the first system confidence percentage and the second system confidence percentage. The visual indication of the decision context information may include a visual indication of the first potential perimeter of the stone and its associated first system confidence percentage, and a visual indication of the first potential perimeter of the stone and its associated second system confidence percentage.

The surgical option is associated with stone removal. The external data stream may include a visualization of a patient's organ. The device may identify, based on the visualization of the patient's organ, a potential perimeter of a stone in the patient's organ. The device may identify a plurality of potential stone removal treatment locations. The device may determine, based on the potential perimeter of the stone in the patient's organ, respective confidence assessments that correspond to the plurality of potential stone removal treatment locations. The device may select a stone removal treatment location from the potential stone removal treatment locations based on their respective confidence assessments. The control signal associated with the surgical instrument may be generated based on the selected stone removal treatment location. The visual indication of the decision context information may include a visual indication of the potential perimeter of the stone in the patient's organ.

Devices and methods for visualizing automated surgical system decisions. An example device may include a processor configured to perform one or more actions. The device may receive an indication of a surgical procedure that involves a first surgical instrument cooperating with a second surgical instrument. The surgical procedure may include a plurality of surgical steps. The device may determine a first candidate action and a second candidate action associated with the first surgical instrument. The first candidate action and the second candidate action may allow the first surgical instrument to complete a first step of the plurality of the surgical steps. The device may determine a first effect, caused by the first candidate action, on the second surgical instrument's ability to perform a second step of the plurality of surgical steps. The device may determine a second effect, caused by the second candidate action, on the second surgical instrument's ability to perform the second step of the plurality of surgical steps. The device may select, based on the first effect and the second effect, an action, from the first candidate action and the second candidate action, for the first surgical instrument to perform. The device may generate a control signal configured to indicate the selected action.

The selected action may be a first action. The control signal may be a first control signal. The surgical procedure may include a third step. The device may determine, based on the selected first action associated with the first surgical instrument, a third candidate action and a fourth candidate action associated with the second surgical instrument. The third candidate action and the fourth candidate action may allow the second surgical instrument to complete the second step. The device may determine a third effect, caused by the selected candidate action associated with the first surgical instrument and the third candidate action, on a third surgical instrument's ability to perform the third step. The device may determine a fourth effect, caused by the selected candidate action associated with the first surgical instrument and the fourth candidate action, on the third surgical instrument's ability to perform the third step. The device may select, based on the third effect and the fourth effect, a second action, from the third candidate action and the fourth candidate action, for the second surgical instrument to perform. The device may generate a second control signal configured to indicate the second action.

The device may determine a parameter change associated with the first effect and the second effect. The device may determine a data type associated with the parameter change. The device may determine a format of a graphical representation of the first effect and the second effect based on the data type. The device may generate the graphical representation based on the determined format. The control signal may be configured to instruct a display to display the generated graphical representation.

The device may determine the data type associated with the parameter change based on one or more of: an absolute change in a parameter over a period of time; a relative change in the parameter over the period of time; data trends of summed data streams of interrelated parameters; or an impact, of the first or second effect, to one or more surgical instruments.

The device may identify a reserved space occupied by a third surgical instrument. Determining the first candidate action and the second candidate action associated with a first surgical instrument may involve determining that the first candidate action and the second candidate action cause the first surgical instrument to remain outside of the reserved space.

The first candidate action may involve placing a port in a first port location on a patient. The second candidate action may involve placing the port in a second port location on the patient. The control signal may be configured to indicate one or more of: a first magnitude of access that a laparoscopic instrument will have to a surgical area if the first port location is used, a first number of orientation possibilities of the laparoscopic instrument if the first port location is used, a second magnitude of access that the laparoscopic instrument will have to a surgical area if the second port location is used, or a second number of orientation possibilities of the laparoscopic instrument if the second port location is used.

The first surgical instrument may include a joint. The first candidate action may involve placing a port in a first port location on a patient. The second candidate action may involve placing the port in a second port location on the patient. The device may determine a first effect associated with the first port location and a second effect associated with the second port location based on at least one of: a position of a health care provider relative to the first surgical instrument, an articulation angle of the joint, a joint length of the joint, or a degree of freedom of the joint.

The device may receive user preference information and a patient position associated with the surgical procedure. The device may determine a surgical constraint based on at least one of the user preference information or the patient position. Selecting the action, from the first candidate action and the second candidate action, for the first surgical instrument to perform, may be based on the surgical constraint.

The first candidate action may involve a first placement of a base associated with the first surgical instrument, or a first movement of the first surgical instrument. The second candidate action may involve a second placement of the base associated with the first surgical instrument, or a second movement of the first surgical instrument.

Devices and methods for visualizing the effect of device placement in an operating room. An example device may include a processor configured to perform one or more actions. The device may receive an indication of a plurality of steps of a surgical procedure. One or more steps in the plurality of steps of the surgical procedure involve use of at least one of a first robotic arm attached to a first base, or a second robotic arm attached to a second base. The device may determine a fixed position of the first base. The device may determine, based on the plurality of steps of the surgical procedure and the fixed position of the first base, that a first candidate position of the second base is associated with a first number of interactions in which the first robotic arm and the second robotic arm will co-occupy space during the surgical procedure. The device may determine, based on the plurality of steps of the surgical procedure and the fixed position of the first base, that a second candidate position of the second base is associated with a second number of interactions in which the first robotic arm and the second robotic arm will co-occupy space during the surgical procedure. The device may select a candidate position for the second base, from the first candidate position and the second candidate position, based on the first number of interactions and the second number of interactions. The device may generate a control signal configured to indicate the selected candidate position for the second base.

The control signal being configured to indicate the selected candidate position of the second base comprises the control signal being configured to indicate one or more of: the first candidate position, the first number of interactions, the second candidate position, the second number of interactions, and a recommendation for the selected candidate position to be used as a fixed position of the second base.

The first robotic arm may be configured to move a first end effector attached to a distal end of the first robotic arm, and the second robotic arm is configured to move a second end effector attached to a distal end of the second robotic arm, each step in the plurality of steps of the surgical procedure is associated with a surgical space internal to a patient. The device may identify a set of candidate positions, comprising the first candidate position and the second candidate position, based on the plurality of steps of the surgical procedure. Each candidate position in the set of candidate positions may allow the first end effector and the second end effector to access the surgical space at a given step in the plurality of steps of the surgical procedure.

On a condition that the first number of interactions is less than the second number of interactions, the device may select the first candidate position. On a condition that the first number of interactions is greater than the second number of interactions, the device may select the second candidate position.

One or more steps in the plurality of steps of the surgical procedure may involve use of a third robotic arm attached to a third base. The device may determine, based on the plurality of steps of the surgical procedure, the fixed position of the first base, and the selected candidate position, that a third candidate position of the third base is associated with a third number of interactions in which the third robotic arm and at least one of the first robotic arm or the second robotic arm will co-occupy space during the surgical procedure. The device may determine, based on the plurality of steps of the surgical procedure the fixed position of the first base, and the selected candidate position, that a fourth candidate position of the third base is associated with a fourth number of interactions in which the third robotic arm and at least one of the first robotic arm or the second robotic arm will co-occupy space during the surgical procedure. The device may select a candidate position for the third base, from the third candidate position and the fourth candidate position, based on the third number of interactions and the fourth number of interactions. The device may generate a control signal configured to indicate the selected candidate position for the third base.

One or more steps in the plurality of steps of the surgical procedure may involve use of a third robotic arm attached to a third base. The device may predict an effect, caused by the selected candidate position for the second base, on placement of the third base, wherein the control signal is further configured to indicate the effect.

The device may receive user preference information. The device may determine a surgical constraint based on the user preference information. The device may select the candidate position for the second base, from the first candidate position and the second candidate position, based on the surgical constraint.

The device may determine a patient position associated with the surgical procedure. The device may determine a surgical constraint based on the patient's position. The device may select the candidate position for the second base, from the first candidate position and the second candidate position, based on the surgical constraint.

Devices and methods for visualizing effects of device placement in an operating room. An example device may include a processor configured to perform one or more actions. The device may receive an indication of a plurality of steps of a surgical procedure associated with a patient. One or more steps in the plurality of steps of the surgical procedure may involve use of a first robotic arm having a first end effector attached and a second robotic arm. The device may identify a first candidate motion and a second candidate motion of the first robotic arm configured to place the first end effector in a target end effector position internal to the patient. The device may determine, for the first candidate motion, a first number of associated interactions in which the first robotic arm and the second robot arm co-occupy space external to the patient during the surgical procedure. The device may determine, for the second candidate motion, a second number of associated interactions in which the first robotic arm and the second robot arm co-occupy space external to the patient during the surgical procedure. The device may select a candidate motion of the first robotic arm, from the first candidate motion and the second candidate motion, based on the first number of interactions and the second number of interactions. The device may generate a control signal based on the selected candidate motion of the first robotic arm.

The device may determine, during a first step in the plurality of surgical procedure steps, a current arm position of the first robotic arm and a current arm position of the second robotic arm that are external to a patient. The device may determine, during a second step in the plurality of surgical procedure steps, the target end effector position of the first end effector, wherein the first candidate motion and a second candidate motion of the first robotic arm are identified based on the current arm positions of the first and second robotic arms and the plurality of steps of the surgical procedure.

The control signal may be configured to indicate the selected candidate motion of the first robotic arm. The control signal may be configured to indicate one or more of: the first candidate motion, the first number of interactions, the second candidate motion, the second number of interactions, a recommendation to move the first robotic arm according to the selected candidate motion, an order in which to perform the selected candidate motion and a motion of the second robotic arm, or a time at which to perform the selected candidate motion.

Each step in the plurality of steps of the surgical procedure may be associated with a surgical site internal to the patient, a second end effector is attached to a distal end of the second robotic arm. The device may identify a set of candidate motions, comprising the first candidate motion and the second candidate motion, based on the plurality of steps of the surgical procedure. Each candidate motion in the set of candidate motions may allow the first end effector and the second end effector to access the surgical site at a given step in the plurality of steps of the surgical procedure.

On a condition that the first number of interactions is less than the second number of interactions, the device may select the first candidate motion. On a condition that the first number of interactions is greater than the second number of interactions, the device may select the second candidate motion.

The target end effector position of the first end effector may be a first position. The device may determine an updated current arm position of the first robotic arm, external to the patient, based on the first robotic arm moving according to the selected candidate motion. The device may determine a second target end effector position of the second end effector, during a third step in the plurality of surgical procedure steps. The second target end effector position may be internal to the patient.

The device may determine, based on the updated current arm position of the first robotic arm, the current arm position of the second robotic arm, and the plurality of steps of the surgical procedure, a third candidate motion of the second robotic arm that will place the second end effector in the second target end effector position. The third candidate motion of the second robotic arm may be associated with a third number of interactions in which the first robotic arm and the second robot arm will co-occupy space during the surgical procedure.

The device may determine, based on the updated current arm position of the first robotic arm, the current arm position of the second robotic arm, and the plurality of steps of the surgical procedure, a fourth candidate motion of the second robotic arm that will place the second end effector in the second target end effector position. The fourth candidate motion of the second robotic arm may be associated with a fourth number of interactions in which the first robotic arm and the second robot arm will co-occupy space during the surgical procedure. The device may select a candidate motion of the second robotic arm, from the third candidate motion and the fourth candidate motion, based on the third number of interactions and the fourth number of interactions. The device may generate a control signal based on the selected candidate motion of the second robotic arm.

One or more steps in the plurality of steps of the surgical procedure may involve use of a third robotic arm. The device may predict an effect, caused by the selected candidate motion of the first robotic arm, on a future motion of a third robotic arm, wherein the control signal is further configured to indicate the effect.

The device may receive user preference information and a patient position associated with the surgical procedure. The device may determine a surgical constraint based on at least one of the user preference information or the patient position. The device may select the candidate motion of the first robotic arm, from the first candidate motion and the second candidate motion, based on the surgical constraint.

The first robot arm may include a plurality of joints configured to move the first robot arm. The device may select, from the plurality of joints, a joint of the first robotic arm to articulate to achieve the selected candidate motion.

Devices and methods for displaying complex and conflicting interrelated data streams. An example device may include a processor configured to perform one or more actions. The device may receive a first biomarker value associated with a first biomarker in a first data stream and a second biomarker value associated with a second biomarker in a second data stream. The device may determine, based on the first biomarker value and the second biomarker value, that a close-loop control condition associated with a control parameter for a surgical device is satisfied. Based on determining that the close-loop control condition is satisfied, the device may determine a control parameter value associated with the surgical device based on the first biomarker value and the second biomarker value. The device may generate a control signal for the surgical device based on the determined control parameter value. The device may receive a third biomarker value associated with the first biomarker in the first data stream and a fourth biomarker value associated with the second biomarker in the second data stream. The device may determine, based on the third biomarker value and the fourth biomarker value, that the close-loop control condition associated with the control parameter for the surgical device is failed. Based on determining that the close-loop control condition is failed, the device may identify an intraoperative metric associated with the first data stream and the second data stream. The device may generate a second control signal configured to display a value associated with the intraoperative metric.

The first biomarker and the second biomarker may be associated with a physiological function of a patient. The device may determine a first status of the physiological function based on the first biomarker value and the second biomarker value. The close-loop control condition may be determined to be satisfied based on the first status of the physiological function being within an expected range. The device may determine a second status of the physiological function based on the third biomarker value and the fourth biomarker value. The close-loop control condition may be determined to be failed based on the second status of the physiological function being outside the expected range.

The device may determine a status type of the second status, wherein the status type indicates at least one of: at least one of the first biomarker or the second biomarker has changed at a rate that is greater than a first threshold, at least one of the first biomarker or the second biomarker has fluctuated a number of times during a time window, wherein the number of times is greater than a second threshold, a difference between the first biomarker and the second biomarker is greater than a third threshold, or a timing delay between a change in the first data stream and a change in the second data stream is greater than a fourth threshold. The intraoperative metric may be identified based on the status type of the second status.

The first biomarker and the second biomarker may be associated with a physiological function of a patient. The device may identify a third biomarker associated with the physiological function of the patient. The device may determine that the third biomarker is capable of impacting at least one of the first biomarker or the second biomarker. Based on the determination that the third biomarker is capable of impacting at least one of the first biomarker or the second biomarker, the device may use the third biomarker as the intraoperative metric.

The device may determine a first control parameter change direction associated with the control parameter based on the first biomarker value. The device may determine a second control parameter change direction associated with control parameter based on the second biomarker value. The close-loop control condition may be determined to be satisfied based on the first control parameter change direction and the second control parameter change direction being the same. The device may determine a third control parameter change direction associated with the control parameter based on the third biomarker value. The device may determine a fourth control parameter change direction associated with the control parameter based on the fourth biomarker value. The close-loop control condition may be determined to be failed based on the third control parameter changing direction and the fourth control parameter changing direction being different.

The device may determine a correlation pattern of the first data stream and the second data stream. The close-loop control condition may be determined to be satisfied or failed based on the correlation pattern.

The first biomarker may be a blood oxygen content. The second biomarker may be a percentage of carbon dioxide in exhalations. The device may determine a correlation pattern of blood oxygen content measurements in the first data stream and percentage of carbon dioxide in exhalations measurements in the second data stream. The close-loop control condition may be determined to be satisfied based on the correlation pattern indicating that the percentage of carbon dioxide in exhalations measurements and the blood oxygen content measurements change at a same rate. The device may generate a visual indication of a slope comparison of the first data stream and the second data stream. The intraoperative metric may include the slope comparison of the first data stream and the second data stream.

The first biomarker may be a blood oxygen content. The second biomarker may be a percentage of carbon dioxide in exhalations. The device may determine a correlation pattern of blood oxygen content measurements in the first data stream and percentage of carbon dioxide in exhalations measurements in the second data stream. The close-loop control condition may be determined to be failed based on the correlation pattern indicating that the percentage of carbon dioxide in exhalations measurements and the blood oxygen content measurements drift apart. Based on determining that the percentage of carbon dioxide in exhalations measurements and the blood oxygen content measurements drift apart, the device may identify a core body temperature of a patient as the intraoperative metric for display.

The device may determine a first pattern of the first data stream. The device may determine a second pattern of the second data stream. The intraoperative metric may include the first pattern of the first data stream and the second pattern of the second data stream.

The device may determine a timing delay between a change in the first data stream and a change in the second data stream. The intraoperative metric may include the determined timing delay between the change in the first data stream and the change in the second data stream.

The device may determine that the first data stream has stopped being received. Based on determining that the first data stream has stopped, the device may include, in the intraoperative metric comprises an option to use simulated data based on a pattern of the first biomarker while the first data stream was being received.

The device may determine a format of a graphical representation of the first data stream and the second data stream based on the intraoperative metric. The device may generate the graphical representation based on the determined format. The second control signal may be configured to instruct a display to display the generated graphical representation.

The second control signal may indicate a prompt or suggestion. The device may receive an input in response to the prompt or suggestion. The device may generate a third control signal for the surgical device based on received response.

Systems, methods, and/or instrumentalities disclosed herein may collect user choices and/or resulting outcomes from surgeries to provide weighted suggestions for future decisions. A system may include a processor. The system may be configured to receive an user input indicating a selection of a procedure from a plurality of procedures and/or a selection of a tactical domain target. The procedure and/or the tactical domain target may be associated with a parameter of a patient. The system may be configured to filter, based on the selection of the procedure, a plurality of surgical elements to obtain a primary surgical element and/or a secondary surgical element associated with the procedure. The primary surgical element may include a plurality of primary control loops associated with an output characteristic of the primary surgical element. The secondary surgical element may include a plurality of secondary control loops associated with an output characteristic of the secondary surgical element. The system may be configured to determine a tactical domain data for the procedure. The tactical domain data may include one or more relationships associated with the primary surgical element, the secondary surgical element, the parameter of the patient, and/or the tactical domain target. The system may be configured to receive a primary control data from the primary surgical element based on a primary control loop from the plurality of primary control loops. The primary control data may include the output characteristic associated with the primary surgical element. The system may be configured to receive a secondary control data from the secondary surgical element based on a secondary control loop from the plurality of secondary control loops. The secondary control data may include the output characteristic associated with the secondary surgical element. The system may be configured to generate a recommendation based on the tactical domain data, the primary control data, and/or the secondary control data. The recommendation may include an indication of an optimized control loop for the primary surgical element during the procedure. The optimized control loop may adjust the output characteristic associated with the primary surgical element to achieve the tactical domain target. The system may be configured to send the recommendation to the primary surgical element. The system may be configured to cause the primary surgical element to adjust the output characteristic associated with the primary surgical element based on the optimized control loop. The primary surgical element may adjust the output characteristic during the procedure to achieve the tactical domain target.

One or more of features may be included. In examples, the parameter of the patient may include at least one of oxygen saturation, blood pressure, respiratory rate, blood sugar, heart rate, a core body temperature and/or a hydration state. The tactical domain target may be a core body temperature setpoint of the patient, the primary surgical element may be a heating blanket, the secondary surgical element may be a ventilator, the output characteristic associated with the primary surgical element may be a heating coil of the heating blanket, the output characteristic associated with the secondary surgical element may be a heating coil to adjust the temperature of air flowing through the ventilator. The recommendation may include the indication of the optimized control loop to be used by the primary surgical element to control the heating coil of the heating blanket to meet the core body temperature setpoint.

The system may be configured to obtain historical data associated with the procedure. The historical data may include historical control data for the primary surgical element and/or for the secondary surgical element. The system may be configured to determine, for the procedure, conflict data. The conflict data may include a determination of a conflict associated with the primary surgical element and/or the secondary surgical element and/or a request for a second user input indicating whether the determination of the conflict occurred during the procedure.

The system may be configured to generate the recommendation further based on a machine learning (ML) model. The ML model may be trained using training data including one or more training data items. A training data item of the one or more training data items may include at least one indication of the historical data associated with the procedure and/or conflict data.

The system may be configured to determine the tactical domain data further based on an ML model. The ML model may infer the one or more relationships associated with the primary surgical element, the secondary surgical element, the parameter of the patient, and/or the tactical domain target. The system may be configured to generate the recommendation based on an ML model associated with the tactical domain data, the primary control data, and/or the secondary control data. The one or more relationships associated with the primary surgical element, the secondary surgical element, the parameter of the patient, and/or the tactical domain target may be determined based on a look-up-table.

A method may include receiving an user input indicating a selection of a procedure from a plurality of procedures, and/or a selection of a tactical domain target. The procedure and/or the tactical domain target may be associated with a parameter of a patient. The method may include filtering, based on the selection of the procedure, a plurality of surgical elements to obtain a primary surgical element and/or a secondary surgical element associated with the procedure. The primary surgical element may include a plurality of primary control loops associated with an output characteristic of the primary surgical element. The secondary surgical element may include a plurality of secondary control loops associated with an output characteristic of the secondary surgical element. The method may include determining a tactical domain data for the procedure. The tactical domain data may include one or more relationships associated with the primary surgical element, the secondary surgical element, the parameter of the patient, and/or the tactical domain target. The method may include receiving a primary control data from the primary surgical element based on a primary control loop from the plurality of primary control loops. The primary control data may include the output characteristic associated with the primary surgical element. The method may include receiving a secondary control data from the secondary surgical element based on a secondary control loop from the plurality of secondary control loops. The secondary control data may include the output characteristic associated with the secondary surgical element. The method may include generating a recommendation based on the tactical domain data, the primary control data, and/or the secondary control data. The recommendation may include an indication of an optimized control loop for the primary surgical element during the procedure. The optimized control loop may adjust the output characteristic associated with the primary surgical element to achieve the tactical domain target. The method may include sending the recommendation to the primary surgical element. The method may include causing the primary surgical element to adjust the output characteristic associated with the primary surgical element based on the optimized control loop. The primary surgical element may adjust the output characteristic during the procedure to achieve the tactical domain target.

One or more of features may be included. In examples, the parameter of the patient may include at least one of oxygen saturation, blood pressure, respiratory rate, blood sugar, heart rate, a core body temperature and/or a hydration state. The tactical domain target may be a core body temperature setpoint of the patient, the primary surgical element may be a heating blanket, the secondary surgical element may be a ventilator, the output characteristic associated with the primary surgical element may be a heating coil of the heating blanket, and/or the output characteristic associated with the secondary surgical element may be a heating coil to adjust the temperature of air flowing through the ventilator. The recommendation may include the indication of the optimized control loop to be used by the primary surgical element to control the heating coil of the heating blanket to meet the core body temperature setpoint.

The method may include obtaining historical data associated with the procedure. The historical data may include historical control data for the primary surgical element and/or for the secondary surgical element. The method may include determining, for the procedure, conflict data. The conflict data may include a determination of a conflict associated with the primary surgical element and/or the secondary surgical element. Conflict data may include a request for a second user input indicating whether the determination of the conflict occurred during the procedure.

The method may include generating the recommendation further based on a machine learning (ML) model. The ML model may be trained using training data including one or more training data items, each training data item of the one or more training data items may include at least one indication of the historical data associated with the procedure and/or conflict data.

The method may include determining the tactical domain data further based on an ML model. The ML model may infer the one or more relationships associated with the primary surgical element, the secondary surgical element, the parameter of the patient, and/or the tactical domain target. The method may include generating the recommendation based on an ML model associated with the tactical domain data, the primary control data, and/or the secondary control data

A system may include a processor. The system may be configured to receive an user input indicating a selection of a procedure from a plurality of procedures, and/or a selection of a tactical domain target. The procedure and/or the tactical domain target may be associated with a parameter of a patient. The system may be configured to determine a tactical domain data for the procedure. The tactical domain data may include one or more relationships associated with a primary surgical element, a secondary surgical element, the parameter of the patient, and/or the tactical domain target. The system may be configured to generate a recommendation based on the tactical domain data. The recommendation may include an indication of an optimized control loop for the primary surgical element. The optimized control loop may adjust an output characteristic associated with the primary surgical element to achieve the tactical domain target. The system may be configured to send the recommendation to the primary surgical element.

Dataflows may be augmented to rebalance the number of unknowns and dataflows. Instead of replacing a smart device, pausing and/or cancelling a procedure, or continuing a procedure with a reduced number of smart devices, a device (e.g., a surgical computing system) may use sensor data from a second smart device to generate control data for a first smart device. In the event that a surgical computing system detects that a first smart device is transmitting erroneous sensor data (e.g., an erroneous dataflow), the surgical computing system may determine that a second smart device is configured to provide sensor data similar to and/or the same as the failed first smart device. The surgical computing system may transmit a configuration message to the second smart device, requesting that the second device send the related sensor data to the surgical computing system. In examples, a surgical computing system may determine that a second dataflow (e.g., including control data, sensor data, and/or dataflow configuration information) may be added to a first dataflow to resolve an issue during a procedure.

A device may include a processor. The device may be configured to receive a first dataflow from a first surgical element. The first dataflow may be associated with a physiological parameter of a patient. The device may determine that the first dataflow from the first surgical element is erroneous. The device may determine a second dataflow associated with a second surgical element. The determination of the second dataflow may be based on an indication of a relational link associated with the second dataflow of the second surgical element, control data for the first surgical element, and/or the physiological parameter of the patient. The device may transmit, to the second surgical element, a configuration message. The configuration message may include an indication that the first dataflow is erroneous and/or a request to configure the second surgical element to send the second dataflow. The device may receive, from the second surgical element, a configuration response comprising the second dataflow. The device may generate control data for the first surgical element based on the second dataflow. The control data may indicate an adjustment to an output characteristic associated with the first surgical element. The device may cause the output characteristic associated with the first surgical element to be adjusted based on the control data.

In examples, the physiological parameter of the patient may be a core body temperature of the patient, the first dataflow may indicate a temperature associated with a heating pad, the second dataflow may indicate an insufflated air temperature measurement from a laparoscopic tool, the output characteristic may be a power level associated with the heating pad, and/or the control data may indicate an adjustment to the power level associated with the heating pad. The device may control the core body temperature of the patient by determining the power level associated with the heating pad based on the insufflated air temperature measurement from the laparoscopic tool.

In examples, the configuration response may include a surgical element ID, an indication that the second dataflow is available, and/or a unit of measure for the second surgical element. The relational link may be determined based on a lookup table (LUT). The LUT may indicate at a relationship between the second dataflow, the control data for the first surgical element, and/or the physiological parameter of the patient. The LUT may include, for the first dataflow and/or the second dataflow, a surgical element ID, a communication protocol, scheduling and frequency information, a destination, security and access control credentials, and a unit of measure for the first dataflow and the second dataflow. The relational link may be determined based on a machine learning (ML) model. The ML model may be trained based on a training data set. The training data set may include a plurality of dataflows and/or a plurality of control data associated with the physiological parameter of the patient.

In examples, the determination that the first dataflow from the first surgical element is erroneous may be based on a determination that control data exceeds a threshold, a determination that the first dataflow is unavailable, or a determination that the physiological parameter of the patient exceeds a patient safety threshold. The device may, in response to the determination that the first dataflow from the first surgical element is erroneous, transmit an interrogation message to the second surgical element. The interrogation message may request dataflow configuration information for the second surgical element and/or an indication of one or more dataflows associated with the second surgical element that may be related to the first dataflow. The device may receive an interrogation response indicating at least one dataflow associated with the second surgical element and/or dataflow configuration information. The dataflow configuration information may include at least one of a communication protocol, a frequency, a destination, or access control credentials.

A device may include a processor. The device may be configured to determine that a first dataflow from a first surgical element is erroneous. The device may determine a second dataflow based on a relational link associated with the first surgical element and a physiological parameter of a patient. The device may transmit, to a second surgical element, a configuration message including a request to configure the second surgical element to send the second dataflow. The device may receive, from the second surgical element, a configuration response including the second dataflow. The device may cause an output characteristic associated with the first surgical element to be adjusted based on control data associated with the second dataflow. The control data may indicate an adjustment to the output characteristic associated with the first surgical element.

A problem-solving level may be determined based on the balance of unknowns and data streams. Instead of replacing a smart device, pausing and/or cancelling a procedure, or continuing a procedure with a reduced number of smart devices, a device (e.g., a surgical computing system) may use sensor data from a second smart device to generate control data for a first smart device. In the event that a surgical computing system detects that a first smart device is transmitting erroneous sensor data (e.g., an erroneous dataflow), the surgical computing system may determine that a second smart device is configured to provide sensor data similar to and/or the same as the failed first smart device. The surgical computing system may transmit a configuration message to the second smart device, requesting that the second device send the related sensor data to the surgical computing system. In examples, a surgical computing system may determine that a second dataflow (e.g., including control data, sensor data, and/or dataflow configuration information) may be added to a first dataflow to resolve an issue during a procedure.

A device may include a processor. The device may be configured to receive a first dataflow from a first surgical element. The first dataflow may be associated with a physiological parameter of a patient. The device may determine based on the first dataflow, that the physiological parameter of the patient exceeds a patient safety threshold. The device may determine a second dataflow associated with a second surgical element. The determination may be based on an indication of a relational link associated with control data for the second surgical element, the first dataflow, and/or the physiological parameter of the patient. The device may transmit, to the second surgical element, a configuration message comprising an indication that the physiological parameter of the patient exceeded the patient safety threshold, and/or a request to configure the second dataflow to receive control data associated with the first surgical element. The device may generate the control data associated with the first surgical element. The control data may indicate an adjustment to an output characteristic associated with the second surgical element. The device may transmit, to the second surgical element, a control message. The control message may include an indication of the control data associated with the first surgical element. The device may cause the output characteristic associated with the second surgical element to be adjusted based on the control data associated with the first surgical element.

In examples, the physiological parameter of the patient may satisfy a threshold if a heart rate exceeds an operational window of 40-120 beats per minute, an oxygen saturation of the patient decreases below 90%, or a core body temperature of the patient is less than 89 degrees Fahrenheit. The configuration message may further include a surgical element ID, a communication protocol, and/or security or access control credentials. The relational link may be determined based on a lookup table (LUT). The LUT may indicate a relationship between the control data for the second surgical element, the first dataflow, and/or the physiological parameter of the patient. The relational link may be determined based on a machine learning (ML) model. The ML model may be trained based on a training data set. The training data set may include a plurality of dataflows and/or a plurality of control data associated with the physiological parameter of the patient.

The device may, based on the first dataflow, determine that the physiological parameter of the patient satisfies a second threshold. The second threshold may be associated with an acceptable operational range. The device may transmit a second configuration message. The second message may include an indication to reconfigure the second dataflow of the second surgical element, to remove the control data associated with the first surgical element. The device may, in response to the determination that the physiological parameter of the patient exceeded the patient safety threshold, transmit an interrogation message to the second surgical element. The interrogation message may request dataflow configuration information for the second surgical element and/or an indication of control data for the second surgical element that is associated with the physiological parameter of the patient. The device may receive an interrogation response indicating at least one physiological parameter of the patient associated with the second surgical element. The dataflow configuration information may include at least one of a communication protocol, a frequency, a destination, or access control credentials.

A device may determine based on a first dataflow, that a physiological parameter of a patient exceeds a patient safety threshold. The device may determine a second dataflow associated with a second surgical element. The determination may be based on an indication of a relational link associated with a first surgical element and/or the second surgical element. The device may generate control data associated with the first surgical element. The control data may indicate an adjustment to an output characteristic associated with the second surgical element. The device may transmit a control message to the second surgical element. The control message may include an indication of the control data associated with the first surgical element. The device may cause the output characteristic associated with the second surgical element to be adjusted based on the control data associated with the first surgical element.

Systems, methods, and instrumentalities are disclosed herein for a (e.g., pre-, in-, and/or post-operative) patient monitoring system. A surgical system may include a processor. A surgical system may include a processor configured to make a determination between two seemingly accurate but conflicting data streams. The processor may be further configured to obtain a first data stream associated with a measurement. The first data stream may be associated with a first control loop of the surgical system. The processor may be further configured to obtain a second data stream associated with the measurement. The second data stream may be associated with a second control loop of the surgical system. The processor may be further configured to determine that the first control loop and the second control loop are diverging. The processor may be further configured to generate a control signal based on the first and second data streams.

The processor may be further configured to obtain a third data stream associated with the measurement. The third data stream may be associated with a third control loop of the surgical system. The generating of the control signal may be further based on the third data stream. The control signal may be a selection of one of the first and second data streams based on a patient risk.

The selection of one of the first and second data streams may further include determining a current physiologic situation associated with a patient. The selection of one of the first and second data streams may further include selecting, between a first control parameter associated with the first data stream and a second control parameter associated with the second data stream, a control parameter based on the current physiologic situation associated with a patient. The data stream associated with the selected control parameter may be selected.

The control signal may be a weighted combination of the first and second data streams. The second data stream may transformed prior to the combination of the first and second data streams. The control signal may be a difference between the first and second data streams. The processor may be further configured to determine a cause of the divergence between the first and second control loops. The control signal may be generated further based on the cause of the divergence.

The processor is further configured to detect a measurement difference between the first data stream and the second data stream. The processor is further configured to compare the measurement difference to a threshold value. The generating of the control signal may be based on the measurement difference being above the threshold value.

The surgical system may be a heating system of a patient. The measurement the first data stream and the second data stream may be associated with may be a temperature of the patient. The generated control signal may be sent to the heating system to change the temperature of the patient.

The surgical system may be configured to identify a disagreement and/or divergence between the first data stream and the second data stream. The surgical system may be configured to determine a cause of the identified disagreement and/or divergence. The data stream may be selected based on the cause of the identified disagreement and/or divergence.

The surgical system may be configured to detect a disagreement and/or divergence between the first data stream and the second data stream. The comparing of the first control parameter and the second control parameter and the selecting of the data stream may be performed based on the detection of the disagreement between the first data stream and the second data stream.

The surgical system may be configured to detect a measurement difference between the first data stream and the second data stream. The surgical system may be configured to compare the measurement difference to a threshold value.

The first data stream and the second data stream may be associated with a control loop of the surgical system.

Systems, methods, and instrumentalities are disclosed herein for a (e.g., pre-, in-, and/or post-operative) patient monitoring system. A surgical system may include a processor. A surgical system may include a processor configured to obtain an input control data stream associated with a measurement. The input control data stream may be associated with a control loop of the surgical system. The processor may be further configured to determine an importance factor of a condition associated with a patient. The processor may be further configured to generate a response reaction based on the input control data stream and the importance factor of the condition associated with the patient. The processor may be further configured to determine a reaction time between an instant of the input control data stream causes a response reaction to be generated. The processor may be further configured to modify the response reaction based on the generated response reaction.

The modification of the response reaction may be an escalation. The modification of the response reaction may be a recession. The reaction time may be based on the importance factor of the condition associated with the patient. The importance factor may be based on a patient risk. The instant the input control data stream is determined when the input control data stream may violate a first threshold associated with the input control data stream. The instant the input control data stream is determined when the input control data stream may satisfy a second threshold associated with the input control data stream.

The control loop of the surgical system may be a closed loop system. The closed loop system of the surgical system may be changed to an open loop system. The processor may be further configured to prevent an anticipated instability from affecting the surgical system based on a change in the first data stream or the second data stream. The surgical system may be a heating system of a patient. The measurement may be associated with the input control data stream is a temperature of the patient. The condition associated with the patient may be a risk of overheating and the importance factor may be high. The response reaction may be generated to reduce the temperature of the patient based on the input control data stream and the importance factor of the condition associated with the patient. The reaction time determined may be the time between an instant of the input control data stream and the instant a response reaction is generated. The response reaction may be modified based on the generated response reaction.

Systems, methods, and instrumentalities are disclosed herein for a (e.g., pre-, in-, and/or post-operative) patient monitoring system. A surgical system may include a processor. The surgical system may obtain a first data stream and a second data stream associated with a same measurement. The surgical system may determine a first control parameter based at least in part on the first data stream. The surgical system may determine a second control parameter based at least in part on a second data stream. The surgical system may compare the first control parameter and the second parameter. The surgical system may select a data stream between the first data stream and the second data stream based on the comparing. The surgical system may generate a control signal based on the selected data stream.

Comparing the first control parameter and the second parameter further may include calculating a first difference between the first control parameter and a current control parameter. Comparing the first control parameter and the second parameter further may include calculating a second difference between the second control parameter and the current control parameter. Comparing the first control parameter and the second parameter further may include selecting a control parameter that is associated with less difference. The data stream associated with the selected control parameter may be selected.

Comparing the first control parameter and the second parameter may include determining a first risk level associated with the first control parameter. Comparing the first control parameter and the second parameter may include determining a second risk level associated with the second control parameter. Comparing the first control parameter and the second parameter may include selecting a control parameter that is associated with a lower risk level. The data stream associated with the selected control parameter may be selected.

Comparing the first control parameter and the second parameter may include determining a first surgical action of the surgical system associated with the first control parameter. Comparing the first control parameter and the second parameter may include determining a second surgical action of the surgical system associated with the second control parameter. Comparing the first control parameter and the second parameter may include comparing the first surgical action and the second surgical action to a predetermined list of preferred surgical actions. Comparing the first control parameter and the second parameter may include selecting a control parameter that is associated with a preferred surgical action. The data stream associated with the selected control parameter may be selected.

Comparing the first control parameter and the second parameter may include determining a current physiologic situation associated with a patient. Comparing the first control parameter and the second parameter may include selecting, between the first control parameter and the second control parameter, a parameter based on current physiologic situation associated with a patient, wherein the data stream associated with the selected control parameter is selected.

The surgical system may identify a disagreement between the first data stream and the second data stream. The surgical system may determine a cause of the identified disagreement. The data stream may be selected based on the cause of the identified disagreement.

The surgical system may detect a disagreement between the first data stream and the second data stream. The comparing of the first control parameter and the second control parameter and the selecting of the data stream may be performed based on the detection of the disagreement between the first data stream and the second data stream.

The surgical system may detect a measurement difference between the first data stream and the second data stream. The surgical system may compare the measurement difference to a threshold value. The comparing of the first control parameter and the second control parameter and the selecting of the data stream may be performed based on the measurement difference being above the threshold value.

The first data stream and the second data stream may be associated with a control loop of the surgical system.

The surgical system may obtain a third data stream associated with the measurement. The surgical system may compare the first data stream and the second data stream to the third data stream. The surgical system may identify, based on the comparing to the third data stream, a data stream consistent with the third data stream. The data stream may be selected based on the identified data stream consistent with the third data stream.

Systems, methods, and instrumentalities are disclosed herein for a surgical system. A surgical system may include a processor. The surgical system may obtain a data stream associated with a measurement from a surgical device. The surgical system may generate a first control signal associated with the surgical device based on the data stream. The surgical system may detect that the data stream is invalid. Upon detecting that the data stream is invalid, the surgical system may determine an approximation factor associated with the data stream. The surgical system may generate a second control signal associated with the surgical device based on the determined approximation factor.

For example, the surgical system may introduce a perturbation to an input signal of the surgical device. The surgical system may receive a value in the data stream upon introducing the perturbation. The surgical system may determine an expected value in the data stream in response to the perturbation. The surgical system may compare the received value to the expected value. The surgical system may assess a validity of the data stream based on the comparing to monitor the data stream.

The surgical system may introduce a perturbation to an input signal of the surgical device. The surgical system may determine an expected control value in response to the perturbation. The surgical system may determine a difference between the expected control value and a normal control value. The approximation factor associated with the data stream may be determined based on the difference between the expected control value and the normal control value. The surgical system may adjust a response of the surgical device based on the approximation factor.

The data stream may be determined to be invalid based on an expected range associated with the measurement. Based on detecting a measured value in the data stream being outside of the expected range associated with the measurement, data stream may be determined to be invalid.

The surgical system may generate a corrected data stream associated with the measurement based on the approximation factor and the data stream. The second control signal associated with the surgical device may be generated based on the corrected data stream.

The surgical system may transform the data stream based on the approximation factor. The second control signal associated with the surgical device may be generated based on the transformed data stream.

The surgical system may detect a measurement difference between the data stream and historic data. The surgical system may compare the measurement difference to an error tolerance threshold value. The detecting that the data stream is invalid may be based on the measurement difference satisfying the error tolerance threshold value.

Determining the approximation factor may further include identifying the surgical device that generates the data stream. Determining the approximation factor may further include obtaining an initial characterization of the surgical device. The approximation factor may be determined based on the initial characterization of the surgical device.

The surgical system may detect a measurement difference between the data stream and historic data. The surgical system may identify a disagreement between the data stream and the historic data. The surgical system may determine a cause of the identified disagreement, and the approximation factor may be determined based on the cause of the identified disagreement.

### BRIEF DESCRIPTION OF THE DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example robotic arm attached to a base.

FIG. 6 illustrates an example operating room arrangement of multiple robotic arms.

FIG. 7 illustrates an example display during gallstone identification.

FIG. 8 illustrates an example display of a sphere of uncertainty.

FIG. 9 illustrates an example combination dual endoscope with visual light and infrared (IR) visualization for subsurface visualization of stones within tissue.

FIG. 10 illustrates an example dual endoscope used in gallstone detection.

FIG. 11 illustrates example joint movements of a robotic arm.

FIG. 12 illustrates an example operating room arrangement of multiple robotic arms.

FIG. 13 illustrates an example display to a user of potential interactions between robotic arms during a surgical procedure.

FIG. 14 illustrates an example display to a user of a decision to reposition a robotic arm to avoid a collision.

FIG. 15 illustrates an example display to a user of optional actions to avoid a collision.

FIG. 16 illustrates an example display to a user of the impact of potential port locations.

FIG. 17 illustrates an example display to a user of the access capabilities of surgical tools based on a port location.

FIG. 18 illustrates an example robotic arm attached to a base.

FIG. 19 illustrates an example operating room arrangement of multiple robotic arms.

FIG. 20 illustrates an example display to a user of the effects of device placement in an operating room.

FIG. 21 illustrates an example device placement during operating room set-up initialization.

FIG. 22 illustrates an example device placement optimization.

FIG. 23 illustrates an example three-dimensional matrix with the different variables associated with the system, arms, devices, etc.

FIG. 24 illustrates example joint movements of a robotic arm.

FIG. 25 illustrates an example operating room arrangement of multiple robotic arms.

FIG. 26 illustrates an example display to a user of areas of potential interactions between surgical devices.

FIG. 27 illustrates an example display to a user of potential interactions between robotic arms during a surgical procedure.

FIG. 28 illustrates an example display to a user of zones of robotic arm movements.

FIG. 29 illustrates example robotic arm movements based on the type of procedure being performed and/or a step of the procedure.

FIG. 30 illustrates an example operating room arrangement of multiple robotic devices.

FIGS. 31A-C illustrate example conflicting decisions based on data from a systemic warming device and data from a smart ventilator.

FIGS. 32A-B illustrate example conflicting decisions based on data from a smoke evacuator, generator, and surgical scope.

FIGS. 33A-B illustrate example threshold options.

FIGS. 34A-B illustrate an example of highlighting segments of high variability.

FIGS. 35A-B illustrate example options for displaying historical data.

FIG. 36 shows an example surgical instrument.

FIG. 37 is a block diagram for applying machine learning to improve artificial intelligence algorithms and/or iterations of learning for artificial intelligence algorithms.

FIG. 38 is an example operational environment in which a surgical computing system may receive control data to determine an optimized control loop for a surgical element.

FIG. 39 is an illustration of an example user interface indicating one or more conflicts.

FIG. 40 is a flow chart of an example optimized control loop selection routine performed by a surgical computing system.

FIGS. 41A-41B illustrate example operational environments including example data paths for one or more dataflows.

FIG. 42 illustrates an example routine for re-balancing the number of unknowns and dataflows.

FIG. 43 illustrates an example communication diagram for re-balancing the number of unknowns and dataflows.

FIGS. 44A-44B illustrate example aspects of an operational environment for re-balancing a temperature measurement and temperature control.

FIG. 45 shows an example computer-implemented surgical system for determining a control signal.

FIG. 46 shows an example computer-implemented surgical system for comparing multiple (e.g., two) differing data streams.

FIG. 47 shows an example flowchart for determining a control signal.

FIG. 48 shows an example computer-implemented surgical system for determining a control signal.

FIG. 49 shows an example computer-implemented surgical system for determining a body temperature management control signal.

FIG. 50 shows an example flowchart for determining a control signal to use for generating a control signal.

FIG. 51 shows an example computer-implemented surgical system for determining a data stream.

FIG. 52 shows an example computer-implemented surgical system with an artificial testing input with a closed-loop control input.

FIG. 53 shows an example flowchart for selecting a data stream to use for generating a control signal.

FIG. 54 shows an example computer-implemented surgical system for determining a data stream is invalid.

FIG. 55 shows an example flowchart for handling an invalid data stream.

### DETAILED DESCRIPTION

A more detailed understanding may be had from the following description, given by way of example in conjunction with the accompanying drawings.

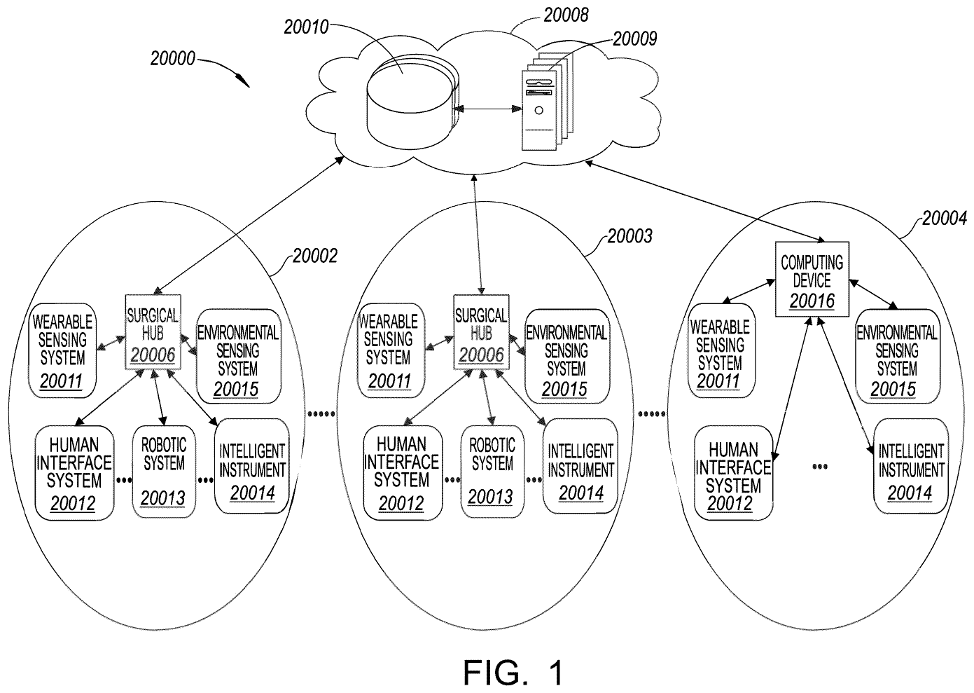

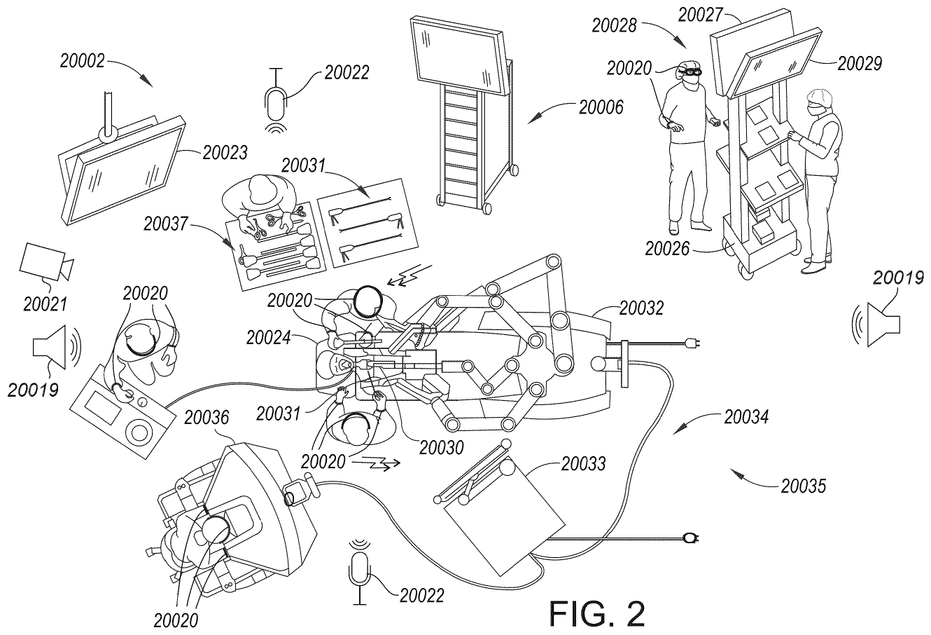

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

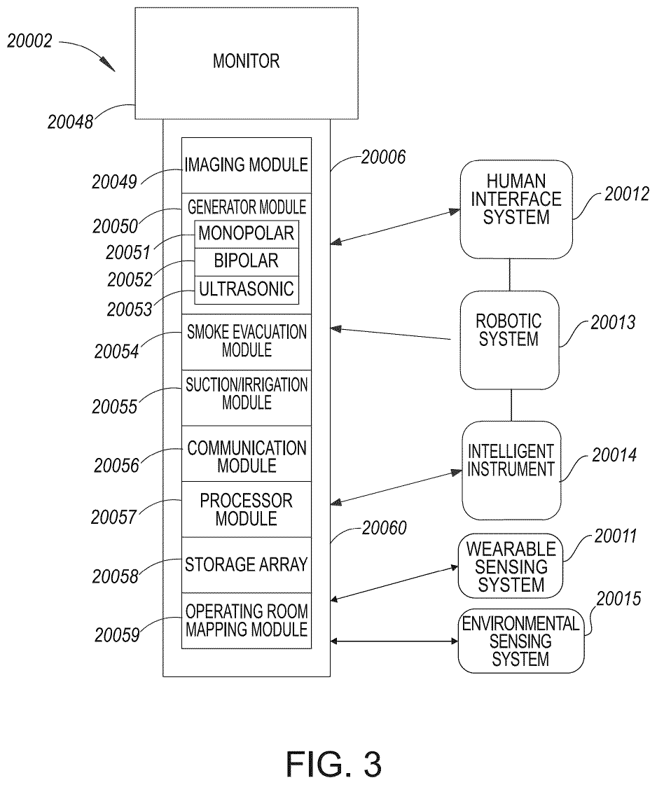

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

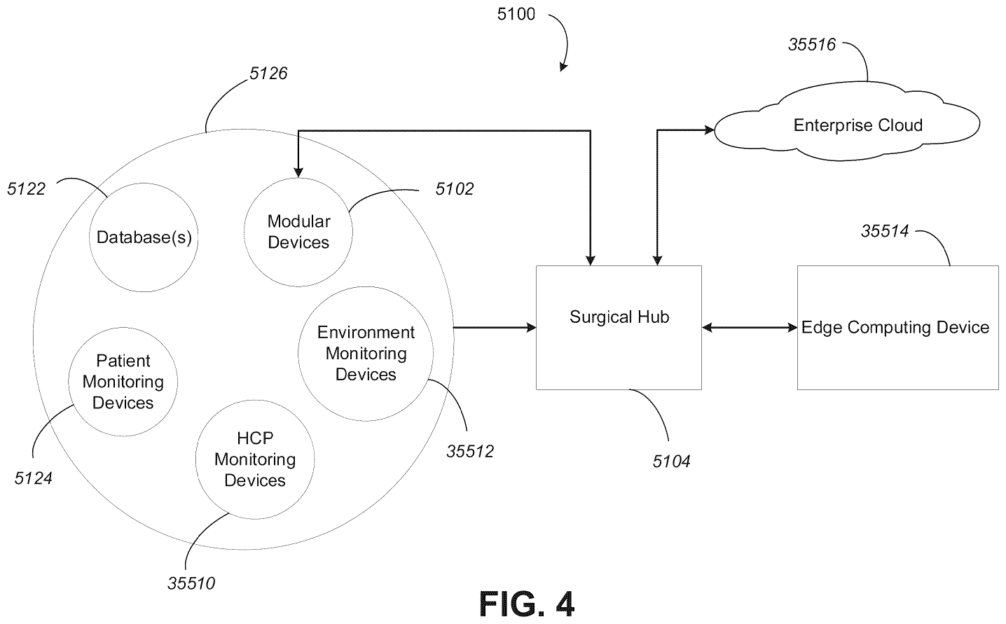

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient moni-toring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

A system may automatically make decisions during a surgical procedure. The system may display a visualization of the system-automated decision and/or information that was used to make the decision.

The system may display a visualization of an internal process of an automated operation of a smart system. The smart system may receive an external data stream from a source external to the surgical system. The system may derive decision contextual information based at least on the external data stream. The system may use the data stream from an externally supplied system to make a decision between at least two choices. The system may select a surgical option associated with a surgical instrument based on the decision context information. The system may generate a visual indication of the decision context information associated with selecting the surgical option. The system may display the decision context information or an explanation of how the decision was made (e.g., to inform the HCP of the options selected from, and why an option was selected). The context of the choice may be a perimeter or margin that the system detects. The internal process may be the probability that the system is correct (e.g., based on highlighted aspects of the image). The internal process may include therapeutic interactions associated with the decision. The system may show how a result of the decision may differ (e.g., sequentially) from the pre-operative (e.g., pre-therapy) expectations. The system may generate a control signal associated with the surgical instrument based on the selected surgical option.

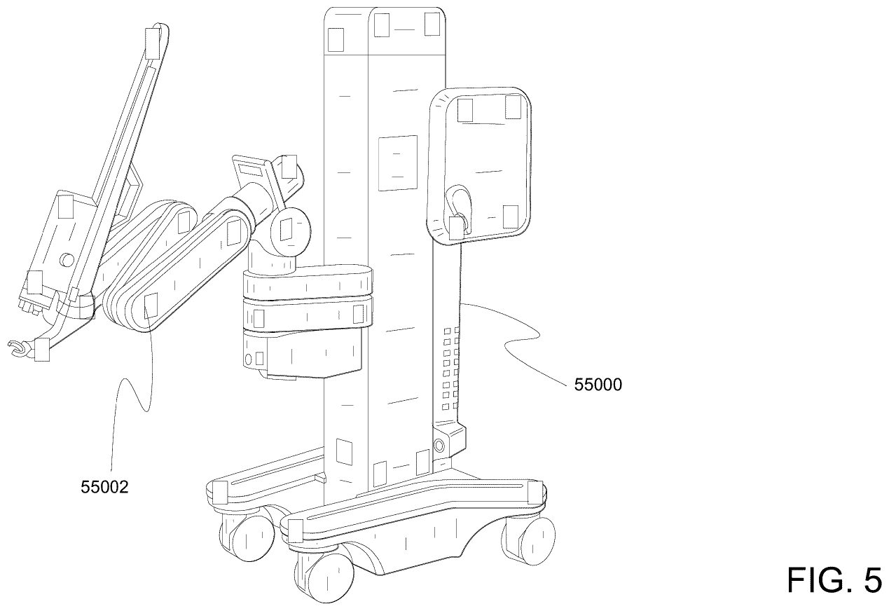

FIG. 5 illustrates an example robotic arm attached to a base 55000. In an example, the system may display (e.g., highlight or otherwise indicate) a recommended joint 55002 to move to place an end effector in the desired location. The system may further display an explanation of how the joint 55002 was selected. For example, the system may illustrate a movement path of the end effector if the joint 55002 is used. In another example, the system may indicate that the space through which the robotic arm will move if joint 55002 is used is smaller than the space the arm would move through if another joint were used.

The system may display outcomes associated with the options. This may improve the HCP's understanding of the algorithm (e.g., decision-making process). The system may demonstrate the result and aspects of the decision-making process leading to the system's decision (e.g., so that the user understands and believes the algorithm has accurately made a decision).

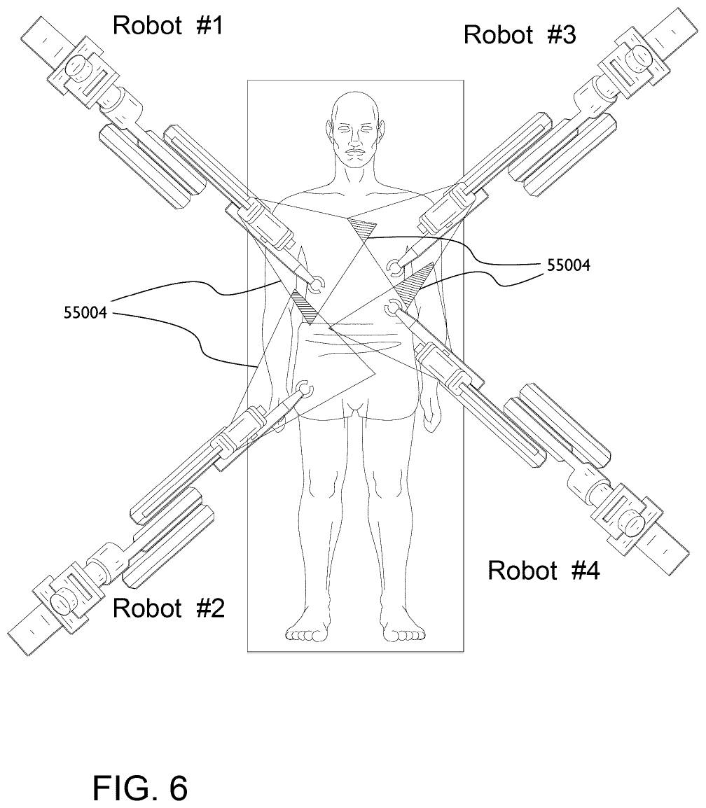

For example, FIG. 6 illustrates an operating room arrangement of multiple robotic arms. As shown, multiple robotic arms may be present in a relatively small space in an OR. The arms may have ranges of motion that overlap (e.g., as shown at 55004). In this case, the arms may collide unless adjustments are made to reduce the potential interactions. For example, one or more of the arms may not be allowed to occupy the overlapping space (e.g., at a given time). As described herein, the system may determine potential robot arm position placement and kinematic forecasting of resulting positions, movements, and/or interactions. The system may determine (and display) options for the HCP to choose from (e.g., and request that the HCP provide input). The system may output a simple, understandable display of context and choices.

In another example, the system may automatically determine which robotic arms to move (e.g., without HCP input). The system may, for example, determine that movement of robotic arm #3 will cause a collision with robotic arm #4. In this case, the system may prevent or limit movement of robotic arm #3. If the system prevents or limits movement of a robotic arm, the system may display the reason(s) for the restriction. For example, as illustrated in FIG. 6, the system may highlight or otherwise indicate the area 55004 in which the robotic arms would collide if movement were not restricted.

The system may obtain a plurality of surgical options associated with the surgical instrument. The system may determine (e.g., based at least on the external data stream) respective system confidence assessments that correspond to the plurality of surgical options. The decision context information may include the respective system confidence assessments that correspond to the plurality of surgical options. The visual indication of the decision context information may include the system confidence assessment that corresponds to the selected surgical option.

The system may indicate a confidence level that the decision made will lead to a certain result. For example, the system may indicate a confidence level of accurately identifying a gallstone or node. The system may display a confidence percentage. The system may display visual cue(s) of the effects of a decision and the confidence level. The system may determine (e.g., based on the external data stream and the selected surgical option) a resultant visualization of the patient's organ. The visual indication of the decision context information may include a visualization of the patient's organ pre-therapy and the resultant visualization of the patient's organ post-therapy.

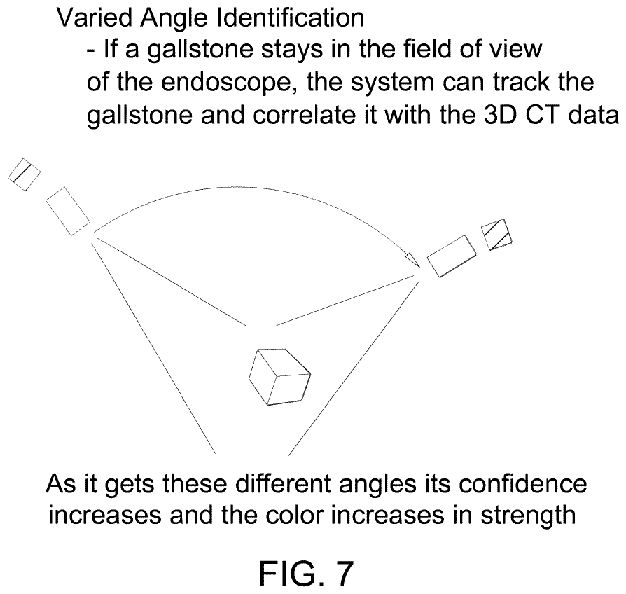

The surgical option may be associated with stone removal. The external data stream may include a visualization of a patient's organ. For example, FIG. 7 illustrates an example display during gallstone identification. As illustrated, the gallstone may be in the field of view of an endoscope. The system may track the gallstone and correlate its location with pre-operative data (e.g., from a three-dimensional CT scan). The endoscope may move around the gallstone to capture views of the gallstone from different angles. As the endoscope obtains the different views, the display may change to reflect the system's confidence in gallstone identification. For example, the color of the gallstone may change or increase in strength/brightness.

The system may identify a potential perimeter of a stone in the patient's organ based on the visualization of the patient's organ. The decision context information may include the identified potential perimeter of the stone in the patient's organ. The visual indication of the decision context information may include a visual indication of the identified potential perimeter of the stone in the patient's organ. For example, the display may have a primary indicator of a location affected by the decision and a secondary indicator of uncertainty (e.g., a potential margin of error). Error bands may vary (e.g., in both positive and negative directions) from event to event. In an example, the width of the indication may be directly correlated to the corresponding error.

The system may identify (e.g., based on the visualization of the patient's organ) a first potential perimeter of a stone in the patient's organ and a second potential perimeter of the stone in the patient's organ. The second potential perimeter may encompass the first potential perimeter. The system may calculate a first system confidence percentage associated with the first potential perimeter and a second system confidence percentage associated with the second potential perimeter. The decision context information may indicate the first system confidence percentage and the second system confidence percentage. The visual indication of the decision context information may include a visual indication of the first potential perimeter of the stone and its associated first system confidence percentage, and a visual indication of the first potential perimeter of the stone and its associated second system confidence percentage.

The system may offset indications on display to mitigate risk correlated to error. The system may have latency in communication, mechanical aspects, and/or measurement uncertainty. For example, as the system works faster, the likelihood of error in what is displayed increases. The system may intentionally offset the data display to combat the increased chance of error. A positional indicator may be shifted to the high end to represent the highest risk possibility to the user and/or patient.

The system may use previously determined data in the confidence prediction. The system may have intrinsic uncertainty when introduced into a new environment and/or operation. As subsequent actions and/or firings are performed by the system, the system may incorporate that data into its confidence predictions. This may cause the system to be more (or less) confident in future decisions.

The system may indicate a percent confidence related to timing.

A secondary data source may be used to build confidence in a primary data source. For example, an endocutter knife may be visualized as it moves from a starting position to the channel. The knife may disappear from view while it transects tissue (e.g., an anvil).

A visual appearance of the display may change based on the confidence level. For example, intensity of visual highlighting may correlate to a higher confidence level. The intensity may be displayed as stepped intensity or a range of intensities.

Visual information (e.g., critical structure detection) may be overlaid onto an augmented reality (AR) display. The information may use fill patterns, weighting, dashes, line segments around a detected object, shape and/or color of a detected object, etc. For example, if a potential object has been identified, a square box may be placed around the object on the display. As confidence in the identification grows, the square box may transform into a fitted shape around the object.

An object that has been identified may be flagged with a yellow box (e.g., indicating caution because it is an unknown object). Once the object has been identified by the system, the object may be flagged with a fitted structure or outline around the object. The object may be flagged as red (e.g., due to its surgical criticality, for example, a critical structure, tumor, etc.).

A sensitivity setting may be adjusted to change how confident the system should be before making and/or displaying a decision. The sensitivity setting may be set by a user. The sensitivity setting may be based on market research.

If multiple objects (e.g., stones, nodes) that have different confidence levels are displayed, the system may indicate the difference by using a visual (e.g., color-based) indicator, shading with different colors, transparency/opaqueness, etc. For example, a partial circumference of stone may be outlined to depict confidence in the stone's size and shape. The system may use different color rings based on the data inputs used. The indication may be a text indicator (e.g., a numerical confidence percentage). The system may display a number the stones detected and a legend. The system may give the user the option to select a stone. For example, the user may be able to hover over (or press and hold) an object (e.g., with a mouse or eye tracking) to display additional information about that object. The information may include a size of the object, a percent confidence in object identification, a likelihood of an event associated with the object (e.g., likelihood for patient to pass a stone), etc.

During pre-operative CT scanning of a patient, gallstones may be visualized (e.g., due to the density differences compared to soft tissue structures). CT systems may be able to produce digital 3D models (e.g., polygonal files, for example, stl format). The 3D model may be funneled into the IR endoscopic computer system (e.g., via the non-proprietary stl format). As the endoscopic camera angle changes in orientation to the object (e.g., stone) in question, the camera may capture a two-dimensional view or shape based on the reflectivity of the subsurface object (e.g., stone). The computer may line up the views of the object (e.g., aligning the real-time view with the CT data). The system may determine a level of confidence based on how well the data matches. The data may be used to notify the surgeon of confidence levels, stone depth, stone count, etc.

Interoperative visualization of subsurface gallstones may be used to ensure that none are missed during the surgical procedure. CT scanning allow for pre-operative visualization, but positions of the objects (e.g., stones) may shift between scanning and the surgical procedure. The combination of the two visualization methods may allow the surgeon to assess real time, intuitive information and mappings (e.g., with increased surgical confidence).

The identification system may utilize one or more of the following properties from the 2D image to match with the 3D object: area, maximum cross-sectional length, the concavity of the perimeter, the convexity of the perimeter, the pointed-ness of perimeter, pre-trained ML models, porosity of the stone/object, topology of the stone, uniformity of the stone surface, length of perimeter, and/or the like.

Foreign bodies may be identified (e.g., in two-dimensional intra-operative imaging) based on a three-dimensional pre-operative model. For example, the system may perform shape-based correlation of a gallstone's location using an infrared endoscope and CT system data. The system may display a computer vision recognized margin of stone (e.g., areas around expected stone center that are more or less likely to include the stone). The system may distinguish between stones and critical structures using different colors/shading/brightness. The surgeon may toggle the view selection to highlight particular structures (e.g., stones view and nodes/other structures view). The system may distinguish between stones and critical structures using identifiers (e.g., vascular identifier, etc.).

The system may indicate an unidentified object or an object identification with low confidence. In this case, the system may prompt the surgeon to identify the objects. The system may utilize the data as an input to an AI/ML feedback loop.

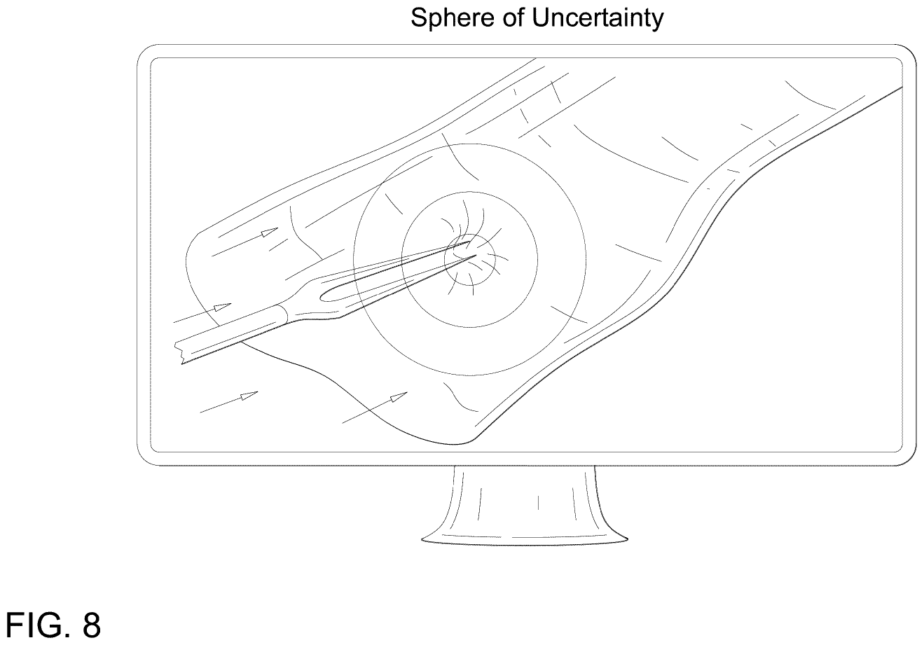

The system may display a sphere of uncertainty. FIG. 8 illustrates an example display of a sphere of uncertainty. As shown, the display may include a color-coded display of uncertainty based on the level of uncertainty or probability of being within a certain distance. For example, the area may be highlighted green if the object is within a first distance (e.g., a millimeter) of that area, highlighted yellow if the object is within a second distance (e.g., 5 millimeters) of the area, and highlighted red if the object is within a third distance (e.g., 10 millimeters) of the area. The sphere of uncertainty may adjust based on patient size, age, etc. Pre-operative CT data may be used to calibrate the sphere of uncertainty for a patient. In this way, the sphere of uncertainty may differ based on patient data.

The sphere of uncertainty may change (e.g., expand and decrease) based on the location in the body and/or device used. For example, if the object is in the lung, the sphere may be smaller than if the object is near the stomach. The sphere of uncertainty may be impacted by nearby critical structures (e.g., carotid arteries, ureter, nerves, etc.).

The sphere of uncertainty may be used to categorize and display data uncertainty (e.g., expected distance from critical structures). After identification, the system may indicate that an object is a stone or critical structure by changing the visual representation of the identified objects (e.g., based on surgical importance). For example, objects may be differentiated using color, line thickness, perimeter of the shape, type of shape (e.g., diamond, square, circle), and/or the like.

The system may change visual indications based on current surgical steps and/or actions. For example, the visual indicator may indicate the direction of removal of a surgical object (e.g., the system can indicate the direction that the kidney stones should be removed by the surgeon based on other surgical criteria). The system may indicate the next action to be taken. For example, if the system identifies multiple kidney stones, the system may identify the kidney stone that makes the most logical sense to remove first (e.g., by highlighting that stone in a distinct color).

The system may select a stone removal treatment location based on the identified potential perimeter of the stone in the patient's organ. The control signal associated with the surgical instrument may be generated based on the selected stone removal treatment location. The visual indication of the decision context information may include a visual indication of the identified potential perimeter of the stone in the patient's organ.

The system may identify (e.g., based on the visualization of the patient's organ) a potential perimeter of a stone in the patient's organ. The system may identify a plurality of potential stone removal treatment locations. The system may determine (e.g., based on the potential perimeter of the stone in the patient's organ) respective confidence assessments that correspond to the plurality of potential stone removal treatment locations. The system may select a stone removal treatment location from the potential stone removal treatment locations based on their respective confidence assessments. The control signal associated with the surgical instrument may be generated based on the selected stone removal treatment location. The visual indication of the decision context information may include a visual indication of the potential perimeter of the stone in the patient's organ.

The system may merge types of visualization. The merged visualization may depend on the overlap and/or density of visualized information. In an example, the system may initially identify several potential objects of interest (e.g., which may be flagged in yellow). The system may identify the individual objects and adjust the color of the label to indicate the objects are surgically critical objects (e.g., to highlight their importance). After the system has identified the objects, the system may group the objects with a similar type and/or nature, and/or objects co-located near one another. The system may flag the group of related objects as a single object (e.g., to reduce visual clutter for the surgeon and/or staff).

If the surgeon proceeds against instructions recommended by the system, the system may perform one or more actions. For example, the system may output surgical reminders for actions, display visual cues for the system, etc. If the surgeon is proceeding in a step or manner that is in conflict with the action suggested by the system, the system may provide visual indicators for the action. For example, the visual indicators may include direct visual cues, such as further illuminating, highlighting, or blinking an area to indicate that another action has been recommended. For example, the displayed image of the first recommended stone for removal may blink to indicate that it should be removed first.

The system may provide indicators (e.g., generalized visual cues) such as blinking the perimeter of the screen in yellow or red to indicate warnings or potential missteps. The system may provide audible or haptic cues to indicate warnings or potential missteps. If a misstep is detected, the system may prevent the surgeon from performing the action.

An AI/ML feedback loop may be used to improve system decision making. For example, if the surgeon deviates from the expected procedure, the system may report the deviation as an input to an AI/ML feedback loop. The AI/ML feedback loop may output an automated surgery report/transcript.

The system may compensate for stone and/or organ shifting between pre-operative imaging (e.g., CT) and intra-operative imaging. The system may anticipate positional shifting of the gallbladder based on patient orientation. The system may utilize pre-preoperative imaging (e.g., CT) and table positions to calculate and estimate gravitational shift. The system may use the pre-operative imaging to create a range of locations (e.g., region of interest) where stones may have shifted. The system may use stones/lymph nodes as markers to distort the pre-operative scan to match patient position.

The system may import previous surgical data to assist in organ shifting predictions. The system may predict how far stones may have moved based on fluid progression and/or natural body processes (e.g., bile movement through the bile duct at a specified rate). The system may utilize previous patient shift data to predict shifting in subsequent patients. The system may assess organ movement of a patient during positioning to determine approximate organ shifting (e.g., using external markers).

The system may indicate if it is unsure if an object is a stone or node. The system may indicate if surgeon input is needed. The system may request surgeon input via a prompt or flashing icon on a screen or AR overlay. The system may adjust the transparency, color, brightness, etc. of objects to show the identification confidence level. For example, non-identified stones (low confidence, or unsure) may appear as a different color than identified stones and/or may appear with a prompt for surgeon input.

The system may display stone margins (e.g., perimeters) that show the center of stone at a higher confidence and show less confidence as the margin is expanded outside the stone (e.g., in a bulls-eye manner). The system may update stone identifier parameters to account for a given case (e.g., based on surgeon inputs). The system may prompt the surgeon that the parameters have been updated. The system may ask the surgeon whether the system should proceed with auto-identification of stones. The system may output an on-screen display of the total quantity of identified masses. For example, the display may indicate auto-identified stones (e.g., “Stones auto-identified: 6”), auto-identified lymph nodes (e.g., “Lymph nodes auto-identified: 4”), and/or unidentified objects (e.g., “Items pending review: 3”).

If the system identifies an object as a stone and the surgeon determines the object was incorrectly identified, the system may display that a surgeon override is detected. The system may ask the surgeon whether the system should report the identified object to a stone identification database. If the surgeon selects yes, the system may send information to the AI/ML cloud. If the surgeon selects no, the system may request whether to include the override in a surgery output report. The system may request that the surgeon indicate a reason for the override. The prompt may be presented to a circulating nurse to allow the surgeon to remain focused on the task at hand. The circulating nurse may record the reason for deviation from system recommendations.

If a stone is removed, the system may turn off the color/transparency indicator for that stone (or change to reflect the surgeon assessment). The system may determine if a non-gallstone was removed. The surgeon may confirm or deny whether the removed item was a stone. The system may request data to determine whether the item removed was a stone.

An in-surgery identification tool may be used to obtain stone identification feedback (e.g., prior to or after removal). For example, the identification tool may use one or more metrics (e.g., hardness, pH, density, tissue impedance, RF device impedance, harmonic tissue detection, etc.).

The system may identify if a gallstone has not (but should be) been removed. The system may overlay a gallstone identification chart from pre-operative and intra-operative imaging. The display may be updated in real time (e.g., change color/transparency after removal in the overlay). For example, after removal, the system may place an x over the stone or change transparency/color on the overlay. The system may use a generative fill (e.g., remove the stone from the pre-operative image to give the surgeon visual input that the stone has been removed). The surgeon may be able to toggle between before/after removal images. If the surgeon progresses past the stone removal, the system may indicate any stones have not been addressed.

At the completion of the surgery, the (e.g., all) stones may be assigned a treatment method (e.g., surgical removal, left to pass naturally, left due to access limitations, ultrasonic emulsification, left because the object was not correctly identified as a stone, etc. A data summary table may record the number of gallstones, the treatment method, relevant metrics, and/or linked pathology.

A smart data collection system may be used for foreign body removal tracking. A volumetric estimate of the foreign bodies may be determined based on pre-operative scanning. Removed foreign bodies may be placed on a tray for weighing (e.g., if typical density of body is known) or into fluid to measure volume. The measurement may be used to compare the volume of foreign body relative to the estimated total volume needed to be removed. If the removed volume is less than intended, the system may use this information to identify fractures of the stones.

A surgeon may gain confidence in the system's ability to make decisions. The system's decision-making approach may differ based on the user/surgeon, whether a device is new or existing, whether a user is new or existing, etc. The display may change as the user changes. Display preferences may follow users. The display may change based on a machine change (e.g., based on data), a number of times the machine has displayed information, surgeon performance; number of steps performed correctly, time between steps, eye tracking (e.g., surgeon not looking at information, machine offers to stop), surgeon input to alter the display, and/or the like.

Machine history may be used to determine an amount of information that the surgeon wants to see. User profiles may be used to set and save settings based on the surgery being performed. Stone identification threshold/sensitivity ratings may change based on the user. A level of detail customized for the user. Surgeon biometrics/preferences may be used in a simulation. For example, the system may store a glove size, establish an OR set-up based on user preference (e.g., OR table height, accompanying positions, etc.), settings for first time users, interns, residents, etc., and/or profiles for the surgery type (e.g., independent of the user).

Based on positional data, the surgeon may be interested in recommendations of angles of approach. The system may use data from other devices to determine angles of approach. For example, the system may use pre-operative imaging, target identification of surgical anatomy, critical structures to avoid, vessels during trocar placement, patient history, surgeon human factors data (e.g., dominant hand, height, vertical position of surgeon shoulders, etc.), patient height, trocar fulcrum, patient factors, table height, surgical position, BMI of the patient, surgical history, adhesions, OR personnel positioning, procedural approach, OR camera data, OR set-up, and/or the like.

The system may display recommendations to the surgeon. For example, the system may display a patient overlay via AR, selection options based on OR tasks prioritization, etc. The surgical planning system may allow a user to populate automated notifications or reminders to occur when a stone is encountered. The system may be used for teaching or educational purposes (e.g., surgical flow, key moment reminders, procedure checklist reminder, etc.).

The system may notify the surgeon if a stone is encountered. This may allow the surgeon to assess the stone prediction accuracy in real time (e.g., as each stone is approached in surgery). This may allow the surgeon to gain confidence in the identification system as an assistant. The system may learn from the surgeon's surgical decisions to improve future identification.

Pre-operative planning and/or imaging may be used by an automated intraoperative notification system. The system may identify and display a stone and its perimeter. If this is the first stone identification in this surgical procedure, the system may apply an ML filter to identify further stones. In another example, for cancer treatment, the system may identify tumors and their sizes. The system may explain the rationale behind identifying an object (e.g., similar size, density, etc.).

The system may read a radiograph to provide a supplemental analysis. For example, the system may identify artifacts created due to a CT machine position. The system may suggest turning the CT machine manually to remove shadow artifacts.

The system may display updates of progress within the procedure. The active information adjustment may indicate that the system is responding to real time behavior. A progress bar or other form of indicator may be displayed onto the screen. The system may be manually or automatically updated with the current procedure tasks or steps. Procedures may have unique listed items like the number of stones or stone pieces left to remove. If a stone fracture is detected, the system may automatically detect the number of pieces created. The indicator system may automatically update the display to indicate the number of pieces created. If the system monitors removal of foreign bodies, the system may automatically update the indicator to reflect the current progress into the procedure. The system may use the tool selection or tool activation patterns to detect the phase of the procedure (e.g., which may be reflected in the indicator system).

The system may display an on-screen procedure status or progression bar. The system may display a consensus of concurrent displays of the same assessment from differing points-of-view or customized for different HCPs. The consensus may be displayed relative to common landmarks or features of the image. These linked or cooperatives areas may be displayed with a common color, shape, digital badge, and/or notation. This may allow the HCP to switch views of the common display and re-orient themselves relative to the new-point-of-view. The images may be alternated to give the user a clear understanding of the different points-of-view and the reference shift.

Consensus references may enable an HCP to move from one view to another and re-orient themselves. For example, the consensus references may include tags or markings fixed in space to create a common reference point, fiducial markings of equipment to indicate orientation and location, and/or a common reference point for multiple images.

HCPs may be better able to re-orient themselves during a procedure due to the ability to change viewpoints. For example, a smart circular stapler (e.g., with an integrated camera) may be inserted into the patient. The staff may shift between the transanal view of the stapler during insertion and a laparoscopic camera view. The staff may be able to re-orient themselves to the procedure because they can quickly switch between different views.

The system may display images related to the movement of the system. The display may show a picture-in-picture image. The user may be able to perform a remote override of what is present on the screens. If multiple displays show different images, notification may display differently on the different screens or may be the same on both screens. Depending on users present, an interest in notifications may change.

Different HCPs may prefer that some data be displayed instead of other data. For example, a radiologist may monitor a cone-beam CT (for identification of tumor margins, instrument location, instrument orientation, critical structure identification, etc.), and the surgeon using an endoscope laparoscope may prefer that the display show data associated with the scope (e.g., to improve local control of the instrument).

HCPs may monitor the same region for different surgical reasons. For example, robotic imaging operated by the robotic control surgeon, and a laparoscope or endoscope being controlled at the table by the surgical assistant or second surgeon may both look at the same local area with differing visualization systems and purposes.

Multiple displays may show different aspects of the same data feed for different HCPs. For example, a back table nurse may look at the surgical procedure flow with emphasis on product usage and stock, and a surgeon may look at a related view of the surgical plan by looking at the instrument functional operation.

Multiple imaging arrays may be used to monitor different perspectives of a coincident location for different aspects of the surgical field and/or view. For example, the system may identify a stone size and bile duct size from pre-operative imaging (e.g., CT) and intra-operative imaging (e.g., EBUS). The system may use both datasets for display and decision making to determine whether a stone is removed or allowed to pass naturally, where the stone should be accessed from (e.g., via duct, or otherwise), etc. The surgeon/OR team may review the EBUS and CT data separately intraoperatively. The data may be presented to different users (e.g., CN sees CT, surgeon sees EBUS), or at different times (e.g., surgeon compares EBUS to CT by opening CT scan data in a minimized image).

Pre-operative and/or intra-operative imaging may be used to determine the cross-sectional area of a bile duct and/or stone size, compare the sizes to determine whether the stone may get stuck in the duct. The system may inform the surgeon of the stone size relative to the duct size (e.g., intraoperatively during interrogation of stone with EBUS). The system may display the size of stone as a percentage of the duct size (e.g., after surgeon uses EBUS on a stone).

The system may indicate a gallstone pass rate confidence using multiple data streams. The system may use a multi-spectral view to differentiate between differing characteristics of tissue. For example, multiple data streams in different light frequencies may be used to make different assessments regarding the same tissue. The data streams may be displayed differently to the OR team (e.g., Surgeon A may see IR data showing deep tissue penetration to ID stones, Surgeon B may see visual light while performing dissection to reach said stones).

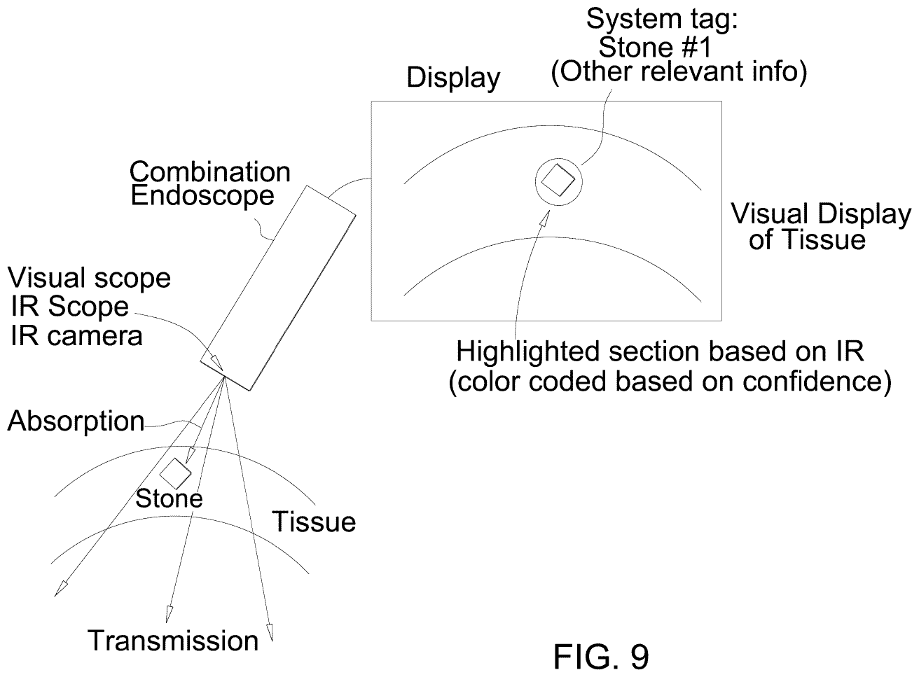

As shown in FIG. 9, a combination dual endoscope may use visual light and infrared (IR) visualization for subsurface visualization of stones within tissue (e.g., IR in the 600-1600 nanometer range may penetrate up to 8-10 mm of soft tissue but not the gallstones themselves). The scope may be used to identify potential stones within the tissue, allowing the surgeon real time feedback on the stone's location. Technology may be combined with other visualization/scanning techniques (e.g., CT, ultrasound, etc.) to increase confidence levels. Data may be sent to a user interface where a visual endoscopic image is displayed with an overlay of the infrared image, as shown in FIG. 9. The color and/or brightness of the detected stone may change based on the system's confidence in correctly identifying the stone. Depending on the IR absorption or transmission in the field of view, the overlay may display a map/information regarding stone location (e.g., with potential locations highlighted based on the system confidence of stone presence).

Users may have custom displays of data (e.g., from an endoscopic multi-band IR camera, an endoscopic IR camera, a data guidance display, etc.). In stone identification, the system may use a fluorescing, radio opaque, or other imaging detector to improve the contrasting of the difference between to the two indistinguishable options.

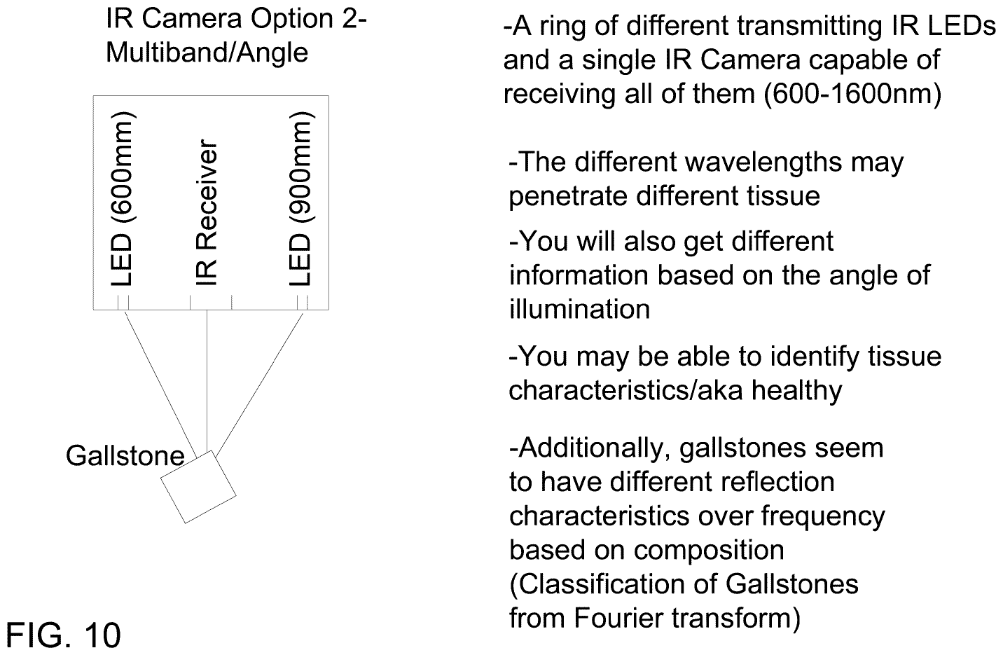

FIG. 10 illustrates another example dual endoscope used in gallstone detection. As shown, the endoscope may have 600 and 900 nanometer wavelength IR LEDs, and an IR receiver. The IR receiver may be capable of receiving the wavelengths of both IR LEDs. The different wavelengths may penetrate different tissues and/or depths. The IR receiver may receive varying information based on the angle of illumination of an area. For example, the endoscope may use the information to identify tissue characteristics (e.g., health, thickness, etc.). The system may use the different wavelengths and angles of illumination to identify foreign bodies, such as gallstones. Gallstones may have different reflection characteristics when illuminated with different frequencies. The reflection characteristics may change based on gallstone composition. The system may use an algorithm to classify objects as gallstones.

To aid in differentiating stones from nodes, the system may use a dye that would react to either the stone or node (e.g., but not the other). Such dye may create additional contrast in pre-operative imaging. The system may classify the likelihood that an object is a stone or node based on statistical size/shape/location of the object (e.g., IE nodes are on average smaller than X dimension, so a larger item is more likely to be a stone).

Foreign body identification may be used in gallstone removal. The system may display an uncertainty of portions of the overall decision. The display may include context to show aspects of which the system is fairly certain and aspects with a higher probability of misidentification (e.g., due to the subjective nature or erroneous implications).

For example, a multi-spectral imaging system with IR and fluorescing capabilities may be used to identify underlying critical structures (e.g., in order to minimize inadvertent collateral damage during dissection). Structures buried within surrounding tissues may obscure the crisp outlines of the critical structure. As the surgeon removes more surrounding tissues, the line defining the edge of the structure may become more defined or thinner.

The zone of the margin on the structure may be estimated using a combination of thermal gradients (e.g., to estimate how deep the structure is buried) and one or more other multi-spectral wavelengths to create a “zone of uncertainty.” The system may communicate the zone of uncertainty to the surgeon. The zone of uncertainty may be defined between the most conservative and most aggressive possibilities of the determined edges of the structure. As the system becomes more certain of the edge location, the zone may change colors, become more defined, or decrease in size (e.g., until specifically fixated around the edge of the structure).

For example, the system may detect a zone of uncertainty around cancer that is being resected. The tumor may have a definitive and/or different structural configuration compared to the surrounding healthy tissues. The zone may be defined as the amount/area of tissue to be removed so that no cancerous tissue is left behind. One or more imaging techniques may be used for intraoperative margin assessments. For example, imaging techniques may include computed tomography (e.g., cone mean CT), elastic scattering spectroscopy (ESS), optical coherence tomography (OCT), tagged florescence, and/or the like. The imaging techniques may have benefits and shortcomings (compared to other techniques) to assess the margin (e.g., until histologic sectioning is done post transection). Inadequate margins may create significant issues in cancer treatment. For example, the occurrence of inadequate margins is roughly 5% in lung cancer treatments, 15-20% in breast, prostate, and colon cancer treatments, and up to 40-60% in GYN cancer treatments. Margin may be a portion of the equation to be solved. The surgeon may balance retention of remaining healthy organ volume, proximity to critical structures, access concerns, dealing with related anatomic structures, and/or the like. These tradeoffs may influence the size of the margin used. With algorithmic interpretation of the imaging and ML and AI assessments of previous surgeries, aspects, and context relative to the current situation, the system may determine a zone of uncertainty on the edge of the impacted tissue and the completely healthy unaffected tissues.

The system may display multiple zones of uncertainty overlayed over each other. Such an overlay may enable the surgeon to choose the zone of uncertainty that is more important, more likely to be incorrect, or more likely to result in the best outcome for the patient. The system may overlay probability average lines overlaid on the zones of uncertainty to show the mean, median, or most likely guess of where the margins end.

If zones overlap in a manner that makes it improbable that there is a delineated line between the zones which can be used for the surgical step, the system or surgeon may select one of the zones to use. In this case, the overlapping zone may be highlighted to request that the surgeon to select a zone (e.g., define which zone is more critical in this situation, more trustworthy, etc.).

A smart system may request (e.g., require) input from a healthcare provider (HCP). The smart system may reduce the number of displayable options (e.g., to a more manageable group from which the HCP can choose).

The system may display multiple HCP choices. The choices may include future actions and/or probabilities of success. The smart system may have operational functions that can interfere (e.g., adjust parameters). If the system is using a data stream from another smart system, the data stream may be used to define at least two options that would result in different operations. The system may display the options to the user. For example, the system may display future steps of use and/or a parameter that is related to the probability of the system operating as desired. There may be multiple levels of choices. For example, the levels may have (e.g., require) varying levels of inputs from the HCP (e.g., surgeon). The levels may request minimal input (e.g., no say) or detailed intervention.

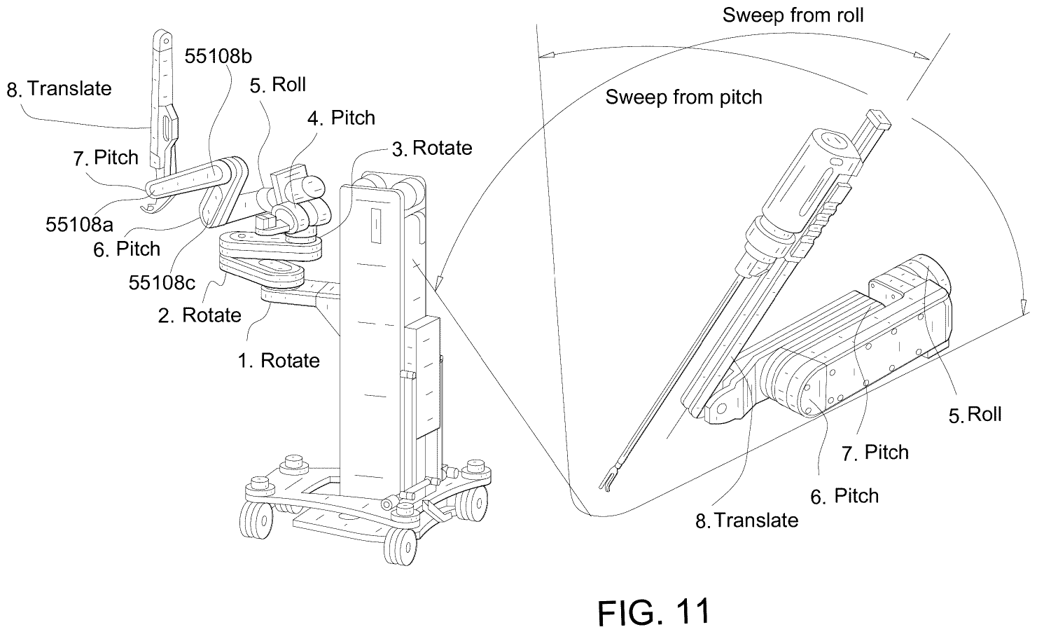

FIG. 11 illustrates example joint movements of a robotic arm attached to a base. As shown, the arm may be able to move in the x, y, and z directions. The joints may control the pitch, roll, and rotation of an end effector attached to the arm. The arm may have a mechanism (e.g., slide) for translation of the end effector. The joints may cause the end effector to make small movements inside the patient (e.g., in the surgical cavity). The arm movements correlated to the small internal movements may be large (e.g., creating a considerable sweep of the arm).

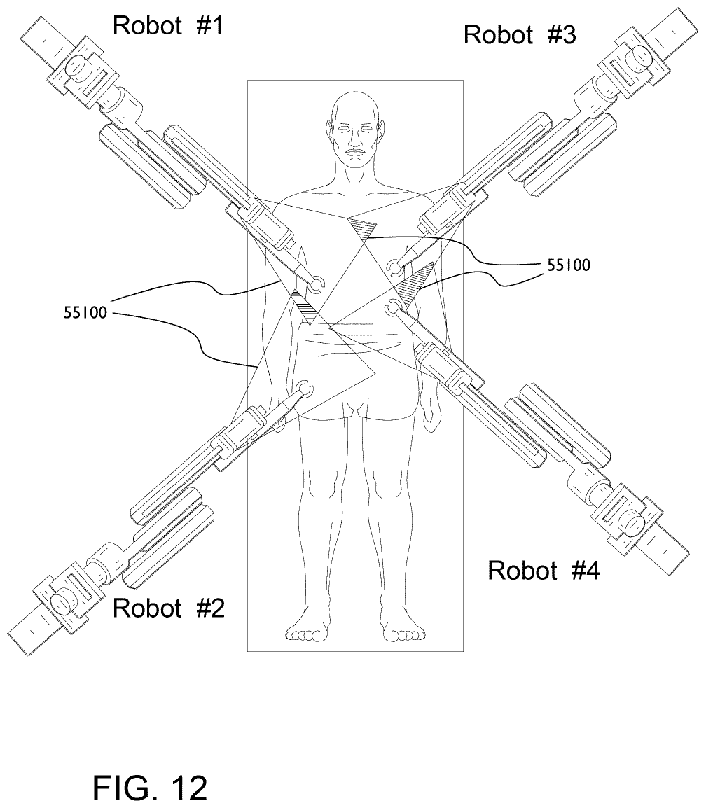

FIG. 12 illustrates an example operating room arrangement of multiple robotic arms. As shown, multiple robotic arms may be present in a relatively small space in an OR. The arms may have ranges of motion that overlap (e.g., as shown at 55100). In this case, the arms may collide unless adjustments are made to reduce the potential interactions. For example, one or more of the arms may not be allowed to occupy the overlapping space (e.g., at a given time). As described herein, the system may determine potential robot arm position placement and kinematic forecasting of resulting positions, movements, and/or interactions. The system may determine (and display) options for the HCP to choose from (e.g., and request that the HCP provide input). The system may output a simple, understandable display of context and choices.

Legibility (e.g., which is traditionally an attribute of written text) may refer to the quality of being easy to read and/or understand. A legible display of motion may involve (e.g., require) a conscious effort to make the diagramming clear and readable to a user (e.g., HCP).

Devices and methods for visualizing automated surgical system decisions. An example device may include a processor configured to perform one or more actions. The device may receive an indication of a surgical procedure that involves a first surgical instrument cooperating with a second surgical instrument. The surgical procedure may include a plurality of surgical steps. The device may determine a first candidate action and a second candidate action associated with the first surgical instrument. The first candidate action and the second candidate action may allow the first surgical instrument to complete a first step of the plurality of the surgical steps. The device may determine a first effect, caused by the first candidate action, on the second surgical instrument's ability to perform a second step of the plurality of surgical steps. The device may determine a second effect, caused by the second candidate action, on the second surgical instrument's ability to perform the second step of the plurality of surgical steps. The device may select, based on the first effect and the second effect, an action, from the first candidate action and the second candidate action, for the first surgical instrument to perform. The device may generate a control signal configured to indicate the selected action.

The selected action may be a first action. The control signal may be a first control signal. The surgical procedure may include a third step. The device may determine, based on the selected first action associated with the first surgical instrument, a third candidate action and a fourth candidate action associated with the second surgical instrument. The third candidate action and the fourth candidate action may allow the second surgical instrument to complete the second step. The device may determine a third effect, caused by the selected candidate action associated with the first surgical instrument and the third candidate action, on a third surgical instrument's ability to perform the third step. The device may determine a fourth effect, caused by the selected candidate action associated with the first surgical instrument and the fourth candidate action, on the third surgical instrument's ability to perform the third step. The device may select, based on the third effect and the fourth effect, a second action, from the third candidate action and the fourth candidate action, for the second surgical instrument to perform. The device may generate a second control signal configured to indicate the second action.

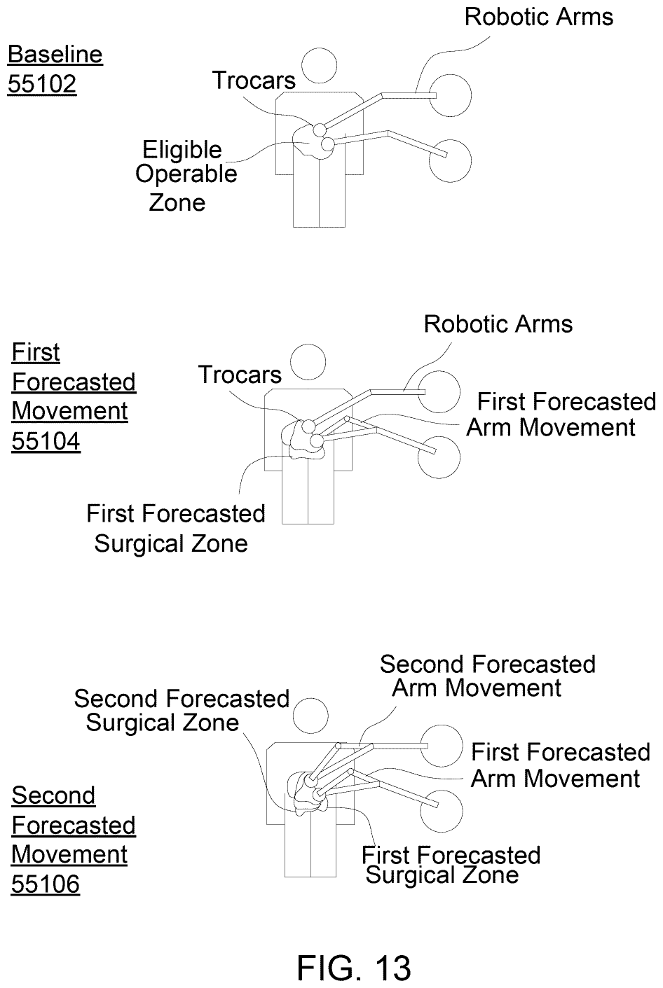

FIG. 13 illustrates an example display to a user of potential interactions between robotic arms during a surgical procedure. In a baseline position 55102 (shown in the top figure), the two robotic arms may be in an initial location. This initial location may be at a time just after the instruments attached to the ends of the arms were inserted into the patient via trocars. Based on this initial position, the system may determine an eligible operable zone. The eligible operable zone may indicate the areas in the patient's body that are accessible to the end effectors without moving arm joints external to the patient's body. The system may outline the eligible operable zone (e.g., within the surgical cavity) in a given orientation. The display may show locations of trocar ports through which the arms are inserted into the patient.

The surgeon may determine to move one of the arms using joints external to the patient (e.g., to access an area not in the eligible operable zone). The first forecasted move 55104 (e.g., first selected action, shown in the middle figure) illustrates an example movement of one of the arms. As shown, the system may update the displayed image to show an updated operable zone (e.g., a first forecasted surgical zone). The system may keep a depiction of the initial positioning of the arm. This may allow the surgeon to better visualize what the movement will look like. The surgeon may similarly move the other arm using joints external to the patient. The second forecasted move 55106 (e.g., second selected action, shown in the bottom figure) illustrates an example movement of the arm. As shown, the system may update the displayed image to show an updated operable zone (e.g., a second forecasted surgical zone). The system may keep a depiction of the initial positioning of the arm. These depictions may allow the surgeon to keep track of the positioning of the arms relative to each other, which may help the surgeon avoid collisions.

Robots may be equipped with means for determining the location of one arm relative to another in three-dimensional space. For example, another may monitor arms and provide relative measurements of one with respect to the other. For example, the system may determine the relative measurements through imaging of the OR. One or more cameras within the OR may be used to generate this information. The cameras may be separate from, or on the robot itself. The system may determine the relative measurements based on magnetic sensors, ultrasonic pinging, etc. This additional data feed may be used to determine the location of the devices relative to each other.

The hub and/or camera system may have to have stored parameter(s) related to the device(s) being tracked and/or capabilities of those device(s). Triangulation of the device position may be used to suggest device motions. For example, the system may use kinematics of the device(s) (e.g., robot arm(s)) and the patient to determine viable movement options. The kinematics may be derived (e.g., on the fly) using visualization. The range of motion (e.g., reach) and balance of the robot may be received from the robot or pre-determined.

Indexing elements (e.g., fiducials) on the arms may enable a separate system to monitor the arms, (e.g., each of the segments of the arms). In some examples, electronic sensors may be used (e.g., rather than fiducials). The electronic sensors may emit a signal that is received by other sensors or a base station. For example, fiducials and/or electronic sensors may be placed at points 55108a-c (e.g., and/or other joints) in FIG. 11.

A virtual fence (e.g., a dynamic virtual fence) may be used to choose and reserve a spatial volume (e.g., adjacent the patient) during the time an HCP (e.g., a surgeon or physician's assistant) intends to occupy the space (e.g., to grant access to the patient without interfering with the flow of the procedure). The device may identify a reserved space occupied by a third surgical instrument. Determining the first candidate action and the second candidate action associated with a first surgical instrument may involve determining that the first candidate action and the second candidate action cause the first surgical instrument to remain outside of the reserved space.

The first candidate action may involve placing a port in a first port location on a patient. The second candidate action may involve placing the port in a second port location on the patient. The control signal may be configured to indicate one or more of: a first magnitude of access that a laparoscopic instrument will have to a surgical area if the first port location is used, a first number of orientation possibilities of the laparoscopic instrument if the first port location is used, a second magnitude of access that the laparoscopic instrument will have to a surgical area if the second port location is used, or a second number of orientation possibilities of the laparoscopic instrument if the second port location is used. The accessory trocar port location may be identified (e.g., during initial robotic port placement or at any other time during the procedure). A virtual fence defining a space the surgeon would occupy when using a device may be defined around the accessory trocar port.

The first surgical instrument may include a joint. The first candidate action may involve placing a port in a first port location on a patient. The second candidate action may involve placing the port in a second port location on the patient. The device may determine a first effect associated with the first port location and a second effect associated with the second port location based on at least one of: a position of a health care provider relative to the first surgical instrument, an articulation angle of the joint, a joint length of the joint, or a degree of freedom of the joint.

The first candidate action may involve a first placement of a base associated with the first surgical instrument, or a first movement of the first surgical instrument. The second candidate action may involve a second placement of the base associated with the first surgical instrument, or a second movement of the first surgical instrument.

The virtual fence may be dynamic in the sense that it can be turned on and off. The dynamic virtual fence may be turned on/off by the surgeon operating the robot. The dynamic virtual fence may be turned on/off by the robot itself (e.g., based on the next anticipated steps of the procedure).

The virtual fence location may be adjusted (e.g., based on the measured angle of the device that is observed to be passing through it by a laparoscopic camera). The laparoscopic camera may be oriented relative to the accessory trocar (e.g., during a setup procedure step) to register a common coordinate system and location. The virtual fence may adjust in size, shape, and/or location while on. If an accessory device is introduced through the trocar, the orientation of the device may be used to adjust the location/size/shape of the dynamic virtual fence. The surgeon operating the robot may adjust the location/size/shape of the dynamic virtual fence based on feedback from the user of the accessory trocar.

Anticipated procedure steps may be created using historical data from procedures with similar characteristics as the current one (e.g., patient factors, disease state, surgeon, device utilization, etc.). Anticipated procedure steps may be determined (e.g., directed) by the surgeon operating the robot.

The dynamic virtual fence may be slowly or instantaneously turned on. The robotic arms may adjust to this space to ensure that the fence is not violated by any portion of the arms. If the fence is turned on instantaneously, the robot may take a short period of time to reposition itself. This can be accomplished without changing the end location of devices attached to the robot arms (e.g., if the arms have sufficient degrees of freedom of motion).

If the robot anticipates that a procedure step is coming, the robot may make choices about how it moves its arms (e.g., to start to create the space prior to it being needed). This may result in less efficient movements leading up to the virtual fence being present. The movements may be accommodated through sufficient motor speed/direction choices, etc.

The device may receive user preference information and a patient position associated with the surgical procedure. The device may determine a surgical constraint based on at least one of the user preference information or the patient position. Selecting the action, from the first candidate action and the second candidate action, for the first surgical instrument to perform, may be based on the surgical constraint.

If the virtual fence has been created, the robot may respond to movement commands from the surgeon to move from location to location (e.g., using control algorithms that account for the constraint of not being able to violate the virtual fence).

For example, in thoracic resection procedures, there may be multiple (e.g., up to three distinct) tissues to staple (e.g., lung parenchyma, major pulmonary vessels, and major pulmonary airways). Specialty staplers may be used for the vessel and airway firings due to their unique access capabilities. Some robotic staplers may not have these access capabilities. The performance of some robotic staplers may be inferior to available handheld options. Handheld staplers may be used in robotic procedures. Space around the patient may be reserved for the HCP (e.g., a surgeon or physician's assistant) to reside to perform the firing (e.g., without interfering with or interference from the robot).

If the vessel and/or airway transection are complete, the reserved space may be removed (e.g., allowing the robot to return to an unconstrained state of movement based on the original algorithms).

The options of kinematic motion may be documented. Ambiguous displays may complicate the easy understanding of the choices the surgeon is making (e.g., in real-time or future steps).

The system may output a predictable display of sequential options. The choice the HCP is making may cause only arm-to-arm interaction outcomes. The choice may affect the orientation and position of the trocar and the internal surgical site. The choice may affect the remaining amount of head rotation or articulation angle of a robotic arm (e.g., beyond what is currently being used). The choice may affect the remaining degrees of freedom the end-effector has to get to locations beyond its current position.

The information being presented to the HCP may be relevant to the decisions being made or actions being taken at a point in time. The screen or display information may not (e.g., should not) include information that is no longer relevant, or a level of detail that is not appropriate.

The display options may help the HCP or operating room (OR) team understand the decisions the robot is making, and assist/control these decisions (e.g., as needed).

The system (e.g., robot) may display the current status of the surgical procedure and/or step. The system may display associated decision(s) and/or movement(s) being made by the system. The display elements may indicate the broader context of the decision(s) within the surgery (e.g., to give the HCP greater confidence and understanding of the decision(s) made by the system).

The system may nest choices within the context of a broader surgical step. For example, the system may provide a series of smaller steps as they relate to an overall larger surgical objective or goal.

The system may display collapsible views of non-active surgical steps.

To provide context, larger segments of the operation may be viewable and/or observable by the surgeon and/or surgical staff. To minimize the display of information (e.g., minimize information overload), non-active segments or broader steps may be collapsed.

The system may output an integrated display of a current autonomous step and upcoming non-autonomous step(s).

The system may provide context about upcoming steps that may require surgical guidance or action by the HCP (e.g., as opposed to autonomous action).

The system may indicate whether the decision is being made autonomously by the system or involves a human decision. The indication may be a light emitting diode (LED) display on the system, a screen display on the system (e.g., for staff), on a surgeon console, a light bar on the equipment/instrument, and/or the like.

The system may display forecasted decision(s). The system may provide an indication (e.g., in advance) of how a decision will be made or forecasted. The indication may be via a series of profiles, standard locations of equipment, imagery or text to provide the HCP or other users of the equipment the related information, and/or the like.

The system may display multiple step kinematic combinations (e.g., with a second variable, for example, probability of unintended interactions) to clearly articulate options to choose from or potential issues. The system may attempt to minimize arm interactions. The system may suggest arm placements (e.g., based on the patient size and orientation). The system may display the benefits/likelihood of arm interactions based on trocar/arm positions. For example, the system may display a topography map of likely interactions. The system may display a procedure map that highlights when and/or where arm interactions are expected to occur.

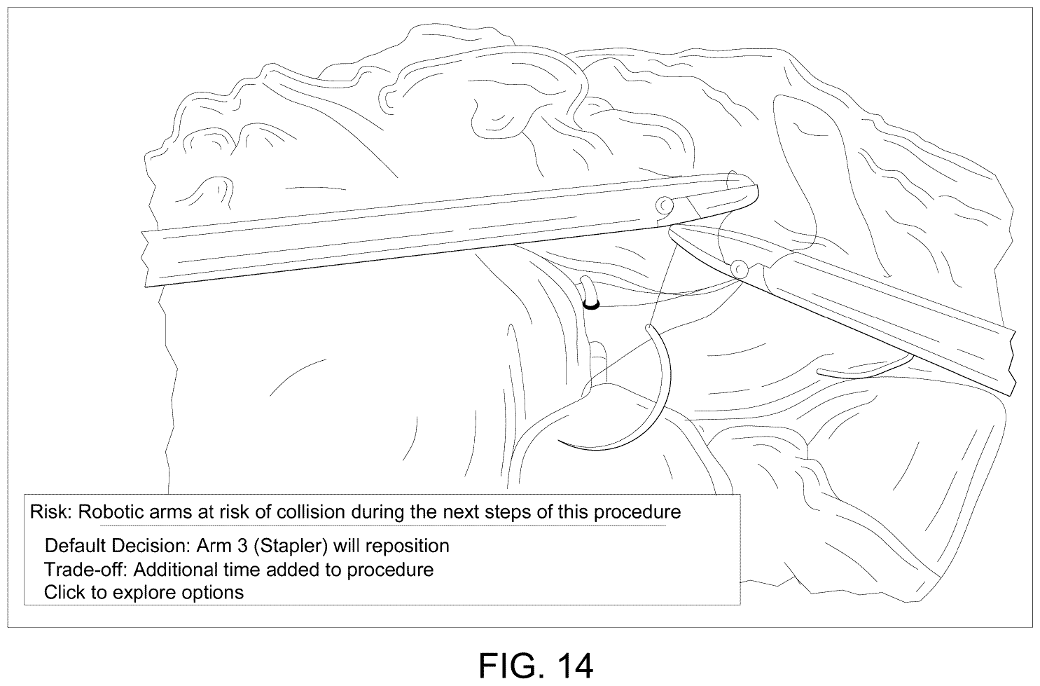

FIG. 14 illustrates an example display to a user of a decision to reposition a robotic arm to avoid a collision. As shown, the display may indicate that the robotic arms are at risk of colliding during one or more of the following steps of the procedure. The system may indicate that a default decision (e.g., if no user intervention is detected) may involve repositioning an arm (e.g., “Arm 3” with a stapler attached). The system may indicate that the default adjustment may increase the overall time of the procedure. The display may include a link that will allow the user to explore additional options (e.g., other than the default). The additional options may similarly include an indication of pros and/or cons associated with the options. For example, an additional option may be to reposition a different arm. The system may indicate that this option will likely cause another collision within the next few steps. These options and associated information may allow the surgeon to make informed decisions about how to proceed.

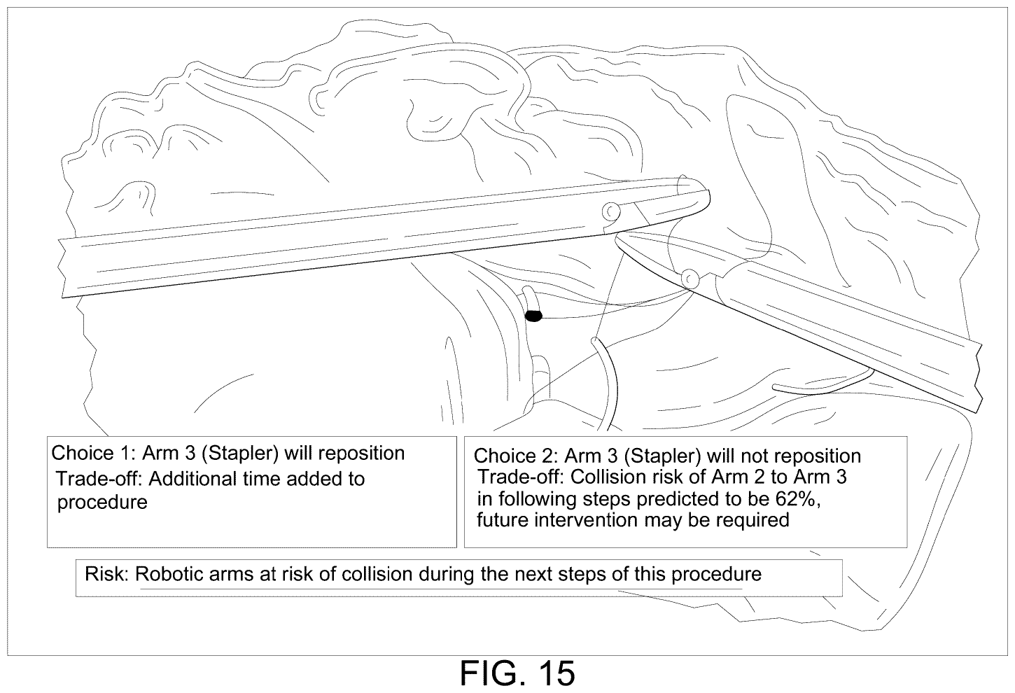

FIG. 15 illustrates an example display to a user of optional actions to avoid a collision. As shown, the display may indicate that the robotic arms are at risk of colliding during one or more of the following steps of the procedure. The system may indicate one or more options (e.g., choice 1 and choice 2 in FIG. 15). The system may indicate that one of the options is a preferred option. As shown, choice 1 may involve repositioning an arm (e.g., “Arm 3” with a stapler attached), which may increase the overall time of the procedure. In choice 2, Arm 3 may not be repositioned (e.g., another arm may be repositioned, or the surgeon may accept the risk of collision). The system may indicate that choice 2 is associated with a 62% collisions risk that will require future intervention. The surgeon may select one of the options or otherwise intervene to avoid the collision.

The system may propose change(s) throughout the procedure. For example, if a collision or interaction is about to occur, the system may determine how the user is notified/what is displayed. For example, the system may indicate options (e.g., whether the system should stop or change directions).

Collision avoidance may depend on the source of the instruction that will cause the collision. The system may determine its reaction to a potential collision based on the method by which it received the instruction that will cause the collision. For example, if the instruction is received (e.g., directly) from the surgeon, the system may allow the movement(s) to proceed (e.g., with a warning indication). An instruction that may (e.g., inadvertently) result in a collision that is created by the system (e.g., the system's control algorithms) may be stopped.

The system may indicate for the HCP to change a robot's position based on which a tool is attached. Coordinated colors and/or blinking on tools and a base may indicate a preferred location.

A system may have an LED at the base of the tool driver assembly (e.g., where it interconnects to the robotic system) and the housing or assembly portion that connects to the tool driver. The system may use color control of the LEDS to indicate the correct housing to the correct tool driver assembly.

For example, during initial setup for a surgical procedure with three tool housings, the tools may be connected to their appropriate housings. There may be three housings and three tools, each with their respective colors matching (e.g., Tool 1: Blue; Tool 2: Yellow; Tool 3: Red; Housing 1: Blue; Housing 2: Yellow; Housing 3: Red). As the procedure progresses, the system may indicate for the HCP to swap one or more tool location(s). For example, the following changes may be made. Tool 1: Blinking Red; Tool 2: Yellow (e.g., no change); Tool 3: Blinking Blue. The base or housing LEDs may not change colors (e.g., stay constant throughout a procedure). The blinking may indicate for the tools to be swapped to the identified new location.

The color of a housing indicator may be determined based on how it is identified in software. For example, in a multi-housing robotic system (e.g., Hugo robot), housing system #1 may (e.g., always) be mapped to ‘Blue’; housing system #2 may (e.g., always) be mapped to ‘Yellow’; and housing system #3 may (e.g., always) be mapped to ‘Red.’ If the mapping of these systems changes, the color identification may change as well.

Multi-factor information may be used to determine whether to switch a tool to another base. The system may display steps and/or instructions on the change to be made (e.g., with visible indications of the change to be made, for example, blinking LEDs on the tool and base).

The system may determine and display options from which the HCP may choose.

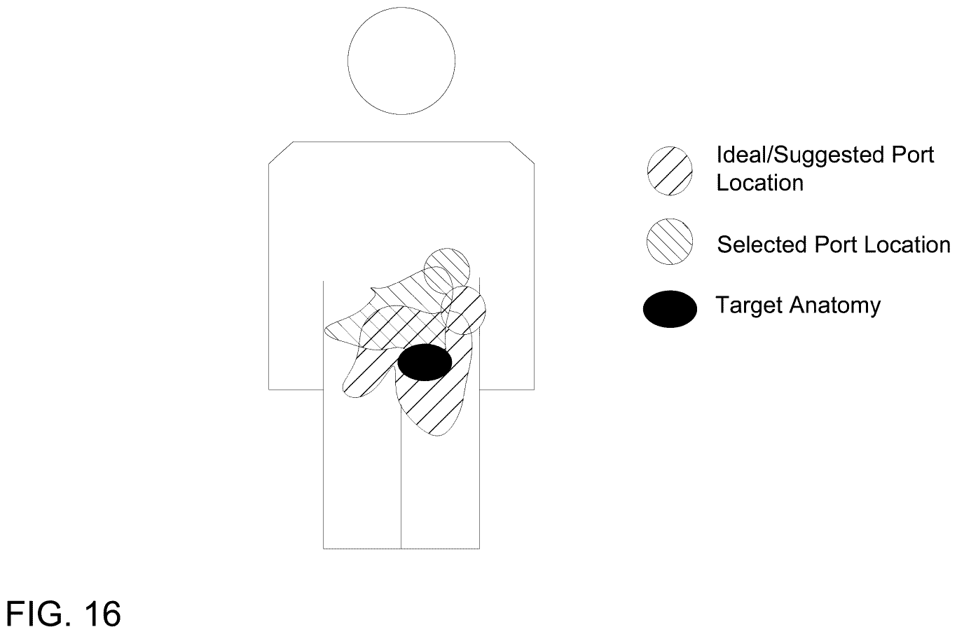

Instrument interactions may be affected by trocar placement and/or HCP position. The system may display the access port location and surgical site accessibility. FIG. 16 illustrates an example display to a user of the impact of potential port locations. As shown, the system may indicate a selected port location, an ideal/suggested port location, and a location of the target anatomy (e.g., to be reached via the port). The display may also show the areas that the end effector will be able to reach depending on which port is used. In the example in FIG. 16, the display allows the surgeon to visualize that the selected port location will not grant as much access to the target anatomy as the ideal/suggested port location would.

The port location of the trocars through the abdomen or thoracic walls may determine the angle of approach to the surgical site within the body. The configurations, articulation angle, joint length, degrees of freedom of the joints, and/or the rotational capabilities of the instruments may determine the viable approach angles and/or orientations of the end-effectors. The ability to visualize the interaction of the instrument choices and the access port locations may enable the surgeon to choose instruments that are capable of accessing the surgical site or what port locations to use (e.g., based on the choice of instruments).

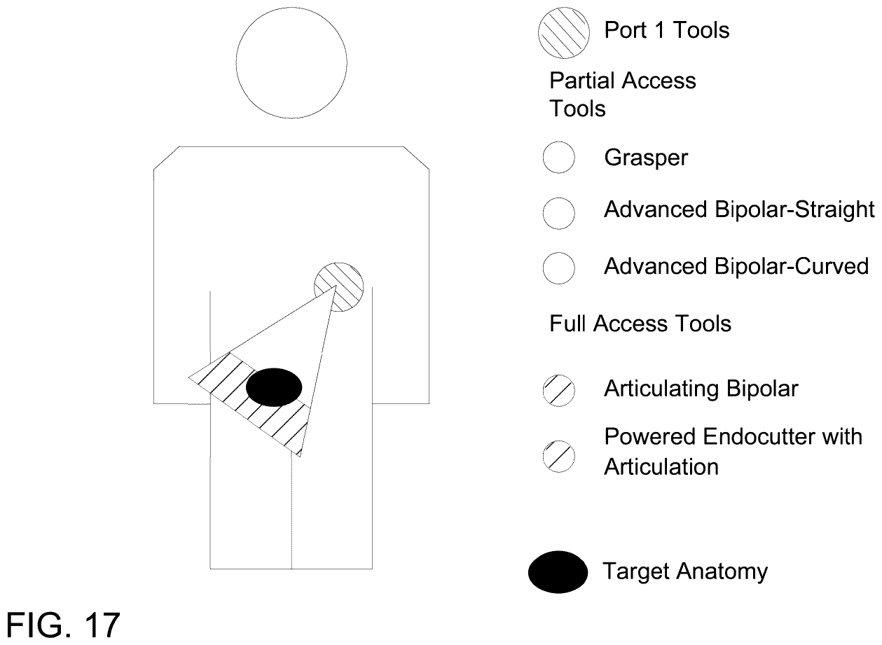

FIG. 17 illustrates an example display to a user of the access capabilities of surgical tools based on a port location. As shown, the system may indicate the areas in which different tools will be able to access via a first port location. The partial access tools (e.g., grasper, advanced bipolar-straight, and advanced bipolar-curved) may be able to reach the target anatomy but will not be able to access all of the target anatomy. The full access tools (e.g., articulating bipolar and a powered endocutter with articulation) may be able to access all of the target anatomy (and perhaps beyond the target anatomy).

A device may have actuation jaws with different configurations. For example, in an endocutter, one jaw may be used to house the cartridge and the staples (e.g., making it twice as large as the other jaw). In an endocutter, one of the two jaws may pivot, and the other jaw may be fixed to the shaft axis. In ultrasonic or bipolar advanced energy devices, the blade and the wave guide may be coupled to the shaft (e.g., or an aspect of the shaft). The clamp arm of the device may be moved relative to the shaft.

The system may determine an approach angle to the local surgical site. The system may determine an orientation (e.g., a preferred orientation) of the end-effector to the anatomic structures of the patient.

For example, in pulmonary artery/pulmonary vein (PA/PV) transection of the lung in a segmentectomy or lobectomy, the surgeon may prefer to have a non-moving jaw and the smallest profile jaw underneath the artery during introduction. This may reduce (e.g., minimize) the likelihood of tears or unnecessary tissue tension of the fragile artery. This may enable the least amount of skeletonization and dissection and may reduce (e.g., minimize) the likelihood of collateral damage. The access orientation of the shaft of the device may be used to position the end-effector perpendicular to the artery and vein (e.g., while avoiding other structures and having only a limited amount of access possibilities through the rib cage).

The system may show the magnitude of the difference of port placement on the instrument access and orientation possibilities (e.g., relative to the choice of differing instruments). The system may display a simulation of port placement relevant to toolset selection for a given placement.

The simulation may allow information to be presented to the surgeon. For example, the system may (e.g., dynamically) present information about different instruments for a given port placement. As the selected placement moves, the information regarding the utilizable tools may shift.

A composite rendering that highlights the differences of the ideal location (e.g., as opposed to the selected location) may be displayed.

The system may present information relative to target anatomy. For example, the system may display a simulation of port placements for a given surgical procedure and anticipated tools. The system may display optional location(s) of port placement based on anticipated variables (e.g., selected surgery and tools) and the impacts of using the location(s). The port placement(s) may reduce (e.g., minimize) robot arm interactions.

The location of the access ports and the instruments may interfere with the local imaging access options. The imaging systems may be affected by the local presence of metals, electromagnetic fields, capacitive coupling, and/or other energy-based interactions between the proximity and magnitude of the interactive effect. For example, the EM sensors (e.g., on the monarch flexible robotic scope) may be affected by the proximity and size of metal objects within the field (e.g., generated by the unit base station). The CT may reflect off of metal objects causing a glare occluding localized visualization. Impendence spectroscopy and/or other conductivity through the tissue may be affected by the presence of metal objects and saline near the imaging location. Capacitive coupling may occur if high levels of energy are moved down a shaft that is closely aligned to another conductive shaft. Capacitive coupling may result in parasitic drains on the sensing and/or unintended currents in the other devices (e.g., and potential burns within the patient).

The system may determine how to display complicated data (e.g., multiple data streams and context) so that the user (e.g., HCP) can make a decision (e.g., give input to the system).

The system may use an adaptive display of the data to visualize trends. The system may adapt how the data is presented to the surgeon (e.g., in real time), for example, based on the data stream(s).

The visualization/presentation of the data may be determined based on relative changes in parameters. For example, related features may be clustered to illustrate patterns around categorical variables. For example, vessel parameters (e.g., size, dissection, etc.) may be located adjacent to one another on the display.

The visualization/presentation of the data may be determined based on changes of parameters over time. For example, a mean and/or range dot plot may be used. An area chart may be used to display relative magnitude over time.

The visualization/presentation of the data may be determined based on interrelated parameters. For example, stacked summary bar charts may be used to illustrate multiple summed data streams (e.g., and the originating data trends). The data may be divided based on tissue type.

The visualized data may include ablation and/or drug delivery, tumor size (e.g., throughout removal), a probe coverage zone, a distance to critical structures, tumor density (e.g., susceptibility to treatment), surrounding tissue damage, and/or the like.

In the case of gallstone removal, the visualized data may include a percentage of successful identification of stones, stone size, percentage of natural passage, and/or the like. In another example, the system may determine that an object is a stone, but its location is somewhere that a stone would not be. In this case, the system may request doctor input to help identify the object. The system may explain why it is unsure of the object's identity (e.g., the location).

The data may be divided based on procedure type. For example, the data may show a percentage of likely robot arm collisions during the procedure.

The system may reduce (e.g., eliminate) noise via the diagramming. For example, rose diagramming may be used. The system may display co-morbidities related to a selected procedure approach.

The system may display breakout diagramming of multiple choices. For example, a sunburst pie chart may be used for relational display of magnitude and frequency of common data points. A percentage breakout of stacked bar charts may be used to display trends. For example, the Y axis may illustrate procedure steps, the X axis may represent time, and different colors may be used to differentiate between laparoscopic and endoscopic device data.

The visualization may be adapted based on smart device capabilities and/or magnitude of requested interpretation. An example miso algorithm may output data to a hub. The system may display tissue creep and/or force stabilization using a first diagramming type, and knife speed force implications may be displayed using a second diagramming type.

The system may output a haptic indication of lack of operation. The system may indicate a reason for the indication (e.g., full articulation giggle, pause giggle, unable to proceed, further required reaction giggle, etc.). The reason may be indicated in a non-visual manner (e.g., based on intensity, duration, and/or frequency of haptic feedback).

The system may adjust a graphical user interface (GUI) on a screen. The adjustment may improve visualization of the context of the data (e.g., the non-obvious relationship between data streams). For example, the magnitude of a displayed event may be adapted (e.g., based on a procedural step and/or the like). The scale of the displayed data may be adjusted. The scale may be adjusted based on a relationship between a localized event compared to an ongoing baseline.

The display may show an anomaly score of harmonic activations. This data may indicate how the device performance changes throughout the life of the device. The data may indicate if an activation seems abnormal. An anomaly score is an example metric that can be calculated by a smart system to characterize device behavior/performance. For example, an anomaly score may be produced by the smart system running an Isolation Forest ML algorithm. An anomaly score of zero or less may indicate that the data point is anomalous.

The device may determine a parameter change associated with the first effect and the second effect. The device may determine a data type associated with the parameter change. The device may determine a format of a graphical representation of the first effect and the second effect based on the data type. The device may generate the graphical representation based on the determined format. The control signal may be configured to instruct a display to display the generated graphical representation.

The device may determine the data type associated with the parameter change based on one or more of: an absolute change in a parameter over a period of time; a relative change in the parameter over the period of time; data trends of summed data streams of interrelated parameters; or an impact, of the first or second effect, to one or more surgical instruments.

A data point in the graphical representation of the data may represent an activation of the device. The graphical representation may be shown as the surgeon progresses through a procedure (e.g., so that the surgeon is able to determine if an activation was flagged as anomalous).

In an example, a patient may be placed into a hypothermic state. The rates of insufflation gases, smoke evacuation, suction irrigation, and/or the like may impact patient temperature. The system may display a rate of change of the patient's body temperature. The system may display data from other systems that might be driving the rate. For example, limits on patient heating and cooling may be relevant to the system's decision to change a rate of heating or cooling.

Although some aspects are described with respect to one or more robotic arms, a person of ordinary skill in the art will appreciate that these aspects may be used for any powered device (e.g., an articulable endocutter, etc.).

Robotic arm placement may be optimized to determine (e.g., and indicate) potential arm interaction(s). The placement may be selected to help mitigate entanglement and/or collisions of the robotic arms.

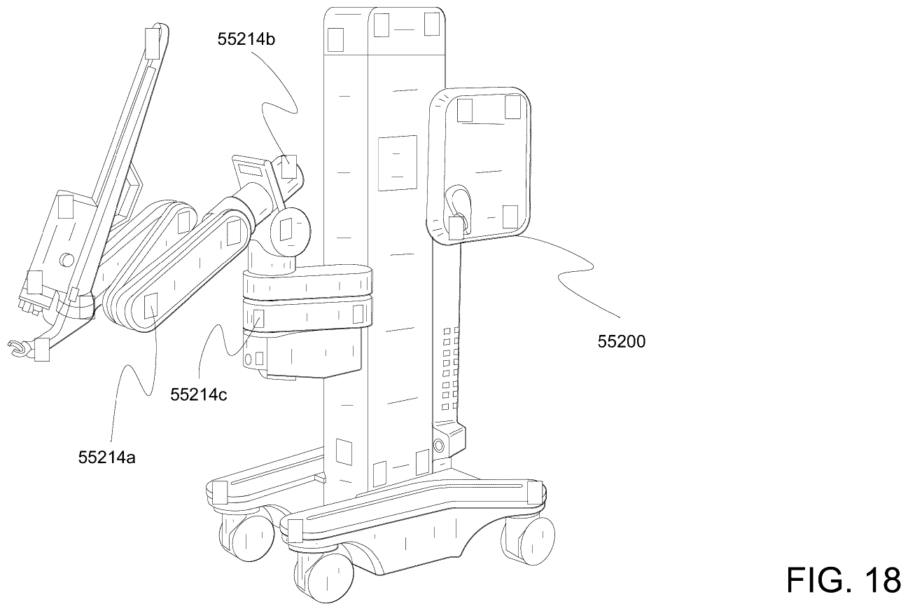

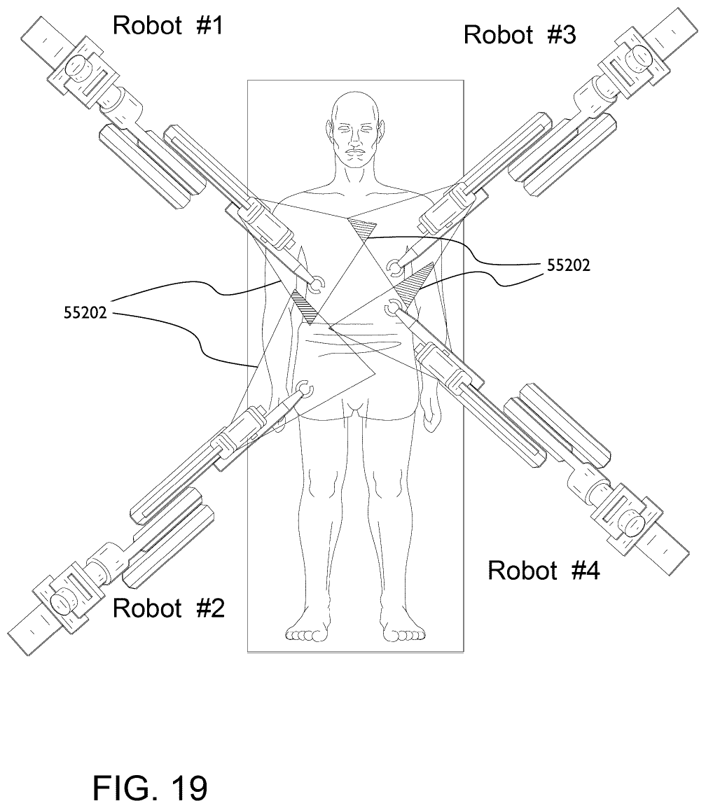

A user (e.g., a health care provider (HCP)) may select the placement of a first surgical instrument (e.g., a base of a robotic arm). FIG. 18 illustrates an example robotic arm 55200 attached to a base. FIG. 19 illustrates an example operating room arrangement of multiple robotic arms. As shown, multiple robotic arms may be present in a relatively small space in an OR. The arms may have ranges of motion that overlap (e.g., as shown at 55202). In this case, the arms may collide unless adjustments are made to reduce the potential interactions. For example, one or more of the arms may not be allowed to occupy the overlapping space (e.g., at a given time). As described herein, strategic placement of the robotic arm bases may help reduce the number of interactions between the arms.

A smart surgical system may determine (e.g., predict) the effect of the placement of the first surgical instrument on the placement of a second surgical instrument. The determination may be based on a predicted progression of a surgical procedure (e.g., where the surgical instruments will be during the steps of the surgical procedure).

The smart system may use the placement of the first surgical device to predict the (e.g., best) placement of the remaining surgical instrument(s). The smart system may display the interaction(s) between the surgical instruments based on the placements. The smart system may display the location(s) of the interaction(s) relative to the surgical site. The smart system may display placement options to the user to allow the user to place or indicate a selected placement location. The smart system may display placement option(s) of other surgical instruments based on the selected location.

The smart system may predict the (e.g., best) placement(s) of the surgical instrument(s) based on the interactions and procedure constraints. The procedure constraints may include the procedure steps, the instruments selected, and the user access specifications (e.g., requirements). The smart system may display placement options to the user. The display may include multiple cascaded placement options. The placement options may be determined based on user prioritization or choices, the layout of the operating room, equipment type/placement, and/or patient positioning. The placement of robotic arm(s) may be determined so that the arm(s) are able to reach the surgical space (e.g., all of the surgical space) inside the patient.

Placement preferences (e.g., from the HCP) may influence criteria for placement determination. Different HCPs (e.g., surgeons) may have varying skillsets, tool preferences, and/or surgical approaches. HCPs may have different approaches to the same surgical procedure. For example, a resident surgeon being overseen by an attending surgeon may use a different surgical approach than an experienced surgeon (e.g., who has performed a large volume of procedures). The criteria for placement determination may change from one HCP to the next.

The system may choose to select device location to allow the device to access the (e.g., all of the) surgical space. The system may select the device location based on one or more constraints (e.g., toolset length, tools to be used, device articulation capabilities, and/or a number of instruments in use, which may be limited due to cavity space, for example). If the device(s) are able to be moved (e.g., manipulated) throughout the procedure, one or more constraint(s) may be flexible.

The device may receive user preference information. The device may determine a surgical constraint based on the user preference information. The device may select the candidate position for the second base, from the first candidate position and the second candidate position, based on the surgical constraint.

The device may determine a patient position associated with the surgical procedure. The device may determine a surgical constraint based on the patient's position. The device may select the candidate position for the second base, from the first candidate position and the second candidate position, based on the surgical constraint.

During a surgical procedure, the movement of end effectors attached to robotic arms may cause arm interactions outside of the patient. The arm interactions may be reduced (e.g., minimized) to avoid collisions and/or entanglements (e.g., that could interrupt the procedure or prevent surgical access). The movement of end effectors may similarly cause interactions between the arms and other objects (e.g., people, stationary devices) in the OR.

The first robotic arm may be configured to move a first end effector attached to a distal end of the first robotic arm, and the second robotic arm is configured to move a second end effector attached to a distal end of the second robotic arm, each step in the plurality of steps of the surgical procedure is associated with a surgical space internal to a patient. The device may identify a set of candidate positions, comprising the first candidate position and the second candidate position, based on the plurality of steps of the surgical procedure. Each candidate position in the set of candidate positions may allow the first end effector and the second end effector to access the surgical space at a given step in the plurality of steps of the surgical procedure.

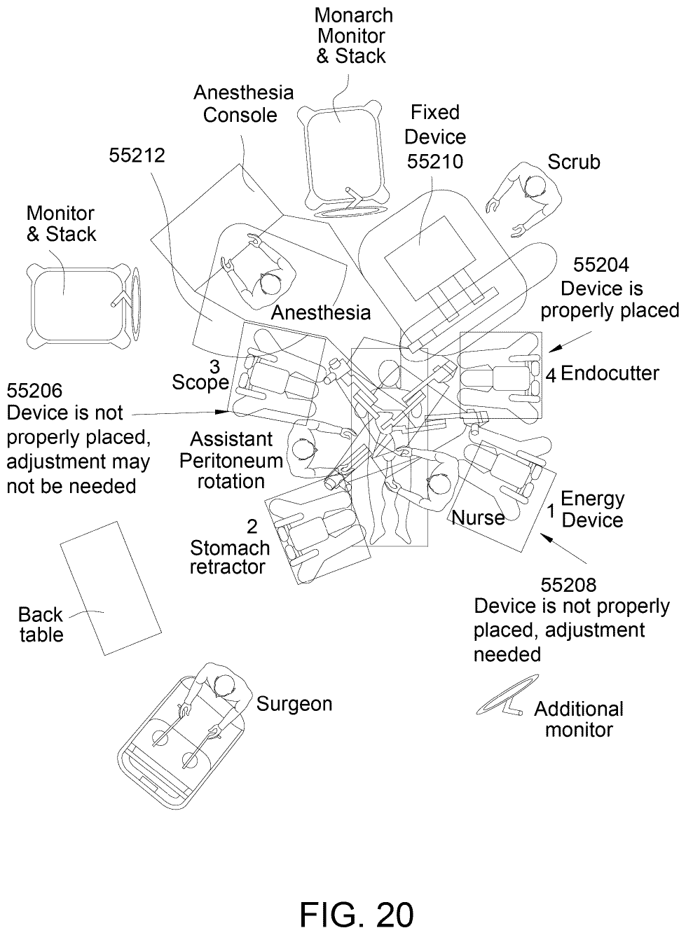

The smart system may predict placement(s) for one or more robotic arms (e.g., the Hugo robot arm). The system may display a recommended placement to the OR team. FIG. 20 illustrates an example display to a user of the effects of device placement in an operating room. For example, as shown at 55204, the system may indicate that a device is properly placed. The system may indicate (e.g., at 55206) that a device is not properly placed, but that the placement is close enough to the recommended placement that adjustment may not be needed. As another example, the system may indicate (e.g., at 55208) that a device is not properly placed and should be adjusted (e.g., because the current placement of the device may cause negative interactions between devices).

FIG. 20 further illustrates an example of a fixed (e.g., non-moving) device 55210. The fixed device 55210 may be a ventilator (e.g., a Monarch smart ventilator). The ventilator may be placed (e.g., and fixed) at the patient's head (e.g., because the ventilator must be attached at the patient's mouth and/or nose). FIG. 20 further illustrates an example area 55212 in which an HCP (e.g., the anesthesiologist) will occupy during one or more parts of the surgery. For example, throughout a surgery, the anesthesiologist may need to have access to the patient's head to properly check and maintain sedation.

Visualization of the placement(s) may differ based on a level of interest that the HCP has in placement prediction. For example, a surgeon performing a procedure that they perform often (e.g., 100 times per month) may want less recommendation for placement (e.g., have less interest in placement prediction). In another example, a surgeon performing a procedure for the first time may prefer heavy intervention (e.g., have a great interest in placement prediction). Intermediate levels of interest may similarly be available. The level of interest may determine an amount of information and/or recommendations displayed to the HCP.

Devices and methods for visualizing the effects of device placement in an operating room. An example device may include a processor configured to perform one or more actions. The device may receive an indication of a plurality of steps of a surgical procedure. One or more steps in the plurality of steps of the surgical procedure involve use of at least one of a first robotic arm attached to a first base, or a second robotic arm attached to a second base. The device may determine a fixed position of the first base. The device may determine, based on the plurality of steps of the surgical procedure and the fixed position of the first base, that a first candidate position of the second base is associated with a first number of interactions in which the first robotic arm and the second robotic arm will co-occupy space during the surgical procedure. The device may determine, based on the plurality of steps of the surgical procedure and the fixed position of the first base, that a second candidate position of the second base is associated with a second number of interactions in which the first robotic arm and the second robotic arm will co-occupy space during the surgical procedure. The device may select a candidate position for the second base, from the first candidate position and the second candidate position, based on the first number of interactions and the second number of interactions. The device may generate a control signal configured to indicate the selected candidate position for the second base.

On a condition that the first number of interactions is less than the second number of interactions, the device may select the first candidate position. On a condition that the first number of interactions is greater than the second number of interactions, the device may select the second candidate position.

The control signal being configured to indicate the selected candidate position of the second base comprises the control signal being configured to indicate one or more of: the first candidate position, the first number of interactions, the second candidate position, the second number of interactions, and a recommendation for the selected candidate position to be used as a fixed position of the second base.

One or more steps in the plurality of steps of the surgical procedure may involve use of a third robotic arm attached to a third base. The device may determine, based on the plurality of steps of the surgical procedure, the fixed position of the first base, and the selected candidate position, that a third candidate position of the third base is associated with a third number of interactions in which the third robotic arm and at least one of the first robotic arm or the second robotic arm will co-occupy space during the surgical procedure. The device may determine, based on the plurality of steps of the surgical procedure the fixed position of the first base, and the selected candidate position, that a fourth candidate position of the third base is associated with a fourth number of interactions in which the third robotic arm and at least one of the first robotic arm or the second robotic arm will co-occupy space during the surgical procedure. The device may select a candidate position for the third base, from the third candidate position and the fourth candidate position, based on the third number of interactions and the fourth number of interactions. The device may generate a control signal configured to indicate the selected candidate position for the third base. The device may predict an effect, caused by the selected candidate position for the second base, on placement of the third base. The control signal may indicate the effect.

Historic data (e.g., an understanding of issues and/or complicated zones) may be used to identify steps and/or situations most affected by the placement selection.

In an example low intervention mode, the smart system may determine to shift boundary condition(s) based on user experience (e.g., increase space/access if system is in training mode).

The system may display example device placements (e.g., layouts) to the user. The layouts may allow the user to select a (e.g., preferred) set up.

During pre-operative planning, the care team (e.g., nurses, surgeon, other doctors, etc.) may perform one or more of the following. The software may have pre-programmed information from clinical knowledge (e.g., for a given surgical procedure, tumor location, or patient characteristics). The system may determine an (e.g., ideal) arm placement based on the information. The software may reside on a robotic system.

A user interface (UI) may display user input options (e.g., patient weight/height/pre-existing conditions/other characteristics, surgeon handedness, tumor scans or location, procedure type, patient orientation during surgery, areas to be accessed during surgery, bed orientation during surgery, number of people in the OR, room size/setup, etc.). The software may be integrated with hospital electronic health record system(s). This integration may allow the system to pull relevant patient data from a database (e.g., to automatically pre-populate procedure, patient, and/or other information). This may reduce the time and effort used for manual data entry. The software may use the information to output options for the configuration of the devices/arms. The system may display the pros and cons of an (e.g., each) option. The system may display expected conflicts/interactions (e.g., at each step). The software may provide a recommendation (e.g., recommended device placement(s)). The surgeon may select device placement(s). The software may have a pre-defined “training mode.” The training mode may be used to recommend device/arm placement if the operating surgeon is a new resident. For example, the training mode may recommend placement(s) to give the surgeon more space or access). The recommended placement(s) may be determined based on HCP input (e.g., clinical advice).

An algorithm may be used to determine recommendations for robotic arm placement in pre-operative planning.

The system may present the surgeon with information or feedback with respect to any non-recommended device placement(s). The feedback may be provided in a graphical format and/or a numerical format. The feedback may be represented in terms of a surgical step, absolute metric, or relative metric.

For example, while positioning the robotic arms before surgery, the system may determine that the current location and orientation of the arms is different from the recommended location/orientation of the arms. The system may display information (e.g., on a surgical screen or tablet), for example, an indication of surgical step(s) that will be affected by the current location/orientation (e.g., if a step will be unable to be completed) and/or a percentage loss in access to the surgical site based on the current location/orientation (e.g., 78% of access compared to 100% access if the recommended placement was used).

The system may display an indication of data sources that are available and/or objects that are in the OR.

The surgical suite floor may have a numbered grid for mapping of device positions. The grid may have six-inch by six-inch squares. The grid may be made with increasing density (e.g., for more accurate placement). The grid may be divided into quadrants (e.g., for basic placement). Software may be used to display a drop down for the HCP to select the surgical procedure being performed and/or available devices. The software may suggest a type and/or number of devices, and/or device placement on the grid. When the devices are placed, an overhead camera may check for placement accuracy (and indicate when the devices are properly placed). During the procedure, if a device moves outside a suggested boundary, the system may provide a warning.

The OR space may be sub-divided into interactive segments. The segments may allow for prioritization or nested movement (e.g., if the OR is reorganized during use).

The system may use a surgical suite grid and algorithm to determine device placement.

A display may show the locations of the connected and non-connected devices in an OR (e.g., to confirm the correct set-up is achieved). The display may have a projector displaying “no access zones” for patient- and/or procedure-specific items. The display may project anticipated staff/personnel locations. If the OR set-up of the equipment is complete, the display may confirm that the connected and non-connected devices are in the correct positions. The display may use a color-coding system to signal if devices are correctly positioned. The display may be used to highlight “no access zones” for a procedure or patient. The display may highlight areas in which personnel are able to be during the procedure. A positioning aid in the OR room may be used to confirm proper device placement.

Robotic arms (and/or other surgical devices) may be positioned (e.g., set up) so as to reduce (e.g., minimize) interactions between with other devices during the procedure.

User inputs may be used to determine (e.g., optimize) robotic arm placement. Factors such as available devices, personnel in the room, patient history, surgeon preference, type of procedure, and/or the like may be inputs to provide the user with a starting point (e.g., to optimize the set-up for additional refinement).

One or more inputs may be used to determine the placement of robotic arms. Device placement and access zones may be determined based on user inputs. The inputs may define “no access zones” (e.g., spaces in which the robotic arms cannot enter).

The inputs may include other devices in the OR (e.g., tables, capital equipment, mayo stands, lighting, etc.). These devices have a defined volume that may be used to calculate available space for the robotic arms. The inputs may include a list of personnel (e.g., nurses, scrub tech, anesthesiologist, surgeons, fellows, etc.) and their anticipated locations in the OR. The inputs may include patient history. For example, high risk factors (e.g., past cardiac events) may cause the system to ensure that the HCP has sufficient access to the chest (e.g., to perform emergency open surgery or to ventilate the patient, etc.). The inputs may include the type of procedure (e.g., and basic patient factors such as size, weight, etc.). For example, in an obese patient, trocars may be higher away from the table, which affects placement of the robotic arms. The inputs may include surgeon preferences (e.g., from previous procedures).

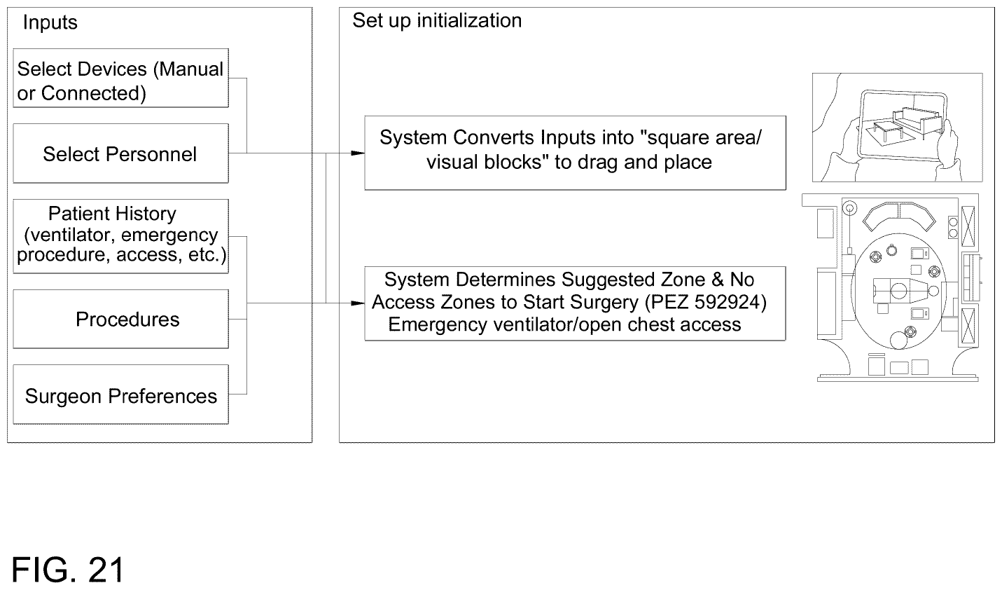

During set-up initialization, a recommended robotic arm placement may be provided to the user. FIG. 21 illustrates an example flow chart for device placement during operating room set-up initialization. As illustrated, the system may receive one or more inputs (e.g., device(s), personnel, patient information, procedure type/steps, surgeon preferences, and/or the like). The system may convert the inputs into a diagram of virtual objects in the OR. The system may indicate recommended device placements and “no access zones.” The system may allow a user to alter the initial diagram. For example, the user may drag and place the virtual objects around the diagrammed room. The system may indicate interactions that would result from the user's changes.

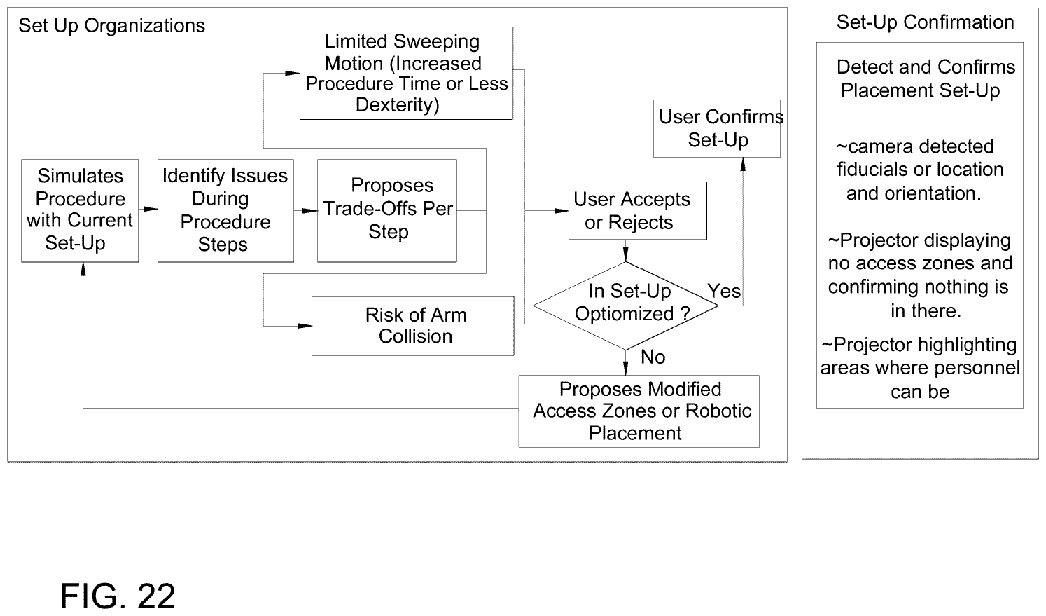

Additional optimization steps may be performed (e.g., after initialization). FIG. 22 illustrates an example flow chart for device placement optimization. As illustrated, the system may simulate the steps of the procedure using the device placements determined during initialization (e.g., including any user adjustments). The system may identify one or more issues/interactions caused by the device placements. The system may indicate trade-offs (e.g., pros and cons) at each step of the procedure. For example, the system may indicate that the device placement will avoid possible interactions at steps 4 and 12, but may create interactions at steps 6 and 19. The system may indicate trade-offs throughout the procedure. For example, the system may indicate that the device placement will reduce the number of interactions by three, but the procedure will take approximately one hour longer than another room setup (e.g., due to the OR staff having to pause and manually rearrange/untangle devices).

The system may indicate options to the user. For example, the system may indicate that limiting the range of motion (e.g., sweeping) of a device would reduce the number of device interactions, but would increase the procedure time or limit the surgeon's ability to access part of the surgical site. The system may indicate the risk of device (e.g., arm) collisions if no preventative action is taken. The system may receive user feedback in response to the options. For example, the user may indicate that they will accept the increased risk of device collisions. In another example, the user may indicate that none of the options are acceptable. In this case, the system may use the feedback to recommend a modified layout of the OR room (e.g., modified device placement) and/or modified access zones for one or more device(s).

If the user selects one of the presented options (e.g., accepts the risk or makes an adjustment), the system may determine that the device placement is finalized. Once the placement is finalized, the system may monitor the device placement and confirm when the devices are correctly positioned. The system may monitor the device placement/orientation by using cameras to track fiducial markers on the devices. The system may display no-access zones in the OR and confirm that no devices are in those zones. The system may also display areas in which the OR staff are able to move (e.g., without coming into contact with surgical device(s)).

Robots may be equipped with means for determining the location of one arm relative to another in three-dimensional space. For example, another may monitor arms and provide relative measurements of one with respect to the other. For example, the system may determine the relative measurements through imaging of the OR. One or more cameras within the OR may be used to generate this information. The cameras may be separate from, or on the robot itself. The system may determine the relative measurements based on magnetic sensors, ultrasonic pinging, etc. This additional data feed may be used to determine the location of the devices relative to each other.

The hub and/or camera system may have to have stored parameter(s) related to the device(s) being tracked and/or capabilities of those device(s). Triangulation of the device position may be used to suggest device motions. For example, the system may use kinematics of the device(s) (e.g., robot arm(s)) and the patient to determine viable movement options. The kinematics may be derived (e.g., on the fly) using visualization. The range of motion (e.g., reach) and balance of the robot may be received from the robot or pre-determined.

Indexing elements (e.g., fiducials) on the arms may enable a separate system to monitor the arms, (e.g., each of the segments of the arms). In some examples, electronic sensors may be used (e.g., rather than fiducials). The electronic sensors may emit a signal that is received by other sensors or a base station. For example, fiducials and/or electronic sensors may be placed at points 55214-c (e.g., and/or other joints) in FIG. 18.

An example smart system interaction architecture is provided herein.

The system may detect (and alert the surgical staff of) missing equipment (e.g., before a procedure begins). Cameras in the OR may publish information to a software system (e.g., that is pre-trained with computer vision object detection on an OR dataset (e.g., with all the common tools, equipment, people, etc.). The software may compare the items in the OR to a checklist (pre-)selected by the surgical staff (e.g., before the procedure). The checklist may be determined based on (pre-)programmed information about tools used for a selected procedure type. The information may be displayed to the staff on a computer/monitor screen or indicated in another way (e.g., audio cues, beeps, etc.). The staff may determine (e.g., and indicate via the UI) whether a tool that was indicated as missing is missing or not. That information may be used to improve the algorithm (e.g., in real time).

OR staff may be identified (e.g., by role). For example, the system may identify bedside OR staff and other/additional staff in the room. OR staff may be identified based on an OR dress code (e.g., latex gloves vs. surgical gloves). Bedside staff may wear a different color for a camera to use in identification. Wearables (e.g., wristbands) for staff members may allow the system to track individuals. OR staff may be identified based on the procedure type (e.g., in procedures where the patient is held in a twilight state, anesthetist/anesthesiologist may be more active near the patient and use more space near a ventilation system). OR staff may be identified based on location (e.g., staff in a sterile field may be differentiated from non-sterile staff). OR staff may be identified using a tracking method (e.g., camera-based tracking). OR staff may be identified using visual/image processing (e.g., IR camera, specified markers/wearables, ME field, etc.).

The OR staff members that are tracked may vary over time. For example, not all staff members may be tracked. For example, to prevent robot arm collisions, the system may (e.g., only) track the people in the space the robot is/may be using. A touch point may be included on a robot arm (or elsewhere). The touch point may be used to inform the system that the individual who interacted with the touch point is to be tracked for collision avoidance.

The system may use the procedure plan/type to predict upcoming movements/interactions.

In an example, a ventilator may be stationary (e.g., have no base movement/tubing adjustability without hardware modification). The system may have access to data from the ventilator, but be unable to modify the position/orientation of the ventilator. The system may highlight a larger collision/no fly zone for such stationary equipment (e.g., compared to a moveable device such as a Hugo robot, where the arms can be manipulated). An HCP may prefer to place equipment with limited mobility near areas used for emergency access (e.g., because the equipment cannot move to block the area).

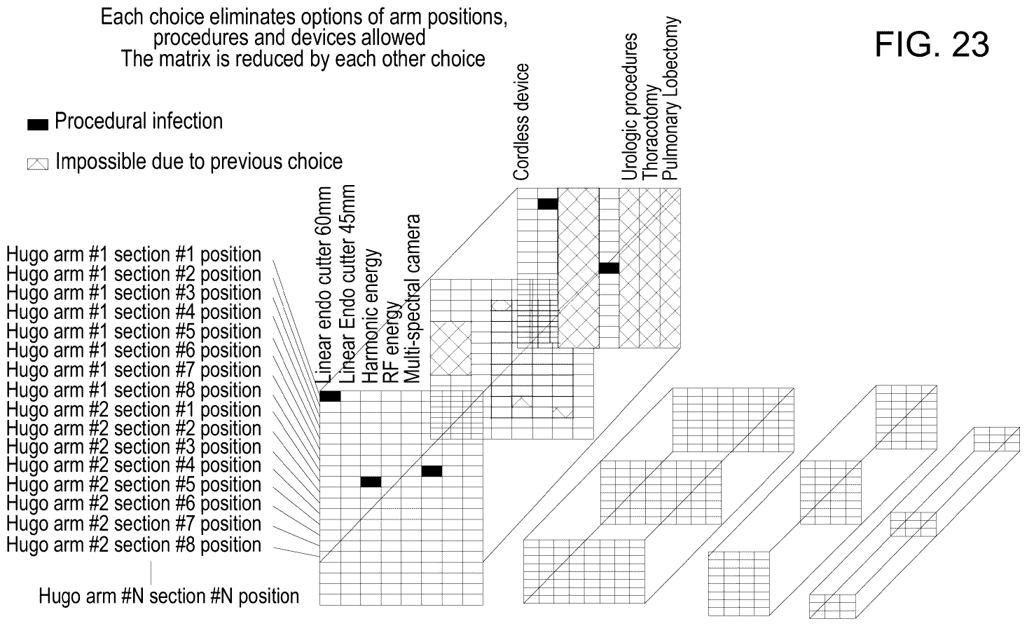

As illustrated in FIG. 23, the system may create a 3D matrix to list the different variables associated with the system, arms, devices, etc. The variable list may include the inputs described herein and/or more specific information (e.g., the parts/joints of a robot arm). For example, a robot arm may have five variables associated with four arm segments and a 360-degree rotatable joint. The variable list may include a variable for the pedestal location of the floor of the OR and/or other joints (e.g., two additional joints, for example, for reach and height). The joint capabilities may determine the pitch, roll, and yaw of the robotic arm system. The variables may be associated with an arm placement (e.g., a single arm placement) within the OR. The number of variables may be multiplied by the number of arm pedestals being used. Additional robotic system consoles, video displays, and/or other capital equipment and devices may become part of the matrix. The number of variables and outcomes/interactions would be improbable (if not impossible) for a human to process. The 3D matrix illustrated in FIG. 23 may whittle down the possible variations.

The surgeon and staff may determine priorities associated with functions and/or parts of surgical equipment (e.g., criticality for a successful outcome). A highest priority device may be placed (e.g., first) and fixed within the OR. The placement may eliminate other equipment from occupying that space. As devices are placed, portions within the matrix may be canceled (e.g., due to conflicts with the already placed devices). For example, if a first robotic arm is placed, the area where the pedestal/base is located and the surrounding arc that the arm will sweep through may not be allowed within the matrix for other equipment. As the OR team begins determining the placement for a second robotic arm, some areas may be excluded within the matrix (e.g., that can be referenced to ensure no unintended interactions take place, for example, the arms running into each other). This process may be repeated as each device is placed and checked off (e.g., until the OR suite is set up).

The OR set-up optimization (e.g., using the matrix) may be sequential (e.g., based on other equipment that has been locked down previously) rather than singular holistic (e.g., presenting all options and proposed locations for all equipment at once). The OR system may select to place an (e.g., only one) item at a time (e.g., rather than try to calculate all possible orientations). For example, the system may (e.g., first) display options for a first robot arm controlling a laparoscope. Once the first robot arm is positioned and locked, the system may prompt the user to place a second robot arm controlling an endocutter (e.g., considering the placement of the first robot arm, and any objects placed at an earlier step or objects that cannot be moved as boundary conditions).

The system may predict placements of devices. The system may display recommended placements to the OR team. The devices may be categorized by movement capability (e.g., a wristed endocutter and a single plane endocutter may have different movement choices). The instruments being used may impact the device placement. The positions of the people in the OR may affect device placement. Steps in which the robot is stopped and steps in which the robot is in use may affect device placement.

Boundary conditions (e.g., including surgical access) may be simulated to improve the likelihood of success of the procedure. An example boundary condition may include the limited articulation of an endocutter. In this case, another device may be used in the endocutter space while the endocutter is not in use.

The system may simulate steps that occur later in the procedure. For example, the system may simulate tumor removal from different port locations.

The system may suggest one or more instruments to use in the procedure. For example, the system may recommend using an ABP device (e.g., instead of an ultrasonic device) due to better robotic access.

The system may use device availability to mitigate future conflicts. For example, the system may suggest using a 45 mm endocutter instead of a 60 mm endocutter. In another example, the system may suggest using a 45 mm energy shaft length instead of a 36 mm energy shaft length. The system may recommend using a harmonic (e.g., non-articulating) device or an articulating device (e.g., an energy device).

The system may suggest one or more device placements based on a surgeon's use of certain procedure technique(s). For example, if a monopolar tip is used, the system may recommend a distance/proximity between the monopolar tip and a smoke evacuator.

Some devices may affect placement of other devices. For example, robotic arm placement may depend on the location(s) of energy generator(s), laparoscopic monitors, OR tower, lighting, suction/irrigation lines, the position of the patient bed, etc.

Some devices may be initially present and removed later in the procedure. Some devices may be introduced during the surgery and left for the remainder of procedure. A CT machine is an example of a temporarily introduced piece of equipment (e.g., introduced and then removed during the surgery). The decision to introduce or remove devices may be planned pre-procedure and/or determined during the procedure. Capital equipment may refer to devices that are present throughout the procedure.

Sterile and non-sterile equipment may be allowed in different areas.

The system may consider limitations for the humans in the room (e.g., line of sight, for example, if the surgeon wants to be able to see a video screen at any time). The system may determine device-free areas (e.g., based on surgeon selection).

Although some aspects are described with respect to one or more robotic arms, a person of ordinary skill in the art will appreciate that these aspects may be used for any powered device (e.g., an articulable endocutter, etc.).

Robotic arm movement may be improved (e.g., optimized) to control interactions between arms outside the body. A system may automate decision making for optimizing arm movements.

On a robotic arm, there may be a large number of options (e.g., infinite options) that can result in locating the end effector in the desired location. Robotic arm movements may be governed by the simplest and/or most efficient way to move the end effector to the desired location inside the patient. This may cause the robotic arms to collide with one another during the surgical procedure (e.g., which may limit the surgeon's access) or to become so entangled that the procedure is stopped to allow the OR team to untangle and reposition the robot arms prior to resuming surgery. In a digitally connected OR, additional data feeds (e.g., external cameras in the OR), beyond just the robotic arm placement, may be used to inform the robot of its external arm locations and assist its ability to prevent collision and/or entanglement via informed decisions of what kinematic movements to make outside the patient (e.g., which joints to move, how much to move, etc.).

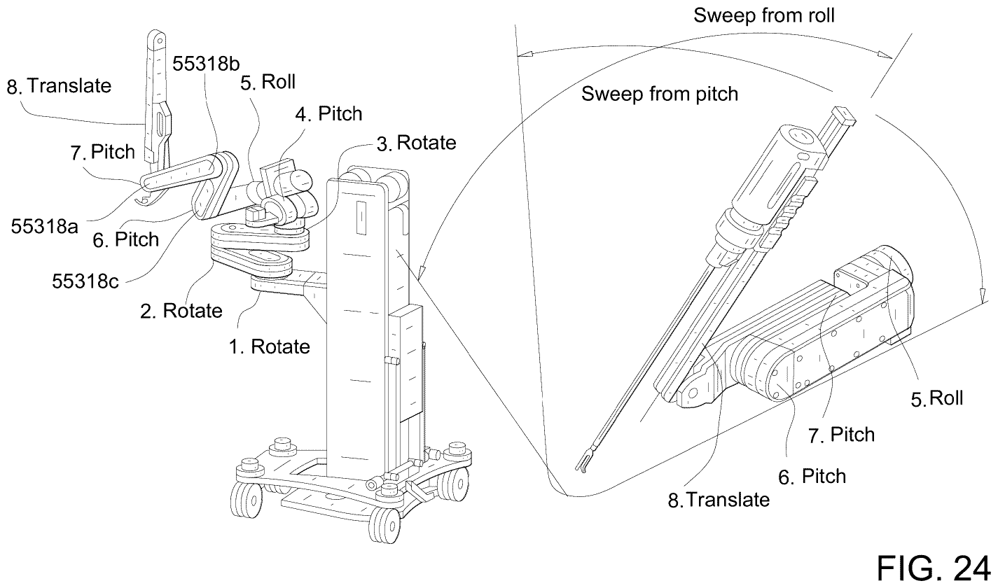

FIG. 24 illustrates example joint movements of a robotic arm attached to a base. As shown, the arm may be able to move in the x, y, and z directions. The joints may control the pitch, roll, and rotation of an end effector attached to the arm. The arm may have a mechanism (e.g., slide) for translation of the end effector. The joints may cause the end effector to make small movements inside the patient (e.g., in the surgical cavity). The arm movements correlated to the small internal movements may be large (e.g., creating a considerable sweep of the arm).

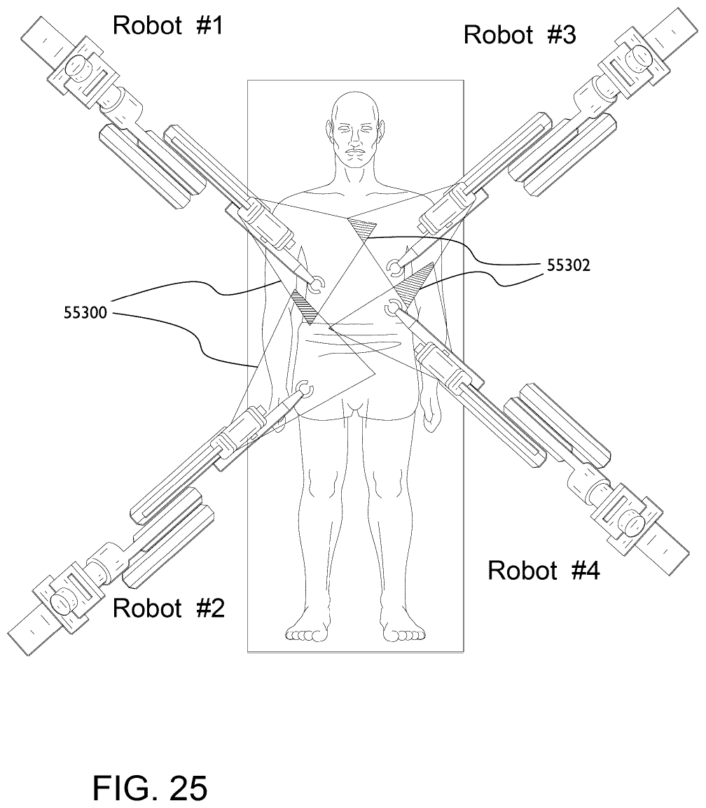

FIG. 25 illustrates an example operating room arrangement of multiple robotic arms. As shown, multiple robotic arms may be present in a relatively small space in an OR. The arms may have ranges of motion that overlap (e.g., as shown at 55300 and 55302). In this case, the arms may collide unless adjustments are made to reduce the potential interactions. For example, one or more of the arms may not be allowed to occupy the overlapping space (e.g., at a given time). As described herein, the system may determine potential robot arm position placement and kinematic forecasting of resulting positions, movements, and/or interactions. The system may determine (and display) options for the HCP to choose from (e.g., and request that the HCP provide input). The system may output a simple, understandable display of context and choices.

Devices and methods for visualizing effects of device placement in an operating room. An example device may include a processor configured to perform one or more actions. The device may receive an indication of a plurality of steps of a surgical procedure associated with a patient. One or more steps in the plurality of steps of the surgical procedure may involve use of a first robotic arm having a first end effector attached and a second robotic arm. The device may identify a first candidate motion and a second candidate motion of the first robotic arm configured to place the first end effector in a target end effector position internal to the patient. The device may determine, for the first candidate motion, a first number of associated interactions in which the first robotic arm and the second robot arm co-occupy space external to the patient during the surgical procedure. The device may determine, for the second candidate motion, a second number of associated interactions in which the first robotic arm and the second robot arm co-occupy space external to the patient during the surgical procedure. The device may select a candidate motion of the first robotic arm, from the first candidate motion and the second candidate motion, based on the first number of interactions and the second number of interactions. The device may generate a control signal based on the selected candidate motion of the first robotic arm.

The system may display choices of powered device joint motion (e.g., based on preferences or concern of where and/or when the devices may interact. The system may monitor the positions and orientation of the portions of the devices outside of the body. The system may monitor the current position of the devices and predicting future positions of the devices based on the surgical tasks or procedure plan. The prediction may be aggregated into options for presentation to the user. The options may indicate interaction location, timing, or magnitude. The system may choose instrument joint motion controls based on the feedback from the user (e.g., what spaces have preferred operational space and/or spaces to keep as clear as possible). The powered devices may be robotic arm assemblies and/or tools. For example, on a condition that the first number of interactions is less than the second number of interactions, the device may select the first candidate motion. On a condition that the first number of interactions is greater than the second number of interactions, the device may select the second candidate motion.

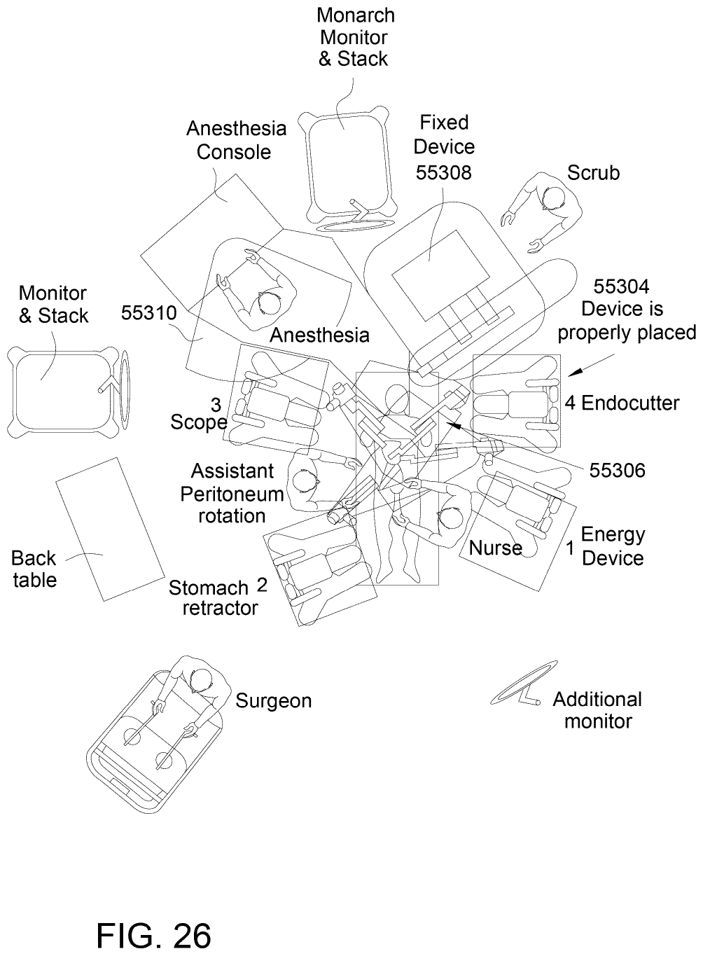

FIG. 26 illustrates an example display to a user of areas of potential interactions between surgical devices. As shown at 55304, the system may indicate that a device is properly placed. The display further shows areas in which multiple robot arms may occupy the same space. For example, as shown at 55306, the arm with endocutter 4 may share an overlapping space with the arm attached to energy device 1. The system may determine sequential movements of the two arms to prevent a collision in the overlapping space. For example, if the endocutter arm is moving through the space to reposition, the energy device arm may remain still. The energy device arm may be moved once the endocutter arm has finished moving and exited the overlapping space.

FIG. 26 further illustrates an example of a fixed (e.g., non-moving) device 55308. The fixed device 55308 may be a ventilator (e.g., a Monarch smart ventilator). The ventilator may be placed (e.g., and fixed) at the patient's head (e.g., because the ventilator must be attached at the patient's mouth and/or nose). FIG. 26 further illustrates an example area 55310 in which an HCP (e.g., the anesthesiologist) will occupy during one or more parts of the surgery. For example, throughout a surgery, the anesthesiologist may need to have access to the patient's head to properly check and maintain sedation.

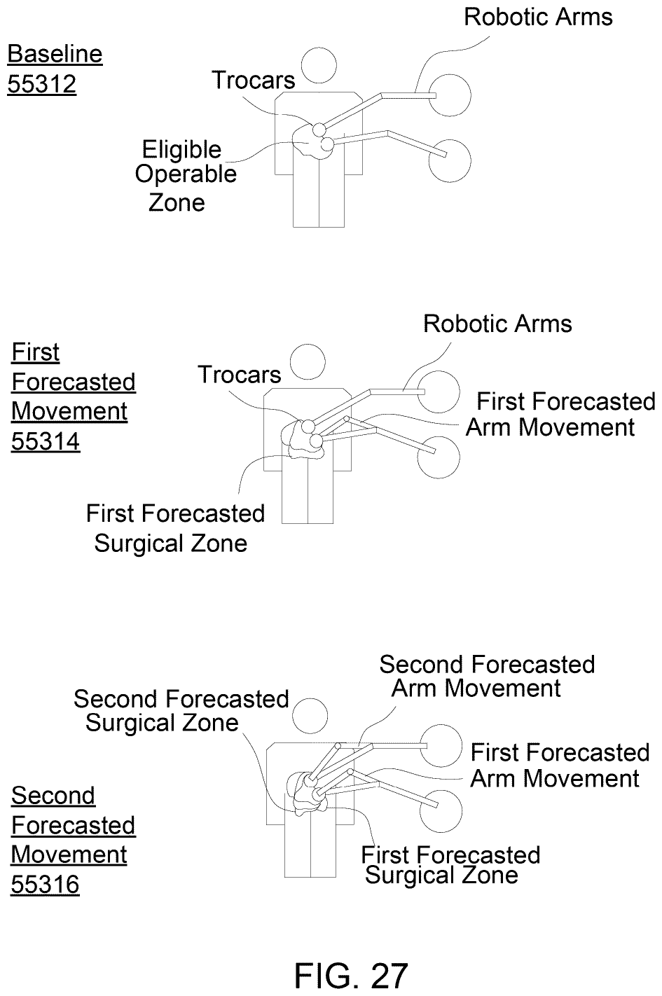

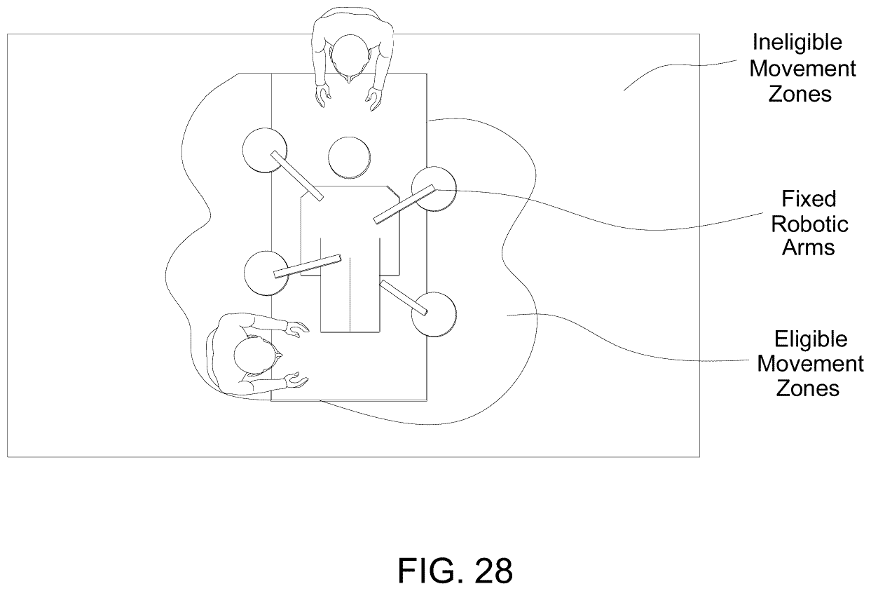

FIG. 27 illustrates an example display to a user of potential interactions between robotic arms during a surgical procedure. In a baseline position 55312 (shown in the top figure), the two robotic arms may be in an initial location. This initial location may be at a time just after the instruments attached to the ends of the arms were inserted into the patient via trocars. Based on this initial position, the system may determine an eligible operable zone. The eligible operable zone may indicate the areas in the patient's body that are accessible to the end effectors without moving arm joints external to the patient's body.

The target end effector position of the first end effector may be a first position. The device may determine an updated current arm position of the first robotic arm, external to the patient, based on the first robotic arm moving according to the selected candidate motion. The device may determine a second target end effector position of the second end effector, during a third step in the plurality of surgical procedure steps. The second target end effector position may be internal to the patient.

The device may determine, based on the updated current arm position of the first robotic arm, the current arm position of the second robotic arm, and the plurality of steps of the surgical procedure, a third candidate motion of the second robotic arm that will place the second end effector in the second target end effector position. The third candidate motion of the second robotic arm may be associated with a third number of interactions in which the first robotic arm and the second robot arm will co-occupy space during the surgical procedure.

The device may determine, based on the updated current arm position of the first robotic arm, the current arm position of the second robotic arm, and the plurality of steps of the surgical procedure, a fourth candidate motion of the second robotic arm that will place the second end effector in the second target end effector position. The fourth candidate motion of the second robotic arm may be associated with a fourth number of interactions in which the first robotic arm and the second robot arm will co-occupy space during the surgical procedure. The device may select a candidate motion of the second robotic arm, from the third candidate motion and the fourth candidate motion, based on the third number of interactions and the fourth number of interactions. The device may generate a control signal based on the selected candidate motion of the second robotic arm.

The surgeon may determine to move one of the arms using joints external to the patient (e.g., to access an area not in the eligible operable zone). The first forecasted move 55314 (shown in the middle figure) illustrates an example movement of one of the arms. As shown, the system may update the displayed image to show an updated operable zone (e.g., a first forecasted surgical zone). The system may keep a depiction of the initial positioning of the arm. This may allow the surgeon to better visualize what the movement will look like. The surgeon may similarly move the other arm using joints external to the patient. The second forecasted move 55316 (shown in the bottom figure) illustrates an example movement of the arm. As shown, the system may update the displayed image to show an updated operable zone (e.g., a second forecasted surgical zone). The system may keep a depiction of the initial positioning of the arm. These depictions may allow the surgeon to keep track of the positioning of the arms relative to each other, which may help the surgeon avoid collisions.

The system may detect sub-optimal equipment set up. The system may be able to project guidance markings on the OR floor and/or equipment to assist OR staff set up the room.

The system may include one or more OR ceiling or boom-mounted overhead cameras to monitor the presence and location of people and equipment in the OR. Cameras may connect with a computer system running computer vision models (e.g., in real time) capable of detecting the position of various OR equipment and people.

The multiple cameras may be mounted at configured distances (e.g., such that the system may utilize information about their relative position to each other and the floor). For example, the cameras may be used to register the measured positions of objects in the camera image within a virtual multi-dimensional reconstruction of the OR.

Robots may be equipped with means for determining the location of one arm relative to another in three-dimensional space. For example, another may monitor arms and provide relative measurements of one with respect to the other. For example, the system may determine the relative measurements through imaging of the OR. One or more cameras within the OR may be used to generate this information. The cameras may be separate from, or on the robot itself. The system may determine the relative measurements based on magnetic sensors, ultrasonic pinging, etc. This additional data feed may be used to determine the location of the devices relative to each other.

The system may utilize knowledge of surgical context for a given procedure (e.g., surgeon, procedure, surgical tool preference card information, etc.) to determine which pieces of equipment should be in place for the surgery (the “Necessary Components”). For example, the system may determine which robotic system components (e.g., robotic arm bases, a flexible robotic system, etc.) to have in the room. Surgical robot systems from different manufacturers may be present in the room.

Components may be the source of collisions and/or setup issues. Components may create access issues for OR staff if not properly positioned.

Object detection algorithms running on the system may be trained to detect the position/location of components and the OR table. The OR table position may be used as a datum or reference location for placement of other large components to define locations for each piece of equipment.

With knowledge of the OR table position and other objects in the multi-dimensional reconstruction, the system may be able to compare the measured positions of components against a pre-configured database of acceptable setup positions (e.g., “Go” and “No Go” positions). Acceptable setup positions may include the position and orientation of components (e.g., robotic arm base components on the floor). Acceptable setup positions may be defined as a polygon shape on the OR floor (e.g., within which the position is acceptable). Orientation may include a range of angles.

If a piece of OR equipment is determined to be in a “No Go” position, the system may alert the user. For example, the user may be alerted based on the system projecting images on the floor of the OR giving visual display of “Go” zones. Projection may be accomplished via one or more ceiling- or boom-mounted projectors that are registered to the same coordinate system as the cameras. A monitor may show the virtual reconstruction of the OR and indicate equipment that is out of position. With the projector system, if a piece of equipment were out of position, the projector may display colors or patterns on the floor or equipment.

Camera localization and detection of robot arms may be used for (e.g., optimal) placement prediction. Hybrid on-screen and off-screen capital placement (e.g., OR set-up) may be performed using OR spotlights/projectors.

Simplified colored light spotlights (e.g., projectors) in multiple colors may be part of the camera hub, or ceiling mounted. The spotlights may illuminate a specified area with a given color (e.g., red). The on-screen display may indicate to the user to place a specified piece of equipment into the illuminated area (e.g., “place endoscope robotic arm in red area”). This may be performed sequentially (e.g., using the same color), or in parallel (e.g., using multiple spotlights of different colors).

If a spotlight is in line with camera, the camera may provide a feedback loop to the spotlight to adjust the color or intensity to visually provide feedback and indicate if an item is placed correctly (e.g., switch from red to green) or to help the OR team to optimize placement (e.g., increase light intensity as position is optimized).

Color selection, commands, and feedback may depend on whether the OR team arranges the OR in series or in parallel.

The system may select robot arm movements (e.g., optimal movements to prevent robot arm entanglement). If a robotic arm interaction is anticipated, the system may perform one or more actions.

This algorithm would be on a processor located somewhere in the OR. The algorithm may subscribe to information from the cameras and/or pre-planning information. From knowledge about the placements/positions of items in the room and appropriate mathematical calculations, the software may predict collisions. The system may recommend changes to arm positions and alert the surgeon (e.g., in real-time).

The algorithm may recommend robotic arm adjustments during surgery. Setting adjustment to one or more robots may be performed to enable continued function on a ‘primary’ arm. Settings may include restricted motion/speed, joint adjustment, yielding to prioritized tasks.

The system may detect an expected collision of robotic arm components outside of the body. The system may include a follower robotic arm and a commanded/active robotic arm. The commanded robotic arm may be (e.g., actively) controlled by the surgeon and may collide with the follower arm outside of the body (e.g., as some end effector positions may cause robotic arm configurations with a large degree of movement, potentially infringing on collision zones or the physical envelope of another arm).

As the commanded arm moves, a system may monitor its arm position. If a collision is anticipated based on the arm position outside of the body between the commanded arm and a follower arm, the follower arm may maintain its end effector position using the degrees of freedom in its wrist (e.g., end effector roll, articulation, etc.). The follower arm may reconfigure its joints out of the body to move the arm linkages out of the way of the actively commanded arm. The surgeon may be notified that the follower arm is in motion due to a collision avoidance maneuver. The movement of the follower arm may have limits. If the collision cannot be avoided by dynamic reconfiguration, the surgeon may be notified that the maneuver is out of range and will not be attempted.

Dynamic reconfiguration maneuvers may be used to avoid robotic arm collisions.

Reconfiguration may involve speed scaling. As a component of a robotic system nears or enters the collision space of another system or obstacle, the controlled speed of the robotic system may be decreased (e.g., dynamically) based on distance from the hazard or potentially colliding object. The slowing of the robotic system may increase as proximity to the colliding object increases (e.g., up to the extent that the robot may cease motion entirely in the direction towards the obstacle). This speed scaling function may be applied to motion in the direction of the obstacle (e.g., exclusively). The speed scaling may prevent collision while maintaining normal operation in safe zones/directions or allow for retreat from the obstacle.

Reconfiguration may involve inertially-weighted motion scaling. The collision space of the obstacle may increase in proportion to the calculated inertia of the robotic arm (e.g., based on current moving speed and payload/weight of arm and end effector tooling). This may create additional reaction and slowing time when the robotic system is less capable of ceasing motion due to inertial loads.

Reconfiguration may involve arrest control. In some cases (e.g., where a collision is imminent or will create a hazard), motion of the robot may be locked out (e.g., entirely) of a specific collision zone (e.g., defined as a radius from an object) to prevent the hazardous situation from occurring. If inertial loads are too great, dynamic braking (e.g., if available) may be (e.g., automatically) implemented to prevent the collision.

The system may detect that a first robot (e.g., Robot A) is expected to collide with a second robot (e.g., Robot B). Robot A may not be fed information or controlled by the global controller that runs the OR vision systems and Robot B. In this case, Robot B may yield to Robot A. The system may stop motion and/or warn the surgeon to move or reconfigure the end effector or arm body. If an arm-based out-of-body collision is expected and Robot A is the moving robot, Robot B may reconfigure (e.g., without moving the end effector) to get as far out of the way as possible.

If Robot B is moving and Robot A is not controlled by the OR system, Robot B may establish zones within which it cannot operate or is slowed due to the presence of Robot A. The slowing or keep out zones may prevent Robot B from contacting Robot A.

The control system may prevent a command to Robot A that would result in a collision as established by the connected OR controller.

Third party robots (e.g., robots of different origin) may interact in an OR. If a collision is expected, the system may notify the surgeon of the upcoming collision. Communication of the notification may be visual and/or non-visual. Non-visual communication may include sensory feedback (e.g., haptic and/or auditory) to create awareness of an approaching collision and/or system adjustment.

A system within the robotic human interface device may have a haptic motor that provides haptic feedback to warn the operator of the robotic system that the end effector or arm structure of a robotic arm is entering within a predefined radius of a person, obstacle, or other robotic arm system in the operating room. This radius may be considered the ‘hazard zone.’ As the distance between the potentially interfering object and the controlled robotic arm within the hazard zone decreases, the haptic and/or auditory feedback may increase proportionally to inform of the increasing risk of proximity/collision. A system within the robotic human interface device may include a system that physically resists manipulation of the human interface device by the operator if the end effector or arm of a robot enters a hazard zone. The system may resist control that would move the robot into a position of proximity or collision risk of another object. The resistance may increase as the distance between the potentially colliding object and the robotic arm decreases. This resistance may increase to the point where a user cannot move the controls into a position that would result in a collision of the controlled robotic arm and another object.

Collision prevention in a robotic system may be based on behavioral and user-feedback.

Short term procedure limitations during steps with less risk may be accepted to prioritize long term collision reduction during subsequent steps with higher surgical risk. If a system (e.g., always) selects the position or movement with the lowest risk of entanglement, the system may create many more risks of entanglements throughout the procedure.

The system may select an option in the beginning of a procedure that creates the risk of entanglement (e.g., but reduces the likelihood of them occurring further on the procedure). In a system that has more control of itself than simple extension/retraction, the system may use that control to help reduce overall entanglement throughout the procedure.

As the mechanical capabilities of the system grows, the system's algorithmic processing and complexity may grow (e.g., to match the scenarios it may accidentally create). Procedure steps (e.g., N steps) may be modeled in succession (e.g., with possible risk entanglements and comparison of overall possible future risk scores).

The device may determine, during a first step in the plurality of surgical procedure steps, a current arm position of the first robotic arm and a current arm position of the second robotic arm that are external to a patient. The device may determine, during a second step in the plurality of surgical procedure steps, the target end effector position of the first end effector. The first candidate motion and a second candidate motion of the first robotic arm may be identified based on the current arm positions of the first and second robotic arms and the plurality of steps of the surgical procedure.

One or more steps in the plurality of steps of the surgical procedure may involve use of a third robotic arm. The device may predict an effect, caused by the selected candidate motion of the first robotic arm, on a future motion of a third robotic arm. The control signal may indicate the effect.

Localized inefficiencies of movement may occur to create overall efficiencies in movement.

The system may recommend using handheld tool(s) to avoid collision. The range of motion/working space used may be reduced with human control. If a tool (e.g., endocutter, clip applier, etc.) is only used once, the system may recommend user intervention to optimize the number of tool exchanges and differing needs of working space for different tool types.

The system may detect a collision using sensors, force transducers, pressure sensors, electrical voltage from drive motors, sounds, and/or accelerometers. These devices may be mounted to the end of arm tool or smart module (e.g., clamped onto tool or built into the tool). These devices may be mounted to each end of a robot arm.

The data may be used in conjunction with a microprocessor to detect a stall, which may indicate a collision. Accelerometers (e.g., any two accelerometers) or other sensor data may be in synch with one another. For example, two acceleration/de-acceleration events (e.g., within microseconds) may indicate a stall or collision between the two arms.

The information may be used by the microprocessor to stop movement of the tools that collided. The information may be used to move the collided tools in a reverse motion to eliminate any collision forces. The information may be used to pause the effected tools. The information may be used to put affected tools in a limp mode where the forces are reduced so that no damage can be done to patient or equipment. The information may be used to send feedback to the surgeon or operating room staff. The information may be used to output alarms (e.g., audible, tactile, haptics, lights, etc.).

Stall detection may trigger corrective reaction. Microphones (e.g., attached to the end of arm tool, for example, clamped onto tool or built in, or to each end of a robot arm) may pick up the sound of collision between tools. The sound may be fed into a microprocessor for analysis. A library of collision sounds may be prerecorded such that the microprocessor can detect which tools collided. The recorded sounds from the microphones may be analyzed and compared with the prerecorded database of collision sounds. The information may be used by the microprocessor to stop movement of the tools that collided, move the collided tools in a reverse motion to eliminate any collision forces, pause the effected tools, put the affected tools in a limp mode where the forces are reduced so that no damage can be done to patient or equipment, send feedback to the surgeon or operating room staff, output alarms (e.g., audible, tactile, haptics, lights) and/or the like.

The likelihood of robotic-human collisions may be reduced. For example, non-contact-based sensing technologies may be used to identify the location of humans in close proximity to the robot. Sensing may be achieved using one the following: a light detection and ranging (LiDAR) scanner (e.g., that emits a laser to measure the distances of surrounding objects), ultrasonic sensors (e.g., that transmit an ultrasound wave that will bounce off an object or obstacle on its path and be detected by the receiver on the sensor, for example, to calculate the distance to objects in the area using time and the speed of sound), capacitance (e.g., electromagnetic fields that can detect human or foreign object presence as a capacitor, for example, similar to a Theremin instrument that can detect the relative location of a human hand between two antenna), a light curtain (e.g., a set of photoelectric sensors that can detect interruption of the signal between two points, a closed Wi-Fi network (e.g., between a Wi-Fi source (router/extender) and receivers (robot arms) to report signal round trip time).

Sensing technology may be agnostic to sensor positioning. Sensors may be embedded on one or more robotic arms and/or in the environment in one or more locations. Sensors may be embedded in a robotic arm in one or more locations.

A sensor or sensors may be placed in a centralized location or dispersed around the room to track the movement of the robotics arms in relation to other objects in the environment (e.g., humans).

A proximity system (e.g., with a controller independent of the robot) may be placed on each robot/robot arm (e.g., regardless of having one arm per robot or multiple arms per robot).

The system may differentiate robotic objects from foreign objects in the field. The robotic system may be aware of the position of each of the arms in the surgical field. The system may calculate whether the object measured in the field is a robotic arm or a foreign object (e.g., surgical assistant, patient, etc.).

The movement (e.g., change in distance) of objects in the field relative to known robotically controlled movement of the arms may be monitored. The movement may inform the system whether the object is part of the robotic system or an object in the field (e.g., surgical assistant, patient, etc.).

For example, if an object moves from one side of the field to the other (but the robotic arm is not moving that far), the system may conclude that the object is foreign body and likely a human.

The effect of sterile drapes (e.g., plastic bags) may be measured and accounted for in the system when measuring the distance between the robot and foreign objects. For example, the capacitance of the bag may be measured nulled from the calculation of whether a foreign object is in the field.

The robot may inform nearby users of movement (e.g., independent of sensors) so that the users can move out of the way of the robot. Movement advertising may be achieved through one or a combination of the following feedback methods: sound (e.g., beeping or other sound localized to the moving robotic arm), light (e.g., illuminating the joints in motion or showing direction of travel through light, for example, turn signals), and/or the like.

Robotic repositioning may be sufficiently slow to allow users time to react and move out of the way or press an emergency stop button on the arm to halt unwanted movement.

Manual override control in the OR (e.g., joint release button, OTP actuator, etc.) may allow surgical staff in the room to move arms or robots in a controlled manner. These techniques may help the system avoid human-robot collisions.

A collaborative multi-system decision may limit portions of device function to prevent inadvertent directed interactions. The collaboration may include an automated cooperative decision by at least two smart devices that reduce the operations of at least one of the two system to intercede before the two systems physically interact (e.g., based on the inputs from the user).

A simulation may be used as an input stream to complex HCP visualizations.

The system may have predictive capabilities to determine a potential collision from analyzing past macro surgical history and real-time pertinent data. The system may detect the potential collision one or more steps ahead of the collision and warn of the potential engagement.

The system may output visualization of upcoming collision. The system may ask the user for input.

Interactive smart systems may enable more understanding within the OR. If conflicts between the systems arise, a decision will be made. The system may convey the conflicts in a manner relevant to the surgeon so they can make a quick informed decision on how to proceed.

Tradeoff factors may be presented for surgeon decision making. Parameters to use for tradeoffs presented to surgeon may include risk (e.g., a risk percentage) of tools touching, an anticipated number of conflicts, downtime (e.g., for the patient post-operation), a number of clutch in/out, procedure duration, an interruption during a critical step, how to minimize the of conflicts (e.g., compared to instances in time), surgeon preference, procedure-specific factors, and/or the like.

The system may create surgeon profiles that defaults setup to selected preferences. The system may identify a first path as the preference (e.g., but does not stop procedure). The profile preferences may be changed by procedure steps. The system may allow the surgeon to select the recommended settings or customize the settings.

The system may inform the user of active decision-making (e.g., with one or more alternate options for the user to select). Parameters may be displayed to a surgeon during a procedure (e.g., when a decision or trade-off occurs based on the connected device set-up). Trade-offs may include time of procedure, movement of robotic arms to adjust positioning, risks of device collision, etc.

Trade-off and decision making may be minimal during the procedure. The display may not (e.g., should never) obstruct the center of the screen. A color indicator around the border of the screen may be used to highlight a decision to be made. A display message box may show a default choice (e.g., if the surgeon chooses to ignore it). The surgeon may have the ability to open the dialog box to view the trade-off scenario. The decision may be limited to two choices.

The space above the patient may not be of equal value (e.g., the space immediately above the trocar is in constant interchanging utilization, but the space above the head or leg is less useful). The system may change choices of robotic joint(s) to intentionally keep free the more important areas (e.g., where potential interaction could occur).

FIG. 28 illustrates an example display to a user of zones of robotic arm movements. The movement zones of individual consoles/arms may be used to predict ideal movement paths. The movement zones may be quantified by tool function, time, or risk associated with collisions. As shown in FIG. 28, there may be an ineligible movement zone. The ineligible movement zone may include areas outside of the reach of the robotic devices (e.g., because the bases holding the robotic arms are fixed), areas that are a threshold distance away from the patient or operating table, areas outside of a sterile barrier, and/or areas in which people or fixed devices will be during the procedure. The movement zones may include eligible movement zones. The eligible movement zones may include areas within reach of the robotic devices, areas within a threshold distance from the patient or operating table, and/or areas within the sterile barrier. The display movement zones may allow the surgeon to visualize limits to the robotic arms' movements.

A small fringe overlap of a movement space may be treated differently than an overlap in a critical section or zone of the instrument. A movement zone may be subdivided to present and map varying risks and importance within it. This subdivision may be performed (e.g., discretely, for example, with zones represented such as with boxes or discrete elements).

Subdivisions may be nested within one another to create context. Subdivisions may be computed (e.g., dynamically with a multitude of factors, resulting in more of a granular or analog subdivision).

Movement zones may be subdivided based on the relative locations corresponding to risk.

Not all movements or actions by a robotic system may make use of the space around them in the same manner. Some actuations or movements by a robotic system may not be significantly impacted if they are stopped or encounter interference.

Due to the temporal nature of actions within a surgery, the system may have a temporal understanding of functionality with movement zones.

The system may assign functionalities to movement zones or movement zone subdivisions. A movement path may be assigned a specific function, or a subdivision may be assigned a function.

Movement zones may be subdivided based on relative location corresponding to function. As the number of pieces of equipment increase, there may be no movement paths with zero risk. The most efficient movement path may incur some additional risk of interference compared to another movement path.

Overall risk of a conflict may be calculated in a variety of ways. For example, the system may calculate the overall overlap of collision space (e.g., the calculation of total area occupied (absolute or relative) of one or multiple movement paths). The system may calculate overlap or infringement of movement zones correlated to their risk. Pass/fail rating, weighted, or other criteria may be applied to the risk that is generated to determine if a path is eligible for movement. Movement paths may be deemed eligible based on overall risk of conflict.

The determination of eligible movement paths may depend on the severity of interference. Movement paths may represent the space that can be occupied.

Overlap or infringement of movement zones may be correlated to their assigned function.

If zones (e.g., representing similar and/or the same functions, or functions that may be co-dependent) are in conflict, it may change the way space is allocated within the room. Zones may have different functions, or work within their subsets of their respective movement spaces, without ever causing a conflict or collision to arise.

Space may be allocated (e.g., temporally) based on movement paths and tasks to be performed. The camera position may impact the simulation in 3D space. The cameras may triangulate robotic position in three dimensions. The hub and/or camera system may have to have stored parameter(s) related to the device(s) being tracked and/or capabilities of those device(s). Triangulation of the device position may be used to suggest device motions. For example, the system may use kinematics of the device(s) (e.g., robot arm(s)) and the patient to determine viable movement options. The kinematics may be derived (e.g., on the fly) using visualization. The range of motion (e.g., reach) and balance of the robot may be received from the robot or pre-determined.

A vision camera system may be placed over the operating room patient. One or more (e.g., at least 4) cameras may be used to see the robot arms and tools (e.g., including trocars). The cameras may gather 3D data by looking at the preprinted fiducials on the robotic arms and tools used by the robot.

CAD data of the arms and tools may be uploaded to further simplify the setup process. For each detected object, a path planning tool may calculate a full path of robot motion (e.g., to efficiently pick and move the tool while avoiding all collisions with the other arms in the system). The anti-collision operation of this system may use AI to adjust the arms and tools to not collide with other tools.

A layout may be presented to the surgeon. The layout may allow the surgeon to see the tools outside the patient on his/her control screen. For example, the display may be similar to a bird's eye view backup camera on a car where they could see an overhead view from above. If a tool collision were to happen, the bird's eye view may allow the surgeon to fix the positioning without addition help from personnel in the room.

The system may allow precise real-time 3D positioning of tools used around the patient. The data may be used for future enhancements (e.g., trocar setup, measurement tools, collision avoidance, big picture views for surgeon, etc.).

Indexing elements (e.g., fiducials) on the arms may enable a separate system to monitor the arms, (e.g., each of the segments of the arms). In some examples, electronic sensors may be used (e.g., rather than fiducials). The electronic sensors may emit a signal that is received by other sensors or a base station. For example, fiducials and/or electronic sensors may be placed at points 55318a-c (e.g., and/or other joints) in FIG. 24.

Fiducials may be printed or marked on the arm, device, etc. Fiducials may be a stick-on label, sterile tape, etc. that a vision system can calibrate and measure (prior to use in a setup step). Fiducials may be pre-printed on device shaft and robotic arms. A clamp-on fiducial device may be added to tool shaft(s).

Patterns sprayed on a device may be used by the vision system to calibrate and measure the tool.

A linear encoder on a trocar may be used to determine axial position of the shaft with respect to trocar reference point. The shaft may be marked (e.g., with 2D bar code with ruler type markings).

A virtual trocar point may be spatially identified using fiducials and a 3D vision camera system. Robotic arm kinematics simulations may provide visualization of choices.

The system may determine alternative choices based on the procedure and the instrument capabilities and options. The system may determine a sharing relationship between two smart drives attempting to utilize the same space simultaneously (e.g., based on pre-determined aspects of the systems and their interaction). An aspect of the interaction may be the intensity or severity of the issue caused by the interaction, risk to the patient, or time criticality of one of the jobs, etc. that would be caused by the interaction. Instruments envelopes of operation may be determined based on risk or function of the instrument in causing collateral impacts to the patient or procedure, complexity of the path for the instrument to undo and take another path to avoid the other instrument, limitation to the instruments' functional capabilities (e.g., articulation angle), ease of display of the alternative options, and/or the like.

The system may determine boundary conditions of the joint movement simulation. The boundary conditions may be static. The system may identify safety limitations (e.g., danger areas for heated devices). The system may avoid critical structures when activating harmonic devices. The system may limit a joint from moving in a direction to ensure the device doesn't move toward a critical structure. If a device has a power cord, joint movement may be adapted to not interfere with cord movement.

One or more aspects may change between procedures. Some OR equipment positions may remain the same (e.g., the OR table, robotic console, ventilator, capital equipment tower, smoke evac, surgical energy, etc.). Some OR equipment positions may be semi-static (e.g., quasi set-in stone). For example, a Hugo robot arm base may be positioned and locked down.

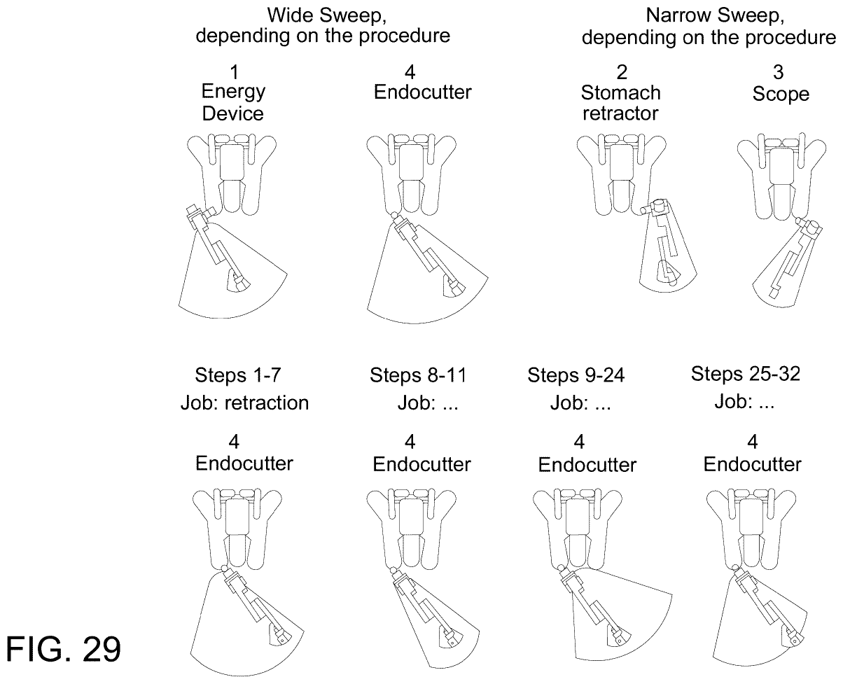

The procedure step may define when a zone is accessible (e.g., go or no-go). FIG. 29 illustrates example robotic arm movements based on the type of procedure being performed and/or a step of the procedure. For example, in certain procedures, arms controlling an energy device and an endocutter may be allowed to perform wide sweeps. For example, the energy device and endocutter may need to reach different areas of the body during the procedure, so they will be given the ability to move over large areas. This may limit the movement of other devices. Arms controlling some other devices (e.g., stomach retractor and scope in FIG. 29) may be allowed to only perform narrow sweeps. For example, the stomach retractor and scope may be moved little or not at all during the procedure (e.g., are mostly static), and won't need the ability to move over large areas. The limited movement may reduce the risk of these arms colliding with other arms (e.g., arms that will perform wide sweeps).

Boundaries may be static or dynamically change based on detected conditions. The system may determine if the plan was changed. The system may monitor for physiologic parameter shifts, user provided input, unexpected tool shifts on an arm, items changing during the procedure, and/or the like to determine changes in the plan.

The surgeon's view may change based on procedure type. For example, the view for mesentery mobilization differs from the view during anastomosis.

A scope may move between trocars (e.g., from trocar 2 to trocar 4). Within a (e.g., single) trocar, the endocutter may access various regions that depend on more proximal joint movement. The system may determine anticipated motion that will occur during the procedure. The distal tool depth and proximity to trocars may alter available space between arms.

The system may recommend a tool length (e.g., that allows a required tool depth). Motion may cause a change in arms/tools. For example, an HCP using a percutaneous retractor that cannot reach tissue may move to other side of the robot.

Unexpected circumstances (e.g., emergency physiologic needs) may occur. For example, an endocutter may misfire or arm motion may be different than initially planned (e.g., and might require human intervention).

Anticipated future steps may be used to determine the current arm movement. For example, at the end of a procedure with expected anastomosis, the surgeon may open space next to a natural orifice for access.

The system may determine whether a joint works better than others for the desired task. The system may determine whether this joint will cause a collision. The system may prioritize end effector position/use. The system may prioritize the number of arms/joints that will move (e.g., the system may avoid a collision by moving 4 arms or the system may create a potential interaction by only moving 1-2 arms). The system may deprioritize factors unrelated to the patient (e.g., stress on the tool).

Height of a tool base may be considered when determining tool position (e.g., impacts user access for exchanges). Tools that are exchanged or replaced may be fully retracted. The access point while the tool is in a fully retracted position may impact the speed of the tool exchange.

Access to distal end may be considered. For example, a scope may be manually wiped off when occluded.

The load on a retractor from the tissue may be translated through the robot arm. The system may determine joint placement to reduce (e.g., minimize) stress on robot arm joints and/or tools for long-term wear reduction. The first robot arm may include a plurality of joints configured to move the first robot arm. The device may select, from the plurality of joints, a joint of the first robotic arm to articulate to achieve the selected candidate motion.

As discussed with respect to FIG. 29, a scope arm may be a quasi-static boundary condition. The surgeon may prefer little to no movement of the scope to maintain a continuous field of view. Endocutter and retractor/energy device arms may have a greater range of motion. The endocutter and retractor/energy device may work cooperatively (e.g., communicating the desired space, and enacting boundary conditions for each other). For example, the retractor (2) may opt to minimize the angle of pitch at a joint (e.g., joint 6) to pull the head of the tool further away from endocutter (4) and energy device (1). Similarly, the energy device may opt to rotate one or more joints (e.g., joints 1, 2, and 3) to move the tool head furthest from the endocutter (4) (e.g., to give the endocutter more working space). The energy device (1) may use distal joints (e.g., roll, etc.) to perform its tasks.

An endocutter may be given maximum joint freedom to optimize the task at hand for critical firings. The endocutter may prioritize proximal joint movement to enable access angles. As shown in FIG. 29, the system may show a user multiple steps for one or more arms (e.g., to enable the user to indicate the most/least important regions of the body). For example, the endocutter may move in a wide sweep one or more times during steps 1-7 of the procedure. However, during steps 8-11, the endocutter may be relatively stationary. This information may allow the surgeon to better plan for when to move other arms. For example, there may be a lower risk of collision if other arms are moved during steps 8-11 than in steps 9-32 (e.g., when the endocutter moves in a wider area).

Each step in the plurality of steps of the surgical procedure may be associated with a surgical site internal to the patient, a second end effector is attached to a distal end of the second robotic arm. The device may identify a set of candidate motions, comprising the first candidate motion and the second candidate motion, based on the plurality of steps of the surgical procedure. Each candidate motion in the set of candidate motions may allow the first end effector and the second end effector to access the surgical site at a given step in the plurality of steps of the surgical procedure.

A static device may remain stationary for most of procedure. A dynamic device may move or be stationary based on the procedure step and/or outcome.

To visualize arm movement from one step to another, different shades within an arm may be used to indicate shifting. The visualization may allow the user to understand positions of multiple arms in different situations.

Other factors (e.g., beyond entanglement) may control the external kinematics of the joints. For example, historic data of collision/interaction, timeliness, sequencing, and/or the like may be used to determine kinematics.

The space used for different joints and/or arms may be kinematically sound. In some cases, if multiple movements are executed at the same time, the movements could cause collisions in the pathway to reach their destination. The system may consider the sequencing of movements during collision avoidance.

Movements may be performed in a specified order (e.g., order of operations, sequenced), or may be performed in parallel. The control signal may be configured to indicate the selected candidate motion of the first robotic arm. The control signal may be configured to indicate one or more of: the first candidate motion, the first number of interactions, the second candidate motion, the second number of interactions, a recommendation to move the first robotic arm according to the selected candidate motion, an order in which to perform the selected candidate motion and a motion of the second robotic arm, or a time at which to perform the selected candidate motion.

Space and time for starting positions, final positions, and/or transit paths may be temporally allocated to those functions and movements.

Entanglement and/or collisions may have degrees of conflict. There may be degrees of conflict that exceed the system's limits. The degrees of conflict may be within the limits that the surgeon and/or staff (e.g., the limits deemed critical). For certain movements or actions, the surgeon may want to push the system beyond what it normally would allow. Two arms or structures may push on one another and create entanglement concerns. That interaction may be advantageous in some way. The surgeon may override collisions or entanglement warnings.

If there is an upcoming instrument exchange, the instrument may be moved so that the tool driver can be manually switched. The system may identify eligible areas for an instrument exchange.

Instrument loading and dimensionality may be a factor for spatial isolation. If an end effector is changed on an autonomous system, an amount of space may be occupied to perform the swap. A very small end effector may not occupy much space and may only involve removing the end effector from the patient by a small amount. A larger end effector may involve fully extracting the current tool from the patient. The system may further retract away from the patient to allow sufficient space for a new tool to be installed into the system.

The system may have knowledge of a current instrument being used, an anticipated next instrument, confirmation of new instrument installation, and/or instrument-conveyed information.

The instrument may exchange information with the robotic system (e.g., without requiring manual information to be provided, such as information of length, type, etc. over an RFID or NFC communication method).

The user may (e.g., manually) input or confirm information regarding the instrument exchange and instrument installation.

Calibration and pre-run activities may be factors for spatial isolation. The instrument may be calibrated. The instrument may occupy a space during calibration. For example, an articulating harmonic device may allow arm movement, articulation of the end effector, and activation of the instrument in-air to confirm that all steps of the exchange have been properly performed (e.g., prior to insertion into the patient). These movements and activation may pose a potential risk to the HCPs and patient if they are in close proximity. The surgeon may ensure they are not co-located to the equipment.

The HCP location may be a factor for spatial isolation. For example, during an instrument exchange, the HCP may not stand in the same location occupied by another individual or piece of equipment. The system may anticipate where people and/or equipment are located, or use data streams (e.g., room cameras or triangulation of other equipment) to determine eligible locations for the instrument swap to occur.

Instrument swap complexity may be a factor for spatial isolation. A highly complicated swap of instruments may utilize additional space to perform the swap (e.g., as opposed to a low complexity swap of instruments).

For example, basic mechanical end effectors may be easily moved into locations that put a slight strain on the HCP (e.g., but allow for the surgery to be performed faster). The strain in this case may not be significant due to the ease of the instrument swap.

A more complicated, electro-mechanical interface with auxiliary connections may be more difficult to swap and may take longer for an HCP to perform. The mild strain that was acceptable for a faster swap may no longer be acceptable. In this case, the robotic system may move the instrument to a more accessible location.

Multi-step instrument exchanges may utilize multiple locations.

Complicated instrument assembly may involve moving the instrument to different locations for different stages of the instrument removal and assembly process (e.g., due to prior instrument removal, new instrument installation, auxiliary connections, electrical RF connections, new instrument calibration, position calibration, confirmation, such as scanning a barcode or button press, and/or the like).

The surgeon may deviate with the instrument swap from the planned instrument exchange (e.g., to use a different length, one of a different personal preference, or due to supply constraints).

The selected space may be (e.g., manually) modified. The new instrument may occupy more space than the system originally anticipated. The system may allow the user to enable new constraints or to manually move the end effector. The device may receive user preference information and a patient position associated with the surgical procedure. The device may determine a surgical constraint based on at least one of the user preference information or the patient position. The device may select the candidate motion of the first robotic arm, from the first candidate motion and the second candidate motion, based on the surgical constraint.

The user may manually override or modify the instrument confirmation. The new instrument may be different from the instrument that the system originally anticipated. This may result in modifications to the system's planned future movements. The system may send a confirmation of the impact to the surgical plan.

Leads and/or chords attached to the patient may be considered when determining device movement. For example, the patient may be attached to an IV, O2 supplementation, an EKG, a blood pressure cuff, energy wires, a monopolar ground pad, etc.

While the surgeon is viewing internal images at the robotic console, the system may display information related to external arm position.

If a procedure change occurs after the plan is determined, the system may provide options to the surgeon (e.g., options regarding how to proceed).

Although some aspects are described with respect to one or more robotic arms, a person of ordinary skill in the art will appreciate that these aspects may be used for any powered device (e.g., an articulable endocutter, etc.).

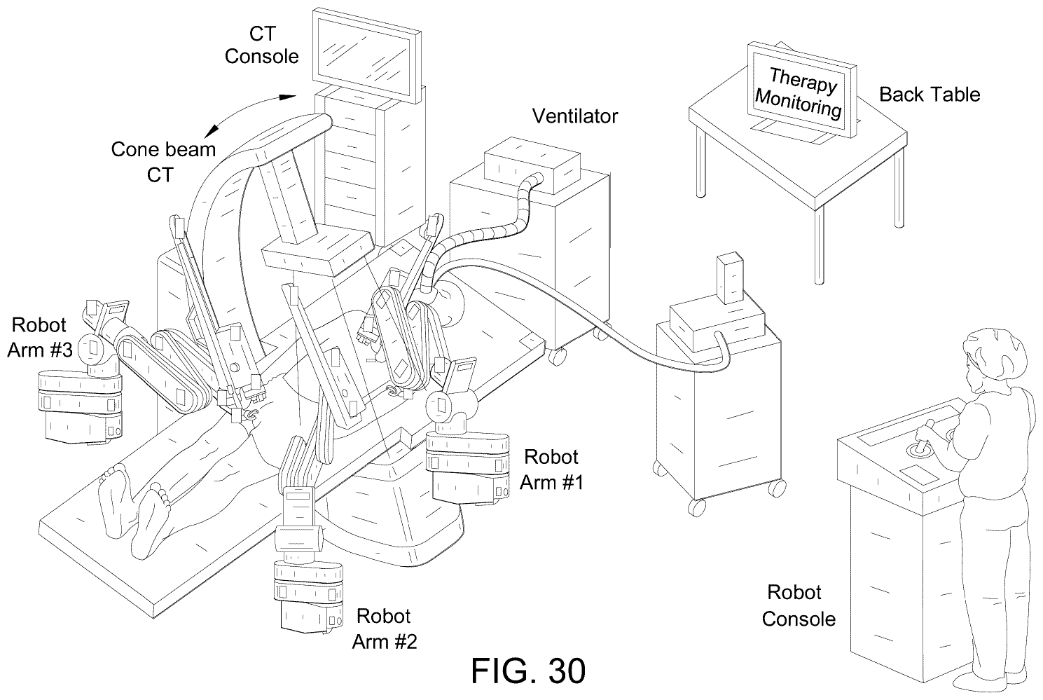

FIG. 30 illustrates an example operating room arrangement of multiple robotic arms and surgical equipment. As shown, multiple robotic arms may be present in a relatively small space in an OR. The arms may have ranges of motion that overlap with each other or other devices. In this case, the arms may collide unless adjustments are made to reduce the potential interactions. For example, one or more of the arms may not be allowed to occupy the overlapping space (e.g., at a given time).

As shown, the operating room may include one or more fixed (e.g., non-moving) devices. For example, the fixed devices may include a ventilator (e.g., a Monarch smart ventilator). The ventilator may be placed (e.g., and fixed) at the patient's head (e.g., because the ventilator must be attached at the patient's mouth and/or nose).

A smart system may display and highlight confounding data to improve feedback provided to a health care provider (HCP). The system may display complex and/or conflicting interrelated data streams to the HCP for input. Multiple monitored patient data streams (e.g., that are related to the same control parameter of a smart system and closed loop on at least one of the biomarkers) may provide inconsistent information about the control patient parameter. More than one of the parameters may be displayed to the user with context (e.g., to enable the HCP to intervene or provide guidance regarding system actions relative to the inconsistency). The related biomarkers may be from multiple smart systems and/or measured in multiple patient locations.

The interrelationship of monitored signals may be (e.g., may appear to be) conflicting or confounded. In this case, the system may not act on the signals without input from the HCP. The system may display and/or highlight combined datasets for the HCP to review. For example, the system may provide the signals and context to the HCP (e.g., so that the HCP may intervene in the decision-making process, if necessary).

An individual data stream may not function as expected. In this case, the system may be unable to proceed with an automated decision. The system may seek input from the surgeon. The system may determine data to display when asking for surgeon input (e.g., requesting that the surgeon confirm if a data stream may be not behaving as expected).

The user (e.g., surgeon) may validate one or more data streams. Data stream(s) may have undefined functionality. The system may have an undefined reaction to the introduction of the data stream(s). Data streams may be configured (e.g., in real time) to allow the user to incorporate new data streams (e.g., and validate the integrity of the data).

The HCP may generate limit(s) on the information displayed. For example, the system may display extended/long term/historic data stream, data relative to predefined limits, etc.

During surgery, the system may monitor patient temperature. If the mean body temperature while under anesthesia drops below 35° C., it may result in vasoconstriction. If the mean body temperature exceeds 37.5° C., it may result in vasodilation. The system may show the temperature of the patient relative to those limits.

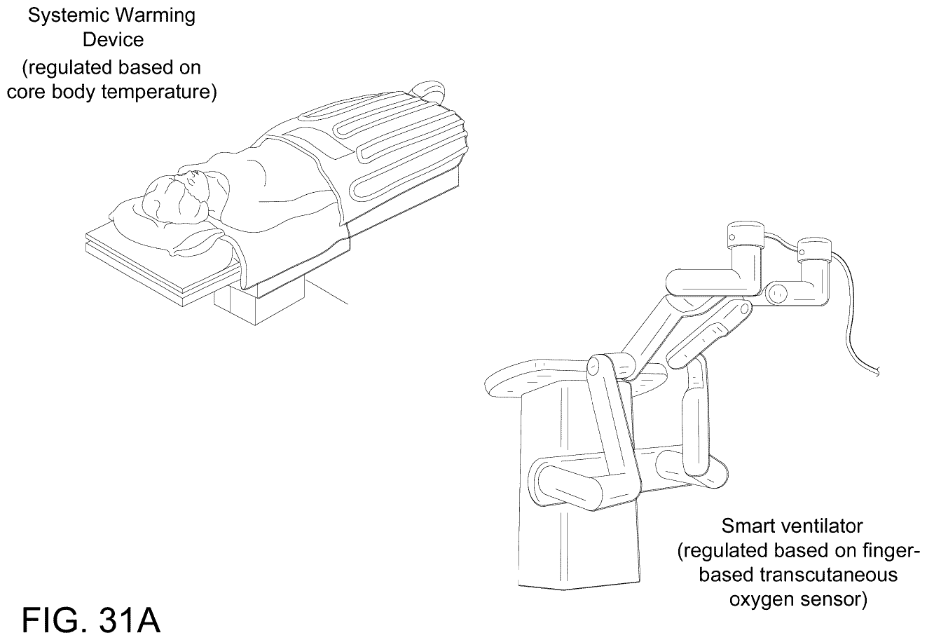

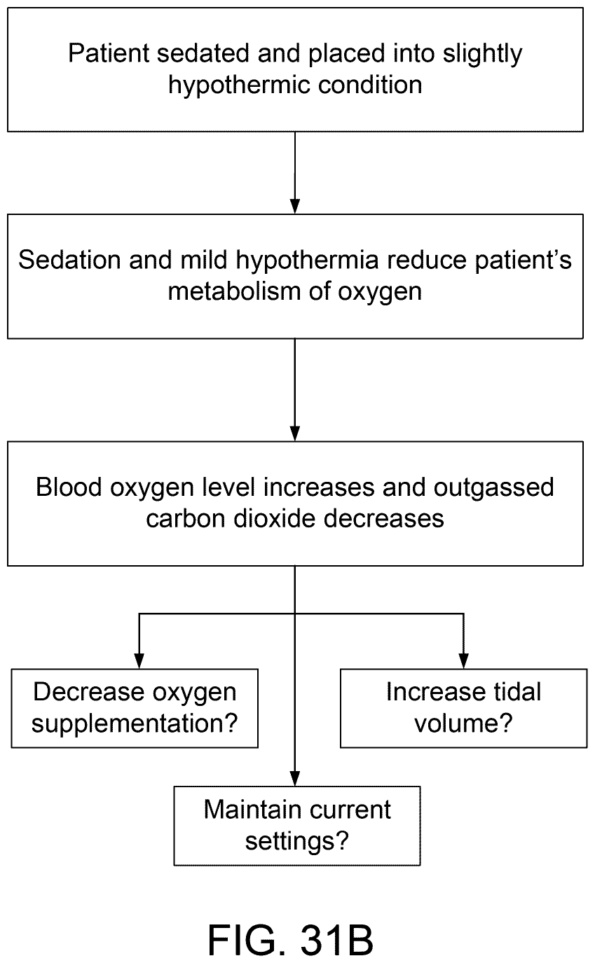

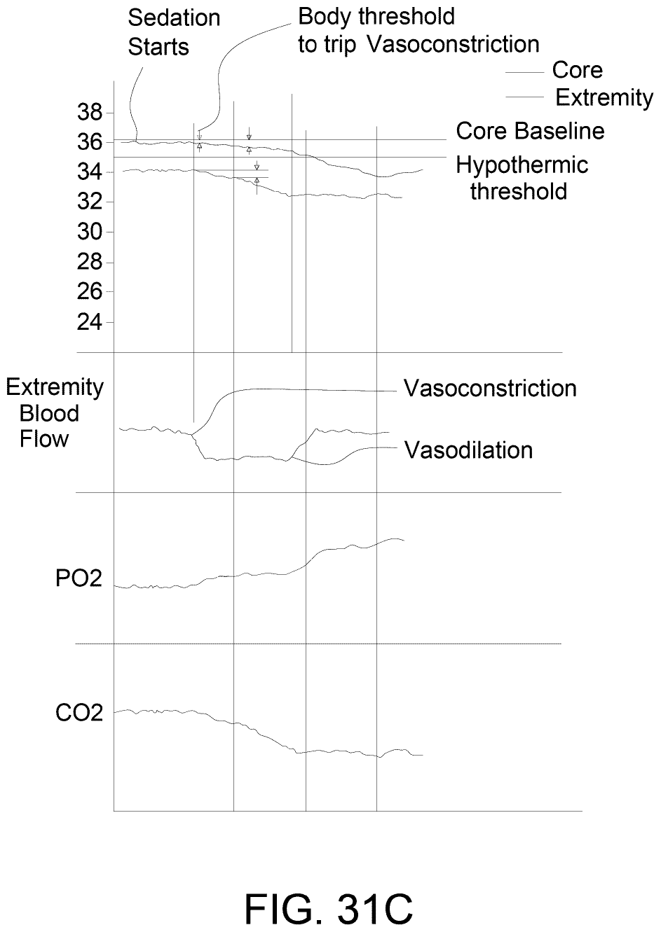

FIGS. 31A-C illustrate example conflicting decisions based on data from a systemic warming device and data from a smart ventilator. As shown in FIG. 31A, the systemic warming device may be regulated based on the core body temperature of the patient. The smart ventilator may be regulated based on finger-based sensor for monitoring transcutaneous oxygen levels. As shown in FIG. 31B, a surgical procedure may involve sedating the patient and placed in a slightly hypothermic state/condition. The sedation and mild hypothermia may reduce the patient's metabolism of oxygen. During the procedure, the blood oxygen level may increase and the outgassed carbon dioxide may decrease. In this case, the system may not know whether to decrease oxygen supplementation or increase tidal volume.

The system's decision may vary based on which temperature reading the system uses. For example, the system may select one option over the other based on the core body temperature reading but may select the other option based on the finger-based oxygen sensor. For example, as shown in FIG. 31C, the extremity temperature may drop below the hypothermic threshold before the core body temperature. The temperature change may (e.g., initially) result in vasoconstriction (e.g., as the body tried to maintain core temperature with the aid of the systemic patient warming). Specifically, the body may vasoconstrict the blood flow to extremities.

As the procedure continues, the patient's core temperature may drop below the hypothermic threshold. The body may reverse the vasoconstriction to a vasodilation state. The vasodilation opens the flow of cold blood to the extremities, which may rapidly increase the core temperature loss. As the patient's core body temperature continues to drop, the patient's blood oxygen level (PO2) may increase and the outgassed carbon dioxide (CO2) may decrease, as shown. If vasodilation occurs and the patient's temperature at the extremities increases, the system may be unsure of whether to maintain or change the oxygen supplementation and/or tidal volume.

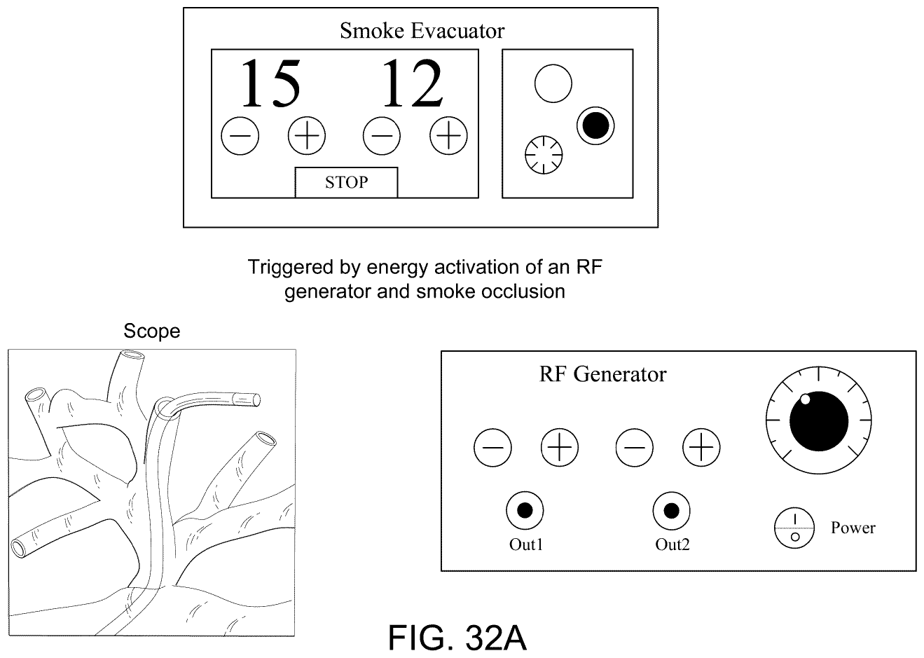

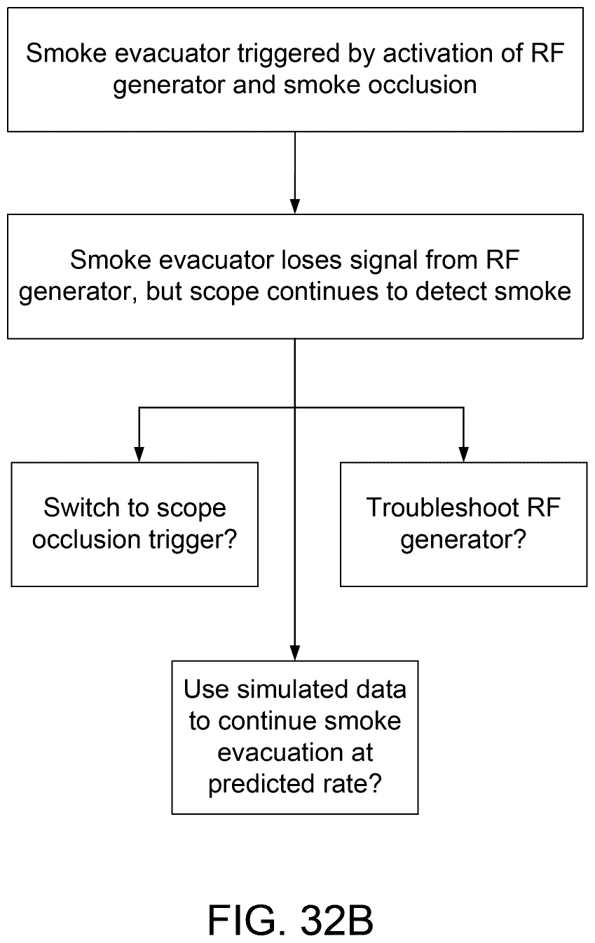

In another example, as illustrated in FIG. 32A a smoke evacuator may be activated by energy activation of an RF generator and/or smoke occlusion detection. The smoke evacuation may remove the smoke to improve visibility and reduce abdominal pressure. As illustrated in FIG. 32B, the smoke evacuator may lose signal from the RF generator (e.g., the cable between the evacuator and generator is unplugged/disconnected). The smoke evacuator may receive data from the scope that continues to indicate the presence of smoke. The smoke evacuator may not know whether to trust the data from the generator or the scope.

The system may determine that the sudden loss of the generator signal is likely due to a mechanical failure (e.g., disconnect rather than abrupt stop in energy during a surgical step). In this case, the system may determine to rely solely on occlusion as an evacuation trigger. In this case, the system may indicate the decision to the surgeon, along with context information (e.g., sudden end of energy activation unlikely at this time). The system may determine that the OR personnel should troubleshoot the generator. In this case, the system may display a warning that the generator may not be acting as expected. The system may determine to use simulated data to continue the smoke evacuation at a predicted rate. For example, if the historical data of smoke evacuation showed a steady decline over the previous 10 minutes, the system may continue to slowly decrease the smoke evacuation at the same rate.

Potentially problematic data may be displayed relative to the limits (e.g., previously established limits). The limits may be empirically set (e.g., based off of the limits of equipment, biological function, or established literature). The limits may be configurable to surgeon preferences. Such limits may help the system identify flawed data. For example, if the patient's temperature reaches over 212° C., it may be highly likely the data source itself may be in error.

The system may flag inaccurate data on a display. The inaccurate information may be flagged (e.g., with a red boundary) and displayed so that the surgeon can monitor the value to make a decision.

The system may display data that is relative to historical zones of interest. The system may subdivide the graphical space (e.g., based on limit(s) and/or other metrics). A graphical representation may have multiple zones. The zones may include limit(s) and/or additional zones that may be of interest to the surgeon.

Zoned data may be correlated to intensity of display graphs. For example, a visual representation of a standard deviation curve may have the intensity of a color correlated to the commonality of a value (e.g., a more common value has higher intensity, and outliers lose coloration).

In another example, a visual representation may overlay a sample standard deviation curve onto a graphical format. The coloration may be utilized to display intensity within the graph.

The system may display one or more versions of correlated data. The system may display predicted data. For example, data may include predicted data values. The predicted data values may be based on one or more models (e.g., human physiology, cause-effect, advanced machine-learning based models, etc.).

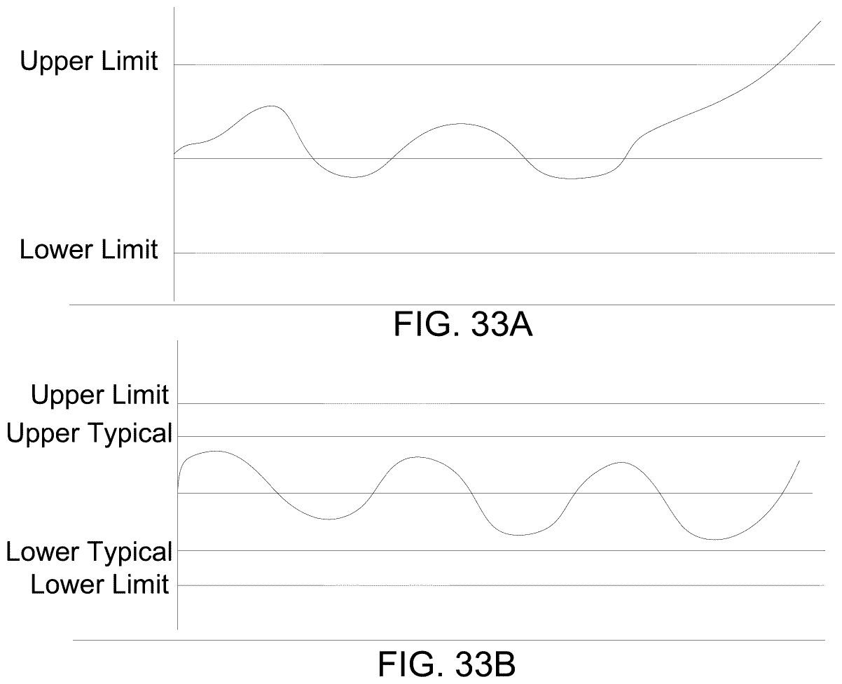

Background information may be used to provide context for decision making. The system may display data within bounds/thresholds. For example, FIG. 33A illustrates an example display of data and associated upper and lower limits. This may enable a user to see changes that don't justify a warning. If the value of the data exceeds the upper limit, as shown, the system may output a warning to the user.

The system may display conflicting data sources to the surgeon. The system may display information from multiple data sources. The user may use the display to understand how the differences may be impacting the data stream.

The system may display the current reading of a data stream. The real-time or current data may be displayed to the HCP at the same time as correlated data is displayed to the HCP.

The duration and history of data display may be configurable. Timeframes and mathematical operations (e.g., average, maximum, minimum, etc.) may be configurable by the surgeon (e.g., to best represent the information they would like to see). For example, the surgeon may request that they system display the current patient temperature and the patient average temperature over the last 10 minutes.

The system may indicate a direction and/or rate of change of a data stream. The system may indicate whether a value is increasing, decreasing, or holding steady. The system may display system behavior and/or changes. The system may indicate a transformation or compensation applied to a data stream (e.g., without showing the raw data).

The system may request user input on a data stream. For example, the system may display prompts or suggestions of how to correct the drop-out in signal. For example, the system may prompt the user for a troubleshooting step for a sensor. In an example, if the system detects that a signal dropped, the system may determine that the cable has likely been disconnected. In this case, the system may prompt the user to reconnect the cable of the system.

The system may display options (e.g., possible options moving forward) from which the user can select. The system may prompt the user with troubleshooting steps and/or actions to be taken (e.g., based on proximity and time to complete each step).

For example, the system may detect a loss in signal based on receiving corrupted or erroneous data (e.g., the voltage on the sensor may be outside the normal range). The system may prompt the user with a series of walkthroughs for how to troubleshoot the issue. For example, the troubleshooting may include checking that the cable is physically connected, checking the connection to the patient, checking that IFU steps were followed (e.g., the patient was shaved, the connection is in the correct location, etc.), replacing the sensor (e.g., if necessary), and/or the like.

The system may indicate (e.g., highlight) impacts from the data loss. The system may indicate the impact that the lost data will have on the system and/or HCP.

The system may be recalibrated using alternate data streams. Humans may monitor data streams. The system may be recalibrated to a new data stream that involves more active human monitoring.

The system may be recalibrated to a new data stream that has an additional error or offset (e.g., while remaining acceptable). In this case, the accuracy or precision that the system provides may be reduced. For example, if the primary patient temperature monitoring system fails, the system may monitor patient temperature through a finger sensor (e.g., which may not provide the same accuracy as the primary sensor). In this case, system accuracy may be reduced. The system may warn the HCP of the accuracy reduction and the change in monitoring method.

Data streams with may not impact the user or procedure. For example, if data is lost, the HCP may manually map a different data source (e.g., so that no additional action is needed).

The user may decide to proceed with the current data stream. A data stream may fall outside of a given range that was enabled but may not be physiologically incorrect. For alarms to be useful, they may be constrained to 95% of the population (e.g., because the other 5% of the population may have physiological traits that fall outside that range). In this case, the system may change a threshold/range to account for the people outside the standard range.

For example, as shown in FIG. 33B, the system may display upper and lower limits (e.g., absolute upper and lower limits), an upper typical limit, and a lower typical limit. The typical limits may show the standard limits (e.g., for 95% of the population). The absolute limits may show the absolute acceptable range of the data (e.g., for 100% of the population). For example, a heart rate monitor may determine that the patient's heart rate is very low. In this case, the system may determine whether the patient has a naturally low heart rate (e.g., below average resting heart rate). If the patient has a naturally low heart rate, the system may forego displaying a warning until the heart rate drops below to absolute lower limit.

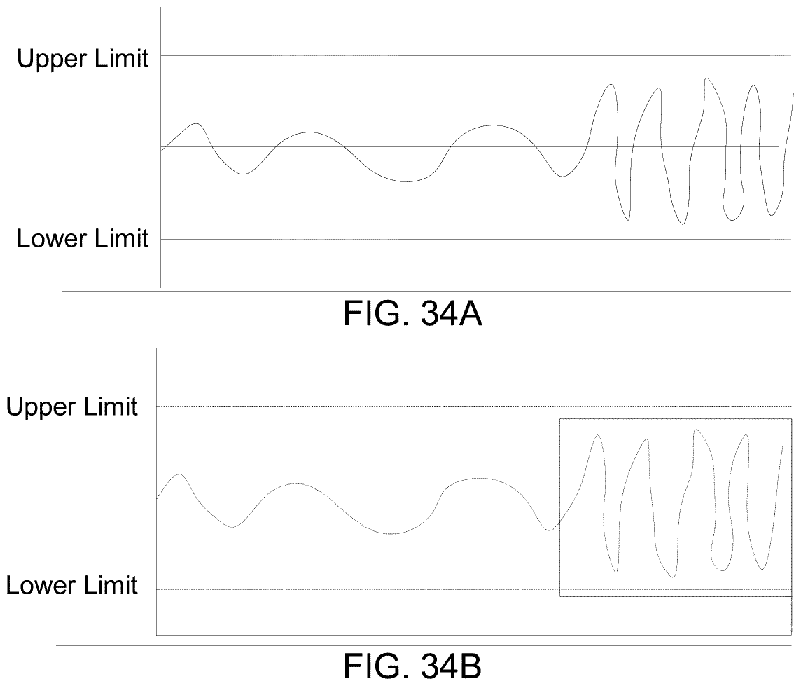

Current data and correlated data may be displayed (e.g., simultaneously). The system may display the data over time, as shown in FIG. 34A. The system may display the current value of the data and the value trending over time. The data may be displayed within a graphical format (e.g., to represent performance of the data). For example, within a monitoring system, the system may (e.g., simultaneously) display the current value of the data stream and historical data (e.g., the immediately or configured historical data) that led to the current value.

The system may display the current value in the context of (e.g., contextualized to) a correlated data stream. For example, data may be displayed for a particular reading in the context of other correlated data. Displaying a data stream may involve displaying (e.g., directly or indirectly) a plethora of (e.g., related) data. The system may utilize a line-graph, scatterplot type format, or other formats. The prior data may be related to other surgeries, or stages of those surgeries.

Data may be displayed alongside a separate (e.g., related or correlated) data stream. The system may highlight a localized data segment within a data stream. For example, if there are substantial variations in the data relative to a prior period of time, the system may display variation relative to historical trending of the data (e.g., to quickly indicate that there may be a problem present).

In an example, a baseline graphical representation may include a high variability segment. The high variability segment may be highlighted to draw attention, as shown in FIG. 34B. Segments (e.g., highlighted segments) may be detected by conventional mathematical means (e.g., measures of statistical variability, maxima or minima values, sudden changes in rate of change of a signal, machine learning, and/or the like).

The system may display historical data. Historical data may be data from a prior event. The historical data may be from a prior surgery of the same patient, performed by the same surgeon, the same hospital system, or large-scale (e.g., nationwide) surgical data. The data may include procedure-specific data, hospital-specific data, surgeon-specific data, patient-specific data, and/or the like.

Historical data may be utilized on a case-by-case basis. Historical data may include aggregated statistics of many people that relate to the current procedure, patient, and/or situation. Historical data may utilize data from within the same surgery.

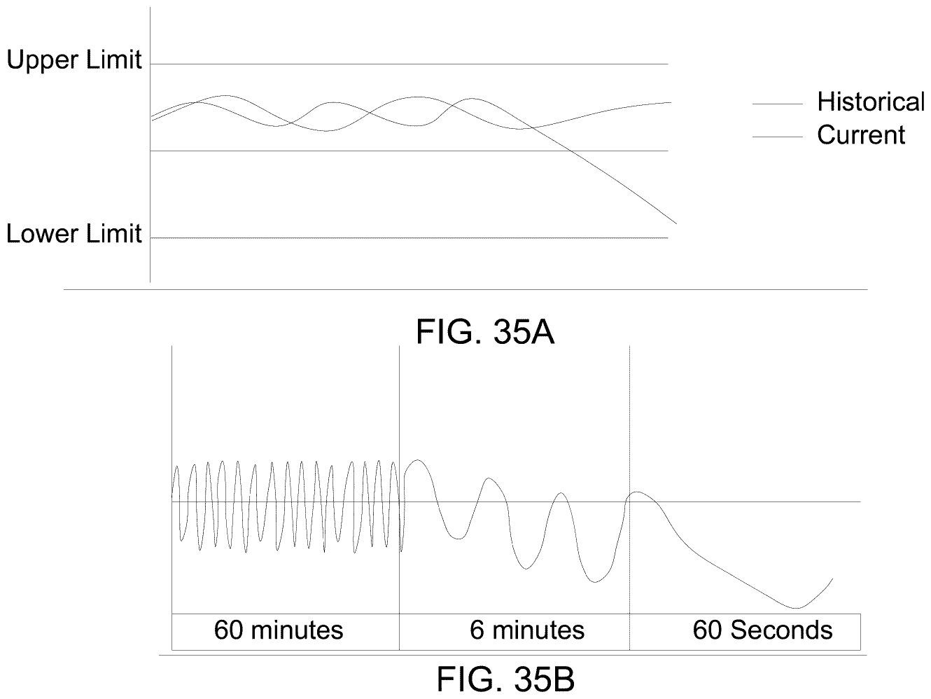

As shown in FIG. 35A, the system may display the historical data overlaid on current data. This may allow the user to quickly visualize discrepancies from the expected data values (e.g., such as the sudden drop in FIG. 35A). Expected data may be defined as data that fits within the limits or physiological ranges for the human body or within the operable ranges of equipment.

The presentation of data may be simplified. For example, the presentation of historical data may be simplified by presenting the data over a logarithmic axis with time. As shown in FIG. 35B, the system may show historical data from the previous 60 minutes, 6 minutes, and 60 seconds of the surgical procedure. In this case, the historical data may show minimums, maximums, and/or averages collapsed relative to time (e.g., so that all data can be shown within a single snapshot). For example, in FIG. 35B, the 60-minute segment appears relatively consistent, the 6-minute segment shows a gradual decline, and the 60-second segment shows a more rapid decline. The system may use waterfall graphs to display data.

The surgeon may manually verify data readings from a sensor (e.g., based on a secondary reading performed by the surgeon). The system may indicate potential locations for sensor applications. For example, if a sensor was placed in the wrong or a subpar location, the system may indicate other (e.g., better) locations to apply the sensor.

The system may propose correction(s) and/or procedure step change(s) based on a data stream. Multiple signals impacting the same control loop may lead the system toward different decisions. If the system cannot reconcile the differences to make an automated decision, the system may seek surgeon input.

The system may use cascading warnings to warn the user. For example, the system may show system interconnections at which the issue cascades from one system to the next.

A first smart system may notify a second smart system that it may be likely to interfere or impact a measurement aspect of the second smart system. The first smart system may sequentially cascade the warnings/notifications to the other systems that are downstream dependent on those monitored aspects (e.g., for closed loop control or transformations).

The system may warn subsequent systems that the data will be affected or missing. The system may supply a replacement value for the missing data. The system may indicate a duration of the effected aspect (e.g., a length of time during which the replacement value is to be used).

The duration of, intensity of, or reaction to the warning may intensify (e.g., based on the issue). For example, if a temperature sensor on a patient warming device is faulty (e.g., the function of the patient warming device to warm the patient is disabled), the patient risk of hypothermia may increase. The system may warn the surgeon of the change. The system may cascade the warning from the patient warming device to the patient transcutaneous temperature sensor or smart ventilator. The warning may let the other devices know that readings and/or patient condition may (e.g., are likely to) change.

The system may output discrete notifications. The smart system (e.g., acting on data feed from another system) may detect that the feed violates one or more of the closed loop envelope operational window(s). The thresholds may be tiered with higher or lower levels (e.g., resulting in more intense notifications or actions as the level increases).

Example discrete electrical failures are described herein. A discrete electrical failure may include hardware failures, PTC resetting events, high signal noise (e.g., reduction of the signal-to-noise ratio), shorting events, unstable power lines/voltage rails, a brown out, a black out, fluctuations, and/or the like.

The system may output notifications related to continuous feed(s). The data stream may have a continuous feed or data. In some examples, the value of the data stream at a given time (e.g., after detection of the issue) may be as critical as the violation of the bounded thresholds. For example, the system may use the dynamic rate of reaction to adjust warnings/notifications (e.g., based on the value's closeness to threshold).

The system may establish a rate of warning escalation for a continuous feed. The threshold and/or rate may be adjustable based on the situation/user. For example, proximity detection of robotic arms may cause different levels of reaction (e.g., depending on tools attached to the arms).

The surgeon or system may adjust inputs based on bandwidth usage. For example, the system may be aware of the available bandwidth and its rate of consumption. The system may determine (e.g., based on other smart systems) whether to reduce its bandwidth consumption.

A physical workspace may share notifications. For example, the system may be aware of the physical constraints of itself and its workspace. The system may determine velocity limits, position limits, acceleration limits, etc. for the workspace.

Warning severity escalation may be in reaction to confusing data streams. The system may combine warnings/notifications from multiple data streams (e.g., to generate a more severe/elevated warning). For example, a visualization system may detect a bleeding event and send a notification to the supervisory smart system. Depending on the level of bleeding, the supervisory smart system may react differently (e.g., elevate the warning response to the user, send request messages to other smart systems to query for more surgical environmental information, etc.).

In another example, the smart system may identify an energy device being activated off screen. The system may send a warning to another smart system that the surgeon is purposefully activating the energy device. In this case, neither system may display the warning. The system may determine a course of action (e.g., system shutdown or notify a user of a warning).

The smart system may determine whether to notify the user based on a known or calculated accuracy of the data. The smart system may average or filter data (e.g., on the fly) to compensate for inaccuracies. The smart system may check multiple sources to understand variations in the data (e.g., to better compensate for the inaccuracies).

Event timing may be used to resolve conflicting data. If the timing of an event is critical, the system may decide to present a warning. The system may pause notification to prevent unwanted movement during critical activation. For example, the system may lock out movement while stapling or sealing. A harmonic device may be allowed to proceed with movement to allow for cutting. During startup, the system may determine that all devices are booted and idle prior to continuing the procedure.

Multiple simultaneous devices may be activated from multiple independent generators. A capacitive grounding pad may be shared by multiple monopolar generators.

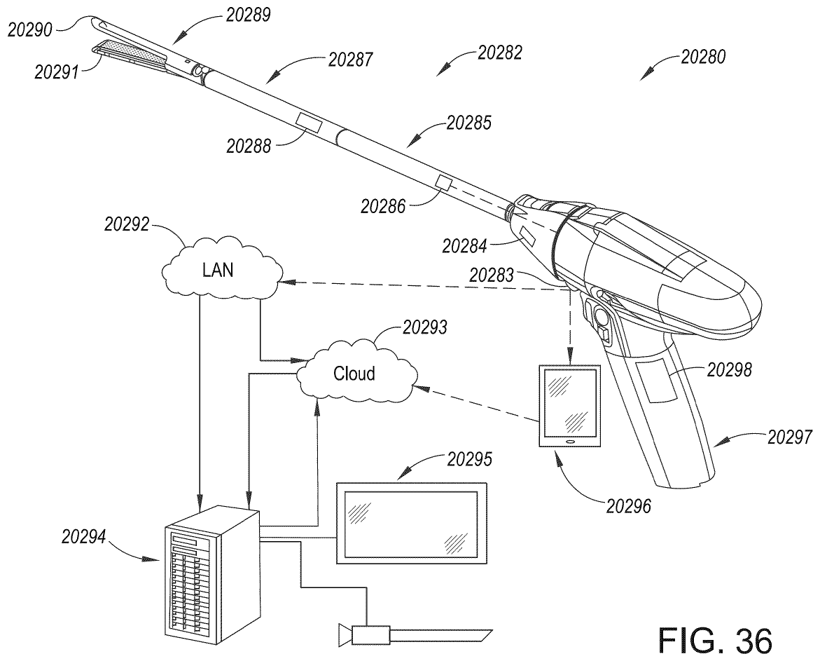

FIG. 36 illustrates an example surgical system 20280 that may include a surgical instrument 20282. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

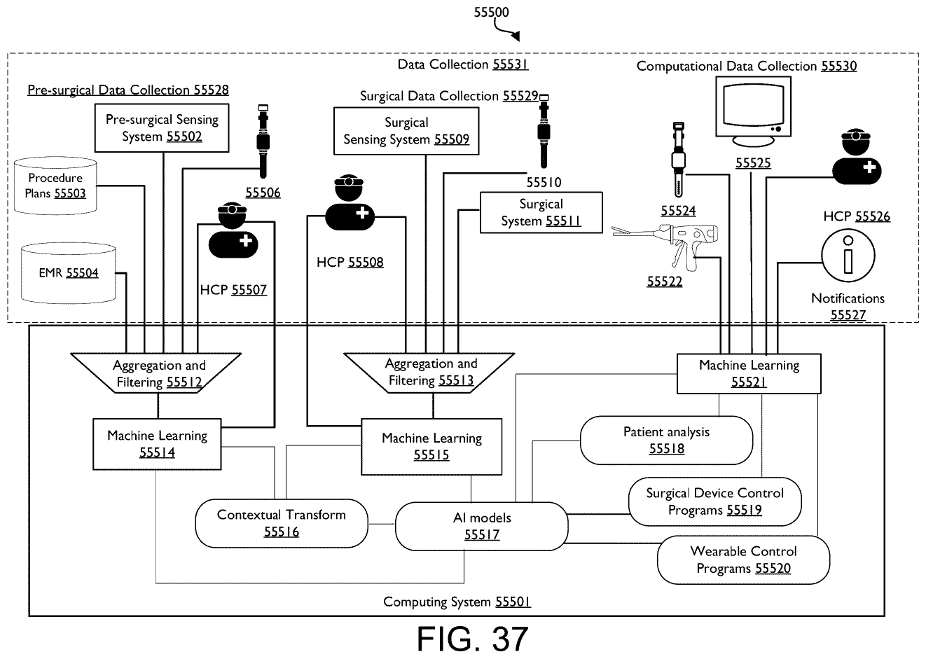

FIG. 37 depicts a block diagram for applying machine learning to improve artificial intelligence algorithms and/or iterations of learning for artificial intelligence algorithms. The computing system 55500 (e.g., surgical computing system and/or surgical computing hub) may include computing hardware including a processor, memory, input/output sub-systems, and the like. The processor may be configured (via application specific hardware, software, firmware, or the like) to transform received data and to derive contextualized for output. For example, the processor may include a microprocessor, a microcontroller, a FPGA, and an application-specific integrated circuit (ASIC), a system-on-a-chip (SOIC), a digital signal processing (DSP) platform, a real-time computing system, or the like. For example, processor may be configured to implement computing functions and/or modules as disclosed herein. For example, the processor may be configured for aggregation and/or filtering 55512, aggregation and/or filtering 55513, machine learning 55514, machine learning 55515, machine learning 55521, contextual transform 55516 (e.g., including real-time intra-operative processing), artificial intelligence models 55517, patient analysis 55518, surgical device control programs 55519, wearable control programs 55520, and the like.

The computing system 55500 may be any device suitable for processing sensor, health record data, user input, and the like, to transform the data and derive computational data for output. The computational output may include a sensor measurement. The computational output may include contextual information or a context, for example, which may include additional information relevant to the present understanding and/or interpretation of the sensor measurement. For example, the context may include pre-surgery and/or pre-therapy baselines. For example, the context may include situational awareness of incorrectly connected and/or incorrectly operating surgical and/or sensing systems. For example, the context may include adjustments to products, surgical plans, and/or margins.

The computing system 55500 may be incorporated into the system 55500 with any method suitable for implementation of the functionality disclosed herein. For example, the computing system 55500 may be incorporated as a stand-alone computing system. For example, the computing system may be incorporated into a surgical hub. For example, the computing system 55500 may be incorporated into a sensing system itself (e.g., sensing both pre-surgical and surgical data and providing contextualized data as an output). For example, the computing system 55500 may be incorporated into a surgical device itself (receiving both pre-surgical and surgical data and providing contextualized data, computational data, and/or alerts as an output).

A data collection, such as data collection 55531 may be provided. Machine learning may use the data collection, such as data collection 55531. Data collection 55531 may be used by machine learning to train, verify, create, and/or determine an ML model.

Data collection 55531 may include one or more data sources. For example, data collection 55531 may include pre-surgical data collection 55529, surgical data collection 55529, and computational data collection 55530. Data collection 55531 may include one or more biomarkers. The one or more biomarkers may come from one or more computing systems, surgical sensing systems, wearable devices, displays, surgical instruments, surgical devices, sensor systems, devices, and the like. The data collection 55531 may include electronic medical records for a patient, data for a patient, data for other patients, data regarding past procedures, data regarding research for procedures, medical data, instructions from a health care provider, plans for a surgery, and the like.

Data collection may include data from a number of different sources. For example, the sources may include procedure plans database 55503, EMR 55504, pre-surgical sensing system 55502, wearable device 55506, data from health care provider 55507, surgical sensing system 55509, HCP 55508, wearable device 55510, surgical system 55511, wearable device 55524, surgical device 55522, human-interface device 55525, data from HCP 55526, and data related to notifications 55527.

Data collection 55531 may include pre-surgical data collection 55528. Pre-surgical data collection 55528 may include data from one or more data sources. Pre-surgical data collection 55528 may include data that is related to a patient that may be recorded prior to a surgery. Pre-surgical data collection 55528 may include one or more biomarkers they may have been recorded for a patient prior to a surgery. For example, a heart rate and blood glucose level for a patient may be recorded for a patient prior to a surgery.

Pre-surgical data collection 55528 may include data from pre-surgical sensing system 55502. Pre-surgical sensing system 55502 may include any configuration of hardware and software devices suitable for sensing and presenting patient parameters and/or biomarkers that may be relevant before, during, or after a surgical procedure. Such a pre-surgical sensing system 55502 may include any of the sensing and monitoring systems disclosed herein, including uncontrolled patient monitoring systems, controlled patient monitoring systems, and the like. For example, pre-surgical sensing system 55502 may include a wearable patient sensor system. The pre-surgical sensing system 55502 may provide data suitable for establishing baselines of patient biomarkers for use in contextual determination during and/or after surgery. The pre-surgical sensing system 55502 may provide data suitable for establishing baselines of patient biomarkers for use in making predications and/or creating computational data.

Pre-surgical data collection 55528 may include data from wearable device 55506. Wearable device 55506 may include any configuration of hardware and software devices suitable for sensing and presenting patient parameters and/or biomarkers that may be relevant before, during, or after a surgical procedure. Such systems may be used by the patient for any amount of time prior to surgery, inside and outside of a medical facility. To illustrate, via an uncontrolled patient monitoring system, the patient may use a wearable heart-related sensor at home for four weeks prior to a surgical procedure. And/or, via a controlled patient monitoring system, a HCP may monitor the same and/or analogous biomarkers using facility equipment during time the patient is prepped immediately before the surgical procedure. For example, the wearable device 55506 may provide data suitable for establishing baselines of patient biomarkers for use in contextual determination and/or for use in creating computational data.

Pre-surgical data collection 55528 may include procedure plans 55503. Procedure plans 55503 may include any data source relevant to a health procedure (e.g., relevant to a health procedure in view of a particular patient and/or facility). Procedure plan 55503 may include structured data indicative of the desired end result, the surgical tactics to be employed, the operation logistics, and the like. Procedure plan 55503 may include an accounting of the equipment to be used and/or the techniques to be used. Procedure plan 55503 may include an order. Procedure plan 55503 may include a timeline. The structured data may include defined fields and/or data tags associated corresponding values. The structured data may include codes associated with surgical steps.

Pre-surgical data collection 55528 may include EMR 55504. EMR 55504 may include any data source relevant to a patient in view of a health procedure, such a surgical procedure. EMR 55504 may include information such as allergies and/or adverse drug reactions, chronic diseases, family medical history, illnesses and/or hospitalizations, imaging data, laboratory test results, medications and dosing, prescription record, records of surgeries and other procedures, vaccinations, observations of daily living, information collected by pre-surgical sensing system 55502, information collected by wearable device 55506, and the like.

Pre-surgical data collection 55528 may include data from a pre-surgical healthcare provider, such as HCP 55507. Data from HCP 55507 may include any data relevant to a pre-surgical sensing system 55502, a patient record, a procedure plan, and the like. Data from HCP 55507 may include data that may be relevant to an operation, configuration, and/or management of a computing system, such as computing system 55500. For example, data from HCP 55507 include feedback that may be provided to a machine learning module, such a machine learning module 55514. The data from HCP 55507 may include manually entering data that may not be received directly for a relevant source (such as manually entering a manually taken biomarker reading, for example).

Data collection 55531 may include surgical data collection 55529. Surgical data collection 55529 may include data from one or more data sources. Surgical data collection 55529 may include data that may be related to a patient that may be recorded during a surgery. Surgical data collection 55529 may include one or more biomarkers they may have been recorded for a patient during a surgery. For example, a heart rate and blood glucose level for a patient may be recorded for a patient during a surgery.

Surgical data collection 55529 may include one or more data sources. Surgical data collection 55529 may include data from surgical sensing system 55509, HCP 55508, surgical system 55511, and wearable device 55510.

Surgical data collection 55529 may include data from surgical sensing system 55509. Surgical sensing system 55509 may include any configuration of hardware and software devices suitable for sensing and presenting parameters patient biomarkers that may be relevant during a surgical procedure. Surgical sensing system 55509 may include the sensing and monitoring systems disclosed herein, including controlled patient monitoring systems, surgeon monitoring systems, environmental sensing systems, and the like.

Surgical data collection 55529 may include data from surgical sensing system 55509. Surgical sensing system 55509 may include any configuration of hardware and software devices suitable for sensing and presenting parameters patient biomarkers that may be relevant during a surgical procedure. Surgical sensing system 55509 may include one or more of the sensing and monitoring systems disclosed herein, including controlled patient monitoring systems, surgeon monitoring systems, environmental sensing systems, and the like.

Surgical data collection 55529 may include data from wearable device 55510. Wearable device 55510 may include any configuration of hardware and software devices suitable for sensing and presenting patent parameters and/or biomarkers that may be relevant before, during, or after a surgical procedure. Such systems may be used by the patient for any amount of time prior to surgery, inside and outside of a medical facility. To illustrate, via an uncontrolled patient monitoring system, the patient may use a wearable heart-related sensor at during a surgical procedure. And/or, via a controlled patient monitoring system, a healthcare provider may monitor the same and/or analogous biomarkers using facility equipment during time of the surgical procedure. For example, the wearable device 55510 may provide data suitable for use in a contextual determination and/or in a creation of computational data.

Surgical system 55511 may include any surgical equipment suitable for providing operative data regarding its configuration, use, and/or present condition and/or status, for example. Surgical system 55511 may include equipment in the surgical theater. Surgical system 55511 may include any equipment employed in the surgical theater as described herein. The surgical system 55511 may include surgical fixtures of a general nature, such as a surgical table, lighting, anesthesia equipment, robotic systems, and/or life-support equipment. Surgical system 55511 may include surgical fixtures that may be related to the procedure at-hand, such as imaging devices, surgical staplers, energy devices, endocutter clamps, and the like. For example, surgical system 55511 may include be one or more of a powered stapler, a powered stapler generator, an energy device, an energy device generator, an in-operating-room imaging system, a smoke evacuator, a suction-irrigation device, an insufflation system, or the like.

The surgical system 55511 may include at least one of a surgical instrument, surgical visualization system, monitor, sound system, energy devices, a wearable, and the like. For example, the surgical system may include a surgical hub. For example, the surgical system may include a surgical stapler. For example, the surgical system may include an endocutter, for example. Data from the surgical instrument may include surgical instrument parameters. The surgical instrument parameters may include surgical instrument power, for example. Data from the surgical visualization system may include location of surgical instruments in relation to a patient surgical site and/or organ. For example, data may include the distance between a surgical stapler and a close vital organ.

Surgical data collection 55529 may include data from a surgical HCP, such as HCP 55508. Data from HCP 55508 may include any data relevant to a surgical sensing system, a wearable device, a surgical system, a machine learning, a patient analysis, a surgical device control program, a wearable control program, a contextual transform, an artificial intelligence model, and the like. For example, HCP 55508 may provide data that may be associated with surgical system 55511, wearable device 55510, surgical sensing system 55509, machine learning 55515, and the like. For example, the HCP 55508 may provide data that may trigger an interaction with the contextual transform 55516 and/or machine learning 55515. The data from HCP 55508 may include manually entering data not received directly for any relevant source (such as manually entering a manually taken biomarker reading, for example).

The data received from the pre-surgical data sources, such as pre-surgical data collection 55528, may be subject to aggregation and/or filtering 55512. Aggregation and/or filtering 55512 may perform pre-processing on data received from pre-surgical data collection 55528. The data received from the surgical data sources, such as surgical data collection 55529, may be subject to aggregation and/or filtering 55513. Aggregation and/or filtering 55513 may perform per-processing on data received from surgical data collection 55529. Aggregation and/or filtering 55512 and aggregation and/or filtering 55513 may be used to prepare and format the data for use by computing system 55500. For example, aggregation and/or filtering 55512 and aggregation and/or filtering 55513 may prepare data to be processed by machine learning 55514, machine learning 55515, machine learning 55521, contextual transform 55516, artificial intelligence models 55517, surgical device control programs 55519, and wearable control programs 55520.

Processing the data received from the pre-surgical data collection 55528 by aggregation and/or filtering 55512 may include filtering (e.g., to select sensor data from the stream of data from pre-surgical sensing system 55502). Aggregation and/or filtering 55512 may use filtering to help reject noise in data from pre-surgical data collection 55502. Aggregation and/or filtering 55512 may use a method to establish a baseline for a biomarker from pre-surgical data collection 55528. Aggregation and/or filtering 55512 may perform time mapping on data from pre-surgical data collection 55528 (e.g., to place received values from different sources in alignment with each other in regard to time). Time mapping may aid in correlation and ratio analysis, which may occur in contextual transform 55516.

Aggregation and/or filtering 55512 may translate data from pre-surgical data collection 55528. The translation of data may include coordinating formats, coordinating data types, translating from one format to another format, translating from one data type to another data type, accounting for a difference between a data source data format, and accounting for a data type expected by another module, such as machine learning 55514. Translating may include translating the data into a format suitable for machine learning, for artificial intelligence models, for patient analysis, for use by a surgical device control program, and/or for use by a wearable control program. Data from the pre-surgical data collection 55528 may be translated into a notification for display, such as display on a human-interface device 55525. Data from pre-surgical data collection 55528 may be translated into a setting for the surgical device 55522. Data from surgical data collection 55529 may be translated into data that may be included and/or used for notifications 55527.

Processing the data received from surgical data collection 55529 by aggregation and/or filtering 55513 may include filtering (e.g., to select sensor data from the stream of data from surgical system 55511). Aggregation and/or filtering 55513 may use a method to establish a baseline for a biomarker from surgical data collection 55529. Aggregation and/or filtering 55513 may use filtering to help reject noise in data from surgical data collection 55529. Aggregation and/or filtering 55513 may perform time mapping on data from surgical data collection 55529 (e.g., to place received values from different sources in alignment with each other in regard to time). Time mapping may aid in correlation and ratio analysis, which may occur in contextual transform 55516.

Aggregation and/or filtering 55513 may translate data from surgical data collection 55529. The translation of data may include coordinating formats, coordinating data types, translating from one format to another format, translating from one data type to another data type, accounting for a difference between a data source data format, and accounting for a data type expected by another module, such as machine learning 55515. Translating may include translating the data into a format suitable for machine learning, for artificial intelligence models, for patient analysis, for use by a surgical device control program, and/or for use by a wearable control program. Data from the surgical data collection 55529 may be translated into a notification for display, such as display on a human-interface device 55525. Data from surgical data collection 55529 may be translated into a setting for the surgical device 55522. Data from surgical data collection 55529 may be translated into data that may be included and/or used for notifications 55527.

Contextual transform 55516 may operate to provide a context for data, such as pre-surgical data collection 55528 and/or surgical data collection 55529. For example, contextual transform 55516 may transform data into contextualized surgical data, which may be included in computational data collection 55530. To illustrate, as an input the contextual transform may receive surgical data that includes, for example, a measurement time, a sensor system identifier, and a sensor value. Contextual transform 55516 may output contextualized surgical data. Contextual transform 55516 may output data that may be modified and/or enhanced by machine learning 55514, machine learning 55515, machine learning 55521, patient analysis 55518, surgical device control programs 55519, wearable control programs 55520, and artificial intelligence models 55517.

Contextual transform 55516 may determine and/or store data that may be related to each other. Contextual transform 55516 may determine how data may be related to each other. For example, contextual transform 55516 may determine that data from surgical data collection 55529 may be related to data from pre-surgical data collection 55529. Contextual transform 55516 may determine a context for the data. Context, for example, additional information relevant to the present understanding and/or interpretation of the sensor measurement.

Computational data collection 55530 may include data that may be generated, created, determined, and/or computed by computing system 55500. For example, computational data collection 55530 may include models output from machine learning, data generated by machine learning, biomarkers processed by computing system 55500, augmented data, predictive probabilities, firmware, firmware updates, parameters for surgical devices, surgical device control program, updates to surgical device control programs, wearable control programs, parameters for wearable devices, parameters for controlling surgical devices, electronic medical records, contextual data, contextual surgical data, notifications, requests for feedback, messages to healthcare providers, and the like.

Computational data collection 55530 may include context, for example, additional information relevant to the present understanding and/or interpretation of the sensor measurement. For example, the context may include pre-surgery and/or pre-therapy baselines. For example, the context may include situational awareness of incorrectly connected and/or incorrectly operating surgical and/or sensing systems. For example, the context may include adjustments to products, surgical plans, and/or margins. Computational data collection 55530 may include data sent to or received from surgical device 55522, wearable device 55524, human-interface device 55525, HCP 55526, and notifications 55527. Computational data collection 55530 may be created, modified, received by and/or sent by machine learning 55514, machine learning 55515, machine learning 55521, contextual transform 55516, artificial intelligence models 55517, patient analysis 55518, surgical device control programs 55519, wearable control programs 55520, and/or any combination thereof.

Computational data collection 55530 may include data that provides a context. The context may include additional information that may place a biomarker into a specific context for the healthcare providers. For example, computational data collection 55530 may include instructions and/or information about a baseline value for a sensor value, an alert of a deviation, relevant information from the patient's record, relevant information to a procedural element of the surgery, surgical device settings, and/or any information the healthcare provider might find relevant to have at the moment of the sensor's measurement itself. The context may be determined by machine learning, such as by machine learning 55514, machine learning 55515, and/or machine learning 55521. Computational data collection 55530 may include one or more data tags. The data tags may include logging data (indicating that that a specific transform or other processing has occurred).

Computational data collection 55530 may include data that may be provided by HCP 55526. For example, HCP 55526 may provide feedback regarding data provided by machine learning 55521. Computational data collection 55530 may include data that may be sent to HCP 55526. For example, HCP 55526 may receive data provided by machine learning 55521. Data from HCP 55526 may include any data relevant to a surgical sensing system, a wearable device, a surgical system, a machine learning, a patient analysis, a surgical device control program, a wearable control program, a contextual transform, an artificial intelligence model, and the like. For example, HCP 55526 may provide data that may be associated with surgical device 55522, wearable device 55524, a patent, human-interface device 55525, notifications 55527, computing system 55500, and/or any combination thereof. For example, the HCP 55526 may provide data that may trigger an interaction with the contextual transform 55516 and/or machine learning 55521. The data from HCP 55526 may include manually entering data not received directly for any relevant source (such as manually entering a manually taken biomarker reading, for example).

Human-interface device 55525 may include any device suitable for producing a perceptible representation of computational data, such as computational data collection 55530. The perceptible representation may include a visual indication, an audible indication, or the like. The human-interface device 55525 may include a computer display. For example, the human-interface device 55525 may include a visual representation including text and/or images on a computer display. The human-interface device 55525 may include a text-to-speech device. For example, the human-interface device 55525 may include synthesized language prompt over an audio speaker. The human-interface device 55525 may communicate the computational data to the surgeon and/or surgical team. The human-interface device 55525 may include and/or be incorporated into any suitable device disclosed herein. For example, the human-interface device 55525 may include and/or be incorporated into a primary display 20023 of FIG. 2.

The notifications 55527 may include any device suitable for generating a perceptible indication that relevant computational data is available and/or has changed. The indication may include a visual indication, an audible indication, a haptic indication, and the like. The notifications 55527 may include non-verbal and/or non-textual indications to represent contextual data is available and/or has changed. For example, the alert system may include audio tones, visual color changes, lights, and the like. For example, the notification may include a haptic tap on a wearable device, such as a smartwatch worn by the surgeon. Notifications 55527 may include computational data, pre-surgical data, surgical data, and/or post-surgical data. Notifications 55527 may include a request from a machine learning algorithm for a HCP 55526 to provide feedback regarding data, a recommendation, an accuracy of an artificial intelligence model, an accuracy of training data, an accuracy of machine learning, a diagnosis, an indication of a problem, data generated by machine learning, a patient analysis, a conclusion regarding a patient analysis, a modification to a surgical device control program, a surgical device control program, wearable control program, any combination thereof, and the like. For example, notifications 55527 may request that HCP 55526 provide feedback regarding a surgical device control program that may be sent to surgical device 55522.

The surgical device 55522 may include any equipment employed for a surgical procedure (such as surgical systems 55511) that may have a configurable aspect to its operation. The configurable aspect of the equipment may include any adjustment or setting that may influence the operation of the equipment. For example, surgical device 55522 may have software and/or firmware adjustable settings. Surgical device 55522 may be hardware and/or structurally adjustable settings. In an example, the surgical device 55522 may report its present settings information to the computing system 55500. In an example, the surgical device 55522 may include an artificial intelligence model that may be deployed by computing system 55500, trained at computing system 55500, modified by computing system 55500, any combination thereof, and the like.

Example device settings for surgical device 55522 may include placement, imaging technology, resolution, brightness, contrast, gamma, frequency range (e.g., visual, near-infrared), filtering (e.g., noise reduction, sharpening, high-dynamic-range), and the like for imaging devices; placement, tissue precompression time, tissue precompression force, tissue compression time, tissue compression force, anvil advancement speed, staple cartridge type (which may include number of staples, staple size, staple shape, etc.), and the like for surgical stapling devices; and placement, technology type (such as energy devices, electrosurgery/laser surgery, mono-polar, bi-polar, and/or combinations of technologies), form-factor (e.g., blade, shears, open, endoscopic, etc.) coaptation pressure, blade amplitude, blade sharpness, blade type and/or shape, shears size, tip shape, shears knife orientation, shears pressure profile, timing profile, audio prompts, and the like for energy devices, for example.

Computational data collection 55530 may include data from wearable device 55524. Wearable device 55524 may include any configuration of hardware and software devices suitable for sensing and presenting patent parameters and/or biomarkers that may be relevant before, during, or after a surgical procedure. Such systems may be used by the patient for any amount of time prior to surgery, inside and outside of a medical facility. To illustrate, via an uncontrolled patient monitoring system, the patient may use a wearable heart-related sensor at during a surgical procedure. And/or, via a controlled patient monitoring system, a healthcare provider may monitor the same and/or analogous biomarkers using facility equipment during time of the surgical procedure. For example, the wearable device 55524 may provide data suitable for use in a contextual determination during and/or after surgery. Wearable device 55524 may include any wearable sensing system 20011 of FIG. 1 as described herein.

Computational data collection 55530 may include a wearable control program that may have been sent by wearable control programs 55520 wearable device 55524. Computational data collection 55530 may include an artificial intelligence model that may be sent to wearable device 55524.

The machine learning module 55514 may perform data preparation as described herein with the pre-surgical data collection 55528 (e.g., a dataset). In an example, the data preparation may further include the machine learning module 55514 receiving input from an HCP 55507 labeling a subset of the data records in the dataset for training a pre-surgical patient analysis model (e.g. a training dataset). The pre-surgical patient analysis model may be stored at and/or or included within AI model 55517. The pre-surgical patient analysis model may be a training data set with supervised machine learning for patient analysis 55518 (e.g., a probability of surgical complications during a surgical procedure). For example, machine learning 55514 may receive data to from pre-surgical data collection 55528 that may be used to train an ML model that may be stored at AI model 55517 and may be deployed at patient analysis 55518.

Those of skill in the art will recognize any suitable machine learning algorithm may be used to build the model 55517. For example, the input from HCP 55507 may include a “high risk” label when a patient's data record from patent records 55504 indicates a risk of surgical complications related to adhesions due to a history of multiple prior colorectal surgical procedures and pre-surgical biomarker measurement data from a pre-surgical sensing system 55502 or a wearable device 55506 indicating a probability of presence of chronic inflammation response. For example, the input from HCP 55507 may include a “medium risk” label when a patient's data record indicates a risk of surgical complications related to adhesions due to a history of multiple prior colorectal surgical procedures without any indication from pre-surgical biomarker measurement data that there is a probability of presence of chronic inflammation response. For example, the input from HCP 55507 may include label “low risk” a when a patient's data record indicates a risk of surgical complications related to adhesions due to a history of a single prior colorectal surgical procedure without any another indication of a probability of adhesion. The labels provided by HCP 55507 may be machine learning 55514 to train one or more models that may be used for patient analysis, modification and/or creation of surgical device control programs, and modification and/or creation of wearable control programs. The model may be stored at AI models 55517 and may be deployed at machine learning 55514, patient analysis 55518, surgical device control programs 55519, and/or warble control programs 55520.

Further, the input from HCP 55507 may include a notification level setting associated with each high-risk label, medium-risk label, or low-risk label. For example, a notification level setting may used by machine learning module 55514 to train an ML model to send a notification to HCP 55507 and/or HCP 55526 when the model may predict a high risk of surgical complication. In an example, a notification level may be used by the model when deployed at machine learning 55514 to send a notification to HCP 55507 when the model predicts a high risk of surgical complication. The HCP 55507 may respond to the notification with feedback, and the model may further be trained using the feedback. In an example, a notification level may be used by the model when deployed at patient analysis 55518 to send a notification to HCP 55526 and/or notifications 55527 when the model predicts a high risk of surgical complication. The HCP 55526 may respond to the notification with feedback, and the model may further be trained using the feedback.

The data preparation may also include the machine learning module 55514 receiving input from an HCP 55507 labeling a second subset of the data records in the dataset for validating an ML model (e.g., a validation dataset) with supervised machine learning.

Machine learning module 55514 may perform model training for the model. Machine learning module 55514 may perform model validation with the validation dataset after model may be deem trained (e.g., when a neutral network-based model's cost function has reached a global minimum).

Upon completing model validation, the machine learning module 55514 may perform model testing using a third subset of the data records in the dataset (e.g., an unlabeled dataset) for testing an ML model. The machine learning module 55514 may send predictions produced by the model to HCP 55507 for verification and/or HCP 55526. For example, the model may predict a high risk of surgical complication and an associated notification level of high-risk surgical complications. The machine learning module 55514 may send the high-risk prediction, notification level of high risk only, and decision points that may have led to such prediction (e.g., from the model 55517 trained with a decision tree machine learning algorithm).

In an example, based on the training dataset labeled by the HCP 55507, the model that may have been trained with a decision tree machine learning algorithm may learn a pattern (e.g., among other patterns) that a high-risk level of surgical complications may correlate with the combination of three or more prior colorectal surgical procedures and pre-surgical measurement data for at least one biomarker associated with a probability of chronic inflammation response (e.g., a high skin conductance level, a low tissue oxygenation level, and the like). Such a pattern may be a decision point in a decision tree algorithm-based model. The machine learning module 55514 may send a decision point along with the high-risk prediction and the notification level of high risk to the HCP 55507 and/or HCP 55526. machine learning module 55514 may send a decision point along with the high-risk prediction and the notification level of high risk to notifications 55527. In response, the HCP 55507 and/or HCP 55526 may provide a response verifying that the prediction is accurate. The verification may contribute to a success metric for meeting an accuracy parameter for deploying the model in a production environment (e.g. may be used on patients without supervision). A response from the HCP 55507 and/or HCP 55526 indicating the predication may be inaccurate may contribute to a failure metric for preventing model deployment due to inaccurate model predictions (e.g. may be used on patients with supervision).

The machine learning module 55514 may output the decision tree from the model. For example, the decision tree may be stored in AI models 55517. The decision tree may be sent to HCP 55507 and/or HCP 55526 to allow the decision tree to be verified holistically as opposed to one predication at a time.

Upon successful model testing with the test dataset, the computing system 55500 may deploy the model to a production environment production. For example, the model may be deployed to machine learning 55514, machine learning 55515, machine learning 55521, and patient analysis 55518. The deployed model 55517 may be further improved (e.g., for patient analysis purposes) in production. For example, patient analysis 55518, machine learning 55514, and/or machine learning 55515 may use feedback from a HCP 55508 to improve the model.

For example, the model may produce false negative predictions and/or false positive predictions. Feedback for such false negative and/or false positive predictions may be sent to the machine learning module 55521. In an example, the model may incorrectly predict a high risk of surgical complication. When the machine learning module 55521 sends an associated notification 55527, which may be sent to HCP 55526, the HCP 55526 may provide a response to the machine learning module 55521 indicating the prediction is a false positive. In such case, the machine learning module 55521 may not update the model threshold for predicting a positive prediction for surgical complications. The model may be stored and/or updated in AI models 55517 such that another deployment of the model may benefit from the feedback improvements.

In an example, the model may incorrectly predict no risk of a surgical complications (e.g., conflict data). The machine learning module 55521 may fail to send a notification, such as notifications 55527, to HCP 55526. HCP 55526 may not be provided with an opportunity to provide feedback. In such a case, the machine learning module 55521 may detect the error by checking the model prediction against the surgical outcome data from surgical system 55511 (e.g., which may be a surgical hub). The machine learning module 55521 may lower the model threshold for predicting a positive prediction for surgical complications to reduce the possibility of predicting false negatives. The model may be stored and/or updated in AI models 55517 such that another deployment of the model may benefit from the feedback improvement and/or detection of the error.

The machine learning module 55515 may perform data preparation as described herein with the surgical data collection 55529 (e.g., a dataset) for creating and training an ML model, which may be a surgical device control program model. The model may be stored and/or deployed at AI models 55517. The model may be deployed at machine learning 55515, machine learning 55521, and/or surgical device control programs 55519. In an example, the data preparation may further include creating a data field and appending it to a (e.g., each) data record in the dataset. The data field may indicate whether there was a surgical complication during a respective surgical procedure derived from surgical data from collected from a surgical system 55511 (e.g., a surgical hub).

The data field may indicate whether an operation of a surgical device or a wearable device may be improved (e.g. the device may have sub-optimally operated). The data field may serve as a desired output label for training the model with supervised machine learning for improving an ML model and/or a surgical device control program that may be determined and/or deployed at device control program 55519 to improve a surgical outcome. For example, the model may be deployed at surgical device control program 55519 and may be used to improve a surgical device program associated with surgical device 55522.

The machine learning module 55515 may perform model training, model validation, model testing for an artificial intelligence algorithm that may be used to create an ML model, such as a decision tree algorithm model. Those of skill in the art will recognize any other suitable machine learning algorithm may be used to build the model. The model may learn a pattern (e.g., among other patterns) that a surgical complication (e.g., a bleeding complication) occurs when a first condition and a second condition occur. The first condition may be that data from surgical sensing system 55509 and/or wearable device 55510 indicates at least one of: heart rate elevated above a threshold A, blood pressure above a threshold B, blood pH below a threshold C, or an edema measurement above a threshold D. The second condition may be that a control program associated with surgical device 55522 (e.g., a linear stapler) may be configured to compress tissue with a compression force below a threshold E. The model may learn another pattern (e.g., among other patterns) that a surgical complication (e.g., a bleeding complication) does not occur when the first condition and the second condition occur. A third condition may be that a control program associated with surgical device 55522 (e.g., a linear stapler) may be configured to compress tissue with a compression force above the threshold E.

Upon model testing using a test dataset, the computing system 55500 may deploy model to a production environment as a part of the machine learning module 55521. For example, the model may be deployed at machine learning 55514, machine learning 55515, machine learning 55521, AI models 55517, patient analysis 55518, surgical device control programs 55519, and/or wearable control programs 55520. During an operation in production, the model may detect a data pattern that the model may have learned during model training. For example, the model may receive input data indicating heart rate elevated above threshold A and indicating that a control program for surgical device 55522 is configured to apply a compression force below threshold E. In response, the model may predict a surgical complication and the machine learning module 55521 may update a deployed model, may update an ML model for generating a surgical device control program, may send updated parameters to the surgical device, or may send an updated surgical device control program to the surgical device to, for example, increase the compression force to be above threshold E.

The machine learning module 55515 may perform data preparation as described herein for creating and training an ML model, which may be an ML model for a wearable device such as wearable device 55510, using the pre-surgical data collection 55528 (e.g., a pre-surgical dataset) and the surgical data collection 55529 (e.g., a surgical dataset). In an example, the data preparation may further include creating a data field and appending it to a (e.g., each) data record in the dataset. The data field may indicate whether there may have been a surgical bleeding complication during a respective surgical procedure derived from surgical data from collected from a surgical system 55511 (e.g., a surgical hub). The data field may serve as a desired output label for training the model with supervised machine learning for adjusting a wearable control program, which may be stored at wearable control program 55520 and may be deployed at wearable device 55524, for improved sensed data relevancy.

For example, the machine learning module 55515 may perform model training, model validation, model testing for an ML model, such as a decision tree algorithm model. Those of skill in the art will recognize any other suitable machine learning algorithm may be used to build the model. The model may learn a pattern (e.g., among other patterns) that a surgical bleeding complication occurs (e.g., at a dissection/mobilization procedure step) when at least two conditions occur. One condition may be that pre-surgical data from pre-surgical sensing system 55502 and/or wearable device 55506 indicates at least one of: heart rate elevated above a threshold A, blood pressure above a threshold B, blood pH below a threshold C, or an edema measurement above a threshold D. Another condition may be that surgical data from surgical sensing system 55509 and/or wearable device 55510 indicates at least one of: heart rate elevated above a higher threshold A′ (e.g., as compared to threshold A), blood pressure above a higher threshold B′ (e.g., as compared to threshold B), blood pH below a lower threshold C′ (e.g., as compared to threshold C), or an edema measurement above a higher threshold D′ (e.g., as compared to threshold D).

The machine learning module 55515 may be configured to send update an ML model for a wearable control program. For example, machine learning module 55515 may update an ML model that may be deployed at machine learning 55514, machine learning 55515, machine learning 55521, wearable control programs 55520, wearable device 55506, wearable device 55510, wearable device 55524 and the like. The machine learning module 55515 may be configured to update a wearable control program that may be stored and/or deployed at wearable control programs 55520, wearable device 55506, wearable device 55510, and/or wearable device 55524. For example, an update may be sent to update the wearable control program of wearable device 55524 (e.g., configured for measuring heart rate, blood pressure, blood pH, and/or edema) when a surgical procedure (e.g., a sleeve gastrectomy procedure) is detected to have entered a dissection/mobilization procedure step. The update to the wearable control program may be to increase the data sampling rate (e.g., from once per minute to once per second). During the model operation (e.g., after model deployment) as a part of the machine learning module 55521 in production, such increased data sampling rate (e.g., during the dissection/mobilization) of biomarker measurement data related to bleeding complications may be sent to the HCP 55508 and/or HCP 55526 (e.g., via device 55525) to equip the HCP 55526 with more relevant data to prevent/mitigate potential bleeding complications.

Patient analysis 55518 may include software that may be used to provide analysis on a patient. For example, the analysis may indicate a probability of a surgical complication, a probability of surgical success, a diagnosis of a disease, a probability of a disease, a probability of a patient recovery, and the like. Patient analysis 55518 may include an ML model. The model may be stored outpatient analysis 55518 and/or deployed at patient analysis 55518. Patient analysis 55518 may include a number of models. For example, patient analysis 55518 may include one model for high blood pressure, a second model for normal blood pressure, and the third model for patients with diabetes. An ML model deployed at patient analysis 55518 may be from machine learning 55514, machine learning 55515, machine learning 55521, and/or AI models 55517. Patient analysis 55518 may include computational data.

Surgical device control programs 55519 may include software that may be used to provide control programs for surgical devices. Surgical device control programs 55519 may include device control programs, such as firmware, that may be stored for surgical devices. Surgical device control programs 55519 may include one or more parameters that may be used to configure, modify, operate, or control a surgical device. Surgical device control programs 55519 may include an ML model. For example, surgical device control programs 55519 may store an ML model that may be used for a surgical device, may deploy an ML model that may be used for a surgical device, or may update an ML model that may be used for a surgical device. An ML model deployed at surgical device control programs 55519 may be from machine learning 55514, machine learning 55515, machine learning 55521, and/or AI models 55517. Surgical device control programs 55519 may include computational data.

Wearable control programs 55520 may include software that may be used to provide control programs for wearable devices. Wearable control programs 55520 may include device control programs, such as firmware, that may be stored for wearable devices. Wearable control programs 55520 may include one or more parameters that may be used to configure, modify, operate, or control a wearable device. Wearable control programs 55520 may include an ML model. For example, wearable control programs 55520 may store an ML model that may be used for a wearable device, may deploy an ML model that may be used for a wearable device, or may update an ML model that may be used for a wearable device. An ML model deployed at wearable control programs 55520 may be from machine learning 55514, machine learning 55515, machine learning 55521, and/or AI models 55517. Wearable control programs 55520 may include computational data.

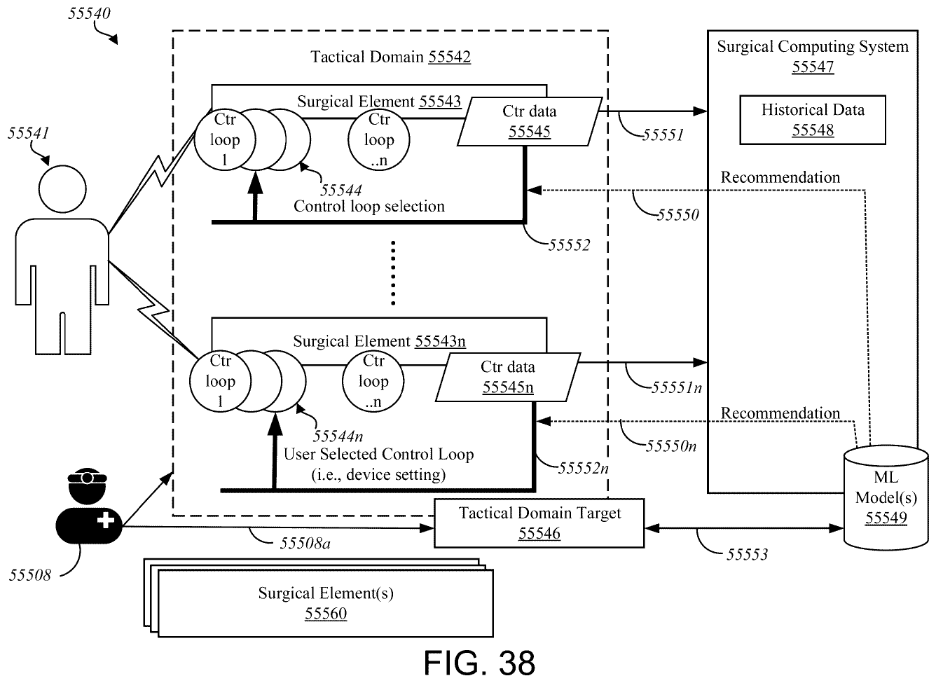

FIG. 38 is an example operational environment 55540 in which a surgical computing system 55547 may receive, among other things, control data 55545 (e.g., via 55551) and/or a tactical domain target 55546 (e.g., via 55553) to determine an optimized control loop 55544 for a surgical element 55543.

The operational environment 55540 may include a tactical domain 55542, a surgical computing system 55547, an HCP 55508, and/or a patient 55541 (although HCP 55508 is depicted in FIG. 38, HCP 55507, and/or 55526 of FIG. 37 may be interchangeably used). An operational environment 55540 (e.g., a tactical domain 55542) may infer one or more relationships between surgical elements 55543, 55543n, HCP 55508 a patient 55541, a tactical domain target 55546, and/or the like. The one or more relationships may be inferred based on for example, control data 55545, 55545n, historical data 55548, recommendation 55550, 55550n, and/or the like to create a tactical domain 55542 (also referred to hereinafter as tactical domain data). In examples, the operational environment 55540 may exclude one or more surgical elements 55560 from a tactical domain 55542 to optimize a procedure and/or efficiently process data.

A tactical domain 55542 may include tools, capabilities, data, and/or resources (e.g., HCP 55508, patient 55541) associated with a procedure. A tactical domain 55542 may include one or more surgical elements 55543, 55543n, configured to communicate with one another and/or with one or more components of an operational environment 55540 and/or a tactical domain target 55546 indicating a setpoint for the tactical domain 55542. Surgical elements 55543, 55543n, may include control loops 55544, 55544n, control data 55545, 55545n, and/or a selector 55552, 55552n.

A tactical domain 55542 may be generated and/or determined based on a ML model 55549 (e.g., of a surgical computing system 55547). A tactical domain 55542 may transmit control data 55545, 55545n (e.g., via 55551, 55551n) and/or a tactical domain target 55546 (e.g., via 55553) to a surgical computing system 55547. A tactical domain 55542 may receive a recommendation 55550, 55550n, associated with a portion of a procedure. The recommendation may be based on control data 55545, 55545n, a tactical domain target 55546, and/or the like. As an example, in response to a received recommendation 55550, a tactical domain 55542 may automatically adjust a control loop 55544 (e.g., via selector 55552) associated with a surgical element 55543, to optimize the performance of the surgical elements 55543, 55543n, and/or to efficiently perform a portion of a procedure (e.g., such that an HCP 55508 does not need to manually adjust the surgical element 55543).

A tactical domain 55542 may be determined and/or generated (e.g., by the surgical computing system 55547) based on one or more relationships associated with surgical elements 55543, 55543n (e.g., control loops 55544, 55544n, and/or control data 55545, 55545n), a surgical computing system 55547 (e.g., historical data 55548 and/or ML models 55549), an HCP 55508, a patient 55541, a tactical domain target 55546, and/or any other component of a operational environment 55540.

One or more relationships may be pre-defined (e.g., via a look-up-table as part of historical data 55548). In examples, a look-up-table may indicate a relationship between a primary surgical element 55543, a secondary surgical element 55543n, and/or a tactical domain target 55546 for a selected procedure. One or more relationships may be determined, for example, in response to a user input (e.g., a user input 55508a by an HCP 55508 via a graphical user interface (GUI), during a procedure and/or selected by an HCP 55508 and stored in historical data 55548 prior to a procedure). As an illustrative example, an HCP 55508 may indicate, in a look-up-table, that a core body temperature of a patient 55541 (e.g., a tactical domain target 55546) may be related to both a ventilator's change of a tidal volume (e.g., a primary surgical element 55543) and/or a heating blanket's power output (e.g., a secondary surgical element 55543a).

In examples, one or more relationships may be determined based on data (e.g., based on one or more components of a operational environment 55540 such as control data 55545, historical data 55548, a tactical domain target 55546, and/or the like). As an illustrative example, a surgical computing system 55547 may determine a relationship (e.g., based on an analysis of historical data 55548) between a core body temperature (e.g., a tactical domain target 55546), a ventilator (e.g., a primary surgical element 55543), and/or a heating blanket (e.g., a secondary surgical element 55543n). The surgical computing system 55547 may determine a relationship for example, based on data indicating that if ventilator increases the tidal volume of air to a patient, a heating blanket's power output may be increased to maintain the patient's core body temperature.

The tactical domain 55542 may receive a user input 55508a from an HCP 55508 (e.g., via 55508a). In examples, a user input 55508a may include an adjustment of a surgical element 55543 setpoint, a selection of a control loops 55544 associated with a surgical element 55543, a selection of a tactical domain target 55546, an indication of one or more steps associated with a procedure, the identification of one or more relationships (e.g., as described herein), and/or the selection, removal, and/or replacement of a surgical element 55543 (e.g., during a procedure). A user input 55508a may be received via a GUI, one or more control inputs (e.g., a knob, a button, and/or the like), and/or a connected device (e.g., a smartphone, wearable electronic device, a computer, and/or the like). In response to a received user input (e.g., an adjustment of a surgical element 55543), a surgical element 55543 may generate control data 55545 and/or a tactical domain target 55546. An adjustment to a surgical element 55543 may cause the control data 55545 and/or a tactical domain target 55546 to be transmitted (e.g., via 55551, 55551n, and/or 55553 respectively) to a surgical computing system 55547.

A surgical element 55543 may include one or more control loops 55544, control data 55545, and/or a selector 55552. Surgical elements 55543 may include (not illustrated in FIG. 38) sensors, instrumentation, and/or tools as described with reference to 20282 of FIG. 36. A surgical element 55543 may be used by an HCP 55508 and/or may operate autonomously to execute one or more actions associated with a procedure. As described herein, a surgical element 55543 may include, for example, robotic surgical systems, navigation systems, smart imaging systems, endoscopic and/or laparoscopic systems, energy scalpels, anesthesia machines, patient monitoring systems (pulse oximeters, blood pressure monitors, EKG monitors, EEG monitors, and/or the like), energy devices (e.g., electrosurgical units, laser surgery systems, and/or the like), infusion pumps, and/or the like. In examples, surgical elements 55543 may be wirelessly connected and/or physically connected (e.g., wired) to a network, a surgical computing system 55547, and/or one or more additional surgical elements 55543n. A surgical element 55543 may send control data 55545 to another computing system, to other surgical elements 55543n, and/or to a surgical computing system 55547 (e.g., via 55551) as described herein.

A tactical domain 55542 may include one or more control loops 55544 associated with a surgical element 55543. A control loop 55544 may change response characteristics of a surgical element 55543 (e.g., a behavior and/or an output characteristic of the surgical element 555343) based on an input from a patient 55541, an HCP 55508, a tactical domain target 55546, a recommendation 55550, 55550n, a network, other surgical elements 55543n, and/or a surgical computing system 55547. A control loop 55544 (e.g., an optimized control loop) may enable the surgical element 55543, an HCP 55508, and/or a patient 55541 to adapt, provide stability and control, improve precision and accuracy, improve consistency, and/or increase efficiency of an output characteristic of the surgical element 55543 during one or more portions of a procedure. As an illustrative example, an optimized control loop 55544 may monitor the force applied to a robotic instrument, and adjust the amount of force applied to prevent excess pressure that may damage tissue (e.g., a force-sensing instrument to measure a force applied during the membrane peeling process of a vitrectomy and membrane peeling).

Throughout a procedure, a tactical domain 55542 (e.g., via recommendation 55550, 55550n) may change and/or adjust an optimized control loop 55544 based on a recommendation 55550, 55550n (e.g., a one or more recommendations 55550 indicating one or more optimized control loops 55544 may be received during a procedure). For example, a recommendation 55550 may indicate a first control loop 55544 for a first portion of a procedure, and a second control loop 55544n for a second portion of a procedure. An optimized control loop 55544 may change, for example, based on a characteristics of a procedure, the patient 55541, the HCP 55508, the performance of a surgical element 55543, and/or the like (e.g., the length of a procedure, the number of complications associated with a procedure, the vulnerability of the patient, a specific hospital, a specific operating room, and/or the like). In examples, an optimized control loop 55544 may be different (e.g., response characteristics of the optimized control loop 55544 may be slower and/or faster) for a patient that is susceptible to hypothermia (e.g., an elderly patient, a pediatric patient, and/or the like) versus a patient that may be more resistant to hypothermia (e.g., a patient with a high body-mass index, patients with high basal metabolic rates, and/or the like).

A tactical domain 55542 may include control data 55545 associated with a surgical element 55543. Control data 55545 may include operational data and/or patient data (e.g., including pre-surgical data collection 55528, surgical data collection 55529, and/or computation data collection 55530 of FIG. 37). Operational data may include information associated with the surgical element 55543 during a procedure (e.g., an output characteristic of the surgical element 55543). For example, operational data may include the speed of a cutting tool, a selected control loop 55544 for a surgical element 55543, a voltage and/or current of an electrosurgical tool, a flow rate for an infusion pump and/or the like. Patient data may include real-time data associated with one or more physiological parameters of a patient during a procedure (e.g., a patient's heart rate, SpO2, temperature, and/or as described herein). Additionally and/or alternatively, control data 55545 may include historical data 55548 (not illustrated as part of surgical element 55543, 55543n), that may be associated with past operational data generated during the same and/or a similar procedure and/or patient data generated during the same and/or a similar procedure (e.g., a specific patient's data for one or more procedures and/or multiple patient's data for one or more procedures).

A tactical domain 55542 may include a tactical domain target 55546. A tactical domain target 55546 may be a setpoint and/or any other measured variable associated with the operational environment 55540 (e.g., associated with a surgical element 55543, a patient 55541, an HCP 55508, and/or the like). In examples, a tactical domain target 55546 may include one or more physiological parameters of a patient 55541 such as a core body temperature, a localized body temperature, oxygen saturation, blood pressure, respiratory rate, blood sugar, heart rate, hydration state, and/or the like. In examples, a tactical domain target 55546 may include a setpoint and/or one or more measured variables associated with a surgical element 55543 such as a voltage and/or current for an electrosurgical tools, flow rate for an infusion pump, power outputs for a laser device, concentration amount for an anesthesia machine, tidal volume for a ventilator, and/or the like. A tactical domain 55542 may receive a tactical domain target 55546 from an HCP 55508 (e.g., via 55508a) and/or from a surgical computing system 55547 (e.g., via 55553). A tactical domain 55542 may determine a tactical domain target 55546 based on one or more surgical elements 55543, 55543n. As an example, an HCP 55508 may select, as a tactical domain target 55546, a temperature setpoint to control the core body temperature of a patient during a procedure.

An operational environment 55540 may include a surgical computing system 55547. A surgical computing system 55547 may include historical data 55548, and/or ML models 55549. A surgical computing system 55547 may receive control data 55545, 55545n (e.g., via 55551, 55551n) from a surgical element 55543, 55543n, and/or a tactical domain target 55546 from a tactical domain 55542 (e.g., via 55553). A surgical computing system 55547 may determine, based on the received control data 55545, 55545n, historical data 55548, and/or a tactical domain target 55546, a recommendation 55550, 55550n for a surgical element 55543 and/or 55543n. A recommendation 55550 may be transmitted wirelessly and/or via a wired connection. A recommendation 55550 may be determined based on one or more ML models 55549 and/or based on one or more relationships as described herein. A recommendation 55550 may include for example, an indication of adjustment and/or a selection of an optimized control loop 55544 associated with a portion of a procedure, an indication of a surgical element 55543 to be used during a portion of a procedure, an indication of a surgical element 55560 to be excluded from a tactical domain 55542, and/or an indication of a conflict associated with one or more surgical elements 55543, 55543n, during a portion of a procedure.

A surgical computing system 55547 may include historical data 55548. Historical data 55548 may include information associated with past recommendations, historical control data from one or more surgical elements 55543, 55543n, data associated with an HCP 55508 (e.g., procedural time, specific surgical elements used by an HCP 55508 during a procedure, and/or the like), historical data 55548 associated with a patient's physiological parameters (e.g., biomarkers), multiple patients physiological parameters associated with a procedure, and/or the like (e.g., including pre-surgical data collection 55528, surgical data collection 55529, and/or computation data collection 55530 of FIG. 37).

A surgical computing system 55547 may include ML models 55549. An ML model 55549 may be trained to achieve a tactical domain target 55546 by generating a recommendation 55550 (e.g., including a selection of an optimized control loop 55544) as described herein. ML model 55549 may generate individual training data items that form training data. Training data items may correspond to a particular portion of a procedure, include historical data 55548, and/or the like. An ML model 55549 may train an ML model to generate and/or determine an output based on control data 55545, based on a portion of a procedure, based on a tactical domain target 55546, and/or the like.

In examples, an ML model 55549 may be trained to predict an optimized control loop 55544, 55544n for one or more surgical elements 55543, 55543n. The optimized control loop 55544 may be determined, such that a tactical domain 55542 achieves a tactical domain target 55546 in an efficient, safe, controlled, and/or economical manner (e.g., during a procedure). The determined optimized control loop 55544 may be sent to a tactical domain 55542 via recommendation 55550, where the tactical domain 55542 (e.g., the surgical element 55543) may automatically adjust a control loop 55544 via selector 55552.

In examples, an ML model 55549 may be trained to determine an optimized surgical element 55543. The optimal surgical element 55543 may be determined for a tactical domain 55542 and/or for use during a portion of a procedure. The recommendation 55550 may be sent to the tactical domain 55542, where the tactical domain may automatically include and/or remove one or more surgical elements 55543, 55543n, and/or 55560 from the tactical domain 55542. In examples, the recommendation 55550 may be sent to an HCP 55508 (e.g., via a GUI), where the HCP 55508 may determine whether to include and/or remove a surgical element 55543 based on the recommendation 55550. As an illustrative example, an ML model 55549 may determine that a specific cutting tool (e.g., a specific serial number) is the most reliable tool from a list of similar cutting tools, that a type (make and/or model) of ventilator provides optimized control during a procedure, and/or the like, and send an indication of the reliable tool, via a GUI, to an HCP 55508.

In examples, an ML model 55549 may be trained to anticipate conflicts between surgical elements 55543, 55543n, and/or the like. An anticipated conflict may be determined for a tactical domain 55542 and/or for portion of a procedure. The recommendation 55550 may be sent to the tactical domain 55542, where the one or more surgical elements 55543, 55543n may resolve the conflict based on the recommendation 55550, 55550n. In examples, an ML model 55549 may provide a recommendation 55550, 55550n to avoid contact between one or more movable arms of a robotic system during a procedure. In examples, the recommendation 55550 may be sent to an HCP 55508 (e.g., via a GUI) where the HCP 55508 may determine how to resolve the conflict, and/or validate whether the conflict actually exists.

Identifying trends associated with ML algorithms and/or ML enhancement of data or displays may be described herein. In examples, the description herein may be used to generate historical data 55548, and/or may be used as training data for a ML models 55549.

Separation of data into known buckets to enable an AI/ML model may be provided. AI/ML identification of visualized shapes may be described herein. Real-time removal of tissues, instrument or organs based on ML identification of the boundaries of the shapes and/or re-insertion of a missing environment may be provided. A real-time projection or by adding a virtual image over real-time imaging based on an ML adaptation of the perspective may be provided. In examples, overlaying an image may provide an ability to separate data into known buckets to enable and support AI/ML—automatic annotation (e.g., a system may label images such that they may be queried by a user). Known buckets can include HCP labels, contextual real-time tagging, boundary flagging around activations, and/or multi-variable flagging.

In examples, an HCP 55508 (e.g., a surgeon) may label one or more images. The system 55547 may utilize surgeon labeled images as a method for bypassing system-driven annotation. HCP 55508 annotation may allow the system 55547 to take advantage of surgeons who prefer to manually annotate images, video, and/o the like.

Contextual real-time tagging may be provided, which may include procedure step integration and/or operating room equipment integration (e.g., surgical element 55543). Procedure step integration may include a camera or system capturing a data (e.g., a video or images). The data may be integrated in with the system 55547 (e.g., a procedural planning system), such that the data (e.g., an image or video) is tagged with metadata relating to the current step or other metrics in real-time as opposed to post-processed. Operating room equipment integration may include a system tagging and/or annotating the data (e.g., video and/or images) with data to show when energy activations occur, and other discrete events within the operating room.

Boundary flagging around activations may be provided herein. In examples, a decoupling of the analysis may allow the ML algorithms (e.g., ML models 55549) to be more specific or constrained. Decoupling may reduce the complexity of ML algorithms (e.g., ML models 55549) and execution time of them.

Multi-variable flagging may be described herein. In examples, the system 55547 may generate flags based on captured data (e.g., video or images). The system 555547 may compile the flags, to detect (e.g., at a later point in time) the contextual annotation. In examples, the system 55547 may detect how bright an image is, clarity of the image, amount of red in the image, instrument detection, and/or the like. The flags may contribute to understanding of the context long term.

Multi-variable flagging may include the identification of liver photons. As an illustrative example, protons versus photons for unresectable hepatocellular carcinoma may be provided. Liver decompensation and overall survival results may include a median follow-up of 14 months. Of 133 patients with a median age of 68 years and/or 75% male, 49 (37%) were treated with proton radiation therapy. Proton radiation therapy may be associated with improved results (adjusted hazard ratio, 0.47; P=0.008; 95% confidence interval [CI], 0.27-0.82). The median results for proton and photon patients was 31 and 14 months, respectively, and the 24-month results for proton and photon patients was 59.1% and 28.6%, respectively. Proton radiation therapy was also associated with a decreased risk of non-classic radiation-induced liver disease (odds ratio, 0.26; P=0.03; 95% Cl, 0.08-0.86). Development of non-classic RILD at 3 months was associated with the worse results (adjusted hazard ratio, 3.83; P<0.001; 95% Cl, 2.12-6.92). There may be no difference in locoregional recurrence, including local failure, between protons and photons.

In examples, a system 55547 may improve data flow and/or storage via network traffic analysis and/or control, adjusting storage rate and/or location, selective inter connectivity, and/or by being situationally dependent. In examples, a system 55547 may shift between real data collection and post-op collection of an autonomous systems to enable collection of more important things (i.e. a surgical element may transmit wirelessly real time or the surgical element may be asked at the end of the procedure to download its dataset).

In examples, quasi-synthetic data may include data that is generally accurate, and/or may execute a function to make the data accurate (e.g., completely right). In some examples, quasi-synthetic data may be generated when/if there is not enough data and/or sensor information present (e.g., a system 55547 may revert to an ML model 555549 for a next action during a procedure). In examples, one or more sensors may be available, and/or the system 55547 may use and ML model 55549 to inform the system and/or an HCP 55508 of a determination for an efficient and/or successful outcome. In some examples, a subjective choice may include synthetic data (e.g., data that is invented/created by something else).

A transfer algorithm adaptation outside real-time may be provided. Solving one or more equations that may be needed and/or introducing equations to the system may be provided herein. Developing an AI/ML model, and/or how one or more unknowns are solved (e.g., based on settings and/or shifting thresholds) may be provided.

For example, patterns in results that a user may select for tactical decisions (e.g., a tactical domain target 55546) for assistance in future decision making may be described herein. Collection of user choices when requested (e.g., required) for automated decision making with resulting outcomes, from multiple surgeries, to provide weighted suggestions for future decisions may be provided. Aggregation of user derived decisions from one or more data streams and the outcomes from patients to form suggestions for future user choices may be provided. The more data streams than closed loop control systems (tactical decisions below) which have more than one choice and were presented to the user for input may be provided. The user may be presented with information including a display of multiple data streams and how the multiple data streams interact (e.g., one or more relationships). The display may present the proposed improvements to the operational algorithms (e.g., a recommendation 55550, 55550n).

ML and/or trending aggregation may be provided herein to obtain strategic decisions. Long term adaptations of the control or display systems via large dataset ML aggregation or trending may be provided herein. The AI/ML model (e.g., ML model 55549) may be acting on a current procedure, and/or on one or more procedures that occur in the future. In examples, there may be instances where ML model 55549 would use data from a current procedure versus from a procedures of a specific type. For example, a sleeve standard of care may include an offset for staple lines of about 20 degrees, to follow greater curvature of stomach. After placing lines, the actual angle of offset may be 10-40 degrees. Surgeries where the actual angle is 25-35 may have the fewest complication instances. As a result, a feedback loop (after a threshold number of procedures) may recommend a 30 deg offset. The placement of staple lines at 30 degrees may be a strategic decision.

Non-real time aggregation pattern identification may be described herein. Adjustments to a procedure recommendation, based on ML datasets from patient data and/or surgeon data may inform a behavior that differs from the standard response (e.g., included as part of historical data 55548).

Data sets may be used to make a strategic decision. A strategic decision may be different than a tactical decision. In examples, comparison of real-time data resulting in a tactical decision over time may be used to make strategic decisions within a single surgery and/or between surgeries. In examples, a tactical decision may come from real time data during a procedure. For example, patient monitoring (a ventilator), the pressure inside tumor/chemo tip, a cone beam CT (e.g., a tumor volume filled), a chemo pump (e.g., drug volume delivered). In examples, the pressure rising within tumor may indicate that a drug delivery may be stopped to prevent spray out.

A system 55547 may compare real time data to a broader intra-surgery database (e.g., historical data 55548). A database may exist. Data may be generated based on a surgeon, one or more procedures (e.g., all procedures) from a piece of equipment, and/or as described herein. In examples, the pressure rising within tumor may indicate that an HCP 55508 may, instead of repositioning probe, ablate instead of delivering chemo (e.g., data from previous procedures informs spray out is bad, and a system may indicate a decision to ablate instead of deliver chemo). In examples, a tumor may have be less homogenous than initially estimated. A system 55547 may generate a new procedure plan based on new information from a patient 55541. In examples, a patient 55541 with a history of cancer may have a limit to an amount of chemo administered. Regardless of tumor saturation, a decision to stop chemo once a threshold volume is achieved may be indicated by the system 55547.

A system 55547 may utilize trends or patterns within and/or between data streams, procedures, patients 55541, and/or physicians to generate strategic decisions (e.g., recommendation 55550). In examples, raw contextual data (e.g., historical data 55548 as described herein) could be used to identify patterns within the data streams, to find coupled feeds that enable the prediction of one feed by the leading feed of another stream. Data may indicate that a current treatment approach is not the best approach (e.g., as a result an HCP 55508 may should change an approach). In examples, an HCP 55508 may have an approach angle and/or a tumor approach direction that may have an implication on a tumor fill. An approach angle may determine whether a procedure is endoscopic and/or trans-parenchymal. In examples, an HCP 55508 may change from irradiation to cryo-ablation due to an issue with chemo delivery (e.g., patient outcomes are the same but a is lower, patient outcomes are the same but a procedure is lower risk, and/or patient outcomes are the same but a surgeon effort is less). In examples, a needle depth and/or drug pressure may be related (e.g., the needle moves beyond 60% depth, a spray out may occur). In examples, a strategic decision (e.g., a recommendation 55550) may include inserting the needle depth to about 30% depth.

There may be a decision that is not based on previous sets of AI/ML data. Previous data set may include strategic implications on one or more steps, procedures, placements of surgical instruments, and/or the like, that may improve surgical outcomes. In examples, where trocars and/or equipment may be placed for efficient use may be determined by AI/ML models. In examples, previous procedure steps may define one or more constraints for a stomach tumor procedure. For example, a tumor may be removed from peritoneum, and/or the amount of a tumor dissected may define an effort requested to remove a segment. In examples, an angle of a sleeve gastrectomy line may be defined where the next staple line will be placed. The system may generate an indication to an HCP 55508, providing a range that the next staple line may be placed at. In examples, a system 55547 may determine, for a tissue tension on a staple line, the amount of mobilization performed to define acceptability of a circular line. In examples, for geometry and/or anatomy, a trocar placement may be based on one or more adhesions. For examples, a trocar placement may be based on a tumor size, a number of needles, one or more feedback loops to fine-tune practice guidelines (e.g., control loops 55544), and/or the like.

Continuous improvement of the ML algorithm based on new incoming data and verification of an AI/ML algorithm (e.g., ML model 55549) may be provided herein. In examples, an HCP 55508 may determine whether to use a first type of energy device or a second type of energy device to clamp and/or fire onto tissue. An algorithm may suggest an approach (e.g., a smaller versus a larger effect on tissue). Data from one or more data streams (e.g., surgical elements 55543) may identify whether the first type of energy device or the second type of energy device is better than an AI/ML preferred technique. In examples, an algorithm may learn based on accepted metrics (e.g., less bleeding, quicker recovery, and/or the like). In examples, a skill assessment of an HCP 55508 (e.g., a surgeon) may be generated (e.g., as part of historical data) and/or compared to provide procedure improvement suggestions.

In examples, a system may examine the trends of the previous procedures (e.g., historical data 55548), looking for patterns in the HCP (e.g., surgeon) techniques, selections of tools and access, anatomic landmark alignment, patient characteristics, patient biomarker reading (e.g., BMI, blood pressure, blood sugar, height, weight, stature, and/or the like) and the outcomes and complication from the previous surgeries, to present the surgeon with a viable choice in real-time and/or to provide context or potential implications of a choice to aid an HCP in their current choice.

A determination of a sleeve pouch size and/or an initiation location of a sleeve may be provided. In sleeve gastrectomy the size, shape, and/or other aspects of the pouch may relate to a patient's outcome, the difficulties of performing the procedure, and/or the probability of post operative complications. If the real-time data streams (e.g., via surgical elements 55543 and/or the like) provide multiple choices for the surgeon for a specific patient being scanned. Results and issues associated with previous surgeries (e.g., either similar patients and/or one or more patients for an HCP, for a surgical center, and/or for a geographic region) may be used to provide the HCP (e.g., the surgeon) with context, predictions, and/or suggestions as described herein. Real-time data streams may relate to the particular constraints of a patient, the approach, the skill level of the HCP, aspects of the general population, techniques, and/or instruments being used. In examples, one or more contributing factors (e.g., criteria that may be included in historical data 55548) may include, for a specific patient and/or for past patients, a stomach thickness, medications, a sleeve starting location from the pyloric sphincter, co-mobilities, a number of intersections and/or an angle size, a pouch size, a boogie size, and/or a uniformity of sleeve size (e.g., the sleeves may be uniform, and/or close to the esophageal sphincter as one moves parallel to the lesser curve, and/or the staple lines around the distance from the incisura-angularis may make the sleeve less than uniform in the distal portion of the sleeve, which may not be desirable), and/or buttress or not. In examples, one or more outcomes may include a durability or weight loss, post operating complications, a time, and/or an impact on patient recovery time.

AI/ML models (e.g., ML models 55549) may have different levels. In examples, ML may include large dataset with limited bounding. In examples an AI/ML model may determine how to parse data. In examples, AI/ML model observes a procedure, a behavior, and/or may preemptively make a change/decision. In examples, a surgical element 55543 may perform a behavior multiple (e.g., 18) times, the AI/ML model may indicate, to the system 55547 (e.g., indicate to the tactical domain 55542 via recommendation 55550) to set up differently based on the behavior. In examples, AI/ML models may include patterns. For an AI/ML model to generate an output (e.g., a recommendation 55550) in real time, verification and validation of an updated model must be achievable against a prior defined data set. In examples, an AI/ML model may be assessed based on new data and/or preexisting data. For example, as an HCP 55508 (e.g., a surgeon) makes decisions, the contextualized data may be incorporated into a dataset from the HCP 55508. This data may be used in training data (e.g., as described herein) to update an AI/ML model. An ML model 55549 may be verified against historical data 55548 which has been previously confirmed. As a result, this ensures that the ML model 55549 continues to meet the verification burden that the model was previously established with, but provides the model flexibility to continue to adapt.

Real time aggregation of HCP 55508 decisions (e.g., surgeon decisions) into a data set for model training and/or verification may be provided (e.g., as part of historical data 55548). In examples, as an HCP 55508 (e.g., a surgeon) makes decisions, contextualized data is incorporated into a dataset from the HCP 55508. This data may be assumed to have a valid output associated with it based on the HCP 55508 decisions. As a result, the data may be utilized in training, updating, or verifying an existing model based on a surgical outcome. As the quantity of surgical decisions becomes large enough, the model may begin to self-train and/or verify itself within its given bounds.

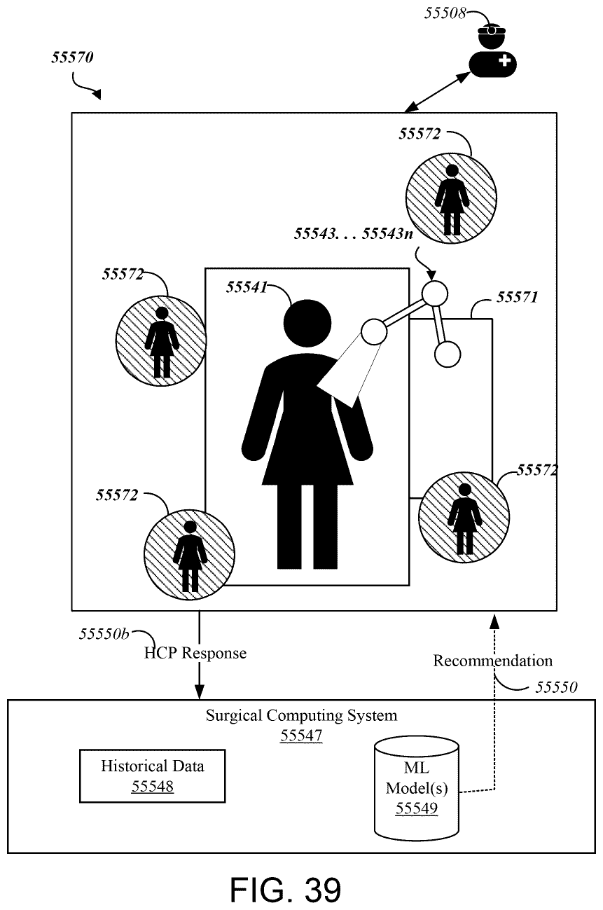

FIG. 39 illustrates an example user interface display 55570 indicating one or more conflicts as determined by a surgical computing system 55547 of FIG. 38. The display 55570 may be provided to an HCP 55508. The display 55570 may notify an HCP 55508 of potential a conflict and/or request that the HCP 55508 resolve a potential conflict associated with a procedure. In some examples, the display 55570 may be a heat map indicating one or more potential conflicts.

The example display 55570 may include a patient 55541, a robotic system 55571, and/or one or more potential conflicts zones 55572. The example display 55570 may be representative of an endoscopic procedure and/or any other procedure, where a robotic system 55571 may be equipped with one or more surgical elements 55543, 55543n (e.g., a camera, a suction tool, a robotic arm, and/or the like).

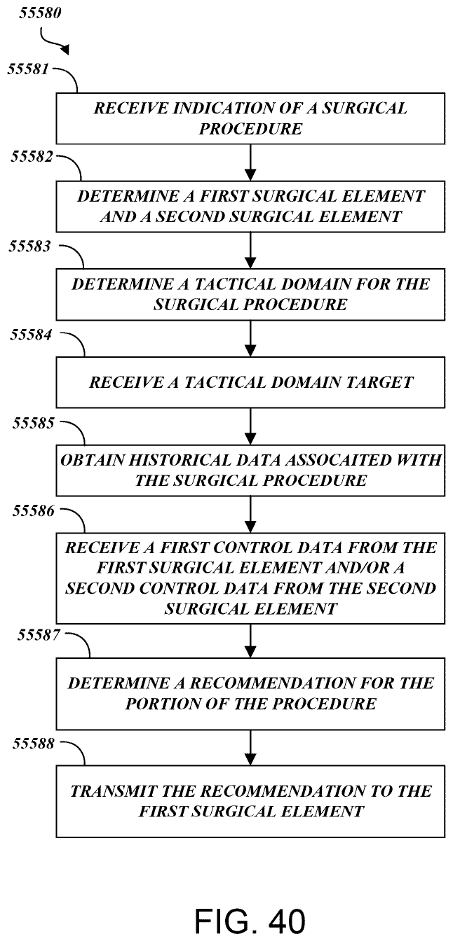

The display 55570 may include one or more conflict zones 55572. The conflict zones 55572 may be as part of a recommendation 55550 generated with reference to block 55587 of FIG. 40. Conflict zones 55572 may indicate a physical conflict area within an operating room, where an HCP 55508 may interfere with a robotic system 55571 during a portion of a procedure and/or where one or more surgical elements 55543, 55543n, may interfere with one another (e.g., if the HCP 55508 were to enter the conflict zone 55572). The conflict zones 55572 may be determined based on historical data, control data, and/or operational data as described herein. In examples, historical and/or control data may include the position, speed, and/or attitudes of surgical elements 55543, 55543n, and/or the movement of HCPs 55508, and/or a patient 55541 over time, during past procedures and/or during a current procedure.

The number and/or position of conflict zones 55572 may be modified based on one or more conditions of a patient 55541, an HCP 555508, surgical elements 55543, 55543n, and/or the like. For example, as a procedure progresses, an ML model 55549 may determine that a location may no longer be a conflict zone 55572 and remove the indication of a conflict zone 55572 from the display 55570. In examples, the display 55570 may receive a user input from an HCP 55508, confirming the validity of one or more conflict zones 55572, and/or acknowledging the existence of a conflict zone 55572. In response to a user input (e.g., via HCP response 55550b), the surgical computing system 55547 may further train an ML model 55549 based on the validity of one or more conflict zones 55572 for a procedure.

As described with reference to FIG. 39, an intra-procedure collision and/or position tracking may be utilized in an ML model (e.g., ML models 55549). In examples, an intra-procedure collision and/or position tracking method (e.g., as provided in display 55570) may define a context and data-focused path planning. As operating rooms become more connected and data centric, data may be collected regarding robotic system 55571 positions, speeds, device attitudes, movement of personnel, and/or knowledge of relevant locations, techniques, and outcomes for each step in the procedure. Data may be used to define a temporospatial heatmap of robotic positions (e.g., as illustrated in display 55570) to show common locations for the robot as they relate to a given procedure and approach.

Common and/or expected robotic locations (e.g., conflict zones 55572) at each given point in time along the course of a procedure may receive a positive numerical offset to define the given point as desirable or an usual position. Positions over time where the robot encounters sensitive and/or irrelevant organ structures, or is close to collision within or outside of the body (e.g., conflict zones 55572) may receive a negative numerical offset (e.g., to define an undesirable position). An outcome of the procedure and/or specific actions therein may be considered to add a multiplicative weighting value to appropriate positions in time (e.g., as part of ML models 55549). In examples, if an operating location on a gallstone removal results in a good outcome such as reduced bleeding (e.g., relative to expected) or good post procedure healing outcomes, the operating location may receive a positive multiplier to indicate that the position and time location were of unique advantage to procedural outcomes. If the operating location is associated with a negative outcome (e.g., like excessive bleeding), a fractional or negative multiplier may be added to reduce the desirability (e.g., fractional) of the operating location in time and/or make the operating location entirely undesirable (e.g., a negative outcome as depicted in conflict zones 55572).

An operating position in time and space (e.g., throughout a procedure) may include a numerical offset (e.g., +6, −2) that may define desirability of operating and/or moving the robotic system 55571 or end effector into that operating position based on frequency or knowledge of a dangerous or uncommon situation. A position may or may not receive a weighting value (a multiplier, i.e. ×0.5 or ×−1.0) that corresponds to patient health outcome of an action performed or related to that position in time. A position may be multiplied by the numerical offset in order to determine a gross overall desirability score for the heatmap (e.g., as provided in display 55570).

Due to the numerical nature of the heatmap (e.g., display 55570), multiple heatmaps may be overlaid via simple addition and/or overlay. Additional robotic systems 55571 in the operating room may increase the number of conflicting robot's desired positions (e.g., conflict zones 55572) and/or may provide a negative offset (e.g., as the likely presence of an additional robotic system 55571 in that space in time may result in possible conflicts and thus a lesser desirability assigned to that position). Multiple heatmaps may be summed to account for multiple robotic paths and/or obstacles or unique considerations within the operating room. The sum of heatmaps may be a net temporospatial heatmap for the robot in question (e.g., as provided in display 55570). Net heatmaps may be overlaid within the HCP's console (e.g., surgeon console) for the benefit of the HCP 55508 to provide additional information or to directly guide the motion of the robot or path plan.

In examples, a system (e.g., surgical computing system 55547) may annotate the number of actual conflicts compared to system predicted number. The system 55547 may predict an anticipated number of conflicts and/or detect a conflict in a procedure. The system 55547 may interrogate an HCP 55508 regarding the magnitude and occurrence of a conflict. The result may be saved in historical data 55548, including the actual and/or predicted or anticipated outcomes to adapt an AI/ML algorithm (ML models 55549) for an optimized prediction.

In examples, a system 55547 may provide a GUI (e.g., display 55570) to an HCP (e.g., a surgeon or other surgical staff) with a sequence of predicted conflicts (e.g., conflict zones 55572). The system 55547 may request that the HCP 55508 determine whether or not the one or more predicted conflicts 55572 occurred, input a number and/or type of actual conflicts, and/or input an adjustment to one or more conflicts to better inform the algorithm in future (e.g. distributed learning). The system 55547 may receive the HCP 55508 input via HCP response 55550b, including patient outcomes to improve predictions of conflicts. In examples, cameras in an operating room may observe and/or respond to a conflict instead of a user (e.g., an HCP 55508).

Adaptation of post-surgical historical data 55548 to reflect new anatomic configurations may be provided herein. In examples, historical data 55548 may include a post-surgical removal of tissues to fit the patient, procedure, outcome with the intervention integrated in the data. In examples pre-op information may be altered once an object is removed by the HCP 55508 (e.g., a surgeon). In examples, (e.g., the removal of gallstones) there may be an instance where an HCP (e.g., the surgeon) overlays a pre-op CT over a real time scope view of the patient. An HCP 55508 may remove the gall bladder via the connection to the common bile duct. Once the gall bladder is removed, the HCP 55508 may return to the bile duct and small intestine to remove remaining stones that were not extracted. If the HCP 55508 is interested in a CT overlay of the gall bladder, the gall bladder may be removed in the CT display so that the system 55547 displays relevant anatomy.

Smart system decisions on subjective interpretation using one or more linked smart data streams may be provided herein. Trends or patterns that may enable smart decision may improve stone identification thru external CT and/or ultrasound. A system may select a main threshold for a stone versus a stone in a gallstone identification. The system 55547 may handle indeterminate shapes and/or probabilities by an image including a dotted outline with the probabilities by identifying and coupling landmarks from multiple images and/or align or distort of the images based on the coupling.

FIG. 40 is a flow chart of an example optimized control loop selection routine 55580 illustratively implemented by a surgical computing system 55547 of FIG. 38. The routine 55580 begins at block 55581.

At block 55581, the surgical computing system 55547 may receive an indication of a surgical procedure. An indication of a surgical procedure may be received from an HCP 55508, a tactical domain 55542, and/or a surgical element (e.g., surgical element 55543 of FIG. 38). In examples an indication of a surgical procedure may be determined by the surgical computing system 55547. An indication of a surgical procedure may include, for example, a specific step, a sub-step, an operation, an act, and/or the like, for a surgical procedure, diagnostic procedure, therapeutic procedure, preventative procedure and/or the like. As a specific example, an indication of a surgical procedure may include administering anesthetics to sedate a patient during an endoscopic procedure and/or preparing an area of a patient's body for an incision.

At block 55582, the surgical computing system 55547 may determine a first surgical element and a second surgical element (e.g., surgical element 55543, 55543n of FIG. 38). The surgical computing system 55547 may determine the first surgical element and/or the second surgical element based on the received indication of a surgical procedure. The surgical computing system 55547 may determine the first and/or second surgical element based on an indication of one or more surgical elements from an HCP 55508, another network, and/or the like.

At block 55583, the surgical computing system 55547 may determine a tactical domain 55542 for the surgical procedure. As described herein, a tactical domain 55542 may include tools, capabilities, and/or resources to conduct a portion of a procedure. A tactical domain 55542 may include a network of surgical elements 55543, 55543n configured to communicate with each other and/or with one or more components of an operational environment 55540. For example, a tactical domain 55542 may include surgical elements 55543, 55543n, control loops 55544, 55544n, control data 55545, 55545n, a tactical domain target 55546, and/or a selector 55552, 55552n of the one or more control loops 55544, 55544n associated with a portion of a procedure. A tactical domain 55542 may be derived (e.g., generated) based on an ML model 55549 (e.g., of a surgical computing system 55547), and/or determined by an HCP 55508.

As described herein, a tactical domain 55542 may be determined and/or generated (e.g., by the surgical computing system 55547) based on one or more relationships associated with surgical elements 55543, 55543n (e.g., control loops 55544, 55544n, and/or control data 55545, 55545n), a surgical computing system 55547 (e.g., historical data 55548 and/or ML models 55549), an HCP 55508, a patient 55541, a tactical domain target 55546, and/or any other component of an operational environment 55540.

As described herein, one or more relationships may be pre-defined (e.g., via a look-up-table as part of historical data 55548). In examples, a look-up-table may indicate a relationship between a primary surgical element 55543, a secondary surgical element 55543n, and/or a tactical domain target 55546 for a selected procedure. One or more relationships may be determined, for example, in response to a user input (e.g., a user input 55508a by an HCP 55508 via a graphical user interface (GUI), during a procedure and/or selected by an HCP 55508 and stored in historical data 55548 prior to a procedure).

At block 55584, the surgical computing system 55547 may receive a tactical domain target 55546. A tactical domain target 55546 may be determined by a tactical domain 55542, an HCP 55508, and/or generated by the surgical computing system 55547. As described herein, a tactical domain target 55546 may be a setpoint and/or any other measured variable associated with one or more surgical elements 55543 and/or a patient 55541. In examples, a tactical domain target 55546 may include one or more physiological parameters of a patient 55541, such as a core body temperature, a localized body temperature, oxygen saturation, blood pressure, respiratory rate, blood sugar, heart rate, hydration state, and/or the like. In examples, a tactical domain target 55546 may include a setpoint and/or one or more measured variables associated with surgical elements 55543 such as a voltage and/or current for an electrosurgical tool, flow rate for an infusion pump, power outputs for a laser device, concentration amount for an anesthesia machine, tidal volume for a ventilator, and/or the like.

At block 55585, the surgical computing system 55547 may obtain historical data 55548 associated with the surgical procedure. The historical data 55548 may include past recommendations, historical control data from one or more surgical elements 55543, data associated with an HCP 55508 (e.g., procedural time, a surgical element 55543 used by an HCP 55508 during a procedure, and/or the like), historical data associated with a patient's physiological parameters (e.g., biomarkers) and/or multiple patients physiological parameters associated with a procedure, and/or the like.

At block 55586, the surgical computing system 55547 may receive a first control data (e.g., control data 55545) from the first surgical element and/or a second control data (e.g., control data 55545n) from the second surgical element. As described herein, first and/or second control data may include operational data, historical data, and/or patient data. Operational data may include information associated with a surgical element during a procedure. For example, operational data may include the speed of a cutting tool, a selected control loop for the surgical tool, a voltage and/or current of an electrosurgical tool, a flow rate for an infusion pump and/or the like. Historical data may include data associated with past operational data generated during similar procedures and/or patient data generated during a procedure (e.g., a specific patient's data for one or more procedures and/or multiple patient's data for one or more procedures). Patient data may include real-time data associated with the health of a patient during a procedure (e.g., one or more physiological parameters of a patient such as a heart rate, SpO2, temperature, and/or the like).

At block 55587, the surgical computing system 55547 may determine a recommendation 55550 for a portion of a procedure as described herein. The recommendation 55550 may include, for example, an indication of an optimized control loop 55544. In examples, ML model 55549 may receive the first and/or second control data, a tactical domain target 55546, historical data 55548, an indication of a portion of a procedure, and/or the like. In response to receiving data, ML model 55549 may generate a recommendation 55550 including a selection and/or indication of a control loop 55544 to be used by a first surgical element during a portion of a procedure.

At block 55588, the surgical computing system 55547 may transmit the recommendation 55550 to the first surgical element. In examples, a recommendation 55550 may cause the first surgical element to automatically select an optimized control loop 55544. Alternatively, the surgical computing system 55547 may provide a recommendation 55550 to an HCP 55508 (e.g., via a GUI). A recommendation 55550 provided to an HCP 55508 may include an indication to select an optimized control loop 55544 and/or an indication to configure a first surgical element based on a portion of a procedure. In examples, an HCP 55508 may determine whether to apply the recommendation 55550 to the first surgical element, to cause the first surgical element to select the optimized control loop 55544. Once the recommendation 55550 is transmitted to the first surgical element, the routine 55580 ends.

An optional routine may be performed by a surgical computing system 55547 to determine a recommendation 55550 for a portion of a procedure including one or more conflict zones 55572 of FIG. 39. In examples, a surgical computing system 55547 may receive a user input indicating a selection of a procedure from a plurality of procedures and/or an indication of a tactical domain target 55546 (e.g., similar to block 55581, and/or block 55584 of FIG. 40). The tactical domain target 55546 and/or the procedure may be associated with a physiological parameter of a patient 55541.

The surgical computing system 55547 may filter, based on the selection of the procedure, a plurality of surgical elements to obtain a primary surgical element 555543 and/or a secondary surgical element 55543n associated with the procedure (e.g., similar to block 55582 of FIG. 40). The primary surgical element 55543 may include a plurality of primary control loops associated with an output characteristic of the primary surgical element 55543, and the secondary surgical element 55543n may include a plurality of secondary control loops associated with an output characteristic of the secondary surgical element 55543n.

The surgical computing system 55547 may determine a tactical domain data (e.g., a tactical domain 55542) for the procedure (e.g., similar to block 55583 of FIG. 40). The tactical domain data may include one or more relationships associated with the primary surgical element 55543, the secondary surgical element 55543n, the parameter of the patient, and the tactical domain target 55546.

The surgical computing system 55547 may receive a primary control data 55545 from the primary surgical element 55543 based on a primary control loop 55544 from the plurality of primary control loops (e.g., similar to block 55586 of FIG. 40). The primary control data 55545 may include the output characteristic associated with the primary surgical element 55543.

The surgical computing system 55547 may receive a secondary control data 55545n from the secondary surgical element 55543n based on a secondary control loop 55544n from the plurality of secondary control loops (e.g., similar to block 55586 of FIG. 40). The secondary control data 55545n may include the output characteristic associated with the secondary surgical element 55543n.

The surgical computing system 55547 may determine conflict data (e.g., one or more conflict zones 55572) for the procedure. The conflict data may include a determination of a conflict associated with the primary surgical element 55543 and/or the secondary surgical element 55543n. The conflict data may include a request for a second user input indicating whether the determination of the conflict occurred during the procedure. The surgical computing system 55547 may generate a recommendation based on the conflict data (e.g., similar to block 55587 of FIG. 40). The surgical computing system 55547 may transmit the recommendation to the primary surgical element 55543 (e.g., similar to block 55588 of FIG. 40.). In examples, the recommendation to the primary surgical element 55543 may be the display 55570 of FIG. 39.

In examples, the surgical computing system 55547 may generate the recommendation further based on a machine learning (ML) model. The ML model may be trained using training data including one or more training data items. Each training data item of the one or more training data items may include at least one indication of the conflict data (e.g., conflict zones 55572).

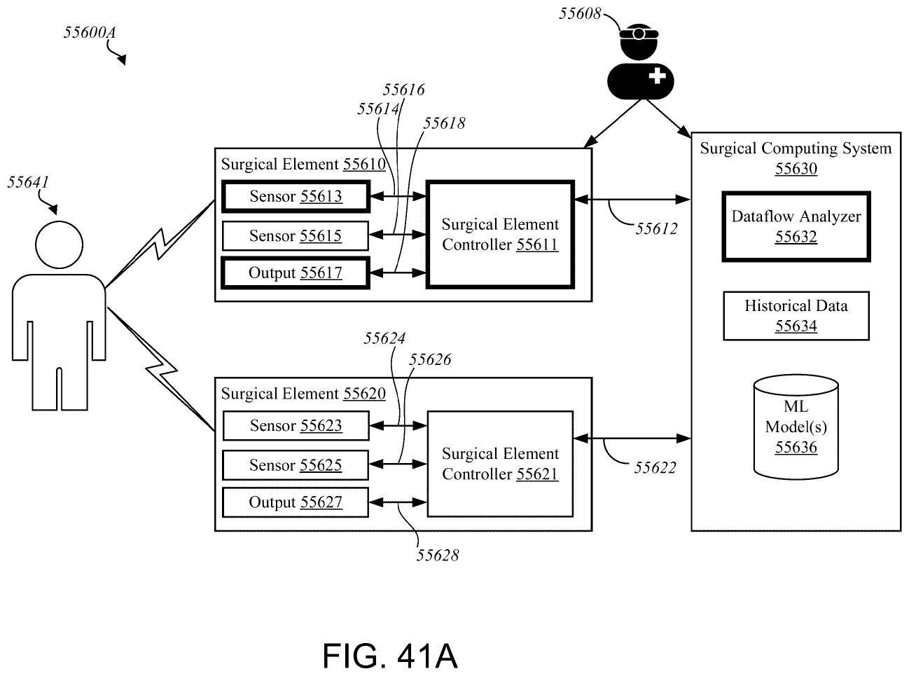

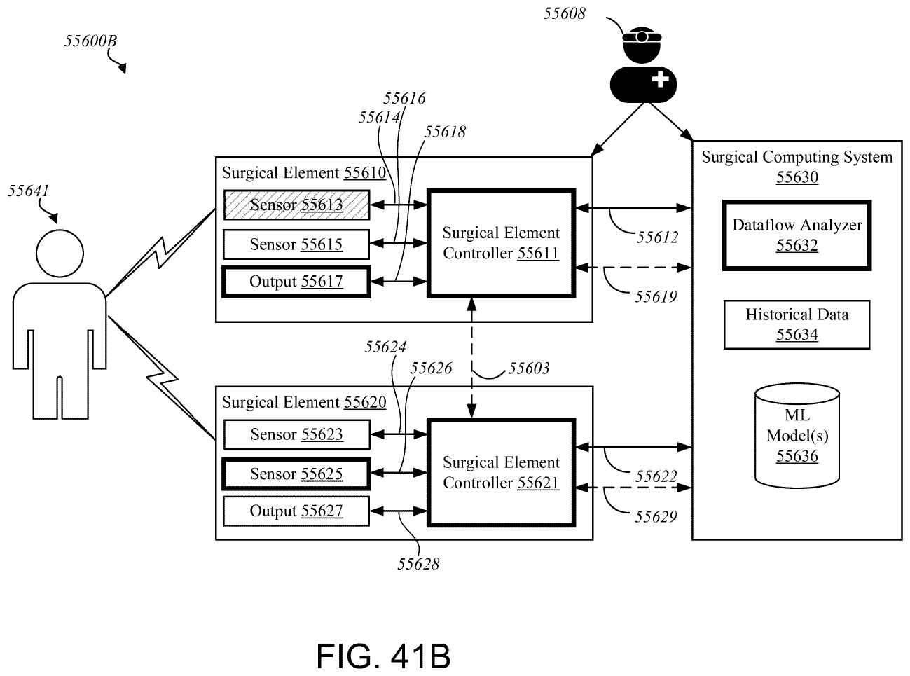

FIGS. 41A and 41B illustrate example operational environments 55600A, 55600B that may rebalance dataflows based on a number of unknowns. In examples, rebalancing dataflows based on the number of unknowns may include determining one or more dataflows to be added and/or included in a system to generate an optimized output and/or to prevent delay during a procedure. A dataflow may include sensor data, operational data, system generated data, control data, dataflow configuration information, and/or the like. The example operational environments 55600A, 55600B may include surgical element 55610, 55620, a patient 55641, a surgical computing system 55630, and/or an HCP 55608. Although operational environment 55600A, 55600B depict two surgical elements 55610, 55620, and/or one surgical computing system 55630, this is not intended to be limiting, as an example operational environment may include any combination of one or more surgical elements, surgical computing systems, HCPs, sensors, outputs, communication paths, and/or the like.

FIG. 41A illustrates an example operational environment 55600A (e.g., a typical control path as indicated with bold boarders). As part of operational environment 55600A, a surgical element 55610 (e.g., via sensor 55613) may provide sensor data (e.g., via 55614) to a surgical element controller 55611. The surgical element controller 55611 may provide a dataflow (e.g., via 55612) to a dataflow analyzer 55632. In response to receiving a dataflow, the dataflow analyzer 555632 may generate and/or send control data to the surgical element controller 55611 (e.g., via 55612). The surgical element controller 55611 may send the control data (e.g., via 55618) to an output 55617 (e.g., to cause the surgical element 55610 to adjust an output characteristic).

FIG. 41B illustrates an example operational environment 55600B (e.g., an alternative control path as indicated with bold boarders), where the surgical computing system 55630 may continue a procedure without the need to pause, cancel, and/or continue a procedure with a reduced number of surgical elements. The surgical computing system 55630 may continue a procedure without interruption by determining that a dataflow (e.g., via 55612) from failed sensor 55613 (e.g., as indicated with shading) is erroneous. The surgical computing system 55630 may communicate with a second surgical element 55620 (e.g., via 55622) to establish a second dataflow from sensor 55625. The surgical computing system 55630 may determine to communicate with a second surgical element 55620 based on sensor a determined relationship between sensor 55625 and/or sensor 55613. The second dataflow may be sent to the surgical computing system 55630 (e.g., via 55629) and/or sent to the surgical element 55610 (e.g., via 55603). The second dataflow may be used to determine control data, which may be sent (e.g., via 55619) to surgical element 55610 and/or determined by surgical element controller 55611. The control data may be sent to output 55617 (e.g., via 55618). Output 55617 may adjust an output characteristic of the surgical element 55610 based on the control data.

A surgical element 55610 may include sensor 55613, 55615, output 55617, and/or a surgical element controller 55611. Surgical element 55610 may be any one of a number of a instruments, devices, and/or the like, that may be present in an operating room during a procedure and/or capable of being connected (wired and/or wirelessly) to a surgical computing system 55630. In examples, surgical element 55610 may be a robotic surgical system, a navigation system, a smart imaging system, an endoscopic and/or laparoscopic system, an energy scalpel, an anesthesia machine, a patient monitoring system (pulse oximeters, blood pressure monitors, EKG monitors, EEG monitors, and/or the like), an energy device (e.g., electrosurgical units, laser surgery systems, and/or the like), an infusion pump, and/or the like.

A patient 55641 may be in communication with one or more surgical elements 55610, 55620 (e.g., sensor 55613, 55615, and/or output 55617, during a procedure). As an illustrative example, a ventilator (e.g., a surgical element 55610) and/or a pulse oximeter (e.g., surgical element 55620) may be attached to a patient during an open-heart surgery. An HCP 55608 and/or a surgical computing system 55630 may interact with one or more surgical elements 55610, to monitor the oxygen saturation of the patient (e.g., via the pulse oximeter), and/or determine a flow rate for a ventilator. In examples, the HCP 55608 may interact with the surgical computing system 55630 and/or surgical elements 55610, 55620 via a user input (e.g., a graphical user interface (GUI), a knob, a button, a smart device a wearable electronic device, and/or the like).

Sensor 55613, 55615 may be any instrument, transducer, and/or the like, configured to measure one or more environmental conditions. In examples, sensor 55613, 55615, may measure physiological parameters of a patient 55641 such as oxygen saturation, blood pressure, respiratory rate, blood sugar, heart rate, a core body temperature and/or a hydration state and/or the like. Sensor 55613, 55615 may measure an environmental condition of an operating room (e.g., the operational environment 55600A, 55600B) such as a room temperature, the number of HCPs 55608 present during a procedure, the position of an HCP 55608 during a procedure, a humidity level, air quality, lighting levels, CO2 levels and/or the like. Sensor 55613, 55615 may measure one or more environmental conditions associated with a surgical element, such as a current, voltage, a pressure, a force applied, a distance, a vibration, an orientation, a flow rate, a status, and/or the like. As an illustrative example, an HVAC system may include a temperature, humidity, and/or air quality sensor, a robotic surgical system may include a force and/or pressure sensor, a depth sensor, a temperature sensor, a blood flow and/or oxygen sensor, a pH sensor (e.g., to measure blood gas), an electrocardiogram (ECG) sensor, a position sensor (e.g., to measure the position of a robotic arm), and/or the like.

Sensor 55613, 55615 may send sensor data (e.g., information associated with a measured environmental condition) to a surgical element controller 55611 (e.g., via 55614, 55616 respectively). Sensor data may be sent based on dataflow configuration information. Dataflow configuration information may include an indication of a surgical element ID, an indication whether a dataflow is available, an update and/or acknowledgement of dataflow configuration information, a unit of measure, a communication protocol (RS-232, Ethernet, TCP/IP, Bluetooth, and/or the like), scheduling and/or frequency information (e.g., a time and/or frequency that sensor data is to be sent), a destination (e.g., port information associated with the surgical element controller 556611), and/or security and/or access control credentials. In examples, an update and/or acknowledgement of dataflow configuration information may indicate that a second dataflow is available if a first dataflow is removed (e.g., taken off-line and/or replaced by the second dataflow on a configured channel), and/or that a second dataflow may be available if a first dataflow and a second dataflow share a channel.

Output 55617 may be any number of devices and/or instruments as part of a surgical element 55610 that may generate an output characteristic based on a received signal. In examples, output 55617 may include, among other components, motors and/or actuators (e.g., as part of a robotic device, a cutting tool, ventilators and/or the like), lighting output devices, GUIs, cameras (e.g., as part of a borescope, IR camera, ultrasound camera, and/or the like), pumps (e.g., as part of smoke evacuation devices, suction devices, anesthesia delivery devices, IV infusion devices and/or the like), and/or lasers (e.g., as part of tissue ablation devices).

Output 55117 may receive control data from a surgical element controller 55611 (e.g., via 55618). In examples, output 55617 may adjust, based on control data received from a surgical element controller 55611, an output characteristic such as the speed of a cutting tool, the power intensity of an electrosurgical tool, a flow rate for an infusion pump, a position of a robotic arm, and/or the like. Although one output 55617 is included in surgical element 55610, it is to be understood that there may be multiple outputs, each receiving control data. As an illustrative example, a robotic system may include multiple outputs 55617 generating any number of outputs including positioning a robot, performing a measurement, capturing an image, creating an incision, and/or the like.

Surgical element 55610 may include a surgical element controller 55611. Surgical element controller may receive sensor data (e.g., via 55614, 55616) from sensor 55613, 55615. Surgical element controller 55611 may transmit control data to output 55617 (e.g., via 55618). A surgical element controller 55611 may communicate, a dataflow (e.g., sensor data, control data, dataflow configuration information, and/or the like) to/from a surgical computing system 55630. In examples, a surgical element controller 55611 may create and/or send a second dataflow (e.g., via 55619) to/from a surgical computing system 55630. In examples, surgical element controller 55611 may configure and/or send a dataflow based on a configuration message. In examples, a surgical element controller 55611 may determine that sensor data may be erroneous (e.g., as describe herein).

A surgical element controller 55611 may receive an interrogation message as described herein. In response, the surgical element controller 55611 may send an interrogation response to the surgical computing system 55630. The interrogation response may include the second dataflow and/or dataflow configuration information such as an indication whether a second dataflow is available an update and/or acknowledgement to dataflow configuration information, (e.g., such as an updated surgical element ID, an updated unit of measure and/or the like). In examples, the interrogation response may indicate that a surgical element 55610 may be configured to transmit a requested dataflow (e.g., that a dataflow is available, that one or more dataflows may be removed (e.g., taken off-line) to send the requested dataflow, and/or the like).

A surgical element controller 55611 may receive a configuration message as described herein. In response, the surgical element controller 55611 may send a configuration response to the surgical computing system 55630. The configuration response may indicate dataflow configuration information, and/or a dataflow of the surgical element 55610 (e.g., a dataflow associated with sensor 55613, 55615, and/or the like).

It is to be understood that surgical element 55620 and/or one or more associated components (e.g., sensor 55623, 55625, output 55627, and/or surgical element controller 55621) may include the same and/or similar interactions, features, functionalities, and/or the like, as described herein with reference to surgical element 55610 and/or one or more components of surgical element 55610 (e.g., sensor 55613, 55615, output 55617, surgical element controller 55611). Surgical element 55620 may be a second surgical element (e.g., separate from surgical element 55610). In examples, communication between one or more components as described with reference to surgical element 55610 may be the same and/or similar to one or more components of surgical element 55620. As an illustrative example, surgical element 55620 may send data to and/or receive data from a surgical computing system 55630 and/or a surgical element 55610, including an interrogation message, an interrogation response, a configuration message, a configuration response, control data, operational data, historical data, sensor data, a dataflow, system generated data, and/or the like (e.g., via 55622, 55624, 55626, 55628, 55629 and/or 55603).

In an illustrative example of two surgical elements as part of an operational environment, a first surgical element 55610 may be a laparoscopic tool including a temperature sensor, whereas a second surgical element 55620 may be a heating pad configured to heat an insufflated cavity. The laparoscopic tool may transmit a dataflow indicating a measured temperature of an insufflated cavity to the surgical computing system 55630, and/or the heating pad may transmit a measured temperature near an insufflated cavity to the surgical computing system 55630. In examples, the laparoscopic tool and/or the heating pad may have the same and/or different configuration information (e.g., the temperatures may not be transmitted at the same data rate, the temperature may not be in the same in the same units, a communication protocol may differ, a frequency of messages may be different, and/or the like).

Operational environment 55600A, 55600B may include a surgical computing system 55630. The surgical computing system 55630 may include a dataflow analyzer 55632, historical data 55634, and/or ML model(s) 55636.

A dataflow analyzer 55632 may include hardware, software, firmware and/or the like, to communicate with surgical element 55610, 55620, and/or an HCP 55608 (e.g., via a GUI), determine dataflow integrity (e.g., whether a dataflow is erroneous), establish relationships between surgical elements 55610, 55620, generate and/or store data (e.g., control data, historical data, sensor data, operational data, system generated data, training data, and/or the like).

A dataflow analyzer 55632 may receive a dataflow from a surgical element 55610, 55620 (e.g., via 55612, 55622 respectively). A dataflow may include sensor data, operational data, system generated data, control data, dataflow configuration information, and/or the like. In examples, dataflow analyzer 55632 may receive sensor data from sensor 55613. The sensor data may include a signal indicating a measurement of an environmental condition during a procedure (e.g., a current, voltage, a pressure, a force applied, a distance, a vibration, an orientation, a flow rate, a status, and/or the like). Sensor data may be configured (e.g., via surgical element controller 55611, 55621) based on dataflow configuration information as described herein. A dataflow analyzer 55632 may generate control data in response to receiving a dataflow. Control data may be sent to surgical element 55610, 55620, via 55612, 55622, to adjust an output characteristic of surgical element 55610, 55620.

Dataflow analyzer 55632 may determine and/or infer one or more relationships (e.g., a relational link) between components of an operational environment 55600A, 55600B. A relationship may be determined and/or inferred based on, for example, compatible sensors between surgical elements 55610, 55620, a determined dataflow that may be needed to resolve an issue during surgery, and/or based on a user input (e.g., by an HCP via a GUI). As an illustrative example, a dataflow analyzer 55632 may receive a first dataflow from a laparoscopic tool (e.g., a dataflow indicating a measured temperature of an insufflated cavity via sensor 55613) and a second dataflow from a heating pad (e.g., a dataflow indicating a measured temperature near an insufflated cavity via sensor 55625). The dataflow analyzer 55632 may determine, based on configuration information sensor data, and/or control data associated with each dataflow, that the two dataflows are related (e.g., a change detected by a first temperature sensor of a second surgical element 55620 may be related to a response and/or a change in control data for a first surgical element 55610). After the dataflow analyzer 55632 determines that one or more aspects of surgical elements 55610, 55620 are related, the dataflow analyzer 55632 may store an indication of the relationship in, for example, historical data 55634 (e.g., as a LUT defining the one or more relationships). Additionally and/or alternatively, dataflow analyzer 55632 may determine and/or provide training data (e.g., including historical data 55634) to ML model(s) 55636, to train an ML model to determine one or more relationships.

A dataflow analyzer 55632 may determine the integrity of a dataflow (e.g., whether sensor data and/or the like, is erroneous). As described herein, erroneous data may be caused by, for example, an incorrectly calibrated sensor, a misplaced and/or improperly installed sensor (e.g., interference by a heat source), calibration drift, contamination, electromagnetic interference, a failed sensor, operator error, misalignment (e.g., in the case of a position sensor), and/or the like.

A dataflow analyzer 55632 may determine that a dataflow is erroneous, for example, if control data exceeds a threshold, based on a determination that the first dataflow is unavailable, a determination that the physiological parameter of the patient exceeds a patient safety threshold, based on a comparison of sensor data to historical data (e.g., past sensor data and/or another data type), and/or based on an output of ML model(s) 55636.

Control data may exceed a threshold if, for example, a signal indicates a higher and/or lower power output level than allowed by a surgical element and/or as indicated by an HCP 55608, a signal associated with control data does not change and/or changes too rapidly within an expected time, and/or the control data indicates incorrect configuration information as described herein. A first dataflow may be unavailable based on, for example, an indication received in an interrogation response and/or in a configuration response, and/or as indicated by an HCP 55608.

A physiological parameter of the patient may exceed a patient safety threshold if, for example, sensor data from a first sensor 55613 and/or a second sensor 55623 are not within a defined range. As an illustrative example, a dataflow analyzer 55632 may receive sensor data from multiple sensors (e.g., a heart rate and a raspatory rate), and determine that, during a procedure when a patient's heart rate increases, the patient's raspatory rate is expected to increase as well. If the raspatory rate does not increase based on a determined schedule, the dataflow analyzer 55632 may determine that one or more sensors are erroneous. In examples, one or more patient safety parameters may be utilized during a procedure (e.g., a first patient safety parameter may be monitored during administration of anesthesia, whereas a second patent safety parameter may be monitored during an incision).

A dataflow analyzer 55632 may determine that a dataflow is erroneous if the dataflow is coming from a source (e.g., a surgical element 55610, 55620) that is not a trusted source and/or trust may not be established. In examples, an untrusted source may be a surgical element 55610, 55620 that may not be associated with a relational link. In examples, a dataflow analyzer 55632 may introduce higher level functional, electrical, or mechanical constraints that, regardless of the dataflow source inputs, ensures the surgical computing system 55630 may not move outside of the higher level constraints (e.g., which may be correlated to physical safety, patient safety, device performance, and/or other intrinsic characteristics).

In examples, a dataflow analyzer 55632 may receive a user input from an HCP 55608 (e.g., via a GUI) to incorporate and/or identify dataflows. A dataflow analyzer 55632 may utilize untrusted data, and/or prompt the user (e.g., HCP 55608) for additional steps, constraints, and/or at multiple stages of a procedure to gain confidence, incorporate as part of training data, and/or reduce overall risk (e.g., by incorporating user feedback to mitigate the risk of the untrusted dataflow).

In examples, a dataflow analyzer 55632 may simulate potential risks associated with incorporation of an untrusted dataflow. A dataflow analyzer 55632 may execute a simulation and/or theoretical exercise of what may occur in the operational environment 55600A, 55600B, and/or to other articulation or handling limits if the dataflow analyzer 55632 were to incorporate the (e.g., new) dataflow. A dataflow analyzer 55632 may execute one or more simulations to anticipate the creation of any new additional risks.

A dataflow analyzer 55632 may be placed into a safe mode or safe space where the dataflow analyzer 55632 may incorporate the new untrusted data and/or test (including physical motion) how that data impacts and/or creates additional risks to the operational environment 55600A, 55600B.

A dataflow analyzer 55632 may monitor untrusted dataflows with relation to trusted dataflows (e.g., dataflows that may have an indicated relationship associated with the operational environment 55600A, 55600B). In examples, a dataflow analyzer 55632 may execute actions (e.g., such as moving an articulation joint or motor) and/or continue to monitor the untrusted data source, as well as other trusted sources that the dataflow analyzer 55632 has access to. The dataflow analyzer 55632 may correlate the performance of the untrusted dataflow to other trusted dataflows.

In examples, a dataflow analyzer 55632 may include one or more methods for handling inherently untrusted data. In examples, dataflow analyzer 55632 may execute a certificate inspection, a safety process, and/or intentionally induce latency and/or a limp mode (e.g., based on dataflow configuration information). In examples, a certificate inspection may include utilizing certificates, encryption keys, and/or other data trustworthiness mechanisms to ensure that data integrity of a dataflow has been preserved. In examples, a safety process may be executed after a trigger event. The safety process may monitor higher level functionality, such that the dataflow is coordinated. In examples, intentionally induced latency and/or a limp mode may include slowing down a speed associated with an output characteristic (e.g., associated with a dataflow) such that the user (e.g., an HCP 55608) may react if the output characteristic starts to misbehave and/or have a chance to correct based on user feedback (e.g., a user input via a GUI), as well as a chance to establish one or more relationships for the dataflow.

As described herein, the dataflow analyzer 55632 may determine to incorporate a second dataflow based on one or more relationships. If a (e.g., new) dataflow is found to be unreliable, the dataflow analyzer 55632 may choose to exclude the dataflow and/or default back to a prior dataflow.

In examples, a dataflow may include degrees of reliability. A dataflow may not be reliable (e.g., 100% reliable) to be used. As an illustrative example, a speed sensor on a piece of equipment may not be calibrated, and as a result, the speed sensor may output an incorrect speed. If the speed is incorrect, dataflow analyzer 55632 may use the dataflow to determine if a device is in motion and/or paused. In examples, erroneous dataflows may be linear and/or non-linear. A dataflow may be reliable within a zone of operation and/or unreliable in other zones (e.g., linearly offset in other zones).

In examples, a dataflow analyzer 55632 may determine the manner of an unreliable and/or dysfunctional dataflow. The manner of dysfunction or unreliability of the dataflow may impact the ability to utilize that dataflow in a capacity.

In examples, dataflow analyzer 55632 may determine the error of the dataflow. A secondary dataflow may be used to quantify an erroneous dataflow. As an illustrative example, a failure of a tachometer may indicate the incorrect speed of a fan. A dataflow analyzer 55632 may use other sensor data (e.g., such as for current or back-emf) to indicate the nature of the failed tachometer (e.g., miscalibration, complete failure, and/or the like).

In examples, a dataflow analyzer 55632 may utilize a known perturbation to determine the level of impact. As an illustrative example, a speed of a fan with a damaged tachometer may have its speed internationally increased to quantify the impact of the failure and/or understand if the tachometer has experienced a complete failure (e.g., no change with increased speed), offset failure, and/or calibration failure.

In examples, depending on the type of reliability issue (e.g., type of failure associated with an erroneous dataflow) with a dataflow, the dataflow may be used as a secondary verification rather than a primary control source (e.g., control with a second dataflow, and verify with a first dataflow). In examples, the first dataflow may be utilized at a (e.g., new) level with an indication, that if changes, may default back to an expected orientation.

A dataflow analyzer 55632 may replace an erroneous dataflow with a second dataflow (e.g., a second dataflow based on a determined relationship as described herein). As illustrated in FIG. 41B, sensor 55613 is shaded to indicate that the dataflow analyzer 55632 determined that a dataflow from sensor 55613 is erroneous. The dataflow analyzer 55632 may (e.g., based on one or more determined relationships) transmit an interrogation message and/or a configuration message to a second surgical element 55620, to establish a second dataflow to compensate for the erroneous data of the first dataflow. An example path of a second dataflow may be indicated by bold boarders associated with sensor 55625, surgical element controller 55621, dataflow analyzer 55632, surgical element controller 55611, and/or output 55617, and/or via 55629, 55619, and/or 55603 of operational environment 55600B. A second dataflow may be transmitted from surgical element 55620 to the dataflow analyzer 55632, and then to the surgical element 55610 (e.g., via 55629, 55619). If the second dataflow is transmitted to the dataflow analyzer 55632, the dataflow analyzer 55632 may use the second dataflow to generate control data for the first surgical element 55610 (e.g., to compensate for the erroneous sensor data of sensor 55613). Alternatively, a second dataflow transmitted from surgical element 55620 to a first surgical element 55610, (e.g., via 55603) may be used by the surgical element 55610 to determine control data associated with output 55617.

A dataflow analyzer 55632 may estimate sensor data associated with a failed sensor 55613. A dataflow analyzer 55632 may analyze control data (e.g., associated with surgical element 55610, 55620), sensor data (e.g., from sensor 55615, 55623, 55625), operational data, historical data, and/or system generated data, to estimate an environmental condition of an operational environment 55600A, 55600B.

A dataflow analyzer 55632 may be able to incorporate (e.g., new) dataflows of which the dataflow analyzer 55632 has knowledge that are available. A dataflow analyzer 55632 may identify available dataflows based on deterministic criteria. Deterministic criteria may include historical prior connections, dataflow status identification, searching, and/or listening. Deterministic criteria may be used in conjunction with and/or complementary to, one or more methods described herein for determining a dataflow.

A dataflow analyzer 55632 may determine a dataflow based on historical prior connections (e.g., as stored in historical data 55634). In examples, dataflow analyzer 55632 may be aware of prior connections (e.g., via historical data 55634, a user input from an HCP 55608, and/or the like), and as a result, utilize a dataflow based on the prior connection.

A dataflow analyzer 55632 may determine a dataflow based on a dataflow status ID (e.g., as part of dataflow configuration information). A dataflow status ID (e.g., a surgical element ID) may be included in metadata. A dataflow status ID may provide a status of one or more dataflows that may be made available (e.g., via communication with one or more surgical elements to allow the dataflow analyzer 55632 to understand one ore more options for incorporation of the dataflow).

A dataflow analyzer 55632 may determine a dataflow based on a distributed data system. A distributed data system may include a list of topics available such as in the LUT described herein (e.g., similar to how a data distribution service network may be constructed). In examples, whether hardcoded or via communication, a data distribution service may have defined knowledge that one or more dataflows exist, and/or the dataflow analyzer 55632 may be able to then subscribe to one or more networks at a given time.

A dataflow analyzer 55632 may search for dataflows. In examples a dataflow analyzer 55632 may actively search (e.g., via an interrogation message described herein) across networks which the dataflow analyzer 55632 may have access to (e.g., wireless, electrical, and/or the like) to try to identify potential sources of information).

A dataflow analyzer 55632 may listen for dataflows. A dataflow analyzer 55632 may actively identify and/or announce that dataflows exist (e.g., such as with advertising data on a wireless beacon, and/or as a result, the dataflow analyzer 55632 may then identify and/or incorporate dataflows).

As described herein, the dataflow analyzer 55632 may transmit an interrogation message. An interrogation message may be sent by the dataflow analyzer 55632 to one or more surgical elements 55610, 55620, and/or to another surgical computing system. The interrogation message may be sent based on one or more determined relationships as described herein. In examples, the interrogation message may be sent by the dataflow analyzer 55632 to create, update, and/or verify one or more relational links (e.g., relationships) as described herein (e.g., with and/or without the detection of erroneous data). In examples, an interrogation message may be sent in response to a determination that a first dataflow is erroneous, based on a predefined frequency and/or time (e.g., as determined by the dataflow analyzer 55632), and/or based on a user input (e.g., by an HCP 55608 via a GUI). An interrogation message may, among other things, request confirmation that a second surgical element 55620 includes a compatible and/or available dataflow.

An interrogation message may include a request for information associated with one or more dataflows of a surgical element 55610, 55620. In examples, an interrogation message may request information associated with a second dataflow from a second surgical element 55620 (e.g., that may be related to a first dataflow of a first surgical element 55610). In examples, a dataflow analyzer 55632 may request, via the interrogation message, that the second surgical element 55620 may determine whether a second dataflow is available. The interrogation message may include information associated with a surgical element 55610, 55620 (e.g., to determine whether a second dataflow is available). In examples, information associated with a surgical element 55610, 55620, may include dataflow configuration information as described herein.

In examples, an interrogation message may be sent by the dataflow analyzer 55632 resolve an imbalance of a closed loop (e.g., to include a number of equations to balance a number of unknowns). As an illustrative example, a smoke evacuation system may be controlled by energy activation to prevent limitations of visibility. During the procedure, the HCP 55608 may be unable to see effectively (e.g., smoke evac may not effectively mitigate the issue). A scope may be added as a dataflow to control the smoke evacuation rate in the smoke evacuator. After the dataflow analyzer 55632 identifies an imbalance (e.g., identifies an erroneous dataflow), the dataflow analyzer 55632 may interact and/or search for a locally available dataflow to be used to resolve the imbalance (e.g., the dataflow analyzer 55632 may request, via an interrogation message, that advanced visualization system (e.g., a scope) provide a stream of occlusion magnitude, to look at the intake of the smoke evaluation for a particle count dataflow. The dataflow may be a particle type visualization (e.g., aerosol versus smoke), and/or an energy device jaw dataflow detection of tissue/body fluid interaction (e.g., whether the smoke evacuation system is immersed in blood, collagen tissue, and/or the like). The advanced visualization system may respond with an interrogation response, indicating a dataflow that may be used.

In response to sending an interrogation message, a dataflow analyzer 55632 may receive an interrogation response. An interrogation response may include dataflow configuration information, including an indication whether a second dataflow is available, an indication of an update to information associated with a surgical element 55610, 55620), and/or an acknowledgement of the information associated with the surgical element. An update to one or more determined relationships may include an indication of a second sensor that may be related to a failed sensor (e.g., sensor 55625 may have be related to sensor 55613 in operational environment 55600B). In examples, the interrogation response may indicate that a surgical element 55610, 55620 may be configured to transmit the requested dataflow (e.g., that a dataflow is available, that one or more dataflows may be removed (e.g., taken off-line) to send the requested dataflow, and/or the like).

A dataflow analyzer 55632 may transmit a configuration message. A configuration message may be sent in response to a received interrogation response, based on a user input (e.g., an HCP 55608 via a GUI), and/or as determined by the dataflow analyzer 55632 (e.g., based on a determination that a dataflow is sending erroneous data and/or the like). A configuration message may request to configure a surgical element based on dataflow configuration information and/or a dataflow associated with a surgical element 55610, 55620. In examples, dataflow analyzer 55632 may instruct a second surgical element 55620 to transmit (e.g., via a configuration message and/or an interrogation message) a configuration response to a first surgical element 55610 (e.g., via 55603).

A configuration message may include a request for sensor data (e.g., as part of a dataflow) based on one or more relationships as determined by the dataflow analyzer 55632, ML model(s) 55636, and/or the like. A configuration message may include information associated with configuring a surgical element 55610, 55620 (e.g., dataflow configuration information). As an example, dataflow analyzer 55632 may send a configuration message to surgical element 55620, to configure a second dataflow (e.g., via 55622 and/or 55629) for receiving sensor data associated with sensor 55625. The configuration message may be sent based on a determination that sensor 55625 is related to failed sensor 55613 and/or that the dataflow analyzer 55632 may need a second dataflow to resolve an issue.

A dataflow analyzer 55632 may receive a configuration response. A configuration response may be received via 55612, 55619, 55622, and/or 55629. A configuration response may include dataflow configuration information, and/or a dataflow associated with a surgical element 55610, 55620. As an illustrative example, a configuration response may include a surgical element ID associated with surgical element 55620, a dataflow protocol associated with surgical element 55620 (e.g., ethernet and/or the like), the frequency that sensor data is sent to the dataflow analyzer 55632, and/or access control credentials associated with sensor 55625, surgical element 55620, and/or the like), and sensor data generated from sensor 55625 (e.g., to be used by the dataflow analyzer 55632 to generate control data for surgical element 55610).

A dataflow analyzer 55632 may utilize an arbitrator (e.g., not depicted as part of operational environment 55600A, 55600B) for connecting one or more surgical elements 55610, 55620. An arbitrator may enable bidirectional communication to be handled between surgical elements 55610, 55620, and/or a surgical computing system 55630 (e.g., may only support communication on a single bus).

In examples, surgical elements 55610, 55620, and/or a surgical computing system 55630 may not be configured for bussed and/or network communications, and/or may be intended for point to point communication (e.g., RS-232). In examples, a message (e.g., a dataflow, a configuration message, an interrogation message, and/or the like) that may be output from at least two surgical elements 55610, 55620, may be transmitted to an arbitrator before being received by the dataflow analyzer 55632. This methodology is not limited to RS-232, and may be employed for other communication modalities (e.g., including wired and/or wireless, Wi-Fi, and/or base station concepts to expand wireless networks). Use of an arbitrator can allow messages to be handled and output in sequential order, without creating message bus conflicts. An arbitrator may utilize any manner of embedded methodologies (e.g., interrupt driven, high speed implementation, and/or the like) to determine the order in which messages on one or more busses are dedicated. The order may become the internal order in which messages are passed through to the output of a surgical computing system 55630. To handle or mitigate potential overflows, messages may be briefly placed into memory such that message data is not lost.

A dataflow analyzer 55632 may generate control data based on a dataflow received from surgical element 55610, 55620, in response to a user input (e.g., by an HCP via a GUI), and/or as determined by the dataflow analyzer 55632. Control data may include information associated with, for example, a control variable setpoint (e.g., associated with a motor speed, a temperature, a flow rate, and/or the like), a current and/or voltage (e.g., associated with an electrosurgical tool, and/or the like), an instruction (e.g., to generate an image from a camera and/or the like), and/or a power level (e.g., of a heating pad, a laser ablation tool, and/or the like). As an illustrative example, control data may include a setpoint for the speed of a cutting tool, a voltage and/or current setpoint of an electrosurgical tool, a flow rate for an infusion pump, a position of a robotic arm, and/or the like.

A dataflow analyzer 55632 may transmit control data (e.g., via 55622, 55629) to a surgical element 55610, 55620, to cause the surgical element 55610, 55620, to adjust an output characteristic associated with output 55617, 55627. The output 55617, 55627, may adjust an output characteristic based on the information associated with a control variable setpoint, a current and/or voltage, an instruction, a power level, and/or the like, as indicated in the control data.

In examples, dataflow analyzer 55632 may instruct a second surgical element 55620 to transmit control data from the second surgical element 55620 to a first surgical element 55610 (e.g., via 55603). In examples, control data may be transmitted (e.g., via 55619) to adjust an output of a first surgical element 55610 based on a dataflow received from a second surgical element 55620 (e.g., as illustrated in operational environment 55600B).

A dataflow analyzer 55632 may generate control data based on multiple dataflows. A dataflow analyzer 55632 may, in response to a user input and/or as determined by the surgical computing system 55630, add a second dataflow to generate control data (e.g., without determining that a first dataflow is erroneous). As an illustrative example, an image camera (e.g., a first surgical element 55610) may transmit a first dataflow to a surgical computing system 55630 with a limited resolution. The limited resolution of the camera may not provide an HCP 55608 with a sufficient image during a procedure. The HCP 55608 may request, via a GUI, that the dataflow analyzer 55632 determine a second dataflow (e.g., based on one or more determined relationships between a first surgical element 55610 and a second surgical element 55620) that may be used with the first dataflow to enhance the resolution of the image. The dataflow analyzer 55632 may request a second dataflow (e.g., via a configuration message as described herein) from, for example, an IR camera (e.g., a second surgical element). The second dataflow may be utilized with the first dataflow to generate control data (e.g., to enhance the output image to the HCP 55608).

A dataflow analyzer 55632 may escalate a determination to include a second dataflow (e.g., whether an erroneous dataflow affects the safety of the patient) to an HCP 55608 and/or to the system 55630. In examples, the surgical computing system 55630 may be unable to generate control data based on one or more dataflows. The dataflow analyzer 55632 may determine that a second dataflow may be added to improve control data in the event that a first dataflow that is monitored, but not utilized, deviates from an expected range.

As an illustrative example, a smoke evacuation device may be controlled by energy activation to clear a field of view for an HCP 55608 during a procedure. If the dataflow analyzer 55632 determines that the smoke evacuation device is not effectively clearing the field of view, the dataflow analyzer 55632 may add a scope occlusion device (e.g., a dataflow analyzer 55632 may add a second dataflow from a scope occlusion device) to control the smoke evacuation rate (e.g., to adjust an output characteristic of the smoke evacuation device).

In examples, the second dataflow may already be monitored (e.g., received by the dataflow analyzer 55632), and/or the dataflow analyzer 55632 may include the existing dataflow from the additional device. As an illustrative example, a smoke evacuation device may be an advanced energy activation device (such as output 55617). A scope may measure a particle count to assess the effectiveness of the smoke evacuation device to clear the field of view. If the particle count is no longer successfully controlled, the dataflow analyzer 55632 may add an existing particle count dataflow from the scope to control smoke evacuation device.

In examples, the second dataflow may not be monitored (e.g., received by the dataflow analyzer 55632), and/or the dataflow analyzer 55632 may automatically include the new dataflow (e.g., according to FIGS. 41A, 41B, 42, 43, 44A, and/or 44B). As an illustrative example, a smoke evacuation device may be controlled by an advanced energy activation device (e.g., output 55617). A scope may measure a particle count to assess the effectiveness of the smoke evacuation device to clear the field of view. If the particle count is not successfully controlled (e.g., a threshold is reached and/or a trigger condition is satisfied), the dataflow analyzer 55632 may initiate infrared monitoring as a second dataflow on the scope to assess and/or monitor particle count. The dataflow analyzer 55632 may utilize the IR data to control the smoke evacuation device.

In examples, a second dataflow may not be monitored, and/or the dataflow analyzer 55632 may request a user input (e.g., via a GUI) to include the second dataflow. In examples, the dataflow analyzer 55632 may generate an indication (e.g., via a GUI) to the HCP 55608, informing the HCP 55608 that a second dataflow may be included (e.g., the dataflow analyzer 55632 may provide the HCP 55608 with an opportunity to prevent the second dataflow from being used). As an illustrative example, a dataflow analyzer 55632 may determine that a robotic device (e.g., surgical element 55610) may not be able to effectively locate a probe tip. The dataflow analyzer 55632 may prompt an HCP 55608 (e.g., via a GUI) to activate a second dataflow associated with, for example, a cone beam CT that is energized and proximate to the patient (e.g., sensor 55625 and/or surgical element 55620 of operational environment 55600B) to locate the probe tip.

In examples, a second dataflow may not be available, and/or may not be enabled by the dataflow analyzer 55632 (e.g., as described with reference to FIGS. 41A, 41B, 42, 43, 44A, and/or 44B). In examples, a dataflow analyzer 55632 may determine that a robotic device (e.g., surgical element 55610) may not be able to effectively locate a probe tip. The dataflow analyzer 55632 may prompt an HCP 55608 (e.g., via a GUI), to connect a cone beam CT that is not in use (e.g., the dataflow analyzer 55632 may determine a relationship between the robotic device and the cone beam CT as described herein), and indicate to the HCP 55608 that a cone beam CT may be connected, positioned, energized and/or activated, to locate the probe tip.

In examples a dataflow analyzer 55632 may determine that a function of a surgical element 55610 may not achieve a desired outcome (e.g., then identify and add a second dataflow to achieve the desired outcome). As an illustrative example, a scope may traverse through a bronchi (e.g., via a robotic device). As the scope progresses further into the bronchial tree, scope position accuracy may decrease. Once position accuracy decreases past a threshold (e.g., a trigger condition is satisfied) a dataflow analyzer 55632 may determine to include additional position information (e.g., a second dataflow) to accurately determine the scope position (e.g., the dataflow analyzer 55632 may add position data from the CT to confirm the robotic device position to drive movement of the scope).

In examples, a dataflow analyzer 55632 may add a dataflow to optimize patient safety. In examples, a dataflow analyzer 55632 may indicate that an additional dataflow may be used to generate a specified output. As an illustrative example, a smoke evacuation device may be controlled by an energy activation device (e.g., output 55617) to clear a field of view for an HCP 55608 during a procedure. If an HCP 55608 is unable to see effectively (e.g., the smoke evacuation device is not effectively mitigating the issue) a scope is added as a dataflow to control the smoke evacuation rate. In examples, a dataflow from energy and/or a dataflow from an insufflation pressure may be used. In examples, the dataflow analyzer 55632 may determine if visual scope output, patient temperature, and/or another sensor may best maintain a field of view for the HCP 55608. In examples, ML model(s) 55636 may be trained to determine an optimized dataflow to be added.

In examples, a dataflow analyzer 55632 may determine that a second dataflow may be included based on ML model(s) 55636. As an illustrative example, a smoke evacuation device may be controlled by an energy activation device (e.g., output 55617) to clear a field of view for an HCP 55608 during a procedure. If an HCP 55608 is unable to see effectively (e.g., the smoke evacuation device is not effectively mitigating the issue) a scope occlusion is added as a dataflow to control the smoke evacuation rate. The dataflow analyzer may use ML model(s) 55636 to predict if/when the scope dataflow is needed (e.g., instead of waiting for an occlusion to limit the ability to function).

A dataflow analyzer 55632 may determine reduce the number of controlled variables (e.g., output 55617, 55627) in response to receiving an erroneous dataflow, and/or the like. In examples, a dataflow analyzer 55632 may limit the impact of an imbalance (e.g., an erroneous dataflow) by reducing the number of unknowns (e.g., reducing the control data associated with adjusting output characteristic(s) such as output 55617, 55627) without providing a second dataflow to create a closed loop (e.g., as indicated in bold boarders as part of operational environment 55600A, 55600B). In examples, one loop may remain closed, and/or a second loop may be allowed to remain open. In examples, a dataflow analyzer 55632 may determine that an erroneous dataflow is less reliable but related. Rather than using a second dataflow, the dataflow analyzer 55632 may use an internal dataflow (e.g., generated as part of system generated data, and/or the like). As an illustrative example, a visual camera may be a surgical element 55610, having a resolution (e.g., 480*620, and/or the like). The resolution may not be sufficient during a procedure. The dataflow analyzer 55632 may include a dataflow from an IR camera to increase the resolution. In examples, the IR camera and the visual camera images may be combined into an image.

In examples, a dataflow analyzer 55632 may utilize ML model(s) 55636 (e.g., a computer vision model). As an illustrative example, the dataflow analyzer 55632 may detect that endocutter connected, and prompt an HCP 55608 (e.g., a surgeon) to enable a perfusion detection model (e.g., the dataflow analyzer 55632 prompts the user to select the endocutter and/or the perfusion model).

As an illustrative example, as a scope is inserted into a bronchi, the EM position reliability may decrease. The dataflow analyzer 55632 may determine that additional position information is needed to ensure proper scope placement. The dataflow analyzer 55632 may add position data from a CT to confirm the position to drive movement of the scope. In examples, as the scope is inserted deeper within the body, the EM may lose accuracy (e.g., conflicts with reality). The dataflow analyzer 55632 may execute reverse kinematics to determine position. As an illustrative example a smoke evacuation may be driven by energy activation to prevent limitations of visibility. The HCP 55608 may be unable to see effectively (e.g., smoke evac is not effectively mitigating the issue). The HCP 55608 may request to change an input for the smoke evac to be dependent on scope occlusion instead of energy activation.

In examples, a dataflow analyzer 55632 may rebalance (e.g., determine a dataflow) by changing the effect of the dataflows. Changing the effect of dataflows may include a different gain factor (e.g., reducing a dataflow with a high level of noise). In examples, a dataflow analyzer 55632 may reduce the gain on a dataflow to improve reliability of the control data.

Historical data 55634 may store and/or include data associated with one or more past procedures. Historical data 55634 may be generated and/or received from surgical elements 55610, 55620, an HCP 55608 (e.g., via a GUI), and/or by the surgical computing system 55630. Historical data 55634 may be used as part of training data (e.g., to train ML model(s) 55636 as described herein). Historical data 55634 may be used by a surgical computing system 55630 to determine one or more relationships associated with surgical elements 55610, 55620). In examples, information associated with historical data 55634 may be included in a LUT to indicate one or more relationships between surgical elements 55610, 55620.

In examples, historical data 55634 may include control data, sensor data, operational data, system generated data, dataflow configuration information, and/or training data. Control data may include information associated with an output characteristic of a surgical element 55610, 55620. Sensor data may include data associated with a measurement of an environmental condition (e.g., one or more sensors) that may have been generated during a procedure. Operational data may include data associated with an HCP 55608 and/or patient 55641, such as historic procedural times, specific surgical elements used by an HCP during a procedure, historical records of a patient's physiological parameters, historical records of multiple patients' physiological parameters associated with a procedure, and/or the like. System generated data may include one or more inferred relationships (e.g., in a LUT), communication messages (e.g., interrogation message/response, configuration message/response, and/or the like), and/or the like, for one or more components of an operational environment. Training data, as described herein, may include data that is used to train an ML model(s) 55636 (e.g., to infer a relationship between components of an operational environment 55600A, 55600B).

In examples, historical data 55634 may include information associated with one or more dataflows. As an illustrative example, historical data 55634 may store information associated with identifying relationships between dataflows (e.g., based on configuration information), such as a surgical element ID, a communication protocol, scheduling and frequency information, a destination, security and access control credentials, a unit of measure, and/or the like.

A surgical computing system 55630 may include ML model(s) 55636. ML model(s) 55636 may receive, as an input, a dataflow, dataflow configuration information, control data, operational data, system generated data, historical data, and/or the like. In response to the received inputs, ML model(s) 55636 may determine one or more relationships associated with surgical elements 55610, 55620, determine whether a dataflow is erroneous, and/or determine an optimized dataflow that may be used to replace an erroneous dataflow and/or may be added to resolve an issue. The ML model(s) 55636 may transmit an output (e.g., a result, a determination, a recommendation, and/or the like) to dataflow analyzer 55632, based on one or more trained models. ML model(s) 55636 may generate individual training data items that form training data (e.g., to train one or more models). Training data items may include a procedure, historical data 55634 (e.g., control data, operational data, sensor data, dataflow configuration information, system generated data and/or the like), physiological parameters of a patient, one or more determined relationships, and/or the like.

ML model(s) 55636 may determine a relational link (e.g., a relationship) associated with a first surgical element 55610 and/or a second surgical element 55620. A relational link may be determined in real-time (e.g., during a procedure), or at another time (e.g., based on historical data 55634 as described herein). A relationship may be determined based on one or more aspects of a surgical element 55610, 55620 (e.g., dataflows, control data, sensor data, operational data, configuration information, and/or the like) as described herein. ML model(s) 55636 may store an indication that one or more aspects of surgical elements 55610, 55620 are related in, for example, historical data 55634 (e.g., via a LUT).

In examples, ML model(s) 55636 may determine that a relationship exists between sensor data associated with a second surgical element 55620 (e.g., sensor 55625) and sensor data associated with a first surgical element 55610 (e.g., sensor 55613). Sensor data may be related if, for example, sensor data shares a similar unit of measure, the sensor data measure a similar physiological parameter of a patient's body, the sensors are proximate to one another during a procedure, and/or the like.

In examples, ML model(s) 55363 may determine that a relationship exists between sensor data associated with a second surgical element 55620 (e.g., sensor 55625) and/or control data generated for a first surgical element 55610 (e.g., control data used to control output 55617). Sensor data associated with a second surgical element 55620 (e.g., sensor 55625) may be related to control data generated for a first surgical element 55610 if, for example, a sensor 55625 measures an output characteristic of the first surgical element 55610 as described herein.

In examples, ML model(s) 55636 may determine that a relationship exists based on dataflow configuration information (e.g., based on a surgical element ID, a communication protocol, scheduling and frequency information, a destination, security and access control credentials, and/or a unit of measure), for a surgical elements 55610, 55620. As an illustrative example, a dataflow analyzer 55632 may transmit to ML model(s) 55636, a first dataflow from a laparoscopic tool (e.g., a dataflow indicating a measured temperature of an insufflated cavity via sensor 55625) and a second dataflow from a heating pad (e.g., a dataflow indicating a measured temperature near an insufflated cavity via sensor 55613 and/or a power level associated with heat generated by the heating pad (e.g., via output 55617)). ML model(s) 55636 may determine that an increase in temperature, as measured by the laparoscopic tool, is caused by the power output associated with the heating pad. ML model(s) may indicate, to dataflow analyzer 55632, that the temperature sensor associated with the laparoscopic tool may be used to control the power output of the heating pad.

ML model(s) 55636 may determine whether a dataflow is erroneous. As described herein, an erroneous dataflow may be caused by, for example, an incorrectly calibrated sensor, a misplaced and/or improperly installed sensor (e.g., interference by a heat source), calibration drift, contamination, electromagnetic interference, a failed sensor, operator error, misalignment (e.g., in the case of a position sensor), and/or the like. ML model(s) 55636 be trained to detect (e.g., determine) that a dataflow erroneous in real-time (e.g., based on operational data, sensor, data, system generated data, and/or control data). In examples, ML model(s) 55636 may determine that a dataflow is erroneous based on a comparison of control data, sensor data, operational data, and/or system generated data to historical data. As an illustrative example, ML model(s) 55636 may determine that a dataflow (e.g., associated with a temperature indication) is erroneous if a first temperature sensor increases while one or more related temperature sensors remain at a constant temperature.

ML model(s) 55636 may determine an optimized dataflow that may be used to replace an erroneous dataflow. ML model(s) 55636 may determine that a dataflow is an optimized dataflow based on dataflow configuration information, a procedure, one or more physiological parameters of a patient, historical data, one or more determined relationships, an input from an HCP 55608, and/or the like. An optimized dataflow may be selected from a plurality of dataflows, to provide sensor data to a dataflow analyzer 55632 that most closely resembles and/or represents sensor data previously sent in an erroneous dataflow.

For example, ML model(s) 55636 may determine that a dataflow (e.g., including sensor data from sensor 55625) may provide stable, reliable, information that may be used by the dataflow analyzer 55632 to generate reliable control data. As an illustrative example, an optimized dataflow may be different (e.g., sensor data from a first temperature sensor may be more accurate than a second temperature sensor) for a patient that is susceptible to hypothermia (e.g., an elderly patient, a pediatric patient, and/or the like) versus a patient that may be more resistant to hypothermia (e.g., a patient with a high body-mass index, patients with high basal metabolic rates, and/or the like).

ML model(s) 55636 may estimate sensor data associated with a failed sensor 55613 during a procedure. In examples, ML model 55636 may receive control data, sensor data, operational data, and/or system generated data, and estimate an environmental condition of an operational environment 55600A, 55600B (e.g., during a procedure) based on previous measurements during similar portions of a procedure.

Example routine for re-balancing the number of unknowns and dataflows may be provided.

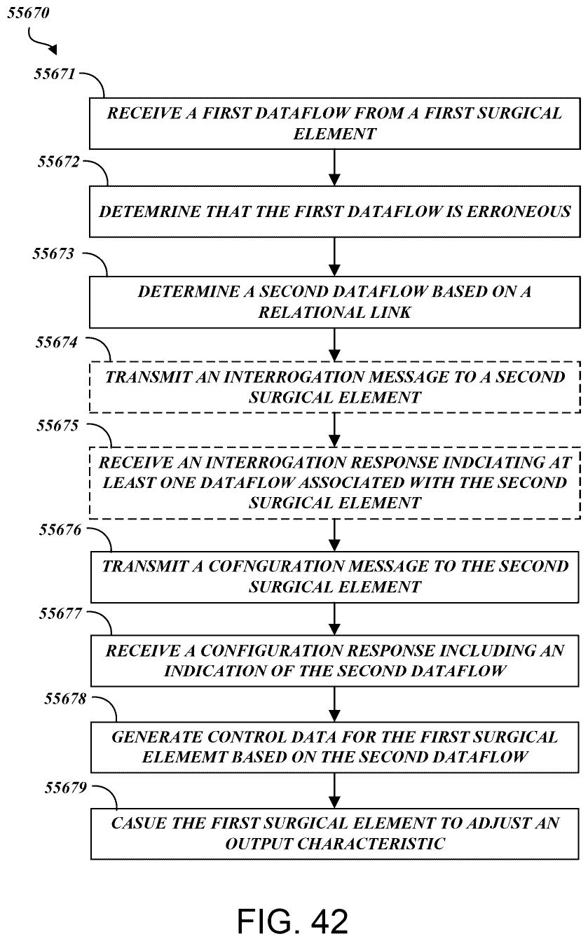

FIG. 42 illustrates an example routine 55670 for re-balancing the number of unknowns and dataflows (e.g., data streams). Example routine 55670 may be executed by, for example, surgical computing system 55630 (e.g., dataflow analyzer 55632) of operational environment 55600A, 55600B. The example routine 55670 begins at block 55671.

At block 55671, a dataflow analyzer 55632 may receive a first dataflow from a first surgical element 55610. The dataflow may include sensor data, operational data, system generated data, control data, dataflow configuration information, and/or the like. In examples, dataflow analyzer 55632 may receive sensor data from sensor 55613. The sensor data may be a signal indicating a measurement of an environmental condition during a procedure (e.g., a current, voltage, a pressure, a force applied, a distance, a vibration, an orientation, a flow rate, a status, and/or the like).

At block 55672, a dataflow analyzer 55632 may determine that the first dataflow is erroneous based on a determination that control data exceeds a threshold, a determination that the first dataflow is unavailable, a determination that the physiological parameter of the patient exceeds a patient safety threshold, based on a comparison of sensor data to historical data (e.g., past sensor data and/or another data type), and/or based on an output of ML model(s) 55636.

At block 55673, a dataflow analyzer 55632 may determine a second dataflow based on a relational link (e.g., a relationship between surgical elements). A relationship may be defined in a LUT, determined by an HCP 55608, and/or determined by ML model(s) 55636 as described herein. As an illustrative example, a dataflow analyzer 55632 may transmit, to ML model(s) 55636, a first dataflow from a laparoscopic tool (e.g., a dataflow indicating a measured temperature of an insufflated cavity via sensor 55625) and a second dataflow from a heating pad (e.g., a dataflow indicating a measured temperature near an insufflated cavity via sensor 55613 and/or a power level associated with heat generated by the heating pad (e.g., via output 55617)). The ML model(s) 55636 may determine that an increase in temperature, as measured by the laparoscopic tool, is caused by the power output associated with the heating pad. ML model(s) may indicate a relationship between the laparoscopic tool and the heating pad to the dataflow analyzer 55632 (e.g., that the temperature sensor associated with the laparoscopic tool may be used to control the power output of the heating pad).

Optionally at block 55674, a dataflow analyzer 55632 may transmit an interrogation message to a second surgical element 55620. As described herein, an interrogation message may request, among other things, confirmation that a second surgical element 55620 includes a compatible and/or available dataflow (e.g., based on a determined relationship).

Optionally, at block 55675, a dataflow analyzer 55632 may receive an interrogation response indicating at least one dataflow associated with the second surgical element 55620. In examples, the dataflow analyzer 55632 may receive an interrogation response indicating that a second dataflow is not available. In examples, an interrogation response may indicate an update to information associated surgical element 55610, 55620, and/or an acknowledgement of the information associated with the surgical element 55610, 55620. An update to one or more determined relationships may include an indication of a second sensor that may be related to a failed sensor (e.g., sensor 55625 may be related to sensor 55613 in operational environment 55600B). In examples, the interrogation response may indicate that a surgical element 55620 may be configured to transmit the requested dataflow (e.g., that a dataflow is available, that one or more dataflows may be removed (e.g., taken off-line) to send the requested dataflow, and/or the like).

At block 55676, a dataflow analyzer 55632 may transmit a configuration message to the second surgical element 55620. A configuration message may be sent in response to a received interrogation response, based on a user input (e.g., an HCP 55608 via a GUI), and/or as determined by the dataflow analyzer 55632 (e.g., based on a determination that a dataflow is sending erroneous data and/or the like). A configuration message may include a request for sensor data (e.g., a dataflow) based on one or more relationships (e.g., as determined by the dataflow analyzer 55632, ML model(s) 55636). A configuration message may include dataflow configuration information as described herein, to configure a surgical element 55620 to transmit a second dataflow.

At block 55677, a dataflow analyzer 55632 may receive a configuration response including an indication of the second dataflow. As described herein, a configuration response may include dataflow configuration information and/or a dataflow associated with a surgical element 55620. Dataflow configuration information may include a surgical element ID, an indication whether a dataflow is available, an update and/or acknowledgement to dataflow configuration information, a unit of measure, a communication protocol (RS-232, Ethernet, TCP/IP, Bluetooth, and/or the like), scheduling and/or frequency information (e.g., a time and/or frequency that sensor data is to be sent), a destination (e.g., port information associated with the surgical element controller 556611), and/or security and/or access control credentials.

At block 55678, a dataflow analyzer 55632 may generate control data for the first surgical element based on the second dataflow (e.g., from the second surgical element). Additionally and/or optionally, a dataflow analyzer 55632 may generate control data based on a user input (e.g., by an HCP 55608 via a GUI) and/or as determined by the dataflow analyzer 55632. As described herein, control data may include information associated with, for example, a control variable setpoint (e.g., associated with a motor speed, a temperature, a flow rate, and/or the like), a current and/or voltage (e.g., associated with an electrosurgical tool, and/or the like), an instruction (e.g., to generate an image from a camera and/or the like), and/or a power level (e.g., of a heating pad, a laser ablation tool, and/or the like).

At block 55679, a dataflow analyzer 55632 may cause the first surgical element to adjust an output characteristic (e.g., based on the transmitted control data). Advantageously, a dataflow analyzer 55632 may send control data to a first surgical element (e.g., that may have a failed sensor), based on a second dataflow to avoid pausing, cancelling, and/or continuing a procedure with a reduced number of surgical elements.

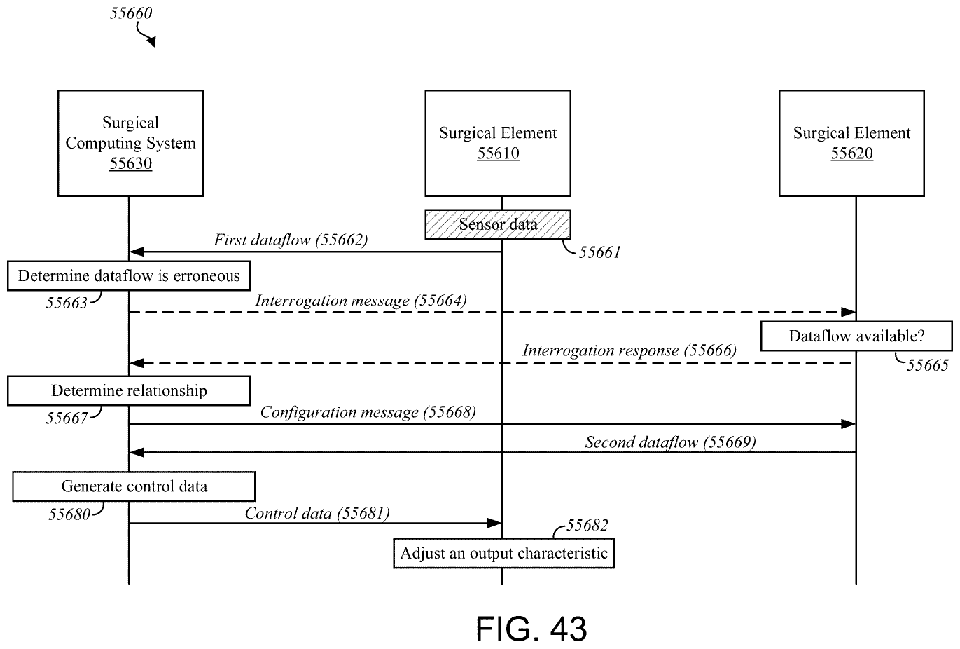

FIG. 43 illustrates an example communication diagram 55660 for re-balancing the number of unknowns and dataflows (e.g., data streams). The example communication diagram 55660 may be described with reference to one or more components of operational environment 55600B, including a surgical computing system 55630, a surgical element 55610, and/or a surgical element 55620. Advantageously, a surgical computing system 55630 (e.g., dataflow analyzer 55632) may use sensor data (e.g., a second dataflow) from a second surgical element to generate control data for the first surgical element (e.g., instead of pausing a procedure to replace the failed surgical element, cancelling a procedure, and/or continuing a procedure with a reduced number of surgical elements). The surgical computing system 55630 may avoid pausing, canceling, and/or compromising safety during a procedure by determining a second dataflow based on one or more associated relationships between surgical elements.

In examples, surgical element 55610 may generate sensor data at 55661. The sensor data may be erroneous (e.g., as indicated in shading). As described herein, erroneous data be caused by an incorrectly calibrated sensor, a misplaced and/or improperly installed sensor (e.g., interference by a heat source), calibration drift, contamination, electromagnetic interference, a failed sensor, operator error, misalignment (e.g., in the case of a position sensor), and/or the like.

Erroneous sensor data at 55661 may be transmitted from surgical element 55610 to surgical computing system 55630 via a first dataflow 55662. A first dataflow may include sensor data, operational data, system generated data, control data, dataflow configuration information, and/or the like. Although FIG. 43 depicts erroneous sensor data at 55661, surgical element 55610 may transmit a dataflow 55662 including erroneous operational data, system generated data, control data, and/or the like.

The surgical computing system 55630 may receive the first dataflow 55662 including erroneous sensor data 55661. The surgical computing system 55630 may determine that a dataflow is erroneous at 55663 based on a determination that control data exceeds a threshold, a determination that the first dataflow is unavailable, a determination that the physiological parameter of the patient exceeds a patient safety threshold, based on a comparison of sensor data to historical data (e.g., past sensor data and/or another data type), and/or based on an output of ML model(s) 55636.

The surgical computing system 55630 may transmit an interrogation message 55664 to surgical element 55620. An interrogation message may be transmitted in response to determining that a dataflow is erroneous. Additionally and/or optionally, an interrogation message may be transmitted in response to a user input (e.g., an HCP via a GUI), and/or as determined by the surgical computing system 55630. As described herein, an interrogation message may request information associated with one or more dataflows of a surgical element 55620. The interrogation message may include dataflow configuration information associated with a surgical element 55610, 55620 (e.g., to determine whether a second dataflow is available).

A surgical element 55620 may receive an interrogation message 55664 and determine if a dataflow is available at 55665. In examples, a surgical element 55620 may determine that a second dataflow is available if a first dataflow is removed (e.g., taken off-line and/or replaced by the second dataflow on a configured channel). In examples, a second surgical element may determine that a second dataflow is available via a second channel and/or determine that a second dataflow may share a channel with a first dataflow.

An interrogation response 55666 may be sent from the surgical element 55620 to the surgical computing system 55630. The interrogation response 55666 may be sent in response to determining whether a dataflow is available at 55665. As described herein, an interrogation response may include an indication whether a second dataflow is available, an indication of an update to information associated with a surgical element 55620, and/or an acknowledgement of the information associated with surgical element 55610, 55620 (e.g., among other dataflow configuration information).

A surgical computing system 55630 may determine a relationship at 55667. In examples, a relationship may be determined based on the received interrogation response 55666. As described herein, a relationship (e.g., between surgical element 55610 and/or surgical element 55620) may be determine based on one or more aspects of a surgical element 55610, 55620 (e.g., dataflows, control data, sensor data, operational data, configuration information, and/or the like), based on data associated with a LUT, and/or based on an output of ML model(s) 55636.

If the surgical computing system 55630 determines that a relationship exists, the surgical computing system 55630 may transmit a configuration message 55668. As described herein, a configuration message may include information associated with configuring a surgical element 55610, 55620. As an illustrative example, dataflow analyzer 55632 may send a configuration message to surgical element 55620, to configure a second dataflow (e.g., via 55622 and/or if determined by the dataflow analyzer via 55629) for receiving sensor data associated with sensor 55625, based on a determination that sensor 55625 is related to failed sensor 55613.

Surgical element 55620 may receive configuration message 55668, and in response, configure the requested dataflow (e.g., select, generate and/or transmit the sensor data requested in the configuration message 55668). In response to receiving the configuration message 55668, surgical element 55620 may send a second dataflow 55682 to the surgical computing system 55630. The second dataflow 55669 may include, among other things, sensor data based on one or more relationships as determined at 55667.

Surgical computing system 55630 may receive the second dataflow 55669, and in response, generate control data at 55680. As described herein, control data may include information associated with, for example, a control variable setpoint, a current and/or voltage, an instruction, and/or a power level. As an illustrative example, control data may include a setpoint for the speed of a cutting tool, a voltage and/or current setpoint of an electrosurgical tool, a flow rate for an infusion pump, a position of a robotic arm, and/or the like.

The surgical computing system 55630 may transmit control data 55681 to a surgical element 55610, to cause the surgical element 55610 to adjust an output characteristic associated with output 55617. The output may adjust an output characteristic at 55682 based on the information associated with a control variable setpoint, a current and/or voltage, an instruction, a power level, and/or the like, as indicated in the control data.

As described herein, the control data 55681 may be generated based on a sensor of a second surgical element (e.g., surgical element 55620), and transmitted to a first surgical element (e.g., surgical element 55610). The control data may be used, for example, to rebalance the number of unknowns and the number of data streams (e.g., dataflows). Surgical element 55610 may receive the control data 55681 and adjust an output characteristic at 55682 as described herein.

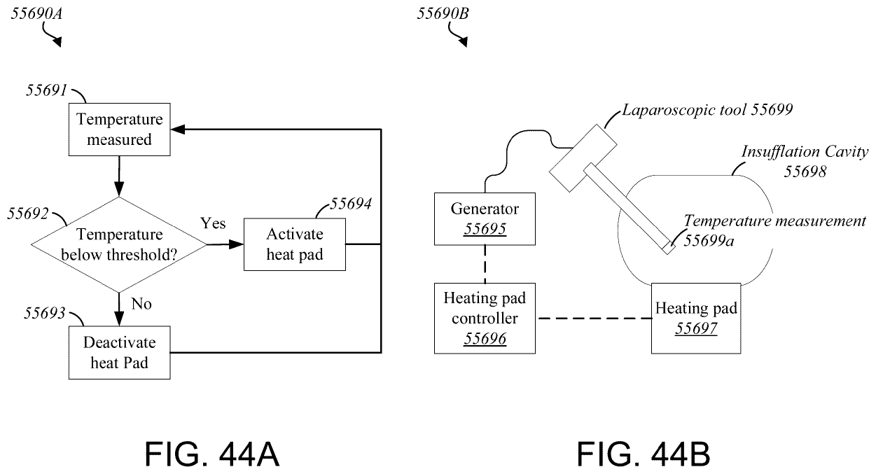

FIGS. 44A and 44B illustrate an example implementation of rebalancing unknowns and dataflows (e.g., data streams) via an insufflated temperature regulation system. Advantageously, routine 55690A and/or environment 55690B may immediately and/or continuously estimate the insufflation cavity temperature, improve heating pad performance and efficiency, and/or minimize patient temperature fluctuations throughout surgery. The following may be described based on a procedure associated with an elderly, low BMI patient undergoing a necessary surgery that is having complications. Temperature measurement 55699a of the laparoscopic tool 55699 may measure the insufflated air temperature of insufflated cavity 55698, and/or proactively the generator 55695 may determine that the patient is at risk of hypothermia without intervention. A warning may be presented (e.g., to an HCP via a GUI) to increase a heat pad setting (e.g., heating pad 55697) or a signal (e.g., control data) may be sent to the heating pad controller 55696, to increase temperature.

A patient may avoid further complication from an already high risk procedure based on the routine 55960A and/or environment 55690B as described herein. In examples, lengthy insufflated surgical procedures may have a risk of causing hypothermia despite the presence of heating pads. During long surgical procedures which may have leaky ports, frequent smoke evacuations, patient response to generate anesthesia, and high gas flow rates, the internal body temperature of the patient may drop to unsafe levels.

To address this issue, supplemental heating in the form of heating pads 55697 may be provided to the patient (e.g., insufflation cavity 55698). To automatically match the patient specific heating needs, a laparoscopic tool 55699 may detect the ambient temperature of the insufflated cavity (e.g., via temperature measurement 55699a). The temperature measurement 55699a may regulate the amount of heating provided to the patient. Heating pads 55697 may be further improved by the temperature measurement 55699a from the energy tool (e.g., heating pad controller 55696). The provided temperature may be used to autoregulate the heat setting of a patient heating pad 55697 to minimize temperature fluctuations throughout a surgery (e.g., autoregulated by the generator 55695).

FIG. 44A illustrates an example routine 55690A for temperature control. FIG. 44B illustrates an example environment 55690B for executing one or more functions associated with routine 55690A. The example environment 55690B may include a generator 55695 (e.g., similar to surgical computing system 55630 of FIG. 41B), a heating pad controller 55696 (e.g., similar to surgical element controller 55611 of FIG. 41B), a heating pad 55697 (e.g., similar to output 55617 of FIG. 41B), an insufflated cavity 55698 (e.g., similar to a patient 55641 of FIG. 41B), and/or a temperature measurement 55699a (e.g., similar to sensor 55625 of FIG. 5B).

Routine 55690A begins at block 55961 where a temperature is measured by temperature measurement 55699a of a laparoscopic tool 55699. The temperature may be measured for an insufflated cavity 55698. In examples, temperature measurement 55699a may include one or more similar features and/or functions as sensor 55625 of operational environment 55600B.

At decision node 55692, a generator 55695 (e.g., a dataflow analyzer 55632) may determine whether the temperature measurement 55699a is below a threshold. A temperature estimator model (e.g., as part of generator 55695) may determine the environmental temperature throughout a procedure. If the insufflation cavity 55698 temperature is lower than a threshold (e.g., an internal body temperature of the patient), the routine may continue to block 55694. If the insufflation cavity 55698 temperature is not below a threshold (e.g., above an internal body temperature of the patient) the routine may continue to block 55963.

At block 55693, generator 55695 may transmit a message (e.g., as part of control data) indicating that heating pad controller 55696 may deactivate heating pad 55697. After heating pad 55697 is deactivated, the routine 55690A may loop back to block 55691 and/or end.

At block 55964, generator 55695 may transmit a message (e.g., as part of control data) indicating that heating pad controller 55696 may activate heating pad 55697. After heating pad 55697 is activated, the routine 55690A may loop back to block 55691 and/or end.

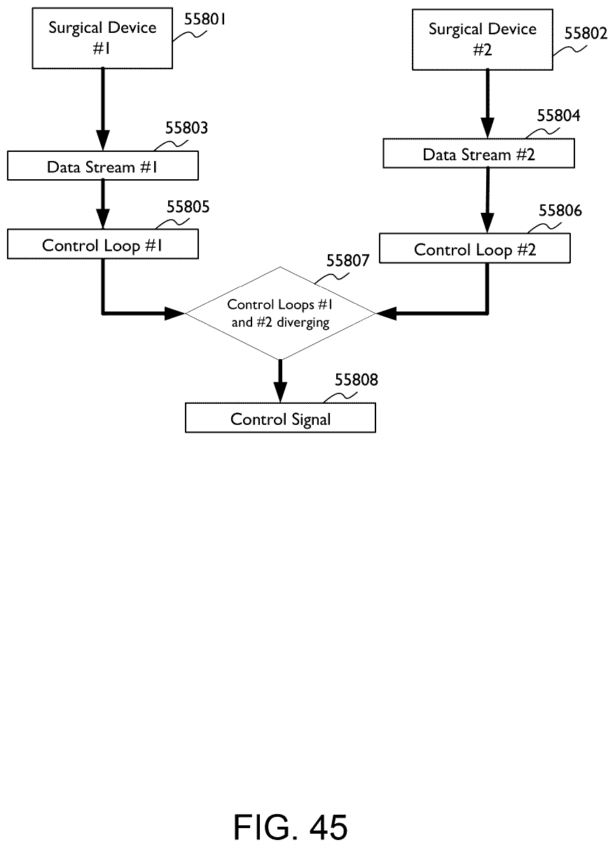

FIG. 45 shows an example computer-implemented surgical system for comparing multiple (e.g., two) differing data streams to determine a control signal for controlling the system. The surgical system may include or may interact with multiple surgical devices such as a first surgical device 55801 and a second surgical device 55802. The first surgical device may include any surgical instrument and/or equipment that may generate relevant surgical data, for example, temperature monitors (e.g., thermometers), pulse oximeters, blood pressure monitors, capnographs, electrocardiograms, spirometers, intraoperative blood glucose monitors, intraoperative fluid monitors, intraoperative nerve monitors, hemoglobin monitors, intraoperative pressure monitors, electroencephalography (EEG) monitors, ultrasounds, electromyography (EMG) devices, dosimeters, or non-invasive cardiac output (NICOM) monitors, and the like. Likewise, the second surgical device may include any surgical instrument and/or equipment that may generate relevant surgical data, for example, temperature monitors (e.g., thermometers), pulse oximeters, blood pressure monitors, capnographs, electrocardiograms, spirometers, intraoperative blood glucose monitors, intraoperative fluid monitors, intraoperative nerve monitors, hemoglobin monitors, intraoperative pressure monitors, electroencephalography (EEG) monitors, ultrasounds, electromyography (EMG) devices, dosimeters, or non-invasive cardiac output (NICOM) monitors, and the like. The first surgical device 55801 may output a data stream (e.g., a first data stream 55803) associated with a measurement (e.g., a temperature, or O2 level) and a first control loop 55805 (e.g., increasing or decreasing a patient temperature using a temperature management system). The second surgical device 55802 may output a data stream (e.g., a second data stream 55804) associated with the same measurement and a second control loop 55806. In some examples, the first data stream 55803 and the second data stream 55804 may provide conflicting data. A disagreement between the first data stream 55803 and the second data stream 55804 may be detected. The control loops may be determined to be diverging 55807. A control signal may be generated 55808.

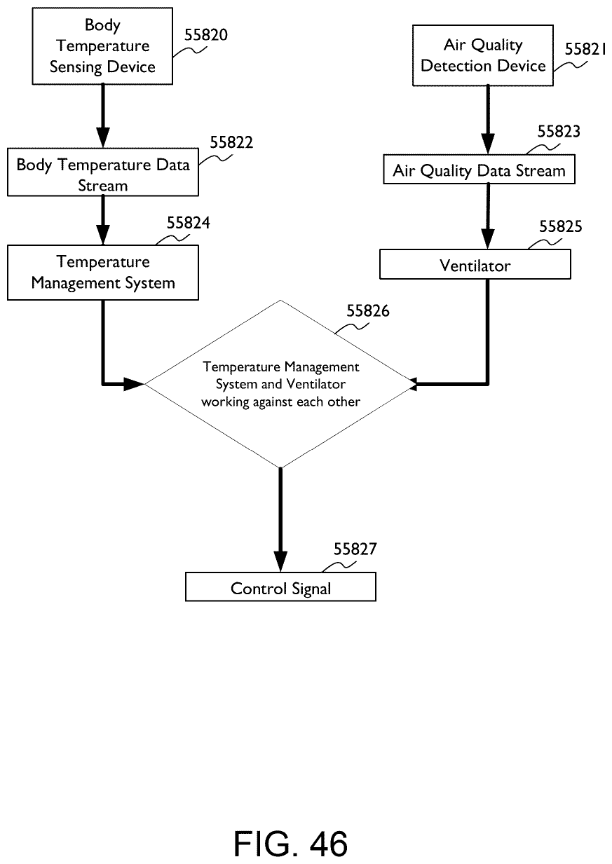

FIG. 46 shows an example computer-implemented surgical system for comparing multiple (e.g., two) differing data streams, including a body temperature data stream 55822 measured by a body temperature sensing device 55820 and an O2 data stream 55823 measured by an air quality detection device 55821.

The temperature data stream, for example, may be obtained via a body temperature measuring device 55820 (e.g., an esophageal temperature probe, nasopharyngeal probe, skin surface thermometer, infrared or tympanic membrane thermometer, etc.). The body temperature data stream may be sent to a temperature management system 55824 that may change the body temperature of the patient (e.g., using forced-air warming systems like a Bair Hugger™, a fluid warming system using intravenous (IV) fluids, blood, or other fluids administered to the patient during surgery, a heated mattress or pads like a HotDog™ patient warming system, water-circulating warming/cooling systems like a Cincinnati Sub-Zero (CSZ) Blanketrol™, intravascular temperature management systems, warming blankets, radiant warming devices, heat and moisture exchange (HME) filters, warming lights, etc.).

The air quality data stream may be measured using capnography, spirometry, oximetry, environmental monitoring systems, airway pressure monitors, real-time gas analysis, or smart ventilation systems, for example. The air quality data stream may be sent to a ventilator 55825 (e.g., an air quality management system including but not limited to a ventilator). Some ventilators may include intraoperative temperature management systems including using heated breathing circuits and/or forced-air warming systems. For ventilators without an integrated intraoperative temperature management system, the ventilator may be operated in tandem with a temperature monitoring system and may work against each other 55826. Ventilators without a temperature monitoring system may lower the temperature of the patient through delivery of dry, cold air through the output of a control signal 55827.

The air being delivered by the ventilator may not cause the body temperature of the patient to drop instantly but with the temperature monitoring system communicating with the ventilator, the temperature monitoring system may engage early to preemptively warm the patient and/or counteract and cooling effects the ventilator has on the body temperature of the patient.

Maintaining a stable, comfortable temperature for the patient may be difficult without the temperature monitoring system and the ventilator being able to communicate. A delay in the feedback control system (e.g., a delay in the cold air being sent to the patient and a measured body temperature of the patient) may have negative effects on the response of the system. The response of the system may include offsetting the control action, oscillations, instability, and/or performance degradation.

Offsetting of a control action may include a delayed response of the temperature monitoring system. For example, if a lower temperature is detected in the body temperature of the patient, the temperature monitoring system may begin warming the patient but because the response of the temperature monitoring system has a delay as well, the total offset of the control action may be much greater than the time a control action may take place if the temperature monitoring system and the ventilator could communicate.

Oscillations and/or instabilities may include the similar concept of the offsetting of the control actions, except, for example, if the body temperature of the patient lowers, the temperature monitoring system may begin to heat. The ventilator may turn off causing the internal body temperature of the patient to stop lowering. The temperature detection device may not detect this change immediately so the temperature monitoring system may continue to heat the patient and may potentially cause the patient to overheat since the body temperature is not lowering from the ventilator anymore. This cycle may continue and cause oscillations in the control signal.

A third data stream associated with the measurement may be obtained, for example, the temperature of the room. The third data stream (e.g., the room temperature) may be associated with a third control loop of the surgical system (e.g., a thermostat that controls the temperature of the room). The generating of the control signal may be further based on the third data stream.

The selection of one of the first and second data streams may include determining a current physiologic situation associated with a patient (e.g., that a patient has hypothermia). A control parameter (e.g., raise the body temperature of the patient using the temperature monitoring system) may be selected based on the current physiologic situation associated with the patient. The data stream (e.g., the body temperature of the patient) may be associated with the selected control parameter is selected. The control signal (e.g., the temperature monitoring system) may indicate to the system to increase the body temperature of the patient using a weighted combination of the body temperature data stream and the O2 data stream. If the second data stream was another temperature data stream instead of the O2 data stream but measured in a different unit of temperature as compared to the first temperature data stream, then the second data stream may be transformed (e.g., the unit may be changed). In examples, the control signal may be the difference between the first and second data streams.

If a cause of the divergence is known, for example, if the body temperature of the patient is drastically rising because the patient is experiencing blood loss, a control signal may take into account this circumstance and indicated to the temperature monitoring system to increase the temperature of the patient. Furthermore, a measurement difference between the first data stream and the second data stream may be calculated and/or compared to a threshold value. For example, if the body temperature of the patient is determined to be only slightly lower than desired, then the body temperature monitoring system may slightly warm the patient.

Surgical devices of the surgical system may need additional input to generate a control signal. Activity of a current system may be measured. Measured activity of the system may be used to determine if a decision is warranted. Measured activity may also be used to determine if a data feed is potentially compromised.

Real-time data and a change of an operating status may be provided. In examples, advanced energy may be being used for the dissection and mobilization of a colon in a cancer surgery. The adhesion and connective tissue mobilization may be achieved through the use of ultrasonic handpiece. An ultrasonic handpiece has the benefit of being able to cut while not causing much collateral thermal damage due to the way it produces local heat. However once mobilized the surgeon may prefer to use a bipolar RF handpiece for coagulation and transection of the mesentery and/or arteries. The energy generator may change from the ultrasonic generator to the RF generator. This change may be an internal operation of the advanced energy generator implies a dramatic change in the way the smoke evacuator need to react to an energy activation, since the RF produces much more smoke and steam during use. This smoke evacuator adaptation of the amount of exhaust air flow relative to the real time monitoring of the generator activation timing may increase the removed liters of gas from the abdomen and therefore also require a subsequent increase in the amount of cold CO2 the insulator pumps in. This change in thermal loading in combination with real-time measurement of core body temperature could induce a very different response request of the patient heating system and its reaction sensitive of it triggers to changes in input thermal load to the patient.

A change in energy modality usage along with real-time core body temperature monitoring may change the magnitude or even need for a changed in decision for closed loop patient heat thresholds.

The current reading may be compared to the same reading earlier in time. This may be to prevent signal dropout. Signal dropout may be caused by wires being disconnected, damaged equipment, physiologic change(s), signal noise, etc. Wires being disconnected may be from the data acquisition device and/or the operating room (OR) equipment and/or leads detached from patient. Damaged equipment may include frayed wires, damaged connectors, etc. Physiologic change may include vasoconstriction effects on the transcutaneous O2, for example. Signal noise may be an effect of damaged equipment or other devices may impact signal. In examples, a cone beam CT may cause issues with other electronic signals.

The trend or pattern of behavior with a previously known behavior may be compared to predict the cause of divergence. Data may go up and down erratically over a large period of time. In examples, the rate at which O2 is supplemented to the patient may be dependent on CO2 offgassing measured at the patient finger, transcutaneously. Supplementation may likely change over time as a result anesthesia inducing a decrease in metabolic activity. In one instance, the patient O2 supplementation may double when compared to the second/30 seconds prior. As it is unlikely for metabolic activity to increase so quickly, this change may trigger the system that a decision is needed to confirm the validity of the datastream.

A reaction may include confirmation of the O2 supplementation compared to the CO2 offgassing (determine if shifts are present in both data streams or isolated to O2). Additional parameters such as patient temperature, tidal volume, O2 blood gas may be used as reference data streams to evaluate if the shift is visible in other patient metrics. In a similar instance, if O2 supplementation were to instantaneously double and then return to the initial level, the system may trigger to monitor or assess that input stream to evaluate the validity.

Response options may be provided to questionable data. In reacting to data, data streams may shift and/or stay at an altered level. The system may identify the cause of the shift. The system may continue to monitor the data, for example, an unexpected discrete shift in an individual data stream may occur.

A data and/or shift may occur. In examples, EKG data may have noise and/or be present at start up. The system may compensate for noise within equipment because procedure has not yet altered patient state. Utilizing a time based aspect of a data stream may provide context of the change. A smart system may know to condense two unrelated data streams into a single composite stream instead of seeking another more useful stream. In examples, ambient room temperature may be measured, a finger transcutaneous temperature may be measure, and an internal body temperature may be measured. A combination of these temperature streams may provide a net difference which may provide new information. The difference may be indicative of vasoconstriction when these 2 metrics don't align.

Visible light and infrared light may be combined from these data streams to get a new image. A whoop band may measure heart rate, temperature, and/or a breathing rate for example. Body ‘strain’ may be calculated to evaluate response to exercise.

In examples, staple firings may include a number of activations, energy per activation, whether the system is smoke evac-ed, etc. A decision to combine this data may be include feedback control dependent on complexity of the system. If the system is unable to seek another stream because of space/capacity, the system may select to use existing streams. Bandwidth may be constrained if no room is available for another input. If data is not coming in quick enough for control loop, speed of execution may drive which data source is useful. Speed of control loop may be measured. As the speed approaches a threshold where the system loses ability to stay deterministic, a data stream may be selected to meet system ability.

Lack of confidence/reliability in ‘more useful’ data stream may occur. This may be due to interference with an existing procedure. Additional time may be needed to obtain separate equipment. Existing space constraints may limit ability to add other equipment. In examples, using existing Monarch ME position instead of adding cone beam CT. User location/current data in view may be provided. In examples, a Garmin watch, a built-in HR monitor vs bluetooth ancillary monitor, etc., may be provided.

If data streams are in different formats, transformation required to combine the data. Some combinations may require alignment. The system may be unable to make a decision. A low patient may have low risk event with incomplete data. A worst case may include an outcome of a ‘delay in surgery’. The system may be able to select either because the patient risk is low. In an example with a high risk patient with bleeding, for example, lack of action may create a high risk event and the system must continue operating normally.

The best function may be prioritized. If the system is unable to make a decision, the system may reduce the number of data streams it is deciding between and focus decision only on the critical data elements. Critical elements may be based on risk profile of patient, surgery type, equipment limitations.

Pre-procedure and/or step priority of available data may be provided. If a decision is needed, the system may ignore limitations of adding new data stream and bring more critical decision data forward, de-prioritizing visualization/collection of background data to get through a step. Time driven in steps where critical structures/high risk failures are present, may be provided. Intentional pause prior to key surgical step may be provided. Data and/or a baseline may be collected in a moment and remove the non-critical streams so that the system is prepared for more critical activity.

The system may pause and alert the surgeon of the need for a decision. If the system is using an automated movement that the surgeon selected, the automated movement may be paused. Slow down activity may be provided.

Without ML/strategic decisions, equations may be more difficult to determine. Key equations may be hard-coded into the system that it abides by. A MISO prediction model may be provided. If the processor is not able to predict the future force, it may not act. The ML process may have a mitigation watch dog to confirm system calculation is done in an appropriate amount of time, if it is unable to, the system may proceed in a non-smart way (e.g., default algorithms). Hard code inferences in may include logic ladders within the software to use specific data to solve unknowns ML would allow the system to use different/other streams.

Predictive resultant simulation may be provided and may trend in the wrong direction. O2 and temperature data may be combined, for example, trending toward an issue. A technique of a surgeon may be based on specific surgeon technique determined for the best outcomes. In examples, harmonic lift may be provided. Types of Simulation include, but are not limited to, the following. Discretely identify multiple options. Multiple sets of data or complex data may be provided including, but not limited to, heart rate, body temperature, O2, etc. Trending of data due to physiologic response may be provided. Context of the data for their specific treatment based on the globally accessible data may be provided. Context relative to an encountered irregularity may be provided.

Predictive simulation may be displayed to the user. Simulation communication-ability and display-ability may be based on device capability and availability. Predictive data may be compared with current patient/procedure information in a simulation driven by historic procedure patterns/behaviors to enable the surgeon to decide. The surgeon may use a predictive intra-op simulation. The surgeon may request additional information once a tumor is reached, for example. In examples, new information may be identified intra-op, the surgeon may adjust simulation inputs/parameters to confirm a procedure approach. For example, maps may be refreshed after you make a turn (if you change path). If surgeon deviates from initial plan, information may be re-run to provide a new path.

Visually evaluating drug diffusion and responding if it does not match an expectation (couple with visualization technique to look at drug presence as input) may be provided. The system may ask the user about simulation findings or it may auto-adjust. In examples, a system may identify a risk reduction opportunity and guide a user to lower risk option.

Display leakage paths/cautionary area may be provided. Simulation to value multiple options including additional data streams may determine which one or combination is most useful to resolve. Determinization between finding more data streams or less unknowns may be provided. In examples, chemo saturation, leakage or spray out conditions may be predicted. Microwave ablation may include, at location X, using simulation to guide position of tip/settings of system prior to ablation. The system may display choices of parameters/recommendations for surgeon activity, ranked by risk/time/patient outcome, etc. As resistance increases to drug insertion in the tumor, either pressure may be increased or the needle may be repositioned. In determining the best option, ease of application may be considered. Also, a potential risk of collateral damage may be considered. For example, which option provides the least likely risk to squirt out, or least risk of over-saturation for example. A magnitude of treated space may be considered including the option providing the least chemo necessary. A highest percent saturated option may include considering between tipping a needle vs an additional insertion. Additional data may be required to understand tumor parameters (ex. density) that drive success of drug diffusion. Additional information, such as lung tissue health, may also inform the impact of tissue damage surrounding the tumor/likelihood of patient recovery. Tumor location may drive that although tipping is the best option, it is not accessible within the given space, for example, Secondary imaging may be utilized to improve probability of correct simulation. A shift may be made from transformed data to non-transformed data for ancillary parameterization of the site or tumor. Tumors may be highly vascularized which means IR imaging may add a layer of detail that is complimentary to imaging the tumor itself.

Other systems may be complimentary. A pre-defined system ‘drum beat’ chosen by the surgeon may drive the pace it is updated. Different data sources may require different time between updates. Data streams may be parsed. For example, breathing every 2 seconds, temperature every 10 seconds, patient table position every 30 minutes, HCP presence/location, etc. Changes in data streams may be used to drive updates. In examples, a tumor fill may be re-run as a simulation constantly once drug delivery begins. Key metrics may drive simulation change, for example, in reverse trendelenberg, a BP may be more sensitive.

The user may know if it's updated/when an indicator on screen illuminates during update, a tone to indicate update in progress, the surgeon may make a manual selection of update via button, the surgeon may choose to rerun the simulation, the simulation may rerun in the background but wait to update until surgeon request, light/brightness of the output (as information ages the content fades) may change, etc. If ML is used, the system may transition from simulation to patient/procedure data. Categories of types of simulation vs real-time data may be provided. All simulation data may be provided. A blend of simulation and real-time data may be provided. For example, map directions (simulation) constantly may be reassessed with real-time data, and be dependent on driver movement/decisions for continuous guidance. In examples, an energy device impedance that algorithm is using may constantly be updated throughout the procedure and may simulate different impedance values to understand impact of impedance on outcome. In examples, after clamping on one vessel with a simulated tissue algorithm, impedance may be used to understand tissue response to energy activation and simulate a range of values to understand likely outcome based on the patient specific metric. All real-time data may be provided. Continuous Simulation may be provided which may include no patient data including the example of a simulation running in the background where updates may be based on a position/procedure step.

Triggers to turn off the simulation may include being outside bounds of simulation, outside OR/simulated space, etc. Outside bounds of simulation may include user deviation from the procedure plan or procedure step introduced that wasn't included in simulation. A high error rate may cause the simulation to no longer providing useful data. The user may turn off the simulation.

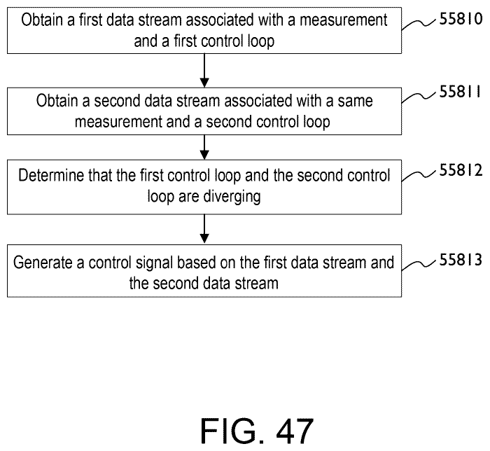

FIG. 47 shows an example flowchart for determining a control signal. At 55810, a first data stream may be obtained. The first data stream may be associated with a first control loop. At 55811, a second data stream may be obtained. The second data stream may be associated with a second control loop. At 55812, the first control loop and the second control loop may be determined to be diverging. At 55813, a control signal may be generated based on the first data stream and the second data stream.

In examples, a determination may be made between two seemingly accurate but conflicting data streams.

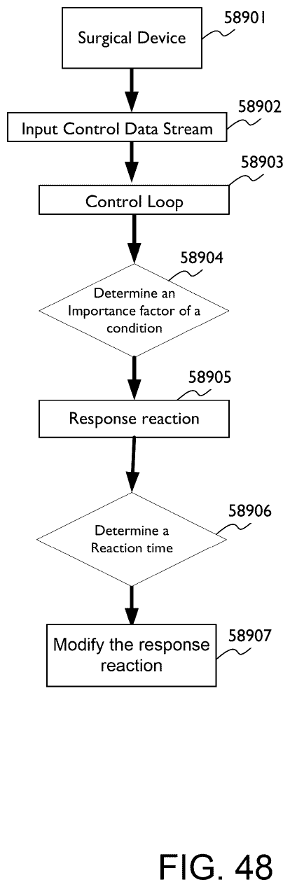

FIG. 48 shows an example computer-implemented surgical system for determining a response reaction and modifying the response reaction. The surgical system may include or may interact with a surgical device 58901. The surgical device 58901 may output an input control data stream 58902. The input control signal may be associated with a control loop 58903. An importance factor of a condition may be determined 58904. A response reaction may be determined and output. A reaction time may be determined 58906. The response reaction may be modified 58907.

The system may not respond to a signal change. If the system is unable to decide what to do, the system may set an alarm condition. The presence of a surgeon may be dependent on the alarm system. The system may actively identify if the surgeon is present. These methods to detect surgeon presence include but are not limited to an eye tracking and/or camera system. If the system requires a response, it may generate an alarm condition.

Alarms for a healthcare professional (HCP) may go beyond the presence of a surgeon. The alarms may call for a scrub tech, an anesthesiologist, etc. Event timestamping and timeliness watchdogs may be provided. Data may be time stamped to understand the timeliness associated with it, and corresponding watchdogs may be implemented. If a watchdog service expires (or is not satisfied), then the system may escalate to a potential action.

Potential actions may be taken without surgeon feedback. Reversion to an immediately prior value may be the action selected, including but not limited to, reversion to a default state, change to a non-clinical state, proceeding with a ‘best’ action, proceeding with a ‘safest’ action, etc. Predefined actions for when a timer expires may be provided. Prior to the surgery occurring, the surgeon or HCP may define what will happen in the event that the surgeon or other HCP is not able to provide feedback in a sufficient amount of time.

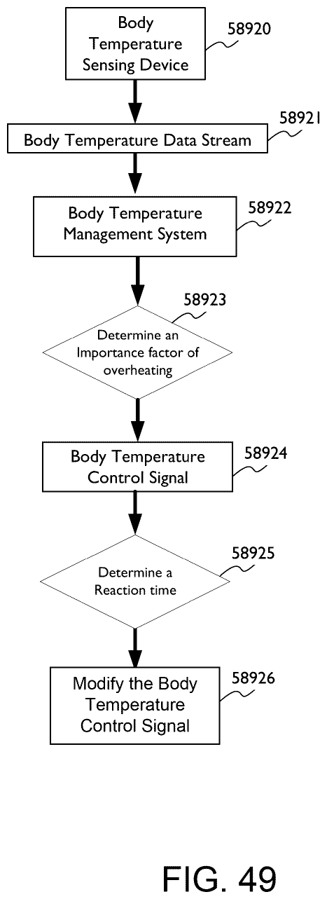

FIG. 49 shows an example computer-implemented surgical system for determining a response reaction in a body temperature control signal 58924 and modifying the body temperature control signal 58924 based on a determined reaction time 58925. A body temperature sensing device 58920 may detect a body temperature of a patient and may output a body temperature data stream 58921 which represents the body temperature of the patient. The body temperature of the patient represented as a data stream may be fed to a body temperature management system 58922. The body temperature control signal may be modified 58926.

The surgical device 58901 may include, but not be limited to, temperature monitors (e.g., thermometers), pulse oximeters, blood pressure monitors, capnographs, electrocardiograms, spirometers, intraoperative blood glucose monitors, intraoperative fluid monitors, intraoperative nerve monitors, hemoglobin monitors, intraoperative pressure monitors, electroencephalography (EEG) monitors, ultrasounds, electromyography (EMG) devices, dosimeters, or non-invasive cardiac output (NICOM) monitors. The surgical device 58901 may output a data stream (e.g., an input control data stream 58902, or a body temperature data stream 58921) associated with a measurement (e.g., a temperature, or O2 level) and a control loop 58903 (e.g., increasing or decreasing a patient temperature using a temperature management system 58922).

The temperature data stream, for example, may be obtained via a body temperature measuring device 58920 (e.g., an esophageal temperature probe, nasopharyngeal probe, skin surface thermometer, infrared or tympanic membrane thermometer, etc.). The body temperature data stream may be sent to a temperature management system 58922 that may change the body temperature of the patient (e.g., using forced-air warming systems like a Bair Hugger™, a fluid warming system using intravenous (IV) fluids, blood, or other fluids administered to the patient during surgery, a heated mattress or pads like a HotDog™ patient warming system, water-circulating warming/cooling systems like a Cincinnati Sub-Zero (CSZ) Blanketrol™, intravascular temperature management systems, warming blankets, radiant warming devices, heat and moisture exchange (HME) filters, warming lights, etc.).

Maintaining a stable, comfortable temperature for the patient may be difficult without the temperature monitoring system and the ventilator being able to communicate. A delay in the feedback control system (e.g., a delay in the cold air being sent to the patient and a measured body temperature of the patient) may have negative effects on the response of the system. The response of the system may include offsetting the control action, oscillations, instability, and/or performance degradation.

The modification of the response reaction may be an escalation. For example, the response reaction as an escalation may be the body temperature management system indicating to raise the body temperature of the patient. The modification of the response reaction may be a recession. For example, the response reaction as an escalation may be the body temperature management system indicating to lower the body temperature of the patient.

The reaction time may be based on the importance factor of the condition associated with the patient, for example, a risk of the patient overheating may be a condition with a high importance. For example, an importance factor associated with overheating may be determined 58923. The importance factor may be based on a patient risk, so for a patient more prone to overheating due to age, previous medical conditions, or the like, the importance factor may be determined to be higher, for example, than a patient who is younger, has no previous medical conditions, etc.

An instant of the input control data stream is determined may be where the input control data stream violates a first threshold associated with the input control data stream, for example, when a body temperature of a patient raises above the critical temperature level determined to cause the patient to overheat. An instant the input control data stream is determined may be where the input control data stream satisfies a second threshold associated with the input control data stream.

The control loop of the surgical system may be a closed loop system or may be changed to an open loop system. An anticipated instability may be prevented from affecting the surgical system based on a change in the first data stream or the second data stream, for example, if a body temperature of a patient is determined to be rising, preemptive action may be taken by the body temperature management system to prevent a case of overheating for the patient.

Primary surgical actions and secondary surgical actions may be provided. A primary surgical action may be defined as a critical surgical step, such as one performed by the surgeon themselves. A secondary surgical action may be one that is supporting primary surgical steps but performed by someone else. This could be control of anesthesia, preparation of devices, etc. In examples, a surgeon may be sitting at the console performing an action, and an arm has been retracted to perform a reload. The reload action still may not be performed within several minutes. In examples, patient temperature may be dropping, and O2 supplementation may slow try to accommodate this. Then the temperature system may drop. The system may hold the level and/or pause until the surgeon is available. Required response times from a surgeon may be variable depending on the type of surgical action. Categorization of these actions may then influence how the system informs and/or responds to the scenario.

Timeliness of response predicted on the severity of the action may be provided. In examples, a scenario in which a critical structure may have been inadvertently damaged may require a much faster HCP response than a scenario where the risk is significantly lower.

A timing of a response based on the response strategy may be provided. Prior decisions and/or surgical goals may have been previously categorized as reactive, proactive, preemptive, predictive, preventative, etc. How the system has categorized these responses may then inform the next steps and timing for the responses, knowing that the overall category it was working within was previously defined.

Minimum required signal change for system action may be provided. Not all stimuli may result in a signal change. Small signals may be filtered out and/or prevent the system from over-compensating to what may be overall small changes within the procedure. The system may identify the signal change, but mitigation may not be activated.

A signal alarm may be provided. If the system fails to respond to a signal change, and the system may internally detect the lack of response, the system may create a signal alarm. The system may fail to identify the signal change. Secondary gating control for the system may be provided. The system may correlate certain procedure steps or segments to other secondary control inputs. These may be a sequence of operations, heartbeat data from equipment, or other controls to ensure the system is operating within compliance. In examples, the surgeon may move to the next surgical step, but there may be no change in any of the system inputs. This may indicate that there may be an error in equipment monitoring.

Timer alarms may be provided. Timer alarms may be preemptive, predictive, preventative, etc. An absence of a change in state may be provided. The system would have expected (based on a secondary, controlling or gating input) that a step should have occurred. Since the step should have occurred, and has not yet, the system should take a pre-emptive step to control for that. In examples, O2 may drop and a bear hugger may be turned on. In examples, in the context of an AFIB and a patient with hypothermia, the system may reduce O2 supplementation to preemptively adjust for metabolic slowing. In examples, moving a stapler to clamp on a stomach, an arm with an endocutter may be approaching a collision/minimum distance with arm holding scope outside of the patient. In examples, a first arm may be within a certain distance of a second arm. The system may pre-emptively move the second arm out of the way so that the first arm does not collide.

A simulation may be provided. In examples, in a pre-procedure trocar placement simulation context, simulation may be used to evaluate robot arm movements required to complete the procedure and provide recommendation for ideal trocar position to avoid collision. Simulation may further be provided to inform which tools to place in each arm to enable access and avoid collision. A surgeon may move a first arm to avoid joints externally and an internal tool of the second arm. A simulation may be run to inform ideal position with maximum available displacement.

An OR space may be moved via a third input (e.g., move the stomach to a more ideal location using arm C with a grasper tool). The system may predict these behaviors outside of using the procedure plan. In examples, a robot arm with an ‘efficiency’ reset may be provided. A complicated procedure step may require extra motion/extension. The robot may continue with the arm fully extended and/or it may preemptively retract the joint to a maximum ‘efficiency’ placement. In examples, data may be fed over time, and a data feed may drift out of range. The system may preemptively restart and/or reset to reduce error. In examples, a computer may restart mid-day. In examples, data reduction may be escalated from 2 to 3 data sources, and at some point the system may reduce the number of streams to avoid overloading the data pipe.

Predefined thresholds of behaviors to determine legitimacy may be provided. The system may monitor the overall status of signals, and/or actions, relative to predefined thresholds. When the signal starts to move outside of those thresholds, it may become an indication that the signal may warrant a pre-emptive response. These thresholds or guard bands (e.g., control points) may not necessarily be the same as critical fault failures but may provide early indications for the system to take action.

Monitoring of the variability of the signal may be provided. Signals may be monitored not just to their absolute value, but to the amount of variability present within their data stream. If the variability increases over time, it may be an indication of equipment failure or other problems within the system that need to be addressed. Signal standard distribution and associated statistical variability may be monitored. Intra-data stream variability, such as point-point variability with distinct datapoints may be provided.

System health monitoring of a characterized system may be provided. A biological passport (e.g., a corollary) and it's use with detecting doping in professional sports may be provided. A characterized system may have a unique and/or distinct number of characteristics to it. A unique profile may be created relative to the characteristics of the specific equipment. Examples of these characteristics may include the amount of force to move certain joints for nominal movements, power consumption in standby, or other indications that fall within the normal operational conditions but are unique to this system. The system may monitor for changes in these unique parameters as a method to predict when an error is occurring within the system, or performance may be slowly degrading over time.

This response may occur for an imbalanced number of unknowns. If an additional unknown is predicted, this response would add an equation before the system is imbalanced. In examples, monopolar application may cause an interference with the EKG, transcutaneous, and other patient attached monitors and may be ignored or put into open loop condition as an anticipation of the upcoming occurrence and the prevention of the occurrence from causing the unbalance or instability all together.

Importance may be high as the interference from the monopolar device may cause false alarms on the EKG for heart stoppage, may interfere with pace maker controls, even may interfere with closed loop ventilator controls. The decisiveness is also of high important as the corrective action needs to be switch to before any of the critical risks to patient health monitor are triggered.

Preventing an anticipated instability from changing the controlled system may be based on a change from the input system (e.g., prevents a change). A proactive response to an external data stream trigger that may affect the operation or stability of a closed loop feedback system due to a potential input instability may be provided. Triggers for detectable events/trends that may generate an anticipatory trigger may be provided.

A rate of change of a signal may preempt a threshold crossing scenario where the derivative (rate of change) of a parameter may indicate a fault or error that may occur prior to an actual threshold boundary being crossed. In examples, a powered endocutter may encounter a stall condition when the force encountered by the drivebar exceeds the capacity of the motor gearbox system to provide sufficient force to continue moving forward. When a true stall is encountered, removal of the endocutter may become more difficult. This stall condition may be detected as well by monitoring the force being current generated in relation to other parameters. During a drive action, the rate of change of force may instead be monitored, and if force is accelerating rapidly, may predict when a stall condition may occur. This may allow the stall condition to potentially be prevented from occurring in the first place, and alert the system that the tissue and/or firing location may warrant further inspection.

Impacts of differing external signal variation may be provided. The signal that the closed loop system is tracking may vary irregularly in both directions from zero. A measurement context to intelligently interpret the signal may be provided. Viable changes, limits and physiology behavior may be used to identify what portions of the signal may be real. O2 requirements for the body for instance may not need second by second gross adjustment. In the case of a noisy signal, the input feed may be smoothed or averaged over a time frame like 1 minute which may remove the instability of the closed loop but not require it to seek another input source. Pacemakers and EKG leads, for example, don't have the freedom to average overtime without effecting the primary short term function of the closed loop system. This example, in contrast, may resolve the input signal irregularities directly rather than using time to average out the overlaid error.

Intermittent signal loss may occur. A signal may remain somewhat stable when active but may have dropouts that induce instability on the closed loop system if every data point is reacted to. Time dependent averaging may be used to smooth the signal, depending on what percentage of the time is drop out. The challenge would be drifting or varying real adaptations could be masked by the drop out timing and the smoothing.

Characterization of the signal loss may be provided. If the loss portion may be easily identified (i.e. zero, +12V or −12V) then these data points may be filtered out and removed leaving just real signal changes and the transition zones. The signal may be smoothed or cleaned with other transformations to lose less data.

In the scenario where the signal is trending toward a threshold, but the threshold has not yet been reached/exceeded, a change in the data stream (trend/slope/rate) may occur. The number of times a triggering event has impacted closed loop behavior in the recent past may be provided. In examples, a moving stapler may clamp on the stomach, an arm with an endocutter may approach a collision/minimum distance with the arm holding the scope outside of the patient. The system may notify/warn surgeon that arm is approaching the threshold. The first arm may be within a certain distance of the second arm. The data frame rate may be increased for resolution of the arm position to better improve the ability of system to avoid collision. The first arm may be within a certain distance of the second arm. The slow speed of the arm movement may prevent over-accelerating the first arm forward into the second arm.

Responses to control signal instabilities may be provided. An upcoming procedural step may impact the data stream. Catching a trigger may help minimize overshoot. An alternative system data feed may be utilized as a means to anticipate the initiation of the interdependent system. In examples, Hugo Robot may be able to solve position of tools within the patient and may know the position of each tower. In order to prevent collision/entanglement the robot may select optimal joint movement to achieve procedural steps. To do so, it may solve for position of the external portion of the arms, which require additional data input via simulation, OR cameras, etc. In examples, in an AFIB context, a patient temperature may begin to drop but CO2 outgassing may be constant. The system may reduce O2 supplementation to preemptively adjust for anticipated reduction of metabolism.

Circumstances when predictive behavior is beneficial may be provided. Physiologic patient changes may make predictive behavior beneficial. In examples, a body is a closed loop system outside of the system closed loop control. This level may allow the system closed loop to adjust as patient parameters shift. Minor changes in the operating procedure may be provided. The surgeon may deviate slightly from a procedure plan, which means the system may accommodate the changes. Major changes in a planned procedure may be provided. Complications or significant deviations may occur during the surgery, and as a result cause a significant change from how the surgery was originally planned. In examples, during a laparoscopic procedure, a surgical error may occur which leads to the surgeon converting the surgery to an open procedure. As the decision is made to convert the procedure to open, the system may move pieces of equipment out of the way, start to disengage pieces of equipment or ensure tools are ready for extraction.

In other situations, equipment failures may occur. A failure in equipment may occur during the procedure, which may cause the system to react to that failure. A current surgeon may be substituted during the procedure. Within a residency program or training program, an attending surgeon and resident/trainee surgeon may alternate portions of the surgery. In examples, a highly skilled surgeon may move through surgery in a much more confident and overall faster pace than a resident or novice surgeon. As a result, the system may anticipate when they are ready to move to the next step. A more novice surgeon may take longer during a surgery and may be more likely to pause or contemplate next moves. As a result, the system may want to be less reactive to avoid presenting the opportunity for confusion.

In examples, 2 closed loop systems may not be dependent from each other, but may impact each other's function. In a predictive kinematic simulation, a patient or robot movement may be optimized for movement efficiency purposes. Simulation of intermediary limbs and potential collisions may be provided. A simulation initiation driven by the system detection of a joint or trunk prior to the end effector may prevent the end effector from reaching its desired location. If preventatively something is determined to be going wrong, a simulation and/or forecast may be used to predict how and/or why. In examples, a robot arm position may require preventative and/or tactical decision for arms. The system may define which positions are/are not satisfactory or are most likely to be satisfactory.

A single quadrant procedure and a multi quadrant procedure may be provided. A robot may move tools to different locations where ideal set up differs. The system may move arms before this. An image of tangled arms vs not tangled arms may help determine if heading in the wrong direction. In comparing a planned simulation to an updated simulation, an initial plan may include actual procedure steps and if the surgeon doesn't follow plan, it may be corrected. Context of the data for the specific treatment may be based on globally accessible data. Context relative to an encountered irregularity may include radiology, histology, etc. Data available to enable a predictive simulation may include patient specific data like medication data, allergies, a non-allergic response to prior medications, current medications and dosages, etc. A response to prior procedures may include a procedure location/known adhesions. Doctor specific responses may include where is the simulation displayed, the preferred language, the preferred simulation display mode, where within the hospital, and device data may include EES owned data, Non-EES owned data, a smart device, non-smart device, historical data, etc. Data categories may be used to perform assessment of the patient, environment, surgeon, etc.

Predictive forecasting may use the data streams at hand and metadata or context to predict the current trend causing a trigger in the future and therefor some action in the present may be necessary. Anticipation of an instability or change based on the input stream that is undesirable and critical to function (prepares for a change) may be provided. Adaption may be a reaction. A monitored parameter within or outside of the system may have single or double end bounded thresholds. Exceeding these thresholds may provoke a response.

In examples, a smoke evacuator activation of an energy device may be provided. The motor on the smoke evacuator may be activated. In example, for an insufflation pump, reduction of the intraperitoneal insufflation pressure may be below a predefined range 5-7 mmHg (Low), with a normal operating range of 12-15 mmHg. In examples, for an endocutter, a motor current may be above an acceptable range within a mechanical lockout zone (e.g., >60 lb within the first 0.250″ of I-beam/knife travel).

In examples, for a bipolar generator, adjustment of the operational voltage level once the tissue impendence may exceed 150 ohms. A bipolar system may run in a constant current mode until it may be run in constant power mode and then in constant voltage mode. This may be due to the tissue impendence and the high levels of current needed when the impendence is low. This may be a response to the generator not the tissue. The generators may not be capable of outputting the power needed in low impedance portions of the tissue impendence curve to run in the more controlled settings.

In examples, for a powered endocutter, powered stapling may use a threshold of motor current or drive bar speed relative to the control system indicated speed as a means to control loading on the end-effector. In the case of a motor current monitored system the current may be a direct reflection of the force/torque the moor is having to overcome. There may be a threshold on the max current level which may be used to trigger a reduction in motor speed which may result in a related reduction since the tissue in the jaws is viscoelastic. There may be a condition with this manner on control on the motor current which may be detrimental if the speed is reduced. The rotor lock condition, which may occur when the motor ceases to spin, may result in the motor current going to its maximum level. A reduction in voltage or current may not change whether the motor was able to spin, and the continued current may heat up the motor causing further reduction in output torque relative to input current. In this case, the motor may need to be shut off entirely, not just slowed down. To determine this, the data feed needed may be the encoder on the motor or the drive rack which measure the advancement rate of the system rather than the current through the motor. This relative adaptation may be triggered based on a secondary monitor of speed and below a certain threshold speed rather than current could be monitored for the critical shut down trigger. The motor current may be used up to a pre-specified max at which point the control input stream would need to be moved from current to speed.

Importance may be low (motor over heating is not a risk to the patient just the device and a delay of the procedure) so timeliness of a fast fuse trigger that is reset able would be fine. It may not cause a cascade of other detrimental issues to the need for a decisive answer is also not high.

Monitored parameters may detect an unacceptable condition in an unexpected portion of the operational step. In examples, for a bipolar generator, low measured bipolar electrode impendence (0-5 ohms) may indicate either immersion in fluid, low impendence tissue, or an electrical short.

A reactive condition may be eliminated, for example, by switching a data source. Short term change in the closed loop-controlled system based on a violation of a threshold monitoring on the input stream may be a triggering event for response. Changes in the data that may impact the control loop (immediate results) include, but are not limited to, crossing a threshold (reactive), undesired patient status/critical event, rate of approaching a threshold, etc. The reaction may be immediate (quick fuse) or may be delayed (slow or delayed fuse) to determine if the response only momentarily violated the threshold and then returned to acceptable levels. The rate may be the rate at which a measure is approaching the trigger threshold. The rate may be the number of times it has approached the threshold. The rate may be based on how close the stream is to the threshold and the relative stability of the measure compared to the volatility of the measure. A related physiologic measure may approach a tipping point on the valid of the control stream so the system changes the data stream it is controlling off of before a threshold is ever exceeded (preventive). In an example, the core body temperature may approach a level that is likely to induce vasoconstriction or vasodilation which may result in an increase or decrease in blood volume per beat which may result in a change in heart rate which is what is being timed for the in-between beat application of energy for destroying the nerves resulting in the irregular heartbeat.

Data shifts may be monitored. Previous trigging events may be translated in larger or smaller results than expected (repetition or time based results). Initial fault response may not correct and/or resolve a data stream. In examples, a patient blood pressure may be elevated, max medication may be given, but a patient may not respond to the dose.

In examples, patient temperature may be low, a bear hugger may be turned on (and functioning properly) but the patient temperature may not increase. The anticipated behavior may not match data stream reaction to the action that was taken. The system and/or device may not function as intended. In examples, energy activation on patient heart, do not see increase in temperature on local cooling system as expected. Change may be anticipated but not shown/experienced in data.

A system and/or device may be operating as intended but may not meet the needs of the patient and/or surgeon. In examples, a local heart may cool on the highest setting and core. In examples, a harmonic blade may squeal. The user experience may be negative but function correctly. Data interaction may not meet expectations. Procedure plan may deviate (implications on future results). A surgeon may behave differently than intended. A device may not match the procedure plan sequence. The system may need to determine if these deviations are acceptable or harmful to control loop.

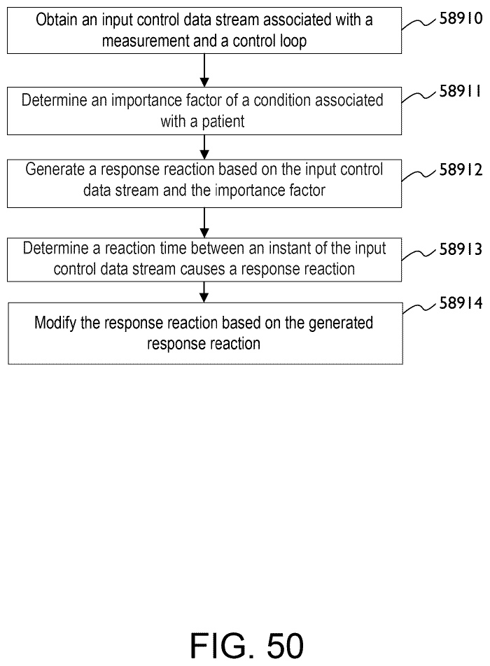

FIG. 50 shows an example flowchart for determining a response reaction and modifying the response reaction. At 58910, an input control data stream associated with a measurement and control loop may be obtained. At 58911, an importance factor of a condition associated with a patient may be determined. At 58912, a response reaction based on the input control data stream and the importance factor may be generated. At 58913, a reaction time between an instant of the input control data stream causes a response reaction may be determined. At 58914, the response reaction based on the generated response reaction may be modified.

In examples, an input control data stream associated with a measurement may be obtained. The input control data stream may be associated with a control loop. An importance factor of a condition associated with a patient may be determined.

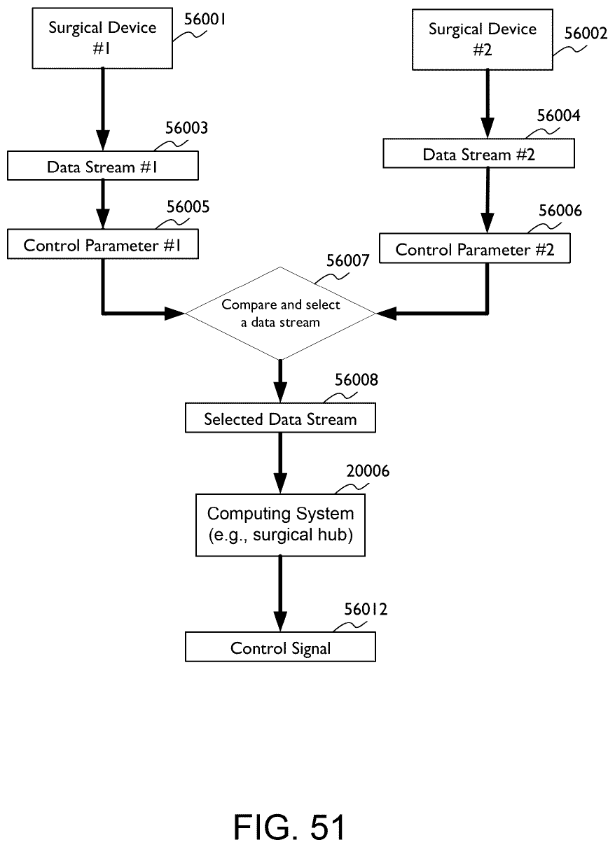

FIG. 51 shows an example computer-implemented surgical system 20006 for comparing multiple (e.g., two) differing data streams to determine which data stream is adequate for controlling the system. The surgical system 20006 may include or may interact with multiple surgical devices such as a first surgical device 56001 and a second surgical device 56002. The first surgical device 56001 may output a data stream (e.g., a first data stream 56003) associated with a measurement 56011. The second surgical device 56002 may output a data stream (e.g., a second data stream 56004) associated with the same measurement 56011. In some examples, the first data stream 56003 and the second data stream 56004 may provide conflicting data. A disagreement between the first data stream 56003 and the second data stream 56004 may be detected.

The surgical system 20006 may select a controlling data stream between the first data stream 56003 and the second data stream 56004 based on their impacts on the control difference. As shown in FIG. 51, a first control parameter 56005 may be determined based on the first data stream 56003, and a second control parameter 56006 may be determined with the second data stream 56004. A risk level (e.g., first risk level) may be associated with the first control parameter 56005, and/or a risk level (e.g., second risk level) may be associated with the second control parameter 56006. For example, a control parameter related to a pulse reading may be associated with a higher risk than a control parameter associated with the temperature of the patient. A control parameter may be selected based on which control parameter has the lower risk level. The data stream associated with the selected control parameter may be selected. For example, a risk level (e.g., first risk level) may be associated with a first control parameter value, and a risk level (e.g., second risk level) may be associated with a second control parameter value. The control parameter value associated with the lower risk level may be selected.

A surgical action (e.g., first surgical action) of the surgical system 20006 may be determined and may be associated with the first control parameter 56005. For example, the first surgical action may be a robotic arm making an incision and may be associated with the blood pressure of the patient. A device may monitor the patient's blood pressure and look for a drop which may suggest internal bleeding.

A surgical action (e.g., second surgical action) of the surgical system 20006 may be determined and may be associated with the second control parameter 56006. The first surgical action and the second surgical action may be compared to a predetermined list of preferred surgical actions. A control parameter may be selected that is associated with a preferred surgical action. The data stream associated with the selected control parameter may also be selected.

In some examples, the first control parameter 56005 and the second control parameter 56006 relate to the same measurement (e.g., same parameter), and the first control parameter may refer to a first control parameter value, and the second control parameter may refer to a second control parameter value.

The first control parameter 56005 may be compared 56007 with the second control parameter 56006. A difference between the first control parameter and the second parameter (e.g., a difference between the first control parameter value and the second control parameter value) may be calculated (e.g., determined). The difference between the first control parameter and the second parameter may represent a discrepancy in a metric, for example, of the first data stream and second data stream. A difference between the first data stream and the second data stream may be determined. The difference between the control parameters and/or the measurement (e.g., a measurement difference) of the data streams may be compared to a threshold (e.g., a threshold value) when determining which to select. For example, if a measurement value is determined to be above the threshold value, the associated control parameter and/or data stream may be selected.

A data stream may be selected 56008 from either the first data stream 56003 or the second data stream 56004, for example, based on the comparison of the first control parameter 56005 and the second control parameter 56006. The selected data stream 56008 may be used to generate control signal(s). For example, the selected data stream may be sent to a computing system (e.g., a surgical hub) 20006. Control signal 56012 may be generated based on the selected data stream 56008.

Determining when a data stream (e.g., a control data stream or first data stream) lacks agreement (e.g., a disagreement) with another data stream (e.g., a second data stream) and/or how to handle the inaccurate (e.g., not chosen) data stream may be provided.

In some examples, a disagreement between the first data stream and the second data stream may be identified, and a cause of the identified disagreement may be identified. The data stream is selected based on the cause of the identified disagreement. For example, a cause of disagreement between the first data stream 56003 and the second data stream 56004 may be identified and/or determined. The cause of disagreement between the first data stream 56003 and the second data stream 56004 may include but not be limited to: an equipment (e.g., device, software, etc.) malfunction; equipment interference; physiologic responses; and perceived data errors. For example, a physiologic situation (e.g., a current physiologic situation) associated with the patient may be determined to be a drop in body temperature. This drop in body temperature may be important for the surgeon to notice as the drop in body temperature may affect other behaviors during the procedure. The selected control parameter, selected between the first and second control parameters, may be based off a physiologic situation of the patient.

In examples, the first data stream and the second data stream may be integrated within a control loop (e.g., closed loop) of the surgical system to continuously adjust and/or refine surgical actions for precise and/or responsive operation.

Equipment (e.g., device, software, etc.) malfunction may include but not be limited to the equipment having a faulty (e.g., bad) connection to a patient, or equipment damage, for example. Equipment malfunction may include situations where the equipment is not functioning as intended, is unresponsive, or is providing inaccurate or inconsistent readings, for example. A faulty connection may be a faulty connection within the equipment itself. A faulty connection within the equipment may include but not be limited to a loose, corroded, worn, or damaged connection, for example. The faulty connection of the equipment may include a faulty connection with a surgical device to the patient. A faulty connection of a surgical device to the patient may include but not be limited to a loose sensor, improper placement, and/or improper materials. Frequent false alarms may cause unnecessary anxiety for both the patient and healthcare providers. Frequent false alarms may lead to alarm fatigue which may cause real emergencies to be overlooked.

In examples, if a connection within a surgical device and/or equipment is incomplete or inadequate, a signal may not appear and/or be inaccurate (e.g., signal drift may occur). For example, signal drift may lead to inaccurate readings, which may result in incorrect data collection and analysis. Signal drift may also undermine the reliability of the device, reducing the trustworthiness of the readings over time and/or may result in lower confidence of the device. Signal drift may require recalibration of the device to maintain accuracy, which is time-consuming and may result in loss of time that is critical to a successful outcome for the patient. However, if signal drift is not noticed by the operator of the device, poor decision-making may occur due to inaccurate data and/or an automated system that relies on this data may lose precision and/or accuracy.

An improper placement of a surgical device on a patient may include the surgical device not being placed on the intended (e.g., correct, required, etc.) anatomy of the patient. If the surgical device is improperly placed on the patient, a signal may appear (e.g., a signal may appear stable). If the surgical device is improperly placed on the patient, a signal that appears may not be accurate. In examples, if a blood pressure cuff is placed on a patient, the blood pressure cuff should be placed (e.g., attached) on the inside of the arm of the patient. If the blood pressure cuff is not placed on the inside of the arm of the patient, the signal may not be accurate.

In examples, a monopolar ground pad may not be properly adhered to a patient. The monopolar ground pad may include an adhesive lead. If the adhesive lead forms a faulty connection with the patient (e.g., is partially connected, or falling off, etc.), a signal may not appear and/or be inaccurate.

In examples, a fingertip pulse oximeter (pulse-ox) may not be properly adhered to a patient. If the fingertip pulse-ox forms a faulty connection with the patient (e.g., is slipping off the patient), a signal may not appear and/or be inaccurate. The pulse-ox may measure the pulse (e.g., pulse rate, or rate of pulse) of the patient. An improperly placed sensor of the pulse-ox might detect incorrect and/or erratic signals, leading to an inaccurate pulse reading.

In examples, a ventilator outlet may form a faulty connection with a patient. If the ventilator outlet forms a faulty connection with the patient (e.g., the outlet is into another closed system), a signal may not appear and/or be inaccurate. If the ventilator outlet forms a faulty connection with the patient (e.g., the outlet is leaking out of the system), no measurement (e.g., measurement of O2, sedation, and/or another gaseous substance) may occur. Faulty connection of the ventilator outlet with the patient may result in errors (e.g., critical errors) in the treatment of the patient, for example, improper oxygenation or inadequate sedation, potentially compromising patient safety and the effectiveness of the ventilation.

If a system includes multiple leads and/or inputs including but not limited to some of the inputs missing and/or not being connected, a signal may not appear and or be inaccurate (e.g., the signal may appear stable but be an incomplete signal). In examples, EKG lead(s) may not be attached properly to a patient. For example, if EKG leads are placed improperly on a patient, it may result in inaccurate readings or misinterpretation of the heart's electrical activity, potentially leading to incorrect diagnoses or missed detection of critical cardiac conditions.

The faulty (e.g., bad) equipment connection may include but not be limited to the equipment having a faulty connection to another piece of equipment. A faulty connection between pieces of equipment may include but not be limited to: incomplete connection(s) and/or attachment(s) between equipment and data source(s) (e.g., the data source(s) of the equipment); or incomplete connection(s) and/or attachment(s) between equipment and shared data sources from (e.g., of) other equipment. For example, faulty connection may lead to inaccurate readings, which may result in incorrect data collection and analysis. Faulty connection may also undermine the reliability of the device, reducing the trustworthiness of the readings over time and/or may result in lower confidence of the device. However, if faulty connections are not noticed by the operator of the device, poor decision-making may occur due to inaccurate data and/or an automated system that relies on this data may lose precision and/or accuracy.

Incomplete connection(s) and/or attachment(s) between equipment and data source(s) (e.g., the data source(s) of the equipment) may include but not be limited to ends (e.g., proximal ends) of wire leads connected to patient may not be fully plugged in to a home unit. An impact to data may occur and/or an intermittent signal or no signal may be produced.

Incomplete connection(s) and/or attachment(s) between equipment and shared data sources from (e.g., of) other equipment may include an impact to data. The data may not be properly shared. The surgical hub 20006 should be able to resolve between original (e.g., correct) acquired data of a first system and partial or missing data that a second system may receive from a bad connection. If there is a disagreement ween the received data of the first system and the second system, resolution options may include but not be limited to: surgical hub 20006 may ignore (e.g., choose to ignore) analog data from the first system to the second system in favor of digital data transfer; surgical hub 20006 may compensate (e.g., choose to compensate) for analog data to help the surgical hub 20006 match an original data source; surgical hub 20006 may prompt a user of the disagreement, and/or indicate system data connection(s) as the source of error (e.g., likely source of error); or the second system may choose to omit data (e.g., the bad, missing, and/or partial data) entirely from the information received from the first system.

Equipment (e.g., device, software, etc.) malfunction may include but not be limited to damage to equipment used for data transport. Damage to equipment used for data transport may include but not be limited to sterilization (e.g., repeated sterilization) of equipment which may affect the data transfer (e.g., data transfer capabilities) of the equipment. The impact of damage to equipment used for data transport may include but not be limited to an impact to data (e.g., noisy signal, no signal, inaccurate signal, etc.).

Damage to equipment used for data transport may include but not be limited to broken and/or damaged electrical equipment being used before the damage is discovered. The impact of damage to equipment used for data transport may include but not be limited to an impact to data (e.g., noisy signal, no signal, intermittent signal, etc.). Damage to equipment may be visible or not visible to an observer. Damage to equipment may lead to delayed diagnostics, incorrect data interpretation, or failure in communication systems and/or device, potentially compromising the overall functionality and reliability of the system and/or device.

Equipment (e.g., device, software, etc.) malfunction may include but not be limited to damage to equipment used for data acquisition. Patient contacting components may be damaged and output an inaccurate signal. The impact of damage to equipment used for data acquisition may include but not be limited to erroneous readings, a data-offset, or an impact to data (e.g., noisy signal, no signal, intermittent signal, etc.).

The cause of disagreement between the first data stream 56003 and the second data stream 56004 may include but not be limited to equipment and/or capital interference. In examples, another device in the operating room may cause interference (e.g., interfere) with the equipment used to gather data having no impact on a signal and/or patient but calibration may be affected. The surgical hub 20006 may be disrupted by large metal objects, for example. If a metal object is passed through a field (e.g., a surgical field) during a procedure, calibration may be affected. Calibration may be compensated while metal is present to avoid calibration interference.

In examples, another device in the operating room may cause interference (e.g., interfere) with the equipment used to gather data. The addition of another data input to a transfer function may result in imbalance to the output data of a transfer function.

The cause of disagreement between the first data stream 56003 and the second data stream 56004 may include but not be limited to physiologic response(s). Physiologic responses may include but not be limited to physiologic responses that reflect a direct change to patient conditions being measured. In examples, a decrease in patient temperature after suction and/or irrigation may be activated for an extended period, which may result in an influx of cold air into a body cavity. Data may respond by showing a change in measured data that directly matches patient conditions and may be attributable to measured inputs.

Physiologic responses may include but not be limited to physiologic responses that cause an indirect change in perceived data by the measurement system. Physiologic responses that cause an indirect change in perceived data by the measurement system may be a result of changes in patient conditions different from those being measured. In examples, effects on data of vasoconstriction on a fingertip pulse-oxygen readings may be shown regarding O2 consumption, etc. A change in measured data may occur that is indirectly a result of patient conditions caused by inputs that may not be directly measured.

The cause of disagreement between the first data stream 56003 and the second data stream 56004 may include but not be limited to perceived data errors. Perceived data errors may include but not be limited to electrical drift. The causes of electrical drift may include but not be limited to resistance, probe age, or a number of sterilizations.

Perceived data errors may include but not be limited to a perception that the data is erroneous but not erratic. In examples, a stable data source may be wrong (e.g., producing incorrect data), but may be position dependent. Perceived data errors may include but not be limited to a flaw in an interface including the purpose and/or actions of equipment (e.g., not an electrical flaw).

Determining when a first data stream lacks agreement with a second data stream and/or how to handle the inaccurate data stream may include but not be limited to logic pathways and/or responses to the identified disagreement. Logic pathways and/or responses to the identified disagreement may include but not be limited to conflicts in interference; choosing which data stream to use; a third data stream being used to confirm an existing closed loop system; prioritization and/or importance; or determining potential causes of errors in the data stream.

Conflicts of interference may include being an assistant and is not expected to be right (e.g., be right all the time) but may avoid being incorrect. Choosing which data stream to use may include using a temperature, for example. Both sources of data may be related at time 0. Both sources may lose relation as the procedure proceeds.

Prioritization of importance may include determining when conflict management does not include the data. Prioritization of importance may include determining whether or not the surgeon should be interfered with. In examples, a surgeon dissecting a first system may use O2 management while a second system may monitor temperature. In the system resolving which of the O2 or temperature to display, the more detrimental (e.g., critical) may be suggested to the surgeon. In examples, a single cause of interference may be provided, which create multiple failures. The (e.g., Only the) more problematic and/or prevalent error may be shown and/or displayed. In examples, a body temperature management may be set to manage the body temperature of a patient based off the patient's heart rate, O2 sensor, patient heating (e.g., body temperature), hypothermia, etc.

Prioritization of importance may include but is not limited to risk management. Other forms of monitoring for conflict management may be provided including monitoring for grandiose errors.

Potential causes of errors in the data stream may be determined. Categories of potential causes of errors in the data stream may be provided and distinguished. If an error in the data stream is one that may be corrected, a method of correcting may be determined. If an error in the data stream is one that may not be corrected, a notice may be provided. A determination of how likely a data stream may be incorrect may be provided. A determination of how often a data stream may be incorrect may be provided.

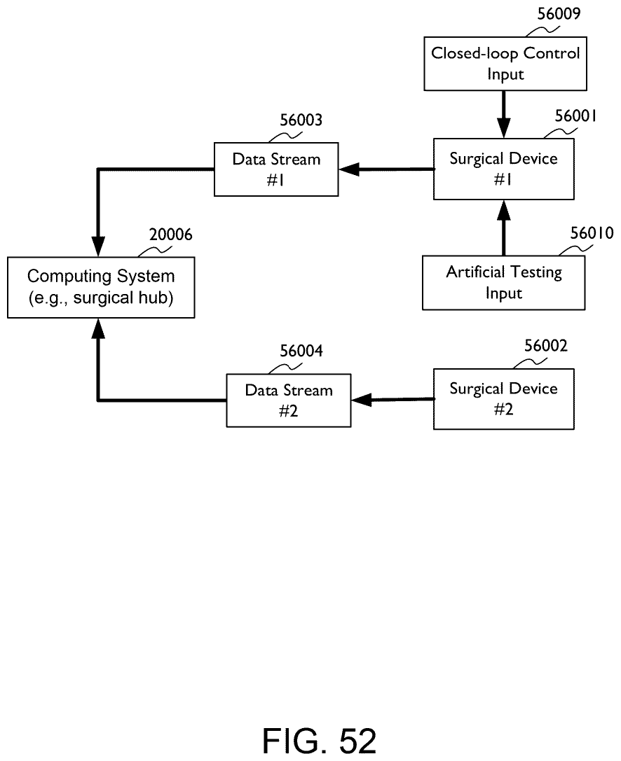

FIG. 52 shows an example computer-implemented surgical system with an artificial testing input with a closed-loop control input for continuously adjusting and/or refining surgical actions for precise and/or responsive operation. The first surgical device 56001 may be associated with a measurement 56011 that may be important to monitor during the surgical actions. An artificial testing input 56010 may be input to the first surgical device 56001 for testing the response of the system, for example. The first surgical device may operate with a closed-loop control input 56009. The second surgical device 56002 may be associated with a measurement (e.g., the same measurement 56011). The first surgical device 56001 may output a data stream (e.g., a first data stream 56003). The second surgical device 56002 may output a data stream (e.g., a second data stream 56004). The first data stream 56003 may be sent to the surgical hub 20006. The second data stream 56004 may be sent to the surgical hub 20006.

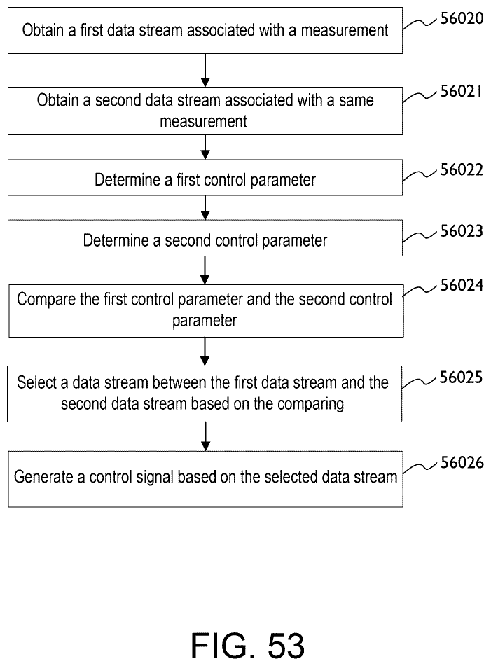

FIG. 53 shows an example flowchart for determining a data stream. At 56020, a first data stream may be obtained. At 56020, a first data stream 56003 associated with a measurement 56020 may be obtained. At 56021, a second data stream 56004 associated with a same measurement 56021 may be obtained. At 56022, a first control parameter 56022 may be determined. At 56023, a second control parameter 56023 may be determined. At 56024, the first control parameter 56005 and the second control parameter 56006 may be compared 56007. At 56025, a data stream may be selected 56008 between the first data stream 56003 and the second data stream 56004 based on the comparing 56007. At 56026, a control signal 56012 may be generated based on the selected data stream 56008.

In a number of examples, the surgical system may obtain a first data stream and a second data stream associated with a same measurement.

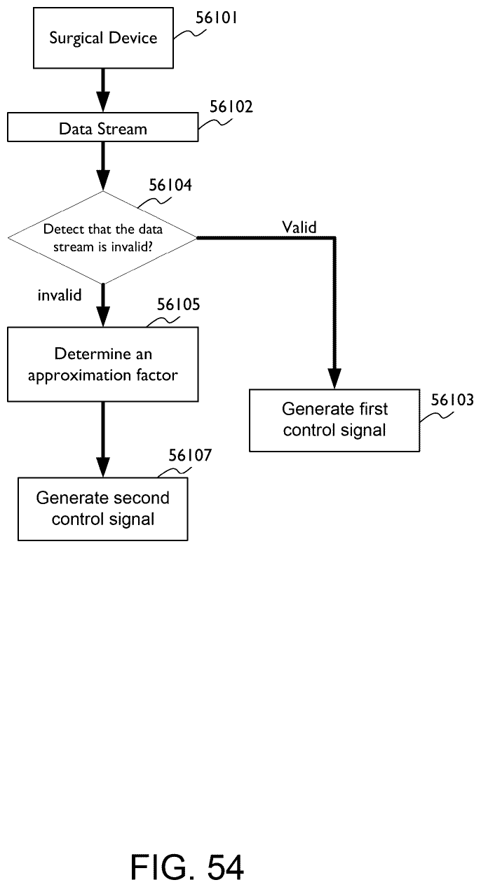

FIG. 54 shows an example computer-implemented method for determining whether a data stream is invalid and generating a control signal. For example, a surgical system may include or may interact with a surgical device 56101. The surgical device 56101 may output a data stream 56102 associated with a measurement. In some examples, the data stream 56102 may provide inaccurate data. At 56104, whether the data stream is invalid may be determined. Based on detecting that the data stream is valid, a first control signal may be generated at 56103. Based on detecting that the data stream is invalid (e.g., determining that the data stream is invalid), at 56105, an approximation factor may be determined. The approximation factor may be used to generate a second control signal at 56107. In some examples, the approximation factor may be combined with (e.g., factored into) the first control signal to generate a second control signal. The first and/or the second control signal may be sent to a surgical device as described herein, a surgical instrument as described herein, or a computing system, such as a surgical hub 20006.

In some examples, the data stream generated from the surgical device may be a single data stream. The data stream may be determined to be invalid and/or misleading the control system to an undesirable outcome. When the data stream is determined to be invalid and/or misleading the control system to an undesirable outcome, the surgical system may identify the issue (e.g., the type of issue), make a correction, and/or determine that the surgical system needs additional information (e.g., sources of information).

The surgical system may identify the (e.g., the type of issue), for example, whether the issue is related to a physiologic change in patient status that invalidates a previously valid data stream and/or whether the issue is related to a signal and/or electrical cause.

In examples, the validity of a data stream may be assessed (e.g., a data stream validity assessment) via intentional disruptions. Intentional perturbations may be introduced into the data stream to assess the validity of the data stream, for example, to assess if the data stream reacts as expected. For example, using an intentional perturbation may show lag in the system or a lack of response. In a closed loop system, an intentional insertion of incorrect data (e.g., an intentional perturbation) into a data stream may be used to monitor a response and adjust the control signal.

The introduced perturbation to an input signal of the surgical device may include data that is testing a known or unknown issue of the surgical system and/or surgical device. The value in the data stream may be received upon introducing the perturbation. An expected value in the data stream may be determined in response to the perturbation. The surgical system may then detect if the data stream is invalid if the perturbation does not cause the expected value to be returned in the data stream.

A single data stream may be misleading the control system to an undesirable outcome. A physiologic change in patient status may invalidate a previously valid data stream. To determine if the data stream is invalid, the data stream may be compared to data at a baseline (ex. procedure initiation) or historic data to confirm measurement is within an expected range (e.g., a normal range). In examples, the data may be correct but not representative of a patient status. When comparing the generated data to the range, a threshold (e.g., an error tolerance threshold value) may be applied to allow for discrepancies. The threshold may be a range, boundary, and/or limit, for example, when determining the validity of a measurement. For example, historic data of a patient may show the average body temperature of the patient within a range. A measured value from a data stream of the temperature of the patient may be determined to be valid if the measured value is within the range of the average body temperature of the patient. In examples, a boundary may be determined at a minimum and/or maximum heartrate. If a measured value from a data stream of the heartrate of the patient is determined to be below the minimum heartrate boundary or above the maximum heartrate boundary, the measured value may be determined to be invalid and alert an HCP. A measurement difference may be determined in addition to determining the validity of a measured value, The measurement difference may highlight the variance of the measured value from the expected value in a data stream. In examples, historic data of a patient may show the average body temperature of the patient within a range. A measured value from a data stream of the temperature of the patient may be significantly below the range of the average body temperature of the patient. In this example, the HCP may take more immediate action to this larger discrepancy. A cause of the identified discrepancy and/or disagreement may be identified. The identified cause of the disagreement may allow the HCP more information for making a decision.

In examples, the cause of the data stream to be invalid may be due to signal and/or electronic malfunctions. Examples of signal and/or electronic malfunctions include interference, signal loss, cross-talk, a distorted and/or weak signal, delay and/or latency, and signal reflection. Interference may include disruption caused by external electromagnetic signals, such as from radios, cell phones, or power lines. Signal loss may include a loss of signal due to low-quality connectivity, physical damage to cables, or other obstructions. Cross-talk may include signals from one communication channel affecting another, causing confusion and/or miscommunication. A distorted signal may include deformation of a signal during transmission, for example, due to noise, faulty hardware, or signal processing. A weak signal may include a reduced signal strength, leading to low-quality reception or functionality. Delay and/or latency may include unintended delay in signal transmission affecting communication, internet speeds, or synchronization in data streams. Signal reflection may include a signal bouncing back to the source or to other devices, causing unintended duplication or overlap.

In examples, a cable detection signal or system may be used to discriminate between hardware issues and potential patient issues. For example, a ground and/or signal interlock may be used. The cable or mechanism may ground out a specific pin on the peripheral for determining if the pin has been disconnected.

In examples, a loopback interlock may be used. The system may provide a loopback mechanism. The capital equipment may provide a signal. The signal may pass through the cable and any peripheral equipment, before returning back to the capital system where it is monitored. If the return signal is not received, then it may be assumed that the signal is lost or corrupted due to an error in the hardware or connection itself.

In examples, active cable pulsing may be used. The cable may generate a specified tone (such as a 1 kHz sine or square wave, for example) in the background, to either be passed on the same wire as the monitored signal, or a distinct wire as the monitored signal (or combination of both). If this signal is not detected by the capital equipment, then it may be an indication that the monitoring peripheral has been disconnected or is faulty.

In examples, a sensing heartbeat system may be used. In a sensing heartbeat system (e.g., smart sensing heartbeat system), the peripheral may send a periodic heartbeat message such as at 1 Hz intervals to broadcast its current status, and that its connectivity status to the surgical system. In examples, a smart system status may be used. For a smart system, the capital equipment or controlling system may query the connected peripheral for its status. The status information of the smart system may be sent (e.g., always be sent) when other pertinent data is sent.

In examples, a calibration jig may be used. A calibration system or setup may be provided. The equipment and/or surgical device(s) may be connected to the calibration system. The connection of the equipment and/or surgical device(s) to the calibration system may produce a known response at different points of the equipment. For example, a pulse oximeter may have a ‘dummy finger’ to provide a predetermined cadence to it. The dummy finger of the pulse oximeter may allow measurements to confirm the system is working correctly.

In examples, in-series sensing system with independent monitoring may be used. The system may provide a mechanism within its own monitoring capabilities to allow a second system to independently monitor it without corrupting or impacting the monitoring of the primary system.

In examples, a voltage readback source may use a calibrated shunt resistor to allow for voltage readings of current (with a known resistance) from a second source. Since both the voltage taps of the shunt resistor and the resistance are known, the system may additionally infer current. The overall impact may be negligible to the primary system. This may allow a second potential monitoring point for a separate system in the event of an error with the primary system.

In examples, bounding of a signal range may be used. A sensor may have the capability to provide a signal over a greater range than is physiologically relevant or capable. As a result, the system may use software or other intelligence to monitor if the signal has exceeded the physiological range and if it is in a zone that may indicate a potential error.

In examples, a sensor may have a range of temperature it can provide back from 0-5V, for example. However, that range of temperature may correlate to 0 degrees Celsius (° C.) to 125° C. We know that the human body will typically remain around 36° C., or about 1.44V on the sensor. We know that if the temperature deviates more than 10° C. in either direction, it may be (e.g., is likely) an indication of a bad reading. The corresponding voltage from the temperature data may correlate to 26° C. (1.04V) or 46° C. (1.84V). As a result, the system may implement thresholds at 1V and 2V to use as a range for a valid signal. Anything outside of those voltages may indicate a signal error.

Incorrect data may be compensated to enable continued system control to correct data stream. In examples, if a data stream is detected and/or determined to be invalid, the data stream may be compensated and/or corrected to be sent to the surgical system.

The surgical system may make a correction to the data stream(s) (e.g., erroneous data stream(s)) to correct for a wrongly corrected data stream. A transformation of the incorrect data stream may be made based on risk associated with the data stream, the surgical device, the measurement, etc. For example, if a measurement is associated with a higher risk of the health of the patient, the transformation associated with that risk may be reduced to avoid exposing the patient to that risk.

Correction factors may be utilized to adjust the signal to correct for a wrongly corrected data stream. Adjustment of the surgical system may be based on an initial characterization of the surgical system. In examples, a system transmitting temperature may be given as a numerical value, without units attached to it. They are transmitted in ° C., but the receiving system expects the temperature to be received in Fahrenheit. As a result, the data stream may be viewed by the system as technically incorrect, although the data may be still seen as valid. The data may be assessed to be in the wrong unit.

Transformation or adjustment(s) of the acceptable boundaries of a data stream may be implemented to correct for a wrongly corrected data stream. If a data stream is incorrect, the boundaries of the data stream may be corrected to include a wider and or narrower range, for example. Responses to the system may be trend based or magnitude based, for example. Data streams that have a variation and/or are still performing consistently, even if the value is incorrect in absolute terms, may have their trending adjustment be implemented.

For example, an approximation factor may be a multiplier that is applied as a transformation to the data stream to generate a corrected data stream. The approximation factor may be an equation that is applied to the data stream to generate a corrected data stream. In an example, an approximation factor may be an equation that generates a data stream in degrees Celsius from a data signal that was received in degrees Fahrenheit. In examples, a patient temperature may be determined to be low using a patient temperature monitor. An initial characterization of the patient temperature monitor may be obtained which shows that the patient temperature monitor is reading the temperature of the patient in degrees Celsius while the data is being interpreted in degrees Fahrenheit. An approximation factor may be applied to the temperature being read in degrees Celsius to alter the data to correspond with degrees Fahrenheit.

Incorrect data streams may be ignored based on risk (e.g., risk associated with the health of the patient). For example, if an incorrect data stream is associated with a higher risk of the health of the patient, then the data stream associated with that risk may be ignored to avoid exposing the patient to that risk.

Intentional calibration of the surgical system may be implemented to correct for a wrongly corrected data stream. Intentional calibration of the surgical system may include white light balancing and/or temperature calibration by a user. In examples, temperature sensors may be applied to the patient. A user (e.g., a surgeon) may select to normalize the surgical system. The system may normalize certain values to associated 100%. It may not matter as a result if the temperature received is received in degrees Fahrenheit or degrees Celsius, as the system could look at the difference in trending or percent difference.

Indirect calibration on realistic bounding or norms may be implemented to correct for a wrongly corrected data stream. In an example, temperature sensors may read in the range of 18-30 degrees. This range may be unreasonable for most operating rooms in degrees Fahrenheit, but perfectly reasonable for degrees Celsius. As a result, the system may make an interpretation that the temperature being received is in degrees Celsius.

The surgical system may determine that the surgical system needs additional information (e.g., sources of information). In examples, the surgical system may be providing a data stream on a probe location. The probe depth within the lung may reduce the accuracy of the location data stream. Additional input or a new data stream may be needed to locate the probe position.

The surgical system may lose confidence in position over time. This may result in the surgical system being unable to reconcile a control signal from the collected data. Trigger events may increase uncertainty of probe position and/or location including but not limited to monopolar activation or a CT machine in the field. Consistent events are tolerable but transient events may be catalogued.

Establish library of events to categorize and communicate events that trigger data uncertainty. Electromagnetic drift against kinematic movement may result in an absolute change and/or a percentage change. Percentage errors in changes in positioning system may be compared to recorded changes in kinematic movement.

A relative error assessment may be implemented for the system triggers when indicating insufficient control data. The system may continually calculate perceived confidence in a location. The perceived confidence may be provided back to the user (e.g., surgeon).

A linear assessment against pre-surgical scans may be implemented for the system triggers when indicating insufficient control data. Computation of forward kinematics against the pre-surgical scans may be used to determine confidence levels, for example.

The surgical system may determine a location is drifting independently. In examples, electromagnetic sensors may be laddered to determine a location is drifting. Laddering of linked electromagnetic sensors, where each sensor can provide data of its current location, may allow a stackup to be performed of the location within the body, and thereby reduce error.

Alternative mechanisms may be utilized for a secondary determination of the position of a device. Alternative mechanisms for a secondary determination of the position of a device may include methods that do not include the use of a Cone Beam CT or surgical system existing electromagnetic navigation system to determine its current location.

Alternative mechanisms for a secondary determination of the position of a device may include utilization of a radioactive or contrast material to help determine the absolute location and be utilized as a beacon.

Alternative mechanisms for a secondary determination of the position of a device may include alternative frequency of communication that is less impacted by the body. In examples, low frequency beacons (such as below 100 MHz) may have better mechanisms of passing through the body, and may be able to determine the location via signal strength, such as triangulation of the signal.

Alternative mechanisms for a secondary determination of the position of a device may include bright light (visible and/or infrared). A high power light may be blinked on for an extremely short duration to avoid thermal injury, but could be picked up and located by multiple cameras in the room that are synchronized to flashing of the light.

Alternative mechanisms for a secondary determination of the position of a device may include an EM sensor in an induced EM field utilized for signal strength to triangulate position. To improve tracking, multiple EM sensors may be placed at fixed intervals along a flexible tube. Each sensors may report its individual position(s), which may be correlated to the previous and/or subsequent sensor (or “local pair”) on the shaft using the fixed distance along the scope as a reference length. By chaining each sensor to its local pair, an overall vector map of the shaft's position and orientation can be reconstructed in space, providing higher fidelity positional tracking and context to the operator. Using a point and its local pair, the system may quantify positional error (or measurement drift) by calculating spatial separation and referencing against the fixed shaft distance. Stacking error from point to point may provide a map of interference in critical areas of operation, potentially allowing for measurement drift compensation, or to calculate a “confidence” value which can be communicated to the operator. An additional embodiment may compare the current detected position to the last known position and/or the “change in commanded position” by the surgical system/s platform. This may be performed on each point to estimate error along the whole shaft.

Alternative mechanisms for a secondary determination of the position of a device may include sound and/or a microphone. The surgical device may use EM waves to locate the beacon on the tip of the endoscope. When the scope is deep within the body cavity, the accuracy of the position may be diminished. A CT scanner may be brought in to act as a secondary source of localization. However, it may interfere with the surgical system's readings. Multiple signals may be used that communicate the same information through different forms as a secondary check while preventing against interference and other issues. In examples, the surgical system may utilize a speaker in the endoscope tip combined with a microphone array in the operating room. During the procedure, the speaker may emit a tone or series of tones at a set frequency (ultrasonic and/or subsonic to prevent the operating room staff from hearing. The microphone array may be set up in a known position around the patient and designed to detect the set frequency of the speaker. Using an array allows for triangulation or other methods of position detection. In addition to an array in the operating room, microphones may be placed at a known position in natural orifices close to the endoscope (e.g., the esophagus), which could improve accuracy. The frequency or sound emitted may need to be chosen so it can penetrate the human body cavity. Since sound acts through a different medium than EM waves, it would have a lower likelihood of interfering with the surgical system position detection while simultaneously providing a secondary check. This same concept of multi-signal inputs may be used for other Interactive Smart Systems in the operating room by utilizing different forms, similar to humans having multiple senses. Utilization of a radioactive or contrast material may help determine the absolute location and be utilized as a beacon.

Alternative mechanisms for a secondary determination of the position of a device may include laparoscopically assisted solutions. Laparoscopically assisted solutions may include a camera for lighting, a magnetic sensor, for example. A separate laparoscopic incision may be used to insert a magnetic sensor, that may exist on the outside of the lung area, but may be used to help confirm the location of the surgical system sensor.

Alternative mechanisms for a secondary determination of the position of a device may include on-patient markers detected by the surgical system and/or may be compared to CT. To improve accuracy of the EM position sensing, a 2D calibration grid may be utilized. A 2D grid of EM sensors may be fixed on the table at known distances and locations from each other. The grid would remain in the same physical location during the course of the procedure. The surgical system may continuously monitor the location of these non-moving, known locations to their true positions. The system may optionally apply the appropriate offset to the system if the position of the sensors displays out of a determined tolerance due to changes to the EM field.

The surgical system may incorporate a new data stream. The surgical system may be able to run with present data. The surgical system may indicate additional data is needed. The surgical system may add additional data from one of the systems. The surgical system may know what other information is needed and/or idea but may have limited bandwidth (e.g., a surgical system may use minimum viable data).

Signal loss or corruption of flexible endoscopic robot position data, for example, may also be provided. The surgical system may monitor data latency and latency variation (e.g., jitter) during a procedure in order to identify when the latency or jitter exceeds a pre-defined threshold at which it has been determined that surgeon performance may suffer.

On-patient markers may be detected by the surgical system and/or compared to a CT. To improve accuracy of the surgical system position sensing, a 2D calibration grid may be utilized. A 2D grid of EM sensors may be fixed on the table at known distances and locations from each other. The grid would remain in the same physical location during the course of the procedure. The surgical system could continuously monitor the location of these non-moving, known locations to their true positions. The system then may optionally apply the appropriate offset to the system if the position of the sensors displays out of a determined tolerance due to changes to the EM field.

A 2D calibration grid for the surgical system position accuracy may include using multiple interactive smart systems to locate oneself within the body. Issues may arise due to difficulties knowing up from down, left from right, on the live endoscope camera, for example. This may force the user (e.g., the surgeon) to rely mainly on known landmarks from CT scans or ultrasounds, which may often be difficult to find. A calibration system may be used at the outset of a procedure to set a global coordinate system that may project onto the endoscope camera live feed. Other smart system screens may remedy this issue.

A 3D calibration grid may be used to aid in endoscope camera orientation. Markers may be placed on the patient pre-operation that may be used for triangulation. They may need to be sensitive to CT and EM energy. At the beginning of a procedure a CT may be performed to relocate the tumor. The markers may be geolocated on the CT image. Measurements may be taken from the markers to the tumor. The CT may be shut off and the EM machine may then be used to sense the markers on the patient. This may map out the location of the markers for the EM sensor. The EM and CT data may be overlayed to calibrate how the EM measurements relate locationally to the CT data. This may be used in a feedback loop with the endoscope to know where the endoscope tip is in relation to the tumor. During the procedure, if locational data is unable to be obtained due to EM sensor dilution, an error may be displayed to the surgeon to let them know the location of the endoscope is not within a pre-defined margin of error, suggesting need for secondary location feedback.

Self-latency monitoring of overlaid laparoscopic video before or during a surgical procedure may be provided. The surgical system may monitor video and/or device data latency and/or latency variation (e.g., jitter) during a procedure to identify when the latency or jitter exceeds a pre-defined threshold at which it has been determined that surgeon performance may suffer. In examples, thresholds may appear to be approximately 160 ms for a total system video latency and approximately 30 ms for a system video jitter.

The system may monitor video and/or device data latency and/or latency variation (e.g., jitter) before the procedure, during a system setup, to allow the surgeon to decide whether or not to proceed with the procedure as planned, or to directly connect the laparoscopic camera system to the surgical monitor (e.g., via a video router), bypassing the surgical system.

The surgical system may mitigate latency and jitter (e.g., excessive latency and jitter) in the event the clinically acceptable thresholds are exceeded. The surgical system may display a notification and/or alert on the surgical monitor overlay informing the surgeon that the acceptable latency and/or jitter threshold has been exceeded. An LED (or similar) indicator on the surgical system or associated hardware may be illuminated. An audible tone may be annunciated by the surgical system, either alone or in conjunction with other notification methods. This tone may be sufficiently loud to be heard above typical levels of background noise in the operating room. The surgical system may re-route the video output so that it bypasses the Hub (directly from laparoscopic camera to surgical monitor). The surgical system may display the real-time latency and/or jitter measurements on the surgical monitor overlay, possibly drawing additional attention to them when they exceed the clinically acceptable thresholds. The surgical system may disable video output to the surgical monitor (possibly with a delay) to ensure the surgeon is not operating using the surgical system in a situation with unacceptable latency or jitter. The surgical system may prioritize certain tasks that may reduce overall system latency. In examples, if video recording is in process, this may be paused since it may not be essential to patient health. In the event of excessive surgical device data latency, the software may display a notification or alert on the surgical monitor informing the surgeon to refer to the surgical device itself for timely information, alerts and alarms.

In examples, the surgical system may have 8 cores and support 16 threads. One of these cores/threads may be dedicated to monitoring latency and jitter so as not to impact the performance of other functions. Latency may be measured based on video and/or data input to the system and video output from the system so just the surgical system's contribution to the overall latency is measured. Time zero (t0) may be defined (for video) as when the video frame grabber or FPGA receives/grabs the video frame and the end-time (tf) may be defined as when the GPU processes/outputs the overlaid frame. Jitter may be defined as the difference between consecutive tf−t0 intervals. For device data, t0 may be defined as when the raw device data message is received which is logged and tf may be defined as for the video latency scenario above.

A spare video input and output of the digital hub may be used to assess latency prior to a surgical procedure. This may be achieved by the surgical system timestamping an incoming video frame from the laparoscopic camera, outputting this frame on a spare video output, and the re-reading the same frame (timestamped) on the spare video input. The difference between the timestamps associated with the successive input frames may closely approximate the latency introduced by the surgical system. By monitoring laparoscopic video latency and latency variation (jitter) prior to and/or during a surgical procedure, the surgical system may identify when these latency measures exceed a clinically acceptable threshold and take action to mitigate any resulting potential harm to the patient. Surgeons may benefit by being alerted of excessive video latency and latency variation before it affects the surgical procedure and leads to patient harm. By monitoring surgical device data latency prior to and/or during a surgical procedure, the surgical system may identify when these latency measures exceed a clinically acceptable threshold and may take action to mitigate untimely surgical device information display on the surgical monitor and any resulting surgeon frustration.

Excessive laparoscopic video latency and video latency variation (e.g., jitter) from the laparoscopic camera to the surgical monitor may adversely impact surgeon performance and lead to patient harm. Excessive surgical device (e.g. electrosurgical generator or surgical instrument) data latency may result in delayed display of relevant device information on the surgical monitor and may result in delayed surgical procedures and surgeon frustration.

A lack of patient information may be resolved at procedure initiation. In an example with an unknown patient, 16% of procedures are emergency compared to planned, and of these, many are time-sensitive and preoperative planning and imaging are not possible. Smart systems may deal with a lack of information. In examples, for a patient with excessive internal bleeding, an ultrasonic evaluation, CT, MRI may not have visibility due to the presence of blood and tissue. A database history may be reviewed. A WBC count may be elevated. When a patient identified as unknown0, a “tag” may be started on them to intentionally collect data from beginning of care to build a history of them compared to big dataset. Unknowns about the patient may be reduced.

Real-time data may inform the next steps to be taken. Patient factors may be determined based on inspection. An analysis of the patient may inform the system on an approach. Demographic data may be available that may indicate a risk profile of the patient. The data may be fed into simulation to approximate the patient risk profile. In examples, in the context of respiration, the heartrate and/or breathing may change erratically or be depressed. If the patient had previous lobectomy, the ventilator may indicate that inhalation and/or exhalation volume representation has a reduced volume. Weight and/or height of the patient may be combined with estimated tidal volume. Scars and/or past procedure markings on surface of the patient may be noted. In the context of imaging, if there is a puncture trauma to a lung lobe, the smart system may indicate regions that are impacted. For heart stents, if the HCP does not have time to run a CT, additional implantables may be detected within the patient.

Biomarkers may be used to trace a patient. Biomarkers available include the retina and thumb print. The system may store de-identified data. Patient records may be found to be most similar to the patient in the OR. Biomarkers may be stored (e.g., keep them separated from PHI information) and may enable the surgeon to confirm the identity of the patient.

A curve (e.g., distribution curve) may be fit to other patients in the OR. Information including, but not limited to, surgical history, patient history, allergies, current medication, and previous disease history, may be needed but not available through a real-time test. When there is insufficient preoperative imaging, intraoperative imaging may supplement. Local site imaging may be communicated to other smart devices using the feed to provide context or orientation origin. Rates of unplanned re-operation have been reported as highly variable in the literature, ranging from 0.8% to 7%. Evaluating and tracking unplanned surgical results on surgical wards may raise awareness of complications and surgical errors.

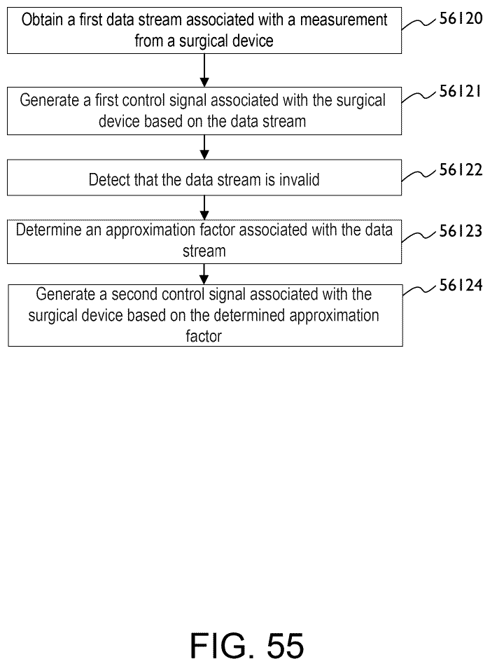

FIG. 55 shows an example flowchart for determining a data stream to be invalid and generating a control signal based on a determined approximation factor. At 56120, a first data stream may be obtained. At 56120, the first data stream may be associated with a measurement from a surgical device. At 56121, a first control signal associated with the surgical device may be generated based on the data stream. At 56122, the data stream may be detected and/or determined to be invalid. At 56123, an approximation factor associated with the data stream may be determined. At 56124, a second control signal associated with the surgical device based on the determined approximation factor may be determined.

Invalid data streams may be detected. An approximation factor may be determined that is associated with the invalid data stream(s).

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

1. A method for controlling multi-system interaction, comprising:
receiving a data stream;
selecting a surgical option associated with a surgical instrument based on the data stream;
generating a control signal associated with the surgical instrument based on the selected surgical option.

2. The method of claim 1, wherein the data stream is an external data stream from a source external to a surgical system, and the method further comprises:
deriving, based at least on the external data stream, decision contextual information, wherein the surgical option associated with the surgical instrument is selected based on the decision context information; and
generating a visual indication of the decision context information associated with selecting the surgical option.

3. The method of claim 1, wherein the surgical instrument is a first surgical instrument, and the method further comprises:
receiving an indication of a surgical procedure that involves the first surgical instrument cooperating with a second surgical instrument, wherein the surgical procedure comprises a plurality of surgical steps;
determining a first candidate action and a second candidate action associated with the first surgical instrument, wherein the first candidate action and the second candidate action allow the first surgical instrument to complete a first step of the plurality of the surgical steps;
determining a first effect, caused by the first candidate action, on the second surgical instrument's ability to perform a second step of the plurality of surgical steps;
determining a second effect, caused by the second candidate action, on the second surgical instrument's ability to perform the second step of the plurality of surgical steps; and
selecting, based on the first effect and the second effect, an action, from the first candidate action and the second candidate action, for the first surgical instrument to perform, wherein the control signal associated with the first surgical instrument is configured to indicate the selected action.

4. The method of claim 1, wherein the surgical instrument is associated with a first robotic arm, and the method further comprises:
receiving an indication of a plurality of steps of a surgical procedure, wherein one or more steps in the plurality of steps of the surgical procedure involve use of at least one of the first robotic arm attached to a first base, or a second robotic arm attached to a second base;
determining a fixed position of the first base;
determining, based on the plurality of steps of the surgical procedure and the fixed position of the first base, that a first candidate position of the second base is associated with a first number of interactions in which the first robotic arm and the second robotic arm will co-occupy space during the surgical procedure;
determining, based on the plurality of steps of the surgical procedure and the fixed position of the first base, that a second candidate position of the second base is associated with a second number of interactions in which the first robotic arm and the second robotic arm will co-occupy space during the surgical procedure;
selecting a candidate position for the second base, from the first candidate position and the second candidate position, based on the first number of interactions and the second number of interactions; and
generating a control signal configured to indicate the selected candidate position for the second base.

5. The method of claim 1, wherein the surgical instrument is associated with a first robotic arm, and the method further comprises:
receiving an indication of a plurality of steps of a surgical procedure associated with a patient, wherein one or more steps in the plurality of steps of the surgical procedure involve use of the first robotic arm having a first end effector attached and a second robotic arm;
identifying a first candidate motion and a second candidate motion of the first robotic arm configured to place the first end effector in a target end effector position internal to the patient;
determining, for the first candidate motion, a first number of associated interactions in which the first robotic arm and the second robot arm co-occupy space external to the patient during the surgical procedure;
determining, for the second candidate motion, a second number of associated interactions in which the first robotic arm and the second robot arm co-occupy space external to the patient during the surgical procedure; and
selecting a candidate motion of the first robotic arm, from the first candidate motion and the second candidate motion, based on the first number of interactions and the second number of interactions, wherein the control signal associated with the first surgical instrument is generated based on the selected candidate motion of the first robotic arm.

6. The method of claim 1, wherein the surgical instrument comprises a surgical device, the data stream is a first data stream, and the method further comprises:
receiving a first biomarker value associated with a first biomarker in the first data stream and a second biomarker value associated a second biomarker in a second data stream;
determining, based on the first biomarker value and the second biomarker value, that a close-loop control condition associated with a control parameter for the surgical device is satisfied;
based on determining that the close-loop control condition is satisfied, determining a control parameter value associated with the surgical device based on the first biomarker value and the second biomarker value;
generating a control signal for the surgical device based on the determined control parameter value;
receiving a third biomarker value associated with the first biomarker in the first data stream and a fourth biomarker value associated with the second biomarker in the second data stream;
determining, based on the third biomarker value and the fourth biomarker value, that the close-loop control condition associated with the control parameter for the surgical device is failed;
based on determining that the close-loop control condition is failed, identifying an intraoperative metric associated with the first data stream and the second data stream; and
generating a second control signal configured to display a value associated with the intraoperative metric.

7. The method of claim 1, wherein control signal is associated with optimizing a selection of a control loop for surgical elements during a medical procedure to achieve safe and reliable outcomes for patients, and the method further comprises:
receiving a user input indicating a selection of a procedure from a plurality of procedures, and a selection of a tactical domain target, wherein the procedure and the tactical domain target are associated with a parameter of a patient;
filtering, based on the selection of the procedure, a plurality of surgical elements to obtain a primary surgical element and a secondary surgical element associated with the procedure, wherein the primary surgical element comprises a plurality of primary control loops associated with an output characteristic of the primary surgical element, and the secondary surgical element comprises a plurality of secondary control loops associated with an output characteristic of the secondary surgical element;
determining a tactical domain data for the procedure, wherein the tactical domain data comprises one or more relationships associated with the primary surgical element, the secondary surgical element, the parameter of the patient, and the tactical domain target;
receiving a primary control data from the primary surgical element based on a primary control loop from the plurality of primary control loops, wherein the primary control data comprises the output characteristic associated with the primary surgical element;
receiving a secondary control data from the secondary surgical element based on a secondary control loop from the plurality of secondary control loops, wherein the secondary control data comprises the output characteristic associated with the secondary surgical element;
generating a recommendation based on the tactical domain data, the primary control data, and the secondary control data, wherein the recommendation comprises an indication of an optimized control loop for the primary surgical element during the procedure, wherein the optimized control loop adjusts the output characteristic associated with the primary surgical element to achieve the tactical domain target;
sending the recommendation to the primary surgical element; and
causing the primary surgical element to adjust the output characteristic associated with the primary surgical element based on the optimized control loop, wherein the primary surgical element adjusts the output characteristic during the procedure to achieve the tactical domain target.

8. The method of claim 1, wherein control signal is associated with optimizing a selection of a control loop for surgical elements during a medical procedure to achieve safe and reliable outcomes for patients, and the method further comprises:
receiving a first dataflow from a first surgical element, wherein the first dataflow is associated with a physiological parameter of a patient;
determining that the first dataflow from the first surgical element is erroneous;
determining a second dataflow associated with a second surgical element, wherein the determination of the second dataflow is based on an indication of a relational link associated with the second dataflow of the second surgical element, control data for the first surgical element, and the physiological parameter of the patient;
transmitting, to the second surgical element, a configuration message, wherein the configuration message comprises an indication that the first dataflow is erroneous and a request to configure the second surgical element to send the second dataflow;
receiving, from the second surgical element, a configuration response comprising the second dataflow;
generating the control data for the first surgical element based on the second dataflow, wherein the control data indicates an adjustment to an output characteristic associated with the first surgical element; and
causing the output characteristic associated with the first surgical element to be adjusted based on the control data.

9. The method of claim 1, wherein control signal is associated with optimizing a selection of a control loop for surgical elements during a medical procedure to achieve safe and reliable outcomes for patients, and the method further comprises:
receiving a first dataflow from a first surgical element, wherein the first dataflow is associated with a physiological parameter of a patient;
determining based on the first dataflow, that the physiological parameter of the patient exceeds a patient safety threshold;
determining a second dataflow associated with a second surgical element, wherein the determination is based on an indication of a relational link associated with control data for the second surgical element, the first dataflow, and the physiological parameter of the patient;
transmitting, to the second surgical element, a configuration message comprising an indication that the physiological parameter of the patient exceeded the patient safety threshold, and a request to configure the second dataflow to receive control data associated with the first surgical element;
generating the control data associated with the first surgical element, wherein the control data indicates an adjustment to an output characteristic associated with the second surgical element;
transmitting, to the second surgical element, a control message, wherein the control message comprises an indication of the control data associated with the first surgical element; and
causing the output characteristic associated with the second surgical element to be adjusted based on the control data associated with the first surgical element.

10. The method of claim 1, wherein the data stream is a first data stream associated with a measurement, the first data stream is associated with a first control loop of the surgical system, and the method further comprises:
obtaining a second data stream associated with the measurement, wherein the second data stream is associated with a second control loop of the surgical system;
determining that the first control loop and the second control loop are diverging; and
generating a control signal based on the first and second data streams.

11. The method of claim 1, further comprising:
obtaining an input control data stream associated with a measurement, wherein the input control data stream is associated with a control loop of the surgical system;
determining an importance factor of a condition associated with a patient;
generating a response reaction based on the input control data stream and the importance factor of the condition associated with the patient;
determining a reaction time between an instant of the input control data stream causes a response reaction to be generated; and
modifying the response reaction based on the generated response reaction.

12. The method of claim 1, wherein the data stream is a first data stream, and the method further comprises:
obtaining a second data stream associated with a same measurement as the first data stream;
determining a first control parameter based at least in part on the first data stream;
determining a second control parameter based at least in part on a second data stream;
comparing the first control parameter and the second parameter;
selecting a data stream between the first data stream and the second data stream based on the comparing; and
generating a control signal based on the selected data stream.

13. The method of claim 1, wherein the data stream is associated with a measurement from a surgical device, and the method further comprises:
generating a first control signal associated with the surgical instrument based on the data stream;
detecting that the data stream is invalid;
upon detecting that that the data stream is invalid, determining an approximation factor associated with the data stream; and
generating a second control signal associated with the surgical instrument based on the determined approximation factor.

14. The method of claim 1, wherein control signal is associated with optimizing a selection of a control loop for surgical elements during a medical procedure to achieve safe and reliable outcomes for patients, and the method further comprises:
determining that a first dataflow from a first surgical element is erroneous;
determining a second dataflow based on a relational link associated with the first surgical element and a physiological parameter of a patient;
transmitting, to a second surgical element, a configuration message including a request to configure the second surgical element to send the second dataflow;
receiving, from the second surgical element, a configuration response comprising the second dataflow; and
causing an output characteristic associated with the first surgical element to be adjusted based on control data associated with the second dataflow, wherein the control data indicates an adjustment to the output characteristic associated with the first surgical element.
